(19) United States
(12) Patent Application Publication
(10) Pub. No.: US 2004/0097566 A1
(43) Pub. Date: May 20, 2004

(54) 2-Substituted thiazolidinone and oxazolidinone derivatives for the inhibition of phosphatases and the treatment of cancer

(76) Inventors: Magnus Pfahl, Solana Beach, CA (US); Hussien A. Al-Shamma, Encinitas, CA (US); Andrea Fanjul Giachino, San Diego, CA (US); David P. M. Pleynet, San Diego, CA (US); Haifeng Bao, San Diego, CA (US); Lyle W. Spruce, Chula Vista, CA (US); Christopher N. Cow, San Diego, CA (US); Catherine Tachdjian, San Diego, CA (US); James W. Zapf, San Diego, CA (US); Torsten R. Wiemann, Encinitas, CA (US)

Correspondence Address: NEEDLE & ROSENBERG, P.C., SUITE 1000, 999 PEACHTREE STREET, ATLANTA, GA, 30309-3915, US

(21) Appl. No.: 10/313,341

(22) Filed: Dec. 6, 2002

Related U.S. Application Data
(60) Provisional application No. 60/337,195, filed on Dec. 6, 2001.

(51) Int. Cl. A61K 31/426; A61K 31/421; C07D 277/32; C07D 263/18; C07D 413/02; C07D 417/02

(52) U.S. Cl. 514/369; 514/376; 548/182; 548/225

(57) ABSTRACT

The present invention relates to certain substituted heterocycles, including 2-substituted thiazolidinone and 2-substituted oxazolidinone compounds. These compounds are useful in the treatment of diseases related to uncontrolled cellular proliferation, such as cancer or precancerous conditions. The compounds are also useful for modulating lipid and/or carbohydrate metabolism, and treating Type II diabetes, hyperglycemia or obesity, and for treating inflammatory diseases such as arthritis.

Some disclosed embodiments of the invention relate to compounds having the structures indicated below, or a pharmaceutically acceptable salt thereof.

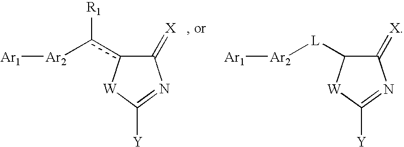

### RELATED APPLICATIONS

This application claims priority to the U.S. Provisional Application Serial No. 60/337,195, filed Dec. 06, 2001, the disclosure of which application is hereby incorporated in its entirety by this reference.

### BACKGROUND OF THE INVENTION

Solid tumors are the leading cause of death attributable to cancers worldwide. Conventional methods of treating cancer include surgical treatments, the administration of chemotherapeutic agents, and recently immune based treatments, which typically involve the administration of an antibody or antibody fragment. Surgical treatments are generally only successful if the cancer is detected at an early stage, i.e., before the cancer has infiltrated major organs. Immune based treatments are subject to problems, including difficulty in targeting antibodies to desired sites, e.g., solid tumors, and host immune reactions to the administered antibody.

The usage of small molecules for the prevention and treatment of cancer has also been reported. Many of the chemotherapeutic treatments available for clinical application today are of limited usefulness because of their non-selective killing and/or toxicity to most cell types. Also, many tumor cells eventually become resistant against the chemotherapeutic agent, thus requiring treatment of such resistant tumors with new agents.

Antiestrogens and antiandrogens for the treatment/prevention of breast and prostate cancer, respectively, are excellent examples of a class of small molecule ligands that function via their influence on nuclear receptor signaling pathways. Small molecules that are useful in the treatment of certain cancers and/or diabetes were disclosed in U.S. patent application Ser. No. 09/655,460 filed Aug. 31, 2000, which is related to PCT International Publication WO 01/16122, published Mar. 08, 2001, and small molecules that are useful in the treatment of certain cancers and/or associated inflammatory diseases were disclosed in U.S. patent application Ser. No. 09/652,810 filed Aug. 31, 2000, and the related publication WO 01/16123, published Mar. 08, 2001.

Additional small molecules that are useful in the treatment of cancers were disclosed in U.S. patent application Ser. No. 10,094,142, filed Mar. 07, 2002, which is related to PCT International Publication WO 02/072009, published Sep. 19, 2002. The disclosures of WO 01/16122, WO 01/16123, and WO 02/072009, and their related United States Patent Applications are hereby incorporated herein by this reference in their entirety including their chemical structural disclosures, and their teachings of the biological activities of their compounds, and methods for their use as pharmaceutical compositions.

Three classes of protein phosphatases have been defined: tyrosine protein phosphatase, serine/threonine protein phosphatase, and dual specificity protein phosphatase. The dual specificity phosphatase dephosphorylates tyrosine as well as serine and threonine residues on the same protein or polypeptide substrate. Cdc25 is a dual specificity protein phosphatase that is believed to be intimately associated with cell growth. Cdc25 controls cell cycle progression by regulating cell cycle transitions at G1/S and G2/M as well as S phase progression. It activates cyclin-dependent kinases (Cdks) by removing inhibitory phosphorylations on Thr14 and Tyr15 residues of Cdks, thereby promoting cell cycle progression. A family of three analogous genes has been identified in humans, termed Cdc25A, Cdc25B, and Cdc25C. Cdc25A and Cdc25B are considered to be oncogenes because overexpression of these two genes has been found in up to 50% of all major human cancers. Overexpression of Cdc25 phophatases can lead to enhanced cancer cell growth. Therefore small molecules that inhibit the action of the Cdc25 phosphatases can inhibit cancer cell growth and can provide a new therapy for the treatment of human cancer when used alone or in combination with other anticancer agents. In general phosphatases play important roles in signal transduction mechanisms. Inhibition of phosphatases can therefore be useful for the control of other diseases such as metabolic disorders including Type II diabetes, or inflammatory diseases such as arthritis.

### SUMMARY OF THE INVENTION

The present invention relates to a series of substituted heterocyclic compounds, including 2-substituted thiazolidinone and 2-substituted oxazolidinone compounds, that show unexpectedly potent anti-cancer activity in vitro and/or in vivo. The novel heterocyclic compounds of the present invention have been unexpectedly found to exhibit potent inhibitory properties against Cdc25, with the effect of inhibiting cancer cell growth, and/or causing the apoptosis of cancer cells. Accordingly, the heterocyclic compounds disclosed herein are useful in the treatment of diseases of uncontrolled proliferation, such as cancer and precancerous conditions, particularly those found in mammals.

Compounds provided herein can be used in the inhibition of certain inflammatory mediators such as, for example, TNF-α and/or nitric oxide synthase (NOS), including the isoforms thereof. Therefore in view of their ability to inhibit both phosphatases and inflammatory mediators such as TNF-α and/or nitric oxide synthase (NOS), the compounds can also be useful for the control of inflammatory diseases such as arthritis.

Compounds provided herein can also be useful for the treatment of certain metabolic disorders including the modulation of carbohydrate and/or lipid metabolism, and/or Type 2 diabetes. Compounds provided herein can be ligands for proteins such as kinases and/or phosphatases that are involved in metabolic disorders.

Some embodiments of the invention relate to methods of synthesizing the compounds disclosed herein.

Compounds provided herein are useful in the treatment of diseases related to uncontrolled cellular proliferation, such as cancer or precancerous conditions. Methods of using such compounds disclosed herein for the treatment of diseases of uncontrolled proliferative diseases in mammals, especially humans, and to pharmaceutical compositions containing compounds thereof are also provided.

In another aspect, this invention relates to the use of the compounds disclosed herein for treating diseases in mammals and/or humans, especially diseases of cellular proliferation, including cancers.

In still another aspect, pharmaceutical compositions are provided for the treatment of diseases of uncontrolled cellular proliferation and cancers comprising a compound disclosed herein as an admixture with one or more pharmaceutically acceptable excipients.

### BRIEF DESCRIPTION OF THE DRAWINGS

FIG. 1 shows selective inhibition of Cdc25 by compound 3 as compared to the dual specificity phosphatase MKP-1.

FIG. 2 shows that compounds 1, 3 and 43 exhibit strong anticancer cell activity against human breast and prostate cancer cells.

FIG. 3 shows that compounds 1, 3 and 43 exhibit strong anticancer all activity against human non-small-cell lung cancer and pancreatic cancer cells.

FIG. 4 shows an example of methods for the synthesis of biaryl intermediates leading to compounds of the invention.

FIG. 5 shows an example of methods for introducing the “Y” group into compounds of the invention.

FIG. 6 shows an example of methods for the synthesis of various heterocycles where W is Oxygen or Sulfur with varying Y groups.

FIG. 7 shows an example of methods for introducing the R₁₀ group into synthetic precursors of the Ar₁ groups of the invention.

FIG. 8 shows an example of methods for the synthesis of intermediates bearing the azaadamantanone and azaadamantanyl group that lead to compounds of the invention.

FIG. 9 shows an example of methods for the synthesis of representative examples of six-membered ring heterocycles as Ar₁.

FIG. 10 shows an example of methods for the synthesis of representative examples of five-membered ring heterocycles as Ar₁.

FIG. 11 illustrates methods for synthesizing compounds of the invention having heteroatomic groups linking the Ar₂ radicals and five membered heterocycles.

FIG. 12 illustrates methods for synthesizing precursors of the benzothiazole compounds of the invention.

FIG. 13 illustrates methods for synthesizing precursors of the benzimidazole compounds of the invention.

FIG. 14 illustrates methods for synthesizing the benzoxazole compounds of the invention.

FIG. 15 shows data on the effectiveness of certain compounds of the invention for killing non-small cell lung cancer cells in vitro as a function of compound concentration.

FIG. 16 shows data on the effectiveness of certain compounds of the invention for killing prostate cancer cells in vitro as a function of compound concentration.

FIG. 17 shows data on the effectiveness of certain compounds of the invention for killing breast cancer cells in vitro as a function of compound concentration.

FIG. 18 shows data on the effectiveness of certain compounds of the invention for killing pancreatic cancer cells in vitro as a function of compound concentration.

FIG. 19 shows data on the effectiveness of certain compounds of the invention for arresting the growth of prostate cancer cells at certain phases of cell growth, in vitro.

FIG. 20 shows data on the effectiveness of compound 43 of the invention for inhibiting the growth of tumors of human prostate cancer cells in athymic nude mice.

FIG. 21 shows data on the effectiveness of compound 81 of the invention for inhibiting the growth of tumors of human prostate cancer cells in athymic nude mice.

FIG. 22 shows data on the effectiveness of compound 81 of the invention for inhibiting the growth of tumors of human non-small cell lung cancer cells in athymic nude mice.

### DETAILED DESCRIPTION

The present invention provides compounds that are useful, for example, to treat diseases of uncontrolled proliferation, for example for the treatment of cancers and precancerous conditions. The present invention can be understood more readily by reference to the following detailed description of preferred embodiments of the invention and the Examples included therein and to the Figures and their previous and following description. It is also to be understood that the terminology used herein is for the purpose of describing particular embodiments only and is not intended to be limiting.

### Definitions

In the specification and Formulae described herein the following terms are hereby defined.

A residue of a chemical species, as used in the specification and concluding claims, refers to the moiety that is the resulting product of the chemical species in a particular reaction scheme or subsequent formulation or chemical product, regardless of whether the moiety is actually obtained from the chemical species. For example, an adamantyl residue in a particular compound has the structure

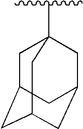

regardless of whether adamantane is used to prepare the compound.

The term “radical” as used in the specification and concluding claims, refers to a fragment, group, or substructure of a molecule described herein, regardless of how the molecule is prepared. In some embodiments the radical (i.e., alkyl) can be further modified (i.e., substituted alkyl) by having bonded thereto one or more “substituent radicals.” The number of atoms in a given radical is not critical to the present invention unless it is indicated to the contrary elsewhere herein.

“Inorganic radicals,” as the term is defined and used herein contain no carbon atoms and therefore comprise only atoms other than carbon. Inorganic radicals comprise combinations of atoms selected from hydrogen, nitrogen, oxygen, silicon, phosphorus, sulfur, selenium, and halogens such as fluorine, chlorine, bromine, and iodine, which can be present individually or bonded together in their chemically stable combinations. Inorganic radicals have 10 or fewer, or preferably 1-4 inorganic atoms as listed above bonded together. Examples of inorganic radicals include, but not limited to, amino, hydroxy, halogens, nitro, azo, thiol, sulfhydril, sulfate, phosphate, and like commonly known inorganic radicals. Inorganic radicals do not have bonded therein the metallic elements of the periodic table (such as the alkali metals, alkaline earth metals, transition metals, lanthanide metals, or actinide metals), except as cations of a pharmaceutically acceptable salt of a compound of the invention having an ionized anionic radical such as a carboxylate, sulfate, phosphate, or the like. Inorganic radicals do not comprise metalloids elements such as boron, aluminum, gallium, germanium, arsenic, tin, lead, or tellurium, or the noble gas elements, unless otherwise specifically indicated elsewhere herein.

“Organic radicals” as the term is defined and used herein contain one or more carbon atoms, and often have hydrogen bound to at least some of the carbons. An organic radical can have, for examples 1-26 carbon atoms, 1-21 carbon atoms, 1-12 carbon atoms, 1-6 carbon atoms, or 1-4 carbon atoms. One example, of an organic radical comprising no inorganic atoms is a 5,6,7,8-tetrahydro-2-naphthyl radical. In some embodiments, an organic radical can contain 1-10 inorganic heteroatoms bound thereto or therein, including halogens, oxygen, sulfur, nitrogen, phosphorus, and the like. Examples of organic radicals include but are not limited to an alkyl, substituted alkyl, cycloalkyl, substituted cycloalkyl, mono-substituted amino, di-substituted amino, acyloxy, cyano, carboxy, carboalkoxy, alkylcarboxamide, substituted alkylcarboxamide, dialkylcarboxamide, substituted dialkylcarboxamide, alkylsulfonyl, alkylsulfinyl, thioalkyl, thiohaloalkyl, alkoxy, substituted alkoxy, haloalkyl, haloalkoxy, aryl, substituted aryl, heteroaryl, heterocyclic, or substituted heterocyclic, wherein the terms are defined elsewhere herein. A few non-limiting examples of organic radicals that include heteroatoms include alkoxy radicals, trifluoromethoxy radicals, acetoxy radicals, dimethylamino radicals and the like.

It must be noted that, as used in the specification and the appended claims, the singular forms “a,” “an” and “the” include plural referents unless the context clearly dictates otherwise. Thus, for example, reference to “an aromatic compound” includes mixtures of aromatic compounds.

Often, ranges are expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint.

The term “alkyl” denotes a radical containing a saturated, straight or branched hydrocarbon residue having from 1 to 18 carbons, or preferably 4 to 14 carbons, 5 to 13 carbons, or 6 to 10 carbons. An alkyl is structurally similar to a non-cyclic alkane compound modified by the removal of one hydrogen from the non-cyclic alkane and the substitution therefore with a non-hydrogen group or radical. Alkyl radicals can be branched or unbranched. Lower alkyl radicals have 1 to 4 carbon atoms. Examples of alkyl radicals include methyl, ethyl, n-propyl, iso-propyl, n-butyl, sec-butyl, t-butyl, amyl, t-amyl, n-pentyl and the like.

The term “substituted alkyl” denotes an alkyl radical analogous to the above definition that is substituted with one or more organic or inorganic substiuent radicals. In some embodiments, 1 or 2 organic or inorganic substiuent radicals are employed. In some embodiments, each organic substiuent radical comprises between 1 and 4, or between 5 and 8 carbon atoms. Suitable organic and inorganic substiuent radicals include but are not limited to hydroxyl, halogens, cycloalkyl, amino, mono-substituted amino, di-substituted amino, acyloxy, nitro, cyano, carboxy, carboalkoxy, alkylcarboxamide, substituted alkylcarboxamide, dialkylcarboxamide, substituted dialkylcarboxamide, alkylsulfonyl, alkylsulfinyl, thioalkyl, thiohaloalkyl, alkoxy, substituted alkoxy, haloalkyl, haloalkoxy, heteroaryl, substituted heteroaryl, aryl or substituted aryl. When more than one substiuent group is present then they can be the same or different.

The term “alkenyl” denotes an alkyl radical as defined above, having 1 to 18 carbons, or preferably 4 to 14 carbons, 5 to 13 carbons, or 6 to 10 carbons which further contains a carbon-carbon double bond. Examples of alkenyl radicals include but are not limited to vinyl, allyl, 2-butenyl, 3-butenyl, 2-pentenyl, 4-methyl-penten-2-yl, 3-pentenyl, 4-methyl-penten-3-yl, 4-pentenyl, 2-hexenyl, 3-hexenyl, 4-hexenyl, 5-hexanyl, 2-heptenyl, 3-heptenyl, 4-heptenyl, 5-heptenyl, 6-heptenyl, and like residues. The term “alkenyl” includes dienes and trienes and other polyunsaturated compounds. The alkenyl radical can exist as E or Z stereoisomers or as a mixture of E or Z stereoisomers. When more than one double bond is present, such as a diene or triene, each double bond can independently exist as E or Z stereoisomers or as a mixture of E or Z stereoisomers with respect to other double bond present in the alkenyl radical.

The term “substituted alkenyl” denotes a alkenyl radical of the above definition that is further substituted with one or more substituent inorganic or organic radicals, which can include but are not limited to halogen, hydroxyl, cycloalkyl, amino, mono-substituted amino, di-substituted amino, acyloxy, nitro, cyano, carboxy, carboalkoxy, alkylcarboxamide, substituted alkylcarboxamide, dialkylcarboxamide, substituted dialkylcarboxamide, alkylsulfonyl, alkylsulfinyl, thioalkyl, thiohaloalkyl, alkoxy, substituted alkoxy or haloalkoxy. In some embodiments, 1 or 2 organic or inorganic substituent radicals are employed. In some embodiments, each organic substituent radical comprises between 1 and 4, or between 5 and 8 carbon atoms. When more than one group is present then they can be the same or different.

The term “alkynyl” denotes a radical containing a straight or branched chain of having 1 to 18 carbons, or preferably 4 to 14 carbons, 5 to 13 carbons, or 6 to 10 carbons, such as ethynyl, 1-propynyl, 2-propynyl, 1-butynyl, 2-butynyl, 3-butynyl, 1-pentynyl, 2-pentynyl, 3-pentynyl, 4-pentynyl, 1-hexynyl, 2-hexynyl, 3-hexynyl, 4-hexynyl, 5-hexynyl and like residues. The term “alkynyl” includes di- and tri-ynes.

The term “substituted alkynyl” denotes a alkynyl of the above definition that is substituted with one or more organic or inorganic radicals, that can include halogen, hydroxyl, cycloalkyl, amino, mono-substituted amino, di-substituted amino, acyloxy, nitro, cyano, carboxy, carboalkoxy, alkylcarboxamide, substituted alkylcarboxamide, dialkylcarboxamide, substituted dialkylcarboxamide, alkylsulfonyl, alkylsulfinyl, thioalkyl, thiohaloalkyl, alkoxy, substituted alkoxy or haloalkoxy residues.

The term “cycloalkyl” denotes a radical containing 1 to 18 carbons, or preferably 4 to 14 carbons, 5 to 10 carbons, or 5 to 6 carbons, such as cyclopropyl, cyclobutyl, cyclopentyl, cyclopentyl, cyclohexyl, cycloheptyl, decahydronapthyl, adamantyl, and like residues.

The term “substituted cycloalkyl” denotes a cycloalkyl as defined above that is further substituted with one or more organic or inorganic groups that can include halogen, alkyl, substituted alkyl, hydroxyl, alkoxy, substituted alkoxy, carboxy, carboalkoxy, alkylcarboxamide, substituted alkylcarboxamide, dialkylcarboxamide, substituted dialkylcarboxamide, amino, mono-substituted amino or di-substituted amino. When the cycloalkyl is substituted with more than one group, they can be the same or different.

The term “cycloalkenyl” denotes a cycloalkyl radical further comprising at least one carbon-carbon double bond, including cyclopropenyl, 1-cyclobutenyl, 2-cyclobutenyl, 1-cyclopentenyl, 2-cyclopentenyl, 3-cyclopentenyl, 1-cyclohexyl, 2-cyclohexyl, 3-cyclohexyl, and like radicals.

The term “substituted cycloalkenyl” denotes a cycloalkenyl residues as defined above further substituted with one or more groups selected from halogen, alkyl, hydroxyl, alkoxy, substituted alkoxy, haloalkoxy, carboxy, carboalkoxy, alkylcarboxamide, substituted alkylcarboxamide, dialkylcarboxamide, substituted dialkylcarboxamide, amino, mono-substituted amino or di-substituted amino. When the cycloalkenyl is substituted with more than one group, they can be the same or different.

The term “alkoxy” as used herein denotes a radical alkyl, defined above, attached directly to a oxygen to form an ether residue. Examples include methoxy, ethoxy, n-propoxy, iso-propoxy, n-butoxy, t-butoxy, iso-butoxy and the like.

The term “substituted alkoxy” denotes a alkoxy radical of the above definition that is substituted with one or more groups, but preferably one or two substituent groups including hydroxyl, cycloalkyl, amino, mono-substituted amino, di-substituted amino, acyloxy, nitro, cyano, carboxy, carboalkoxy, alkylcarboxamide, substituted alkylcarboxamide, dialkylcarboxamide, substituted dialkylcarboxamide, alkylsulfonyl, alkylsulfinyl, thioalkyl, thiohaloalkyl, alkoxy, substituted alkoxy or haloalkoxy. When more than one group is present then they can be the same or different.

The term “mono-substituted amino” denotes an amino (—NH₂) group substituted with one group selected from alkyl, substituted alkyl or arylalkyl wherein the terms have the same definitions found throughout.

The term “di-substituted amino” denotes an amino substituted with two radicals that can be same or different selected from aryl, substituted aryl, alkyl, substituted alkyl or arylalkyl wherein the terms have the same definitions found throughout. Some examples include dimethylamino, methylethylamino, diethylamino and the like.

The term “haloalkyl” denotes a alkyl radical, defined above, substituted with one or more halogens, preferably fluorine, such as a trifluoromethyl, pentafluoroethyl and the like.

The term “haloalkoxy” denotes a haloalkyl, as defined above, that is directly attached to an oxygen to form a halogenated ether residue, including trifluoromethoxy, pentafluoroethoxy and the like.

The term “acyl” denotes a radical of the formula —C(O)—R that comprises a carbonyl (C═O) group, wherein the R radical is an organic radical. Acyl radicals often contain 1 to 8 carbon atoms. Examples of acyl radicals include but are not limited to formyl, acetyl, propionyl, butanoyl, iso-butanoyl, pentanoyl, hexanoyl, heptanoyl, benzoyl and like radicals.

The term “acyloxy” denotes a radical containing 1 to 8 carbons of an acyl group defined above directly attached to an oxygen such as acetyloxy, propionyloxy, butanoyloxy, iso-butanoyloxy, benzoyloxy and the like.

The term “aryl” denotes an unsaturated and conjugated aromatic ring radical containing 6 to 18 ring carbons, or preferably 6 to 12 ring carbons. Many aryl radicals have at least one six-membered aromatic “benzene” radical therein. Examples of such aryl radicals include phenyl and naphthyl.

The term “substituted aryl” denotes an aryl ring radical as defined above that is substituted with or fused to one or more organic or inorganic substituent radicals, which include but are not limited to a halogen, alkyl, substituted alkyl, haloalky, hydroxyl, cycloalkyl, substituted cycloalkyl, cycloalkenyl, substituted cycloalkenyl, amino, mono-substituted amino, di-substituted amino, acyloxy, nitro, cyano, carboxy, carboalkoxy, alkylcarboxamide, substituted alkylcarboxamide, dialkylcarboxamide, substituted dialkylcarboxamide, alkylsulfonyl, alkylsulfinyl, thioalkyl, thiohaloalkyl, alkoxy, substituted alkoxy or haloalkoxy, aryl, substituted aryl, heteroaryl, heterocyclic ring, substituted heterocyclic ring radical, wherein the terms are defined herein. Substituted aryl radicals can have one, two, three, four, five, or more substituent radicals. The substituent radicals can be not be of unlimited size or molecular weight, and each organic radical can comprise 15 or fewer, 10 or fewer, or 4 or fewer carbon atoms unless otherwise expressly contemplated by the claims

The term “heteroaryl” denotes an aryl ring radical as defined above, wherein at least one of the carbons of the aromatic ring has been replaced with a heteroatom, which include but are not limited to nitrogen, oxygen, and sulfur atoms. Heteroaryl radicals include 6 membered aromatic ring radicals, and can also comprise 5 or 7 membered aromatic rings, or bicyclic or polycyclic heteroaromatic rings as well. Examples of heteroaryl radicals include pyridyl, bipyridyl, furanyl, and thiofuranyl residues. Further examples of heteroaryl residues which can be employed in the chemical structures of the invention include but are not limited to the residues exemplified below:

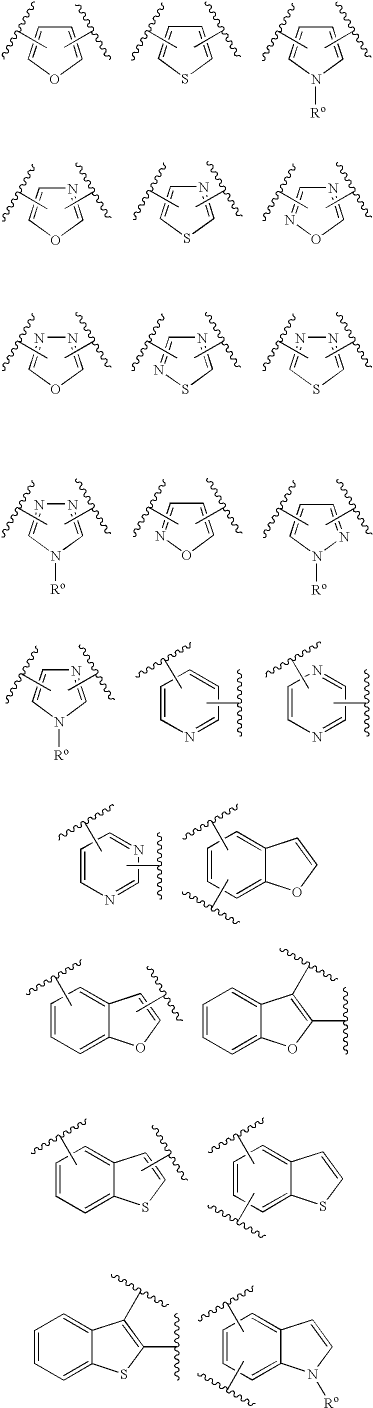

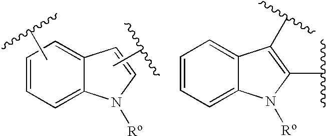

wherein R^{o} can be hydrogen, alkyl, substituted alkyl, aryl, substituted aryl, and the like. It is to be understood that the heteroaryl radicals can optionally be substituted with one or more organic or inorganic substituent radicals bound to the carbon atoms of the heteroaromatic rings, as described hereinabove for substituted aryl radicals. Substituted heteroaryl radicals can have one, two, three, four, five, or more substituent organic or inorganic radicals, in a manner analogous to the substituted aryl radicals defined herein. The substituent radicals cannot be of unlimited size or molecular weight, and each organic substituent radical can comprise 15 or fewer, 10 or fewer, or four or fewer carbon atoms unless otherwise expressly contemplated by the claims.

The term “halo,” “halide,” or “halogen” refers to a fluoro, chloro, bromo or iodo atom or ion.

The term “thioalkyl” denotes a sulfide radical containing 1 to 8 carbons, linear or branched. Examples include methylsulfide, ethyl sulfide, isopropylsulfide and the like.

The term “thiohaloalkyl” denotes a thioalkyl radical substituted with one or more halogens. Examples include trifluoromethylthio, 1,1-difluoroethylthio, 2,2,2-trifluoroethylthio and the like.

The term “carboalkoxy” refers to an alkyl ester of a carboxylic acid, wherein alkyl has the same definition as found above. Examples include carbomethoxy, carboethoxy, carboisopropoxy and the like.

The term “alkylcarboxamide” denotes a single alkyl group attached to the amine of an amide, wherein alkyl has the same definition as found above. Examples include N-methylcarboxamide, N-ethylcarboxamide, N-(iso-propyl)carboxamide and the like. The term “substituted alkylcarboxamide” denotes a single “substituted alkyl” group, as defined above, attached to the amine of an amide.

The term “dialkylcarboxamide” denotes two alkyl or arylalkyl groups that are the same or different attached to the amine of an amide, wherein alkyl has the same definition as found above. Examples of a dialkylcarboxamide include N,N-dimethylcarboxamide, N-methyl-N-ethylcarboxamide and the like. The term “substituted dialkylcarboxamide” denotes two alkyl groups attached to the amine of an amide, where one or both groups is a “substituted alkyl”, as defined above. It is understood that these groups can be the same or different. Examples include N,N-dibenzylcarboxamide, N-benzyl-N-methylcarboxamide and the like.

The term “alkylamide” denotes an acyl radical attached to an amine or monoalkylamine, wherein the term acyl has the same definition as found above. Examples of “alkylamide” include acetamido, propionamido and the like.

The term “heterocycle” or “heterocyclic”, as used in the specification and concluding claims, refers to a radical having a closed ring structure comprising 3 to 10 ring atoms, in which at least one of the atoms in the ring is an element other than carbon, such as, for example, nitrogen, sulfur, oxygen, silicon, phosphorus, or the like. Heterocyclic compounds having rings with 5, 6, or 7 members are common, and the ring can be saturated, or partially or completely unsaturated. The heterocyclic compound can be monocyclic, bicyclic, or polycyclic. Examples of heterocyclic compounds include but are not limited to pyridine, piperidine, thiophene, furan, tetrahydrofuran, and the like. The term “substituted heterocyclic” refers to a heterocyclic radical as defined above having one or more organic or inorganic substituent radicals bonded to one of the ring atoms.

The term “carboxy”, as used in the specification and concluding claims, refers to the —C(O)OH radical that is characteristic of carboxylic acids. The hydrogen of the carboxy radicals is often acidic and (depending on the pH) often partially or completely dissociates, to form an acid H+ion and a carboxylate anion (—CO₂⁻), wherein the carboxylate anion is also sometimes referred to as a “carboxy” radical.

The term “nitrile”, as used in the specification and concluding claims, refers to a compound having a —CN substituent radical wherein the carbon is triply bonded to the nitrogen atom.

The term “alkylsilyloxy”, as used in the specification and concluding claims, refers to a radical of the formula -0-SiR₁R₂R₃ wherein the R₁,R₂, and R₃ groups are independently hydrogen or organic radicals, wherein the organic radicals preferably contain from one to ten carbon atoms.

The term “alkylene” as used herein refers to a difunctional saturated branched or unbranched hydrocarbon chain containing from 1 to 36 carbon atoms, and includes, for example, methylene (—CH₂—), ethylene (—CH₂—CH₂—), propylene (—CH₂—CH₂(CH₃)—), 2-methylpropylene [—CH₂—CH(CH₃)—CH₂—], hexylene [—(CH₂)₆—] and the like. “Lower alkylene” refers to an alkylene group of from 1 to 6, more preferably from 1 to 4, carbon atoms.

The term “cycloalkylene” as used herein refers to a cyclic alkylene group, typically a 5- or 6-membered ring.

The term “arylalkyl” defines an alkylene as described above which is substituted with an aryl group that can be substituted or unsubstituted as defined above. Examples of an “arylalkyl” include benzyl, phenethylene and the like.

### Compounds

Some disclosed embodiments of the invention relate to compounds of the Formula (I):

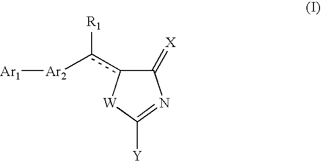

wherein:

(a) Ar₁ is an aryl, substituted aryl, heteroaryl, or substituted heteroaryl;

(b) Ar₂ is an aryl, substituted aryl, heteroaryl, or substituted heteroaryl;

(c) R₁ is hydrogen, hydroxy, alkoxy, alkyl, or substituted alkyl;

(d) - - - represents a bond present or absent;

(e) W is S or O;

(f) X is S or O; and

(g) Y is

(i) an organic radical comprising 1 to 15 carbon atoms,

(ii) an —S—R₂ or -0-R₂ radical wherein the R₂ radical comprises 1 to 10 carbon atoms; or

(iii) an —NR₃R₄ radical wherein R₃ and R₄ are

a. independently hydrogen, hydroxyl, amino, or an organic radical comprising 1 to 15 carbon atoms, or

b. R₃ and R₄ together with the nitrogen form a heterocycle, or substituted heterocycle comprising 1 to 15 carbon atoms;

or a pharmaceutically acceptable salt thereof.

The compounds of Formula (I) comprise 2-substituted heterocyclic moieties having 5-membered heterocyclic rings with the structure:

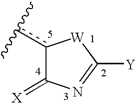

wherein X and W can independently be sulfur or oxygen. Such heterocyclic moieties are referred to as thiazolidinone (when W=sulfur) or oxazolidinone (when W=oxygen) moieties. The heterocyclic moieties of the invention also have a “Y” substituent bound to the carbon at the 2-position of the heterocyclic ring, as will be further described hereinbelow. Therefore, the substituted heterocycles of Formula 1 can be referred to as, for example, 2-substituted-thiazolidinone or 2-substituted-oxazolidinone compounds.

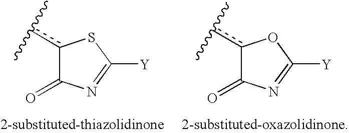

The 2-thiazolidinone or 2-oxazolidinone moieties of Formula (I) are connected, via a single or double bond, to a bridging carbon atom, that is in turn bonded to the Ar₂ radical. Nevertheless, the use of a carbon atom to connect the 2-thiazolidinone or 2-oxazolidinone moieties to the Ar₂ is not believed to be critical to the invention, and the bridging carbon atom can be replaced with a heteroatom (such as nitrogen, oxygen, sulfur, or the like) or heteroatomic group (such as a sulfoxide, sulfone, or the like), to produce useful compounds within the scope of the invention, as will be further described herein.

The bridging carbon atom illustrated in Formula (I) is connected to the R₁ substituent radical, which can be hydrogen or another organic or inorganic substituent radical. The R₁ radical should not be so large as to inhibit the binding of the compound to the target receptor proteins, and therefore preferably contains less than 10 non-hydrogen carbon atoms or heteroatoms. In some embodiment, the R₁ radical has 1 to 10 or 1 to four carbon atoms. In certain embodiments, the R₁ radical is hydrogen, hydroxy, alkoxy, alkyl, or substituted alkyl radical. In some embodiments R₁ is hydrogen, a alkyl or a substituted alkyl. In another embodiment, R₁ is hydrogen or a lower alkyl.

The compounds of Formula (I) have an Ar₁ radical that is an aryl, substituted aryl, heteroaryl, or substituted heteroaryl radical, as defined elsewhere herein, and is connected by a carbon-carbon bond to the Ar₂ radical. The substituted aryl and substituted heteroaryl radicals can have 1 to 5 R₁ₓ organic or inorganic substituent radicals, wherein x is 0 to 4, that are bound to ring carbon atoms of Ar₁. The R₁ₓ radicals can be bound to any ring carbon atom, in any position relative to the bond to the Ar₂ radical and in any position with respect to each other. Suitable R₁ₓ radicals can be independently selected and include but are not limited to hydrogen, halogen, alkyl, substituted alkyl, haloalkyl, alkenyl, substituted alkenyl, alkynyl, substituted alkynyl, cycloalkyl, substituted cycloalkyl, cycloalkenyl, substituted cycloalkenyl, heterocyclic, substituted heterocyclic, alkoxy, substituted alkoxy, hydroxyl, acyl, amino, monosubstituted amino, di-substituted amino, carboxy, carboalkoxy, nitrile, alkylcarboxamide, substituted alkylcarboxamide, dialkylcarboxamide, substituted dialkylcarboxamide, haloalkoxy, alkylsilyloxy, heteroaryl, substituted heteroaryl, aryl, or substituted aryl radicals. In many embodiments, the R₁ₓ radicals each comprise 1 to 12 carbon atoms, 1 to 10 carbon atoms, or 1 to 4 carbon atoms.

Although not wishing to be bound by theory, the Ar₁ radical together with its substituent radicals are preferably of a size that is sufficiently small as to allow the Ar₁ radical to substantially fill, yet fit within the binding regions of the target phosphatases. Therefore, in many embodiments, the Ar₁ radical, together with all its substituent R₁ₓ radicals, comprises between 4 and 30 carbon atoms, or between 5 and 25 carbon atoms, or between six and 20 carbon atoms.

In many embodiments of the invention, the anti-cancer activity of the compounds of the invention can be substantially and unexpectedly improved if at least one of the R₁ₓ radicals is a bulky (i.e. sterically demanding) substituent radical bonded to a carbon of the aromatic ring of the Ar₁ radical. Those of ordinary skill in organic chemistry are aware of many types of bulky substituent radicals. One type of bulky substituent radical has the following formula;

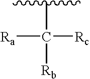

wherein Rₐ, R_{b}, and R_{c} are independently or together hydrogen, or an inorganic or organic radical, with the proviso that no more than one of Rₐ, R_{b}, and R_{c} are hydrogen, so that the bulky substituent radical has a branched structure at the central carbon atom of the radical.

One or more of Rₐ, R_{b}, and R_{c} can be a heteroatom such as oxygen, nitrogen, sulfur, phosphorus, or the like, or a organic radical having heteroatoms therein, such as alkoxy, mono or di-substituted amino groups and the like. These branched substituent radicals have a secondary or tertiary carbon atom bonded to the carbons of the Ar₁ ring. In some embodiments, Rₐ, R_{b}, and R_{c} can be an alkyl, substituted alkyl, cycloalkyl, substituted alkyl, heterocyclic or substituted heterocyclic radical.

Examples of such branched substituent are the isopropyl, 2-methylpropyl, cyclopentyl, and cyclohexyl radicals shown below.

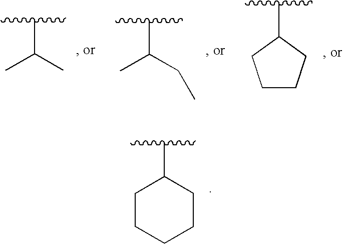

In some embodiments none of Rₐ, R_{b}, and R_{c} are hydrogen, and therefore a tertiary carbon atom is bonded to the aryl or heteroaryl ring. In some embodiments Rₐ, R_{b}, and R_{c} are alkyls that each comprise 1 to 4 carbon atoms. Examples of such tertiary alkyl substituents include radicals such as

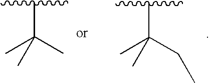

Two or three of the Rₐ, R_{b}, and R_{c} radicals of the branched radical can be bonded together to form cyclic, bicyclic, polycyclic, heterocyclic, alicyclic, aryl, or heteroaryl rings. The Rₐ, R_{b}, and R_{c} radicals can in some embodiments be bonded to additional organic or inorganic substituent groups. Examples of such branched radicals having cyclic radicals including

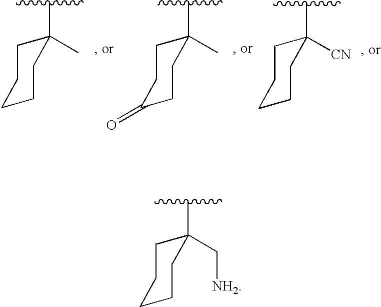

The branched substituent radical can be a substituted “adamantyl” radical of the Formula (VIIIa):

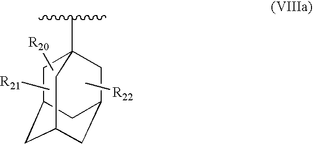

wherein:

R₂₀, R₂₁ and R₂₂ can be hydrogen, an inorganic radical, or an organic radical at any position on the adamantyl radical. In some embodiments, R₂₀, R₂₁ and R₂₂ are independently selected from hydrogen, halogen, alkyl, hydroxy, carboxyl, alkylcarboxamide or dialkylcarboxamide radicals. In one embodiment the branched substituent radical is a substituted cycloalkyl of Formula (VIIIa) wherein R₂₀, R₂₁ and R₂₂ are hydrogen, such that the substituted cycloalkyl is an unsubstituted adamantyl radical of Formula (Vie):

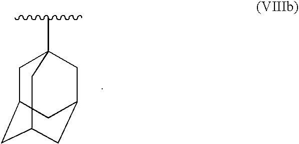

In another embodiment the branched substituent radical is a substituted adamantyl radical of Formula (VIIIa) wherein R₂₀ is a fluorine. In another embodiment, the branched radical is a radical of Formula (Voice):

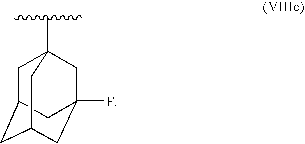

Some embodiments of the invention relate to compounds of Formula (I) wherein the branched substituent radical is a substituted heterocyclic radical of the Formula (Vied):

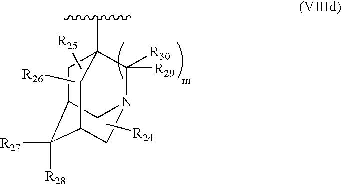

wherein:

m is 0 or 1;

R₂₄, R₂₅ and R₂₆ can be attached to any carbon on the substituted heterocyclic radical except for the carbons bearing R₂₇ and R₂₈ or R₂₉ and R₃₀ and are independently hydrogen, halogen, alkyl, hydroxy, carboxyl, alkylcarboxamide or dialkylcarboxamide;

R₂₇ and R₂₈ are independently hydrogen, halogen, or hydroxy; or R₂₇ and R₂₈ together form a carbonyl radical;

R₂₉ and R₃₀ are independently hydrogen; or R₂₉ and R₃₀ together form a carbonyl radical.

In one embodiment the branched substituent radical is a substituted heterocyclic radical of Formula (Vied) wherein m is 0; R₂₄, R₂₅ and R₂₆ are hydrogen; R₂₇ and R₂₈ are each hydrogen or R₂₇ and R₂₈ together form a carbonyl radical of the following formulas:

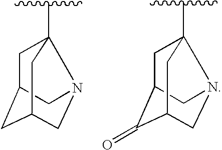

In one embodiment, the branched radical is a substituted heterocyclic radical of Formula (VIIId) wherein m is 1, R₂₄ and R₂₅ are independently an alkyl, R₂₆ is hydrogen and R₂₇ and R₂₈ are each a hydrogen or R₂₇ and R₂₈ together form a carbonyl of the for following formulas:

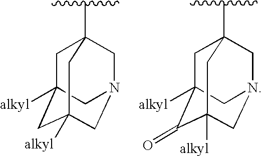

In one embodiment, the branched substituent radical is a substituted heterocyclic radical of Formula (VIIId) wherein m is 1; R₂₄, R₂₅ and R₂₆ are hydrogen; R₂₇ and R₂₈ are hydrogen or R₂₇ and R₂₈; and R₂₉ and R₃₀ together form a carbonyl of the following formulas:

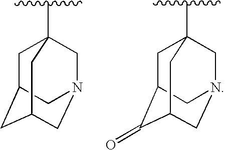

In certain embodiments, the branched substituent radical for Ar₁ is a t-butyl, a 2-methylpropyl, a phenyl, a 2-pyridyl, a 3-pyridyl, a 4-pyridyl, a 1-alkylcyclohexyl, azaadamantyl, azaadamantone-yl or an adamantyl radical.

Although the R₁ₓ and/or bulky substituent radicals of Ar₁ can be bonded to any position of the Ar₁ ring, in some embodiments the bulky substituent radical has a “meta” orientation relative to the substitution of the Ar₂ ring. Some embodiments of the invention relate to compounds wherein Ar₁ is a meta-substituted benzene radical of Formula (II):

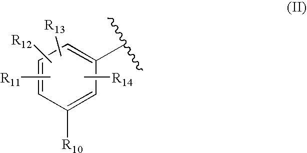

wherein:

R₁₀ is not hydrogen, and can be the branched substituent radicals as 5 disclosed above, or is an inorganic radical, or an organic radical having 1 to 15 carbon atoms. Examples of suitable inorganic or organic radicals include hydroxy, a halogen, alkyl, substituted alkyl, haloalkyl, thioalkyl, thiohaloalkyl, alkylsulfonyl, alkylsulfinyl, alkoxy or substituted alkoxy, haloalkoxy, alkenyl, substituted alkenyl, cycloalkyl, substituted cycloalkyl, heterocyclic, substituted heterocyclic, mono-substituted amino, di-substituted amino, alkyl carboxamide, substituted carboxamide, dialkylcarboxamide, substituted dialkylcarboxamide, heteroaryl, substituted heteroaryl, aryl, or substituted aryl; and

R₁₁, R₁₂, R₁₃ and R₁₄ are independently selected from hydrogen, inorganic radicals, or organic radicals having 1 to 15 carbon atoms which optionally have from one to ten non-hydrogen atoms. Examples of suitable inorganic and organic substituent radicals include but are not limited to a halogen, alkyl, substituted alkyl, thioalkyl, thiohaloalkyl, alkylsulfonyl, alkylsulfinyl, alkenyl, substituted alkenyl, alkynyl, substituted alkynyl, cycloalkyl, substituted cycloalkyl, heterocyclic, substituted heterocyclic, haloalkyl, haloalkoxy, alkoxy, substituted alkoxy, hydroxyl, acyl, amino, monosubstituted amino, di-substituted amino, carboxy, carboalkoxy, nitrile, alkylcarboxamide, substituted alkylcarboxamide, dialkylcarboxamide, substituted dialkylcarboxamide, haloalkoxy, alkylsilyloxy, heteroaryl, substituted heteroaryl, aryl, or substituted aryl radicals. In some embodiments, at least one of R₁₁, R₁₂, R₁₃ and R₁₄ are not hydrogen. In some preferred embodiments, R₁₁ is a hydroxy radical, preferably bonded ortho to R₁₀ and para to the Ar₂ radical.

Alternatively, some embodiments of the invention relate to compounds of Formula (I) wherein Ar₁ is a substituted aryl radical of Formula (II) and:

R₁₀ is a bulky organic substituent radical, a branched radical as describe above, or an alkyl, substituted alkyl, cycloalkyl, substituted cycloalkyl, heterocyclic, substituted heterocyclic, heteroaryl, substituted heteroaryl, aryl, or substituted aryl radical;

R₁₁ is hydrogen, alkoxy, substituted alkoxy, hydroxyl, carboxy, carboalkoxy, alkylcarboxamide, substituted alkylcarboxamide, dialkylcarboxamide, substituted dialkylcarboxamide, haloalkoxy or alkylsilyloxy; and

R₁₂, R₁₃ and R₁₄ are independently hydrogen, halogen, alkyl, substituted alkyl,-alkenyl, substituted alkenyl, akynyl, substituted alkynyl, cycloalkyl, substituted cycloalkyl, heterocyclic, substituted heterocyclic, alkoxy, substituted alkoxy, hydroxyl, acyl, amino, mono-substituted amino, di-substituted amino, carboxy, carboalkoxy, nitrile, alkylcarboxamide, substituted alkylcarboxamide, dialkylcarboxamide, substituted dialkylcarboxamide, haloalkoxy, alkylsilyloxy, heteroaryl, substituted heteroaryl, aryl, or substituted aryl.

In some of the above embodiments, R₁₁ is para to the Ar₂ ring and ortho to the R₁₀ substituent, so as to form Ar₁ radicals having the structure:

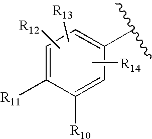

wherein the R₁₀, R₁₁, R₁₂, R₁₃ and R₁₄ groups are one of the substituent groups defined above. In certain embodiments, R₁₀ is a branched substituent as disclosed above and R₁₁ is a hydroxy, or alkoxy group. In certain preferred embodiments, R₁₁ is hydroxy and R₁₃ and R₁₄ are hydrogen, to give a radical having the formula

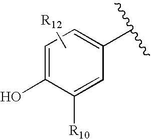

In some embodiments of Ar₁ radicals related to the embodiments above, R₁₁ and one of the R₁₂, R₁₃ and R₁₄ radical together form an additional ring fused to the aromatic ring of the above compounds, so as to form a bicyclic Ar₁ radical, having the generic structures shown below:

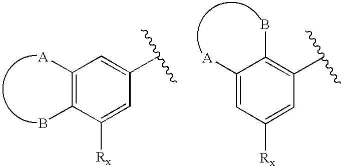

wherein Rₓ can be hydrogen, an inorganic radical, or an organic radical comprising 1 to 15 carbon atoms, and A and B are optional heteroatoms independently selected from the group consisting of —O—, —N—, —NR₄13 , and —S—. In many embodiments, Rₓ is a branched radical as discussed above. The additional ring can comprise a cycloalkyl, a cycloalkenyl, a partially or completely saturated heterocyclic, or a heteroaryl ring. It is to be understood that for the purposes of this document, when the additional ring of the fused ring structures shown immediately above is a cycloalkyl or cycloalkenyl ring, the delocalized carbon-carbon double bond that is part of the benzene ring of is not to considered to be relevant to the definition of the additional ring as a “cycloalkyl” or “cycloalkenyl” ring. In many embodiments, the additional ring has 5, 6, 7, or 8 ring atoms, including the 2 carbon atoms of the benzene ring fused thereto. The additional ring can optionally be substituted with 1, 2, 3, 4 or 5 inorganic or organic substituent radicals.

In some embodiments, the bicyclic Ar₁ radicals can have structures such as

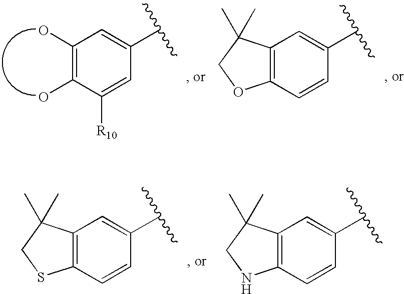

In some embodiments, the bicyclic Ar₁ radicals can have an additional ring that is heteroaromatic, and forms, for example, benzofurans, benzothiophenes, and the like. Therefore, in some embodiments, Ar₁ is a bicyclic heteroaromatic group having the generic formula:

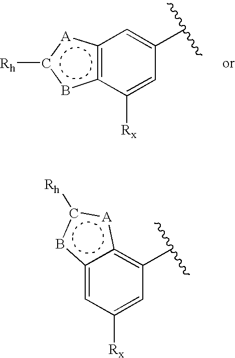

wherein A and B are independently selected from the group consisting of —O— —N—, —NR_{x′}—, and —S—; Rₓ can be independently selected from hydrogen, an inorganic radical, and an organic radical comprising 1 to 15 carbon atoms, or the branched radicals described above; C is carbon; at least one of A or B is —N—; and Rₕ is selected from the group consisting of hydrogen, —SH, —NH₂, or a organic radical having 1 to 7 carbon atoms and optionally one to three heteroatoms selected from the group consisting of O, S, N, and halogens.

In some embodiments, Ar₁ is benzoxazole group having the formula

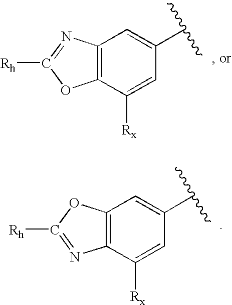

In some embodiments, Ar₁ is benzothiazole group having the formula

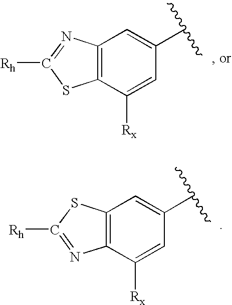

In some embodiments, Ar₁ is benzimidazole group having the formula

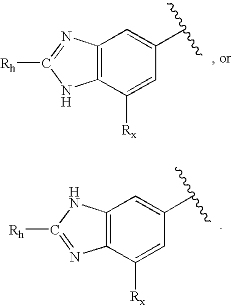

In the above embodiments relating to fused heterocyclic Ar₁ radicals comprising benzoxazole, benzothiazole, and benzimidazole radicals, beneficial results can be obtained if Rₓ is one of the branched radicals described hereinabove. For example, in some embodiments, compounds containing Ar₁ radicals of the following structures can be useful

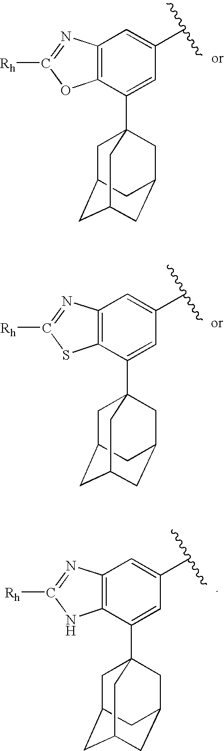

In some embodiments Ar₁ is a 3-adamantyl-phenyl, 3-adamantyl-4-hydroxy-phenyl or 3-adamantyl-4-hydroxy-5-fluoro-phenyl radical having the structure:

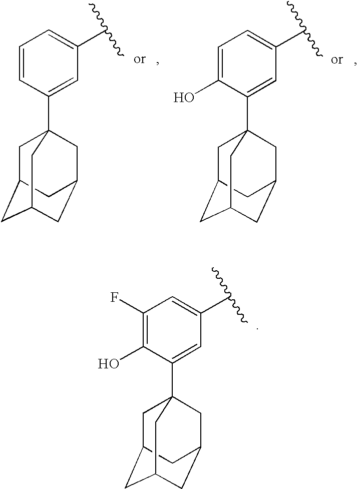

In other embodiments Ar₁ has the structure:

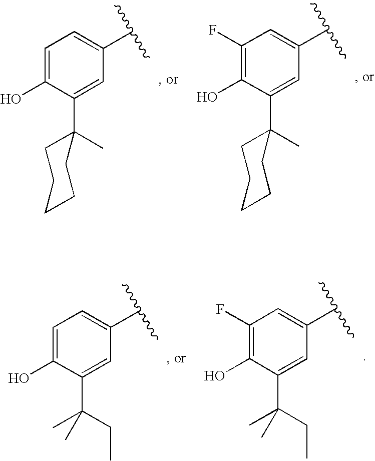

Compounds of Formula (I) wherein Ar₁ is a heteroaryl or substituted heteroaryl radical can have, for example, Formula (III):

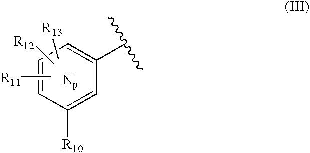

wherein the R₁₀, R₁₁, R₁₂, and R₁₃ groups are as defined above, and

N is a ring atom at any position not substituted with R₁₀, R₁₁, R₁₂, R₁₃, or Ar₂ residues;

p is 1, 2 or 3,; and

R₁₀, R₁₁, R₁₂, and R₁₃ have the same definitions described herein.

Some embodiments of the invention relate to compounds of Formula (III) wherein Ar₁ is a substituted heteroaryl radical of Formula (IV) or (V):

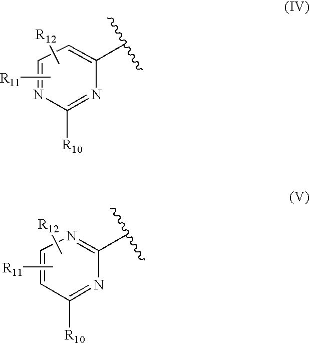

wherein R₁₀, R₁₁, and R₁₂ have the same definitions described hereinabove.

Some embodiment of the invention relate to compounds of Formula (I) wherein Ar₁ is a heteroaryl or substituted heteroaryl radical of the Formula (VI):

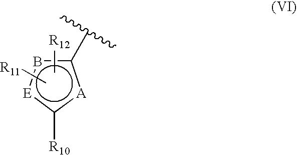

wherein:

A, B, and E are independently O, S or N; and

R₁₀, R₁₁, and R₁₂ have the same definitions described hereinbove.

Illustrative embodiments of such heteroaryl or substituted heteroaryl radicals are provided below in Formula (VIIa), (VIIb), (VIIc), (VIId) or (VIIe):

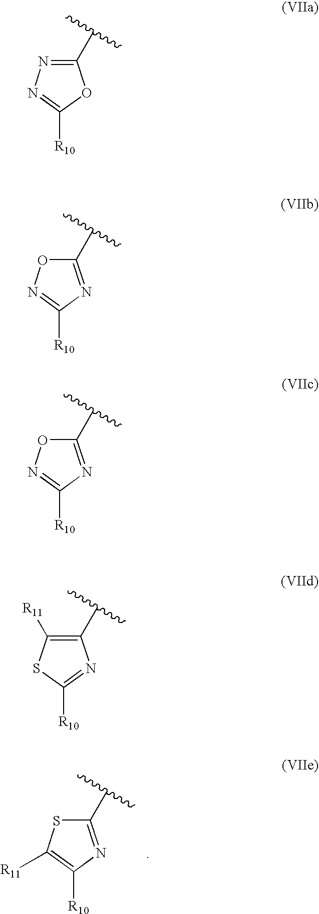

wherein R₁₀, R₁₁, and R₁₂ have the same definitions described hereinbove.

The compounds of Formula (I) comprise an Ar₂ radical which is an aryl, substituted aryl, heteroaryl, or substituted heteroaryl radical, as defined elsewhere herein. Ar₂ is connected by a single carbon-carbon bond to the Ar₁ radical and an atom that links the Ar₂ radical to the five membered heterocyclic radical. In many embodiments, the linking atom is a carbon atom and bears an R₁ substituent. In other embodiments, the linking atom is a heteroatom, as described hereinbelow.

The Ar₂ radicals can be substituted aryl and substituted heteroaryl radicals having 1, 2, 3, 4, or more organic or inorganic substituent R₃ₓ radicals bound thereto, wherein x is an integer from 1-9. The R₃ₓ radicals can have any orientation relative to the Ar₁ radical and any orientation with respect to each other. Although not wishing to be bound by theory, the Ar₂ radical and its substituent radicals must be of a size that is sufficiently small so as to allow the compounds of the invention to fit within the binding regions of the target phosphatases. Therefore, in many embodiments, the Ar₂ radical, together with all its substituents, comprises between 4 and 30 carbon atoms, or between 5 and 25 carbon atoms, or between six and 20 carbon atoms.

In one embodiment of the invention Ar₂ is a substituted benzene radical of Formula (IXa):

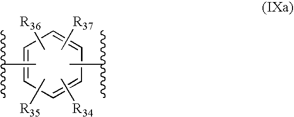

wherein:

R₃₄, R₃₅, R₃₆ and R₃₇ are independently selected from hydrogen or an organic or inorganic radical. In some embodiments, the R₃₄, R₃₅, R₃₆ and R₃₇ radicals can independently comprise between 1 and 10 non-hydrogen atoms, or between 1 and 3 non hydrogen atoms, selected from halogens, —O—, —S—, and —N—. In many embodiments the organic substituent radicals for Ar₂ have from 1 to 8 or from 1 to 4 carbon atoms. Examples of suitable R₃₄, R₃₅, R₃₆ and R₃₇ radicals include hydrogen, alkyl, haloalkyl, haloalkoxy, substituted alkyl, alkenyl, substituted alkenyl, alkynyl, substituted alkynyl, cycloalkyl, substituted cycloalkyl, heterocyclic, substituted heterocyclic, alkoxy, substituted alkoxy, hydroxyl, acyl, amino, mono-substituted amino, di-substituted amino, carboxy, carboalkoxy, alkylcarboxamide, substituted alkylcarboxamide, dialkylcarboxamide, substituted dialkylcarboxamide, haloalkoxy, heteroaryl, substituted heteroaryl, aryl, substituted aryl; or two adjacent groups together with the aromatic ring form a cycloalkyl, substituted cycloalkyl, cycloalkenyl or substituted cycloalkenyl optionally comprising 1 or 2 heteroatomic residues selected from O, S, NH, N-alkyl and N-substituted alkyl residues.

In another embodiment, Ar₂ is a meta- or para-substituted benzene radical of the Formula (IXb) or Formula (IXc):

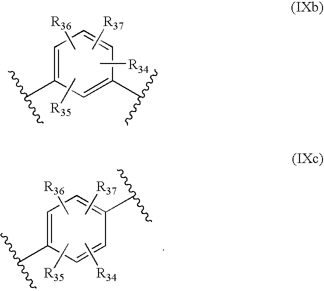

wherein R₃₄, R₃₅, R₃₆ and R₃₇ have the same definitions as described hereinabove.

In some embodiments, Ar₂ is a meta-substituted benzene radical having the formula

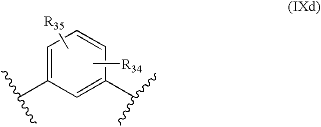

wherein R₃₄ and R₃₅ are as defined hereinabove. In some beneficial embodiments R₃₄ and R₃₅ are independently selected from hydrogen, hydroxy, halogen, alkyl, haloalkyl, 1 5 alkoxy, or haloalkoxy. In many embodiments, at least one of R₃₄ and R₃₅ is hydrogen, hydroxy, or fluorine. In many embodiments, the alkyl, haloalkyl, alkoxy, or haloalkoxy radicals have from 1 to 4 carbon atoms.

In one embodiment, Ar₂ is a benzene radical wherein two adjacent substituent radicals together with the aromatic ring form a heterocycle comprising 2 oxygen atoms having the formulas:

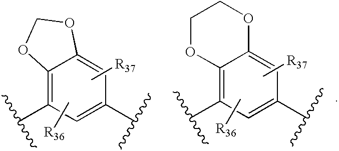

wherein R₃₆ and R₃₇ have the same definitions as described hereinabove.

In some embodiments the Ar₂ heteroaryls are a pyridine radical having the structure

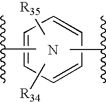

wherein the nitrogen atom is at any unsubstituted ring position and R₃₄, R₃₅ are independently selected and are defined as described hereinabove. In some beneficial embodiments R₃₄ and R₃₅ are independently selected from hydrogen, hydroxy, halogen, alkyl, haloalkyl, alkoxy, or haloalkoxy.

In many embodiments, Ar₂ is a meta-substituted pyridine radical having the structure

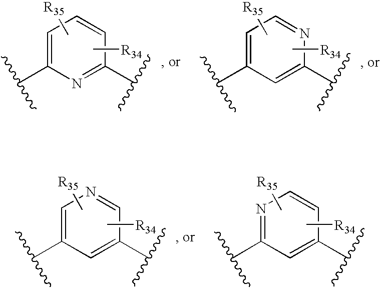

wherein R₃₄ and R₃₅ are as defined hereinabove. In some beneficial embodiments R₃₄ and R₃₅ are independently selected from hydrogen, hydroxy, halogen, alkyl, haloalkyl, alkoxy, or haloalkoxy. In many embodiments, at least one of R₃₄ and R₃₅ is hydrogen, hydroxy, or fluorine. In many embodiments, the alkyl, haloalkyl, alkoxy, or haloalkoxy radicals have from 1 to 4 carbon atoms.

In certain embodiments, the Ar₂ pyridine radical is pyridine radical with a meta geometry having the structure

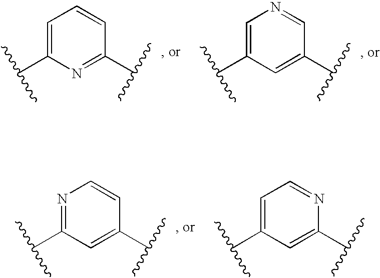

Some embodiment of the invention relate to compounds of Formula (I) wherein Ar₂ is a heteroaryl or substituted heteroaryl radical of the Formula (VI):

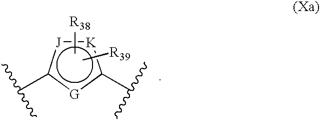

wherein:

G, J, and K are independently C or CH, O, S, N, NH or N-alkyl; and wherein R₃₈ and R₃₉ are independently selected from hydrogen or an organic or inorganic radical comprising between 1 and 10 non-hydrogen atoms. Examples of suitable R₃₈ and R₃₉ radicals include hydrogen, alkyl, substituted alkyl, alkenyl, substituted alkenyl, alkynyl, substituted alkynyl, cycloalkyl, substituted cycloalkyl, heterocyclic, substituted heterocyclic, alkoxy, substituted alkoxy, hydroxyl, acyl, amino, mono-substituted amino, di-substituted amino, carboxy, carboalkoxy, alkylcarboxamide, substituted alkylcarboxamide, dialkylcarboxamide, substituted dialkylcarboxamide, haloalkoxy, heteroaryl, substituted heteroaryl, aryl, substituted aryl. In some embodiments, at least one, or at least two of G, J, and K are C or CH.

Some examples of radicals of the Formula (Xa) wherein one of G, J, or K are S, O, or NR_{N}, and wherein NR_{N} is hydrogen, an alkyl, substituted alkyl, or haloalkyl, are exemplified by Formulas (Xaa), (Xab), (Xac), (Xad), (Xae), and (Xaf):

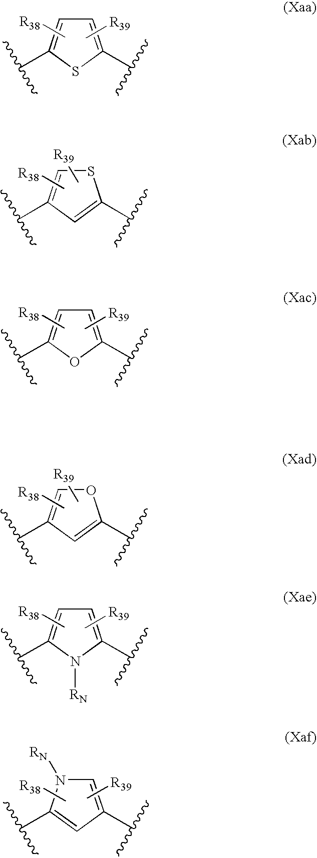

In some embodiments the Ar₂ heteroaryls are an unsubstituted furan or thiofuran radical having the formula

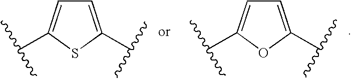

Some embodiments of the invention relate to bicyclic aryl or heteroaryl Ar₂ radicals of the Formulas

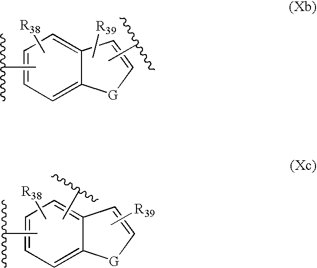

wherein:

G is C or CH, O, S, N, NH or N-alkyl; and R₃₈ and R₃₉ are independently selected and are hydrogen or an organic or inorganic radical comprising between 1 and 10 non-hydrogen atoms. Examples of suitable R₃₈ and R₃₉ radicals include hydrogen, alkyl, substituted alkyl, alkenyl, substituted alkenyl, alkynyl, substituted alkynyl, cycloalkyl, substituted cycloalkyl, heterocyclic, substituted heterocyclic, alkoxy, substituted alkoxy, hydroxyl, acyl, amino, mono-substituted amino, di-substituted amino, carboxy, carboalkoxy, alkylcarboxamide, substituted alkylcarboxamide, dialkylcarboxamide, substituted dialkylcarboxamide, haloalkoxy, heteroaryl, substituted heteroaryl, aryl or substituted aryl radicals.

Some embodiments of the invention relate to compounds when Ar₂ is a substituted heteroaryl radical of the Formula (Xb) wherein G is either NH or N-alkyl, of Formula (Xba) or (Xbb):

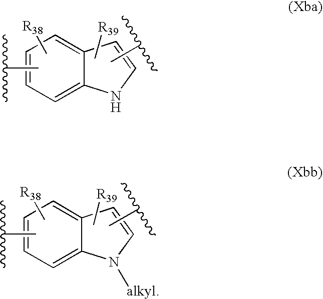

In some embodiments of Formula (I), - - - represents a bond present. When - - - is present, both E and Z configurations of the double bond, or a mixture of both olefin geometries are within the scope of the invention. Therefore the compounds of Formula (I) wherein - - - is present include compounds of Formulas (XIa):

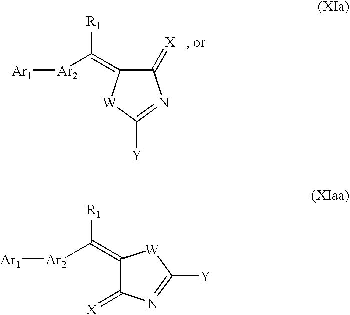

It is to be understood that for the purposes of this document, including the description and claims, if a chemical drawing shows only one of the two E or Z isomers as shown above, it should be presumed that either of the illustrated E or Z isomers, or a mixture of the two E and Z isomers is intended unless it is otherwise clear to the contrary from the context or claims. In experimental practice, especially as shown in the examples below, mixtures of the isomers are sometimes obtained, although one isomer can substantially predominate over the other isomer in many actual experiments. In the examples below, the chemical drawings illustrate the E or Z isomers that was experimentally observed to predominate.

In some embodiments, - - - represents a bond absent and the resulting compound is represented by Formula (XIb):

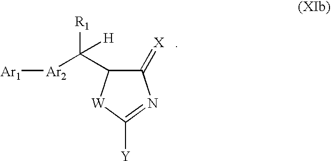

wherein R₁ can be hydrogen, hydroxy, alkyl or substituted alkyl. In some preferred embodiments, R₁ is hydrogen.

The compounds of Formula (I) have a non-hydrogen substituent at the 2-position of the 2-thiazolidinone or 2-oxazolidinone moieties, as discussed hereinabove. Y can comprise

(i) an organic radical comprising 1 to 15 carbon atoms, or 1 to 10 carbon atoms, or 1 to 6 carbon atoms;

(ii) an —S—R₂ or -0-R₂ radical wherein the R₂ radical comprises 1 to 15 carbon atoms, 1 to 10 carbon atoms, or 1 to 6 carbon atoms; or

(iv) an —NR₃R₄ radical wherein R₃ and R₄ are

a. independently hydrogen, hydroxyl, amino, or an organic radical comprising 1 to 15 carbon atoms, or 1 to 10 carbon atoms, or 1 to 6 carbon atoms; or

b. R₃ and R₄ together with the nitrogen form a heterocycle, or substituted heterocycle comprising 1 to 15 carbon atoms, or 1 to 10 carbon atoms, or 1 to 6 carbon atoms;

In some embodiments, Y is an alkyl, substituted alkyl, aryl, substituted aryl, heteroaryl, or substituted heteroaryl radical. The aryl, substituted aryl, heteroaryl or substituted heteroaryl radicals can have the Formulas (XIIIa), (XIIIb) or (XIIIc):

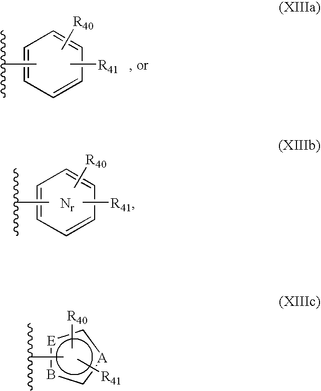

wherein:

A, B, and E are independently O, S or N;

N is a ring nitrogen;

r is the number of aromatic ring nitrogens and is 1, 2 or 3; and R₄₀ and R₄₁ are independently hydrogen, alkyl, substituted alkyl, alkenyl, substituted alkenyl, alkynyl, substituted alkynyl, cycloalkyl, substituted cycloalkyl, heterocyclic, substituted heterocyclic, alkoxy, substituted alkoxy, hydroxyl, acyl, amino, mono-substituted amino, di-substituted amino, carboxy, carboalkoxy, alkylcarboxamide, substituted alkylcarboxamide, dialkylcarboxamide, substituted dialkylcarboxamide or haloalkoxy.

The “Y” radical can also be an —S—R₂ or -0-R₂ radical wherein the R₂ radical comprises 1 to 15 carbon atoms. In some embodiments, Y is an-SR₂ radical, wherein R₂ is alkyl, substituted alkyl, cycloalkyl, or substituted cycloalkyl radical.

The “Y” radical can also be an —NR₃R₄ radical wherein R₃ and R₄ are independently hydrogen, hydroxyl, or an organic radical comprising 1 to 15 carbon atoms, 1 to 10 carbon atoms, 1 to 6 carbon atoms, or 1 to 4 carbon atoms. The R₃ and R₄ radicals can be selected independently, and examples of suitable radicals include but are not limited to alkoxy, substituted alkoxy, alkyl, substituted alkyl, cycloalkyl, substituted cycloalkyl, heterocyclic, substituted heterocyclic, amidine, substituted amidine, urea, substituted urea, amino, substituted amino, amide alkyl, amide substituted alkyl, amide aryl, amide substituted aryl, amide heteroaryl, amide substituted heteroaryl, acyl alkyl, or acyl substituted alkyl radicals. In some embodiments of the invention Y is —NR₃R₄ wherein R₃ and R₄ are independently hydrogen, alkyl, substituted alkyl.

In some embodiments of the —NR₃R₄ radical, R₃ and R₄ together with the nitrogen form a heterocycle, or substituted heterocycle comprising 1 to 12 carbon atoms, 3 to 10 carbon atoms, or 3 to 8 carbon atoms. In some embodiments, the heterocycles are partially or completely unsaturated. In some embodiments, the heterocyclic ring can comprise 4, 5, 6, 7, or 8 ring atoms, of which at least 2 ring atoms are carbon, at least one ring atom is nitrogen, and the remaining ring atoms can optionally comprise one or more additional heteroatoms such as nitrogen, oxygen, sulfur, phosphorus, and the like. In some preferred embodiments, the heterocyclic ring has 4, 5, or 6 ring atoms, that may optionally have one, two, or more substituent radicals thereof. Examples of suitable substituent radicals include but are not limited to halogen, hydroxy, amino, alkoxy, substituted alkoxy, alkyl, substituted alkyl, cycloalkyl, substituted cycloalkyl, heterocyclic, substituted heterocyclic, carboxy, haloalkyl, haloalkoxy, amidine, substituted amidine, urea, substituted urea, substituted amino, amide alkyl, amide substituted alkyl, amide aryl, amide substituted aryl, amide heteroaryl, amide substituted heteroaryl, acyl alkyl, or acyl substituted alkyl radicals.

Examples of suitable saturated heterocyclic Y groups include:

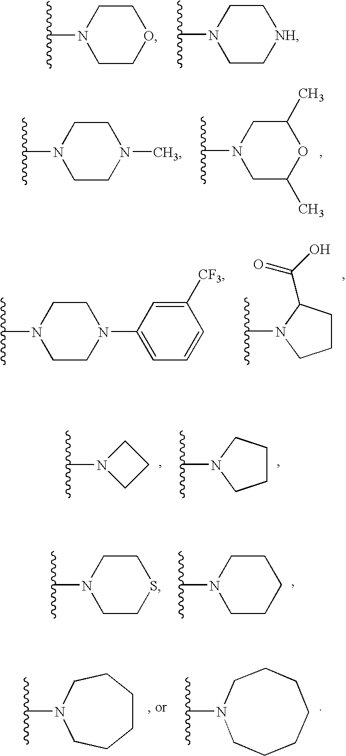

In some embodiments of the invention, the nitrogen atom of the Y radical can have other substituents, exemplified by the formulas:

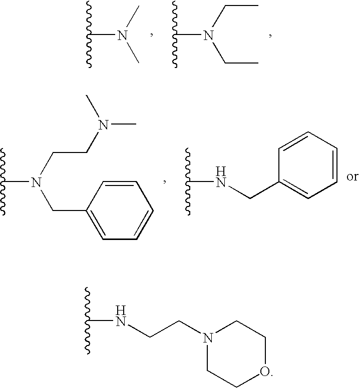

In some embodiments of the invention Y is —NR₃R₄ wherein R₃ and R₄ are independently hydrogen, heterocycle, hydroxyl, amidine, alkoxy, urea or amino. In some embodiments of the invention Y is represented by the formulae:

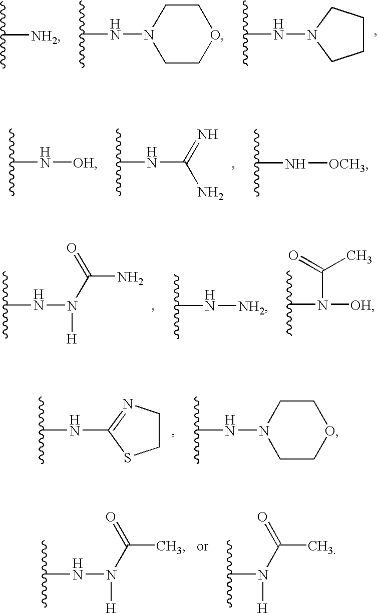

In some embodiments of the invention W is S (i.e., sulfur) and X is O (i.e., oxygen) to form a 2-substituted thiazolidinone or 2-substituted thiazol-4-one, both terms have the same meaning as used herein, and is represented by Formula (XIIa):

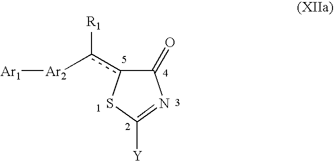

In some embodiments of the invention W is O (i.e., oxygen) and X is O (i.e., oxygen) to form a 2-substituted oxazolidinone and the compounds of the invention are represented by Formula (XIIb):

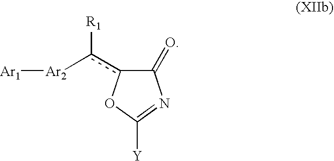

The heterocyclic residues disclosed herein can simultaneously exist in various tautomeric forms. It is understood that all tautomers are within the scope of the invention.

The compounds disclosed in WO 02/072009 have the structure

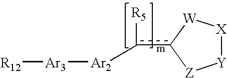

wherein:

(a) Ar₃ is an aromatic ring residue having the formula:

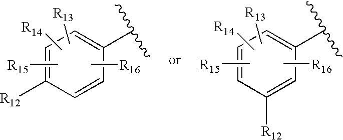

wherein

(i) R₁₂ is an alkyl or a substituted alkyl residue comprising 6 to 18 carbon atoms; or a cycloalkyl, a substituted cycloalkyl, a heterocyclic, a substituted heterocyclic, a heteroaryl, a substituted heteroaryl, an aryl or a substituted aryl residue comprising 5 to 18 carbon atoms, and

(ii) R₁₃, R₁₄, R₁₅ and R₁₆ are independently selected from hydrogen, a hydroxyl, an amino residue; an alkyl or a substituted alkyl residue comprising 6 to 18 carbon atoms; or an alkenyl, a substituted alkenyl, an alkynyl, a substituted alkynyl, a cycloalkyl, a substituted a cycloalkyl, a heterocyclic, a substituted heterocyclic, an alkoxy, a substituted alkoxy, an acyl, a mono-substituted amino, a di-substituted amino, a carboxy, a carboalkoxy, a nitrile an alkylcarboxamide, a substituted an alkylcarboxamide, a dialkylcarboxamide, a substituted dialkylcarboxamide, a haloalkoxy, a triorganosilyloxy, a heteroaryl, a substituted heteroaryl, an aryl, or a substituted aryl residue comprising 5 to 18 carbon atoms or two of R₁₃, R₁₄, R₁₅ and R₁₆ together form an alkylene-dioxy substituent ring; and

(iii) Ar₃ and R₁₂ do not together form a substituted or unsubstituted 5,6,7,8-tetrahydro-2-napthyl residue, a substituted or unsubstituted 1,2,3,4-tetrahydro-6-quinolinyl residue, or a substituted or unsubstituted 1,2,3,4-tetrahydro-7-quinoxalinyl residue;

(b) Ar₄ is an unsubstituted aryl, a substituted aryl, a heteroaryl or a substituted heteroaryl residue comprising 5 to 18 carbon atoms;

(c) R₅ is hydrogen, hydroxy, alkyl or substituted alkyl;

(d) - - - represents a bond present or absent;

(e) m is the integers 0 or 1; and

(f) W, X, Y and Z form a residue of formula:

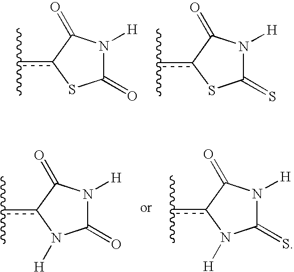

The compounds of the present invention do not comprise five-membered heterocyclic rings having the structure

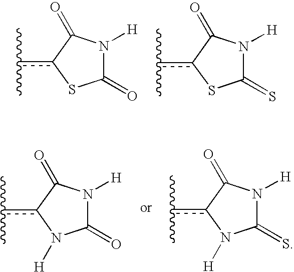

It should be noted that in some cases, the compounds disclosed in WO 02/072009 can be used as synthetic precursors for the compounds of the present invention. The compounds disclosed in WO 02/072009 wherein the five-membered heterocycle is a rhodanine ring, i.e. wherein the five-membered heterocycle is

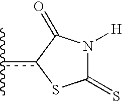

can be reacted with an amine or an alkylating agent to introduce the “Y” groups onto the heterocycle and generate compounds of the present invention.

In some embodiments, the invention relates to compounds having the structure

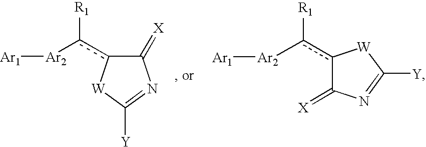

wherein:

a) Ar₁ has 4 to 30 carbon atoms and is an aryl, substituted aryl, heteroaryl, or substituted heteroaryl radical;

b) Ar₂ has 4 to 30 carbon atoms and is an aryl, substituted aryl, heteroaryl, or substituted heteroaryl radical;

c) R₁ is hydrogen, hydroxy, alkoxy, alkyl, or substituted alkyl;

d) - - - represents a bond present or absent;

e) W is —S— or —O—;

f) X is —S— or —O—; and

g) Y is an organic radical comprising 1 to 15 carbon atoms;

or a pharmaceutically acceptable salt thereof, wherein the Ar₁, Ar₂, and other terms are defined hereinabove.

In other embodiments, the invention relates to a compound having the structure

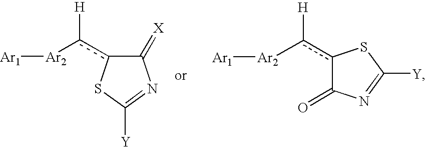

wherein:

a) Ar₁ has six to twenty carbon atoms and has the structure

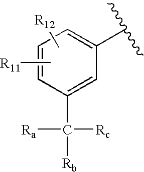

wherein Rₐ, R_{b}, and R_{c} are independently selected from hydrogen, an alkyl, substituted alkyl, cycloalkyl, substituted alkyl, heterocyclic or substituted heterocyclic radical; or wherein two or three of the Rₐ, R_{b}, and R_{c} radicals together form a bicyclic, polycyclic, heterocyclic, alicyclic, aryl, or heteroaryl ring; with the proviso that no more than one of Rₐ, R_{b}, and R_{c} are hydrogen; and R₁₁ and R₁₂, are independently selected from organic or inorganic substituent radicals;

b) Ar₂ has six to twenty carbon atoms and has the structure

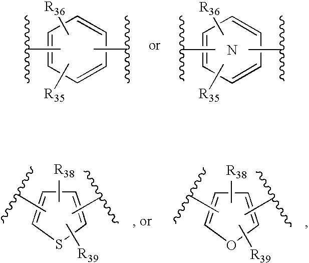

wherein R₃₅, R₃₆, R₃₈, and R₃₉ are independently selected from hydrogen, an inorganic radical, or an organic radical having from 1 to 6 carbon atoms;

c) - - - represents a bond present or absent; and

d) Y is an —NR₃R₄ radical wherein R₃ and R₄ together with the nitrogen form a heterocycle, or substituted heterocycle comprising 1 to 12 carbon atoms;

or a pharmaceutically acceptable salt thereof.

In other embodiments, the invention relates to a compound having the structure

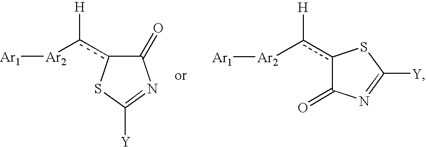

wherein:

a) Ar₁ has the structure

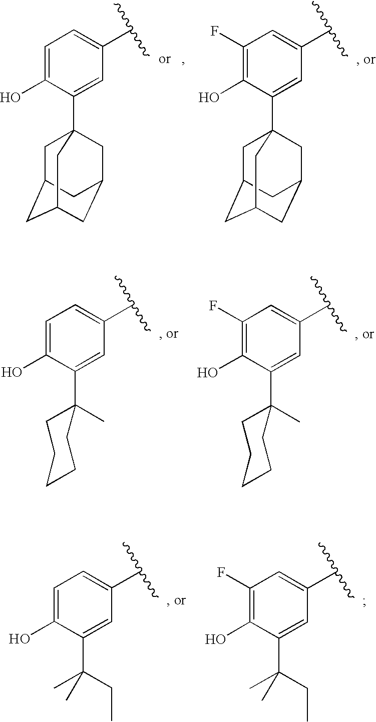

b) Ar₂ has the structure

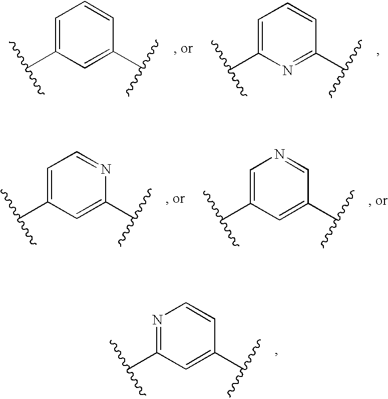

or the structure

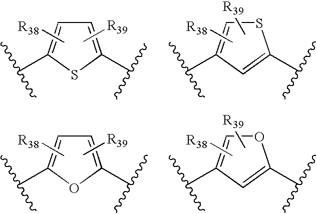

wherein the R₃₈ and R₃₉ radicals are independently selected from hydrogen, halogens, or organic radicals having 1 to 6 carbon atoms,

c) - - - represents a bond present or absent; and

d) Y has the structure

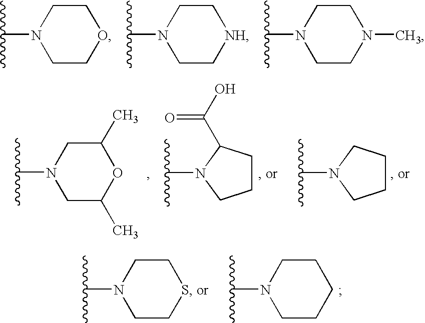

or a pharmaceutically acceptable salt thereof.

2. In the embodiments described above, the Ar₂ ring and the five membered heterocycle are linked by a linking carbon atom having an R₁ substituent. In some embodiments of the present invention, the carbon atom having an R₁ substituent can be replaced with an appropriate heteroatomic linking group. Therefore, in some embodiments, the invention relates to a heteroatom-linked compound having the structure

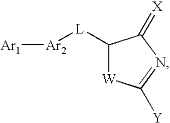

wherein:

a) Ar₁ has 4 to 30 carbon atoms and is an aryl, substituted aryl, heteroaryl, or substituted heteroaryl radical;

b) Ar₂ has 4 to 30 carbon atoms and is an aryl, substituted aryl, heteroaryl, or substituted heteroaryl radical;

c) L is a heteroatomic linking group selected from —O—, —NR_{L}, —S—, —S(O)—, and-S(O)₂—, wherein R_{L} is hydrogen or an organic residue;

d) - - - represents a bond present or absent;

e) W is —S— or —O—;

f) X is —S— or —O—; and

g) Y is an organic radical comprising 1 to 15 carbon atoms;

or a pharmaceutically acceptable salt thereof, wherein Ar₁, Ar₂, and Y can be any of the embodiments defined above.

In such embodiments, the heteroatom-linked compounds can have structures which include

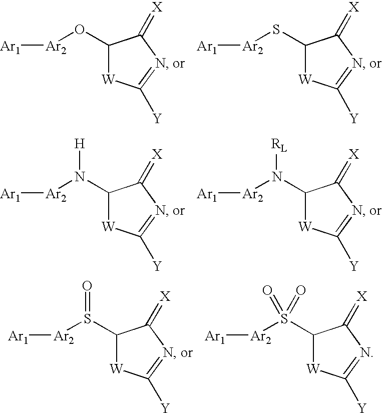

Preferably, R_{L} is hydrogen, a lower alkyl, or a hydroxyalkyl group.

It is understood that when a chiral atom is present in a compound disclosed herein that both enantiomers, racemic mixtures and mixtures of enantiomeric excess are within the scope of the invention. As defined herein, racemic mixture is an equal ratio of each of the enantiomers, whereas an enantiomeric excess is when the percent of one enantiomer is greater than the other enantiomer, all percentages are within the scope of the invention. Furthermore, when more than one chiral atom is present in a compound then the enantiomers, racemic mixtures, mixtures of enantiomeric excess and diastereomic mixtures are within the scope of the invention.

The compounds disclosed herein can also include salts of the compounds, such as salts with cations, in order to form a pharmaceutically acceptable salt. Cations with which the compounds of the invention can form pharmaceutically acceptable salts include alkali metals, such as sodium or potassium; alkaline earth metals, such as calcium; and trivalent metals, such as aluminum. The only constraint with respect to the selection of the cation is that it should not unacceptably increase the toxicity. Also, one or more compounds disclosed herein can include salts formed by reaction of a nitrogen contained within the compound, such as an amine, aniline, substituted aniline, pyridyl and the like, with an acid, such as HCl, carboxylic acid and the like. Furthermore, all possible salt forms in relationship to the tautomers and a salt formed from the reaction between a nitrogen and acid are within the scope of the invention.

The present invention also provides, but is not limited to, the specific compounds set forth in the Examples set forth below, and a pharmaceutically acceptable salt thereof.

### Making the Compounds of the Invention

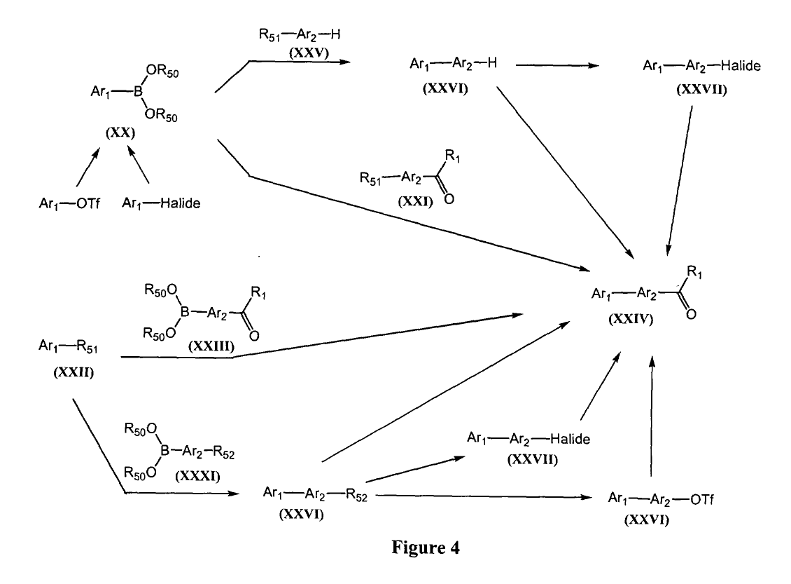

Various synthetic methods can be employed in the synthesis or production of the compounds disclosed herein. A representative set of synthetic pathways for compounds of the invention having carbon atoms connecting Ar₂ and the 5-membered heterocyclic rings is shown in FIGS. 4, 5, 6, 7, 8, 9 and 10. A group of methods for synthesizing carbonyl-containing synthetic precursors of such compounds is shown in FIG. 4. One approach involves coupling a boronic acid precursor of the Ar₁ ring, (having Formula (XX), R₅₀═H) with a carbonyl-containing aryl halide precursor of Ar₂ (having Formula (XXI), wherein R₅₁═Br) to give biaryl (XXIV) that is substituted with a carbonyl group, such as a formyl group (i.e., R₁═H). Alternatively, a boronic acid precursor of Ar₁ such as(XX) can be coupled with aryl halide (XXV), such as when R₅₁═Br, to give biaryl (XXVI) that is subsequently formulated or acrylate using techniques known in the art, such as the Voltmeter or the Vilsmeier-Haack reaction, the Gatterman reaction, the Duff reaction, the Reimer-Tiemann reaction or a like reaction, to give the desired carbonyl containing biaryl compound (XXIV). The use of Biaryl forming or coupling reactions, such as that described for the formation of Biaryl (XXIV) and (XXVI) can also be conducted using boronic esters, such as where R₅₀ together with the boron form a pinacol borate ester (formation of pinacol esters: Ishiyama, T., et al., J. Org. Chem. 1995, 60, 7508-7510, Ishiyama, T., et al., Tetrahedron Letters 1997, 38, 3447-3450; coupling pinacol esters: Firooznia, F. et al., Tetrahedron Letters 1999, 40, 213-216, Manickam, G. et al., Synthesis 2000, 442-446; all four citations incorporated herein by reference). In addition to when R₅₁ is Br, R₅₁ can also be I, Cl or triflate (derived from a phenol and triflic anhydride or similar agent).

Biaryl (XXVI) can also be acylated, for example by the Friedel-Crafts acylation reaction (using an acid chloride or the like) to give biaryl (XXIV) where R₁ is not hydrogen. In an alternative manner, biaryl (XXVI) can be formylated by first performing a halogenation step to give biaryl (XXVII), such as a bromination, followed by a halogen-metal exchange reaction using an alkyl lithium and reaction with DMF or equivalent known in the art to give biaryl (XXIV) where R₁ is H.

Alternatively, the coupling of the Ar₁ and Ar₂ groups to make a biaryl can take place between a halogenated aryl precursor for Ar₁ (XXII), such as where R₅₁═Br, and a boronic acid precursor of Ar₂ (XXIII, R₅₀═H) to give the above mention biaryl (XXIV). Also, aryl (XXII) can be coupled with boronic acid (XXXI), R₅₂═H, to give biaryl (XXVI). In another method, aryl (XXII) can be coupled with boronic acid (XXXI), where R₅₂ is a hydroxyl or a protected hydroxyl such as a t-butyl dimethylsilyloxy group, to give biaryl (XXVI). Biaryl (XXVI), R₅₂=hydroxy or protected hydroxyl group, can be converted to a triflate (XXVI) and subsequently allowed to react with carbon monoxide in the presence of a Pd catalyst to give biaryl (XXIV). Such catalytic carbonylation reactions can be adapted so as to produce a formyl group (R₁═H) by concurrent use of a trialkylsilane, such as triethylsilane, or can be adapted to produce an ester (in the presence of alcohols) or carboxylic acid (in the presence of water). The esters or acids can be converted to a formyl through a reduction step (e.g., DIBAL) and an oxidation step (e.g., PCC and like reagents). Employing the same strategy as described above, biaryl (XXVI), R₅₂═H, can be converted to biaryl (XXIV) through (XXVII).

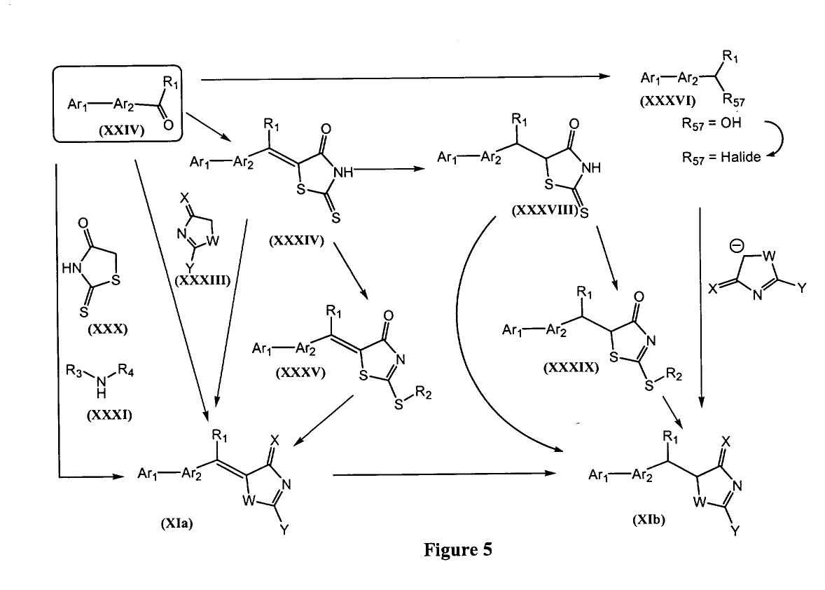

The carbonyl group of biaryl (XXIV) can subsequently be condensed with an appropriate 5-membered heterocycle possessing an active methylene moiety. A representative set of such reactions are found in FIG. 5. One method is the condensation of the carbonyl group of biaryl (XXIV) with 2-thioxo-thiazolidin-4-one (XXX, also known as rhodanine in the art) in the presence of amine (XXXI) bearing at least one hydrogen, with the rhodanine, in an appropriate solvent such as, for example, toluene, to give heterocycle (XIa, wherein Y is NR₅₅R₅₆). This method provides compounds of the invention wherein the - - - is present thus representing the presence of a double bond, i.e. a benzylidene compound. In an alternative method, benzylidene compounds of the invention can be prepared by the condensation of the carbonyl group of biaryl (XXIV) with a 5-membered heterocycle (XXXIII) that has previously been substituted with the desired “Y” group, to give heterocycle (XIa). Alternatively, heterocycle (XIa) can be prepared in a step-wise manner, the carbonyl of biaryl (XXIV) can be condensed with 2-thioxo-thiazolidin-4-one (XXX, rhodanine) under Knoevenagel conditions to give 5-membered heterocycle without a “Y” radical (XXXIV). Heterocycle (XXXIV) can be calculated on a sulfur atom to give new heterocycle with a “Y” group comprising an R₂—S-group (XXXV, wherein R₂ has the meaning as described for compounds of Formula (I) hereinabove), which can be in turn reacted with an amine (XXXI) to heterocycle (XIa). These methods utilize a reaction known in the art as a Knoevenagel condensation as described by Tietze and Beifuss, Comprehensive Organic Synthesis (Pergamon Press), 2:341-394, (1991), incorporated herein by reference.

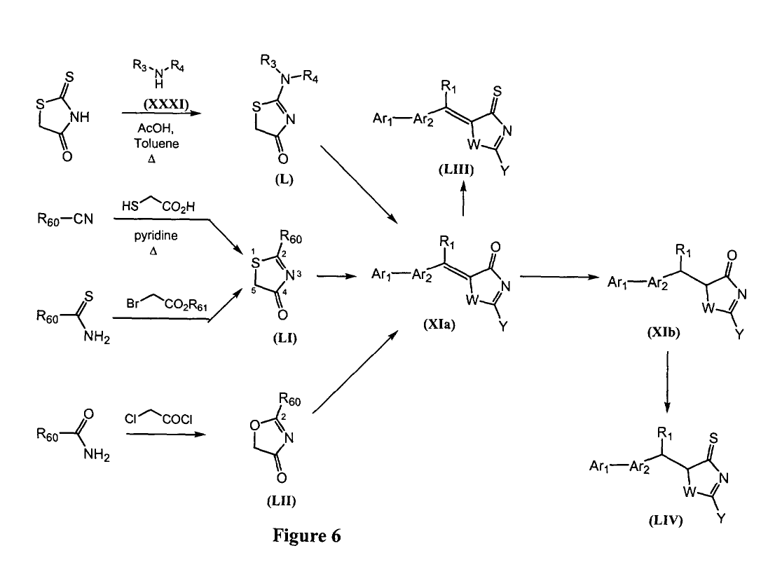

The 2-substituted heterocycle (XXXIII) wherein Y has the same definition as described hereinabove (shown in FIG. 5), can be separately prepared and utilized in the preparation of the compounds of Formula (I). Several illustrative methods for the synthesis of 2-substituted heterocycles from the genus (XXXIII) are shown in FIG. 6. One method uses rhodanine and an appropriate amine, such as amine (XXXI) under Knoevenagel-like conditions to give heterocycle (L). This 2-substituted heterocyclic compound can be further purified or used directly in the next step for condensation with biaryls having carbonyl groups, or other linking group precursors, to give, for example heterocycle (XIa).

A method for the preparation of heterocycle from genus (XXXIII) having a carbon-based radical at the C(2) position (LI) is also shown in FIG. 6. This method can employ a wide variety of nitrites (wherein R₆₀ is a group containing 1 to 10 carbon atoms), by treating them with mercaptoacetic acid in a solvent, such as pyridine, with heat to give heterocycle (LI), Sadek, et al. Synthesis, 1983, 739-791; Sowellum, et al. Pharmazie, 1988, 43, 533-534; Abdel-Latif, et al. Pol. J. Chem., 1991, 65, 1043-1048; these three citations incorporated herein by reference. Alternatively, a thioamide (wherein R₆₀ is a group containing 1 to 10 carbon atoms) can be allowed to react with bromoacetic acid, ester or acid bromide (i.e., R₆₁═H, alkyl, bromide) to also give heterocycle (LI), Kerdesky, et al. J. Med. Chem., 1991, 34, 2158-2165; Okawara, et al. Chem. Pharm. Bull., 1985, 33, 3479-3483, both citations incorporated herein by reference. Five-membered heterocycles wherein W is oxygen and having formula (LII) can be prepared by using a variety of amides (wherein R₆₀ is a group containing 1 to 10 carbon atoms) in the presence of chloroacetyl chloride, Rao et al. J. Chem. Soc. D, 1970, 1622; Kelly, et al. J. Org. Chem., 1996, 61, 4623-4633, both citation incorporated herein by reference.

As described above, the 2-substituted heterocycles [i.e., (L), (LI) or (LII)] have active methylene groups that can be condensed in the presence of base catalysts with the carbonyl of biaryl (XXIV) to give the benzylidene compounds (XIa) of the invention. The carbon-carbon double bonds of these benzylidene compounds can then be reduced to the benzyl compound (XIb) of the invention.

An additional method of preparation is available for compounds of the invention is where X is sulfur. As shown in FIG. 6, compounds such as (XIa) and (XIb) can be further reacted to give thiones (LIII) and (LIV) respectively by methods known in the art. Such methods include, but are not limited to, Lawesson's Reagent, P₂S₅ and the like.

As shown in FIG. 5, the carbonyl group of biaryl (XXIV) can also be reduced, such as with sodium borohydride, diisobutyl alumium hydride, or the like, to give benzyl alcohol (XXXVI, R₅₇═OH) and converted to benzyl bromide (XXIX, R₅₇═Br) with HBr or some other method known in the art, such as PPh₃/CBr₄ or converted to another leaving group, such as, for example, mesylate or iodide. Benzyl bromide (XXXVI, R₅₇═Br) or a like compound is allowed to react with the anion(s) of heterocycle (XXXIII) to give biaryl (XIb). Heterocyclic compounds in which anion or anions can be generated include but are not limited to heterocycles of the Formula (XXXVIIIa) or (XXXVIIIb):

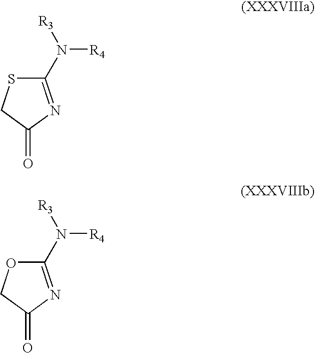

Alternatively, biaryl (XIb) can be prepared by a reduction of benzylidene (XXVIII), using methods known in the art, such as hydrogen in the presence of Pd/C, Mg/MeOH, LiBH₄ in THF/pyridine and the like. In still another method benzylidene (XXXIV) can be reduced, such as for example, with hydrogen in the presence of Pd/C, Mg/MeOH, LiBH₄ in THF/pyridine and the like, to give heterocycle (XXXVIII) and subsequently allowed to react with an amine, such as amine (XXXI), to give heterocycle (XIb); or heterocycle (XXXVIII) can be S-alkylated in a manner described herein to give heterocycle (XXXIX) and subsequently allowed to react with an amine, such as amine (XXXI), to give heterocycle (XIb).

One embodiment of the invention relates to the processes for making compounds of Formula (I) which comprises coupling two aromatic rings to give a biaryl wherein one of the aryl rings contains a carbonyl moiety, preferably an aldehyde.

Coupling of two aryl rings can be conducted using an aryl boronic acid or esters with an aryl halide (such as, iodo, bromo, or chloro), triflate or diazonium tetrafluoroborate; as described respectively in Suzuki, Pure & Applied Chem., 66:213-222 (1994), Miyaura and Suzuki, Chem. Rev. 95:2457-2483 (1995), Watanabe, Miyaura and Suzuki, Synlett. 207-210 (1992), Littke and Fu, Angew. Chem. Int. Ed., 37:3387-3388 (1998), Indolese, Tetrahedron Letters, 38:3513-3516 (1997), Firooznia, et. al., Tetrahedron Letters 40:213-216 (1999), and Darses, et. al., Bull. Soc. Chim. Fr. 133:1095-1102 (1996); all incorporated herein by reference. According to this coupling method, precursors such as (XX) and (XXI) can be employed:

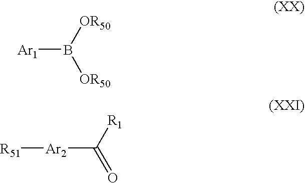

where R₅₀ is hydrogen, alkyl, or R₅₀ together with the boron and the two oxygens form a heterocycle, such as a pinacol group, and R₅₁ is a halide (such as, iodo, bromo, or chloro), triflate or diazonium tetrafluoroborate. Alternatively, it is understood that the coupling groups can be reversed, such as the use of (XXII) and (XXIII), to achieve the same coupling product:

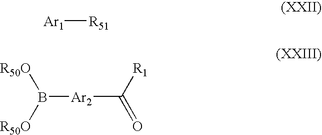

where R₅₀ and R₅₁ have the same meaning as described above. The above mentioned precursors can be prepared by methods readily available to those skilled in the art. For example, the boronic ester can be prepared from an aryl halide by conversion of the corresponding aryl lithium, followed by treatment with a trialkyl borate, such as triisopropyl borate and the like. Alternatively, the boronic ester can be hydrolyzed to the boronic acid for coupling.

The coupling reaction can also be conducted between an aryl zinc halide and an aryl halide or triflate. Alternately, the coupling reaction can also be executed using an aryl trialkyltin derivative and an aryl halide or triflate. These coupling methods are reviewed by Stanforth, Tetrahedron 54:263-303 (1998) and incorporated herein by reference. In general, the utilization of a specific coupling procedure is selected with respect to available precursors, chemoselectivity, regioselectivity and steric considerations.

The biaryl intermediates having a carbonyl group, such as compound (XXIV) of FIG. 5, can be subsequently condensed with an active methylene compound to produce the desired final product heterocycles (XIa). The condensation can be accomplished in a step-wise manner, wherein the active methylene compound is a rhodanine compound (compound (XXX). The product of the initial condensation, such as, for example, a 5-benzylidene-2-thioxo-thiazolidin-4-one or a 5-benzylidene-2-thioxo-oxazolidin-4-one compound, is subsequently condensed with an amine (XXXI) to introduce the “Y” group onto the heterocycle.

Therefore, in one embodiment, the invention relates to a method for making a 2-substituted benzylidene compound having the structure

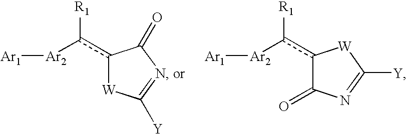

wherein:

a) Ar₁ has 4 to 30 carbon atoms and is an aryl, substituted aryl, heteroaryl, or substituted heteroaryl radical;

b) Ar₂ has 4 to 30 carbon atoms and is an aryl, substituted aryl, heteroaryl, or substituted heteroaryl radical;

c) R₁ is hydrogen, hydroxy, alkoxy, alkyl, or substituted alkyl;

d) - - - represents a bond present or absent;

e) W is —S— or —O—; and

f) Y is an —NR₃R₄ radical wherein R₃ and R₄ are independently selected from the group consisting of hydrogen, hydroxyl, amino, and an organic radical comprising 1 to 15 carbon atoms;

wherein the method comprises

g) providing a 5-benzylidene-2-thioxo-thiazolidin-4-one or a 5-benzylidene-2-thioxo-oxazolidin-4-one compound having one of the structures

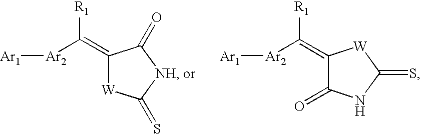

h) and reacting the 5-benzylidene-2-thioxo-thiazolidin-4-one or 5-benzylidene-2-thioxo-oxazolidin-4-one compound with an amine having the formula HNR₃R₄, to give at least some of the 2-substituted benzylidene compound.

Alternatively, the rhodanine compound and the amine can be introduced concurrently in a single pot reaction, as is illustrated in the various examples below.

Therefore, in another embodiment, the invention relates to a method for making a 2-substituted benzylidene compound having the structure

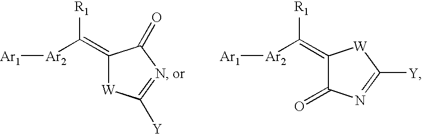

wherein:

a) Ar₁ has 4 to 30 carbon atoms and is an aryl, substituted aryl, heteroaryl, or substituted heteroaryl radical;

b) Ar₂ has 4 to 30 carbon atoms and is an aryl, substituted aryl, heteroaryl, or substituted heteroaryl radical;

c) R₁ is hydrogen, hydroxy, alkoxy, alkyl, or substituted alkyl;

d) - - - represents a bond present or absent;

e) W is —S— or —O—; and

f) Y is an —NR₃R₄ radical wherein R₃ and R₄ are independently selected from the group consisting of hydrogen, hydroxyl, amino, and an organic radical comprising 1 to 15 carbon atoms;

wherein the method comprises

g) providing a carbonyl compound having structure

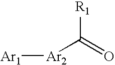

h) and condensing the carbonyl compound with 2-thioxo-thiazolidin-4-one or with 2-thioxo-oxazolidin-4-one in the presence of an amine having the formula HNR₃R₄, to give at least some of the 2-substituted benzylidene compound.

Alternatively, the methylene compound can be a heterocycle into which a “Y” group has already been introduced, such as a heterocycle of Formula (XXXVIII) to give a benzylidene compound of Formula (I) where - - - is a bond.

Compounds wherein the “Y” radical is sulfur based radical can be prepared, for example, by alkylating the sulfur of a rhodanine derivative. The sulfur based Y radical can be displace by an amine if desired.

Condensation of the biaryl carbonyl containing derivatives (e.g., FIG. 5, compound (XXIV)) with a suitable active methylene compound can be accomplished by the use of methods known in the art. For example, the biaryl carbonyl product from the coupling reaction can be condensed with an active methylene compound to give a benzylidene compound of Formula (I) (i.e., - - - is a bond) as described by Tietze and Beifuss, Comprehensive Organic Synthesis (Pergamon Press), 2:341-394, (1991), incorporated herein by reference. It is understood by those of skill in the art that intermediates having hydroxyl groups bound thereto can be formed during condensation of a biaryl carbonyl containing derivative and an active methylene compound, as shown below.

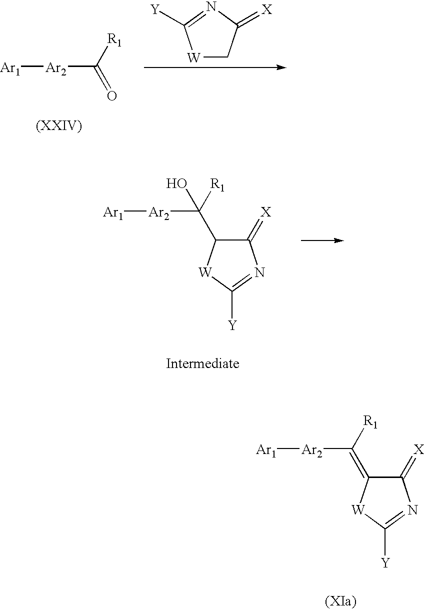

The hydroxyl groups of such intermediates are often eliminated (as water) during the condensation reaction, to form the desired benzylidene compound. Nevertheless, the conditions of the reaction can be modified for the isolation or further use of hydroxyl containing intermediates, and such embodiments are within the scope of the invention. Effective catalysts for the condensation can be selected from ammonia, primary, secondary and tertiary amines, either as the free base or the amine salt with an organic acid, such as acetic acid. Examples of catalysts include pyrrolidine, piperidine, pyridine, diethylamine and the acetate salts thereof. Inorganic catalysts can also be used for the condensation. Inorganic catalysts include, but are not limited to, titanium tetrachloride and a tertiary base, such as pyridine; and magnesium oxide or zinc oxide in an inert solvent system. This type of condensation can be strongly solvent-dependent and it is understood that routine experimentation may be necessary to identify the optimal solvent with a particular catalyst, preferable solvents include ethanol, tetrahydrofuran, dioxane or toluene; or mixtures thereof.

In an optional step, the benzylidene compounds wherein the double bond is present can be reduced to give a compound of Formula (I) where - - - is absent, i.e., a benzyl compound having the structure

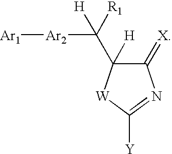

The reduction of the carbon-carbon bond of the benzylidene compound to give the reduced and/or hydrogenated benzyl compound can be accomplished by many methods known of those of ordinary skill in art, such as catalytic hydrogenation, reduction with reducing metals such as sodium or zinc in the presence of protic solvents, or via hydride reducing agents such as borohydrides, etc.

In some embodiments described above the invention relates to a method of making a heteroatom-linked compound having the structure

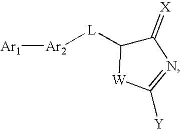

wherein L is a heteroatomic linking group selected from —O—, —NR_{L}, —S—, —S(O)—, and-S(O)₂—, wherein R_{L} is hydrogen or an organic residue.

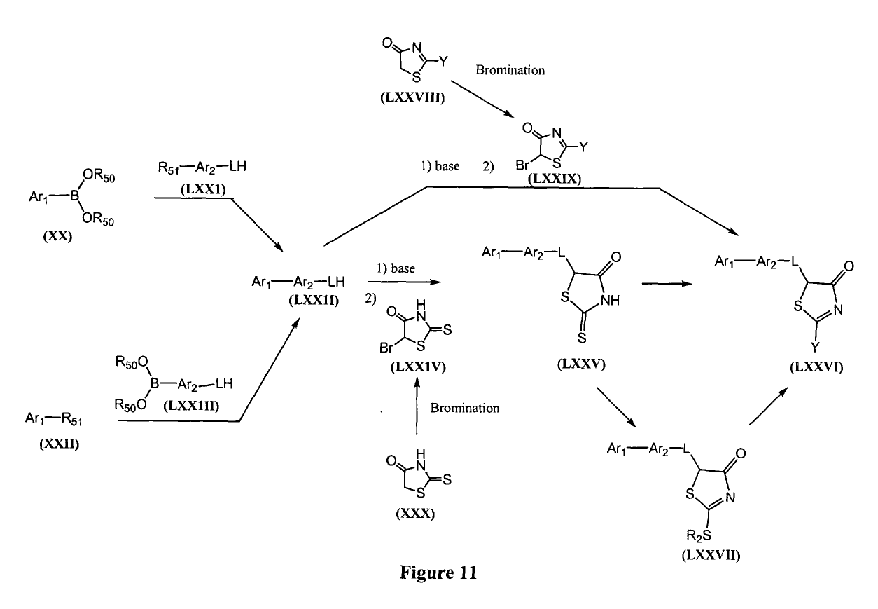

Methods for making the heteroatom linked compounds shown above are illustrated in FIG. 11. Precursor biaryl compounds having the structure Ar₁—Ar₂-LH (see FIG. 11, compound LXXII, wherein L is —O—, —S—, and —NR_{L}) can be prepared, for example, by coupling a boronic acid precursor of Ar₁, such as for example Formula (XX), with an appropriate precursor of Ar₂ that has a “L” heteroatom substituent in a form suitable for coupling to the five membered heterocycles of the invention. Examples of such compounds are the R₅₁—Ar₂-LH compounds having formula (LXXI) in FIG. 11, where R₅₁ is preferably a halide or tosylate, and L is —O—, —S—, and —NR_{L}. Compound (XX) can be coupled with compound (LXXI) to give biaryl (LXXII). Biaryl (LXXII) can be prepared alternatively by the coupling of boronic acid (LXXIII) with aryl halide (XXII), as also shown in FIG. 11. Methods of synthesis for wide variety of substituted aromatic precursor compounds for Ar₁ and Ar₂ are disclosed elsewhere herein, or are well known to those of ordinary skill in synthetic organic chemistry arts.

Precursors of the five membered heterocycles of the invention suitable for coupling with compound (LXXII) can be prepared by bromination of an active methylene position. For example, 5-Bromo-2-thioxo-thiazolidin-4-one (LXXIV) can be prepared by bromination of rhodanine (XXX) as described by Pujari, J. Sci. Ind. Res. 14B:398 (1955), then coupled with compound (LXXII) in the presence of base, as described by Zask et al., J. Med. Chem. 33:1418-1423 (1990) to give the heterocycle (LXXV). Heterocycle (LXXV) can be further reacted with an amine to give the desired 2-substituted heterocycle (LXXVI) as described hereinabove.

Therefore, in some embodiments, the invention relates to method of making a heteroatom-linked compound having the structure

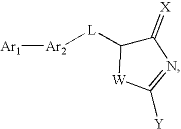

wherein:

a) Ar₁ has 4 to 30 carbon atoms and is an aryl, substituted aryl, heteroaryl, or substituted heteroaryl radical;

b) Ar₂ has 4 to 30 carbon atoms and is an aryl, substituted aryl, heteroaryl, or substituted heteroaryl radical;

c) L is a heteroatomic linking group selected from —O—, —NR_{L}, —S—, wherein Ris hydrogen or an organic residue;

d) - - - represents a bond present or absent;

e) W is —S— or —O—;

f) X is —S— or —O—; and

g) Y is an NR₃R₄ radical comprising 1 to 15 carbon atoms;

wherein the method comprises

i) providing a heteroatom linked precursor compound having structure

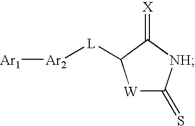

j) and condensing the heteroatom linked precursor compound with an amine having the structure HNR₃R₄;

k) to give at least some of the heteroatom-linked compound, or a pharmaceutically acceptable salt thereof.

Alternatively, a sulfur heterocycle (LXXV) can be alkylated to give new heterocycle (LXXVII), which in turn is converted to derivative (LXXVI) as described hereinabove.

In yet another method, biaryl (LXXII) can be coupled directly to a halogenated heterocycle such as (LXXIX), which can be obtained by bromination of the previously 2-substituted heterocycle (LXXVIII), to afford heterocycle (LXXVI). Therefore, in some embodiments, the invention relates to method of making a heteroatom-linked compound having the structure

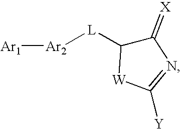

wherein:

a) Ar₁ has 4 to 30 carbon atoms and is an aryl, substituted aryl, heteroaryl, or substituted heteroaryl radical;

b) Ar₂ has 4 to 30 carbon atoms and is an aryl, substituted aryl, heteroaryl, or substituted heteroaryl radical;

c) L is a heteroatomic linking group selected from —O—, —NR_{L}, —S—, —S(O)—, and-S(O)₂—, wherein R_{L} is hydrogen or an organic residue;

d) - - - represents a bond present or absent;

e) W is —S— or —O—;

f) X is —S— or —O—; and

g) Y is an organic radical comprising 1 to 15 carbon atoms;

wherein the method comprises

l) providing a heteroatom substituted biaryl compound having structure

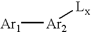

wherein Lₓ is —OH, —NHR_{L}, or —SH;

m) and condensing the heteroatom substituted biaryl compound with a 2-substituted-5-halogenated-five membered heterocycle having the structure

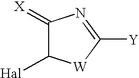

wherein Hal is a halogen,

n) to give at least some of the heteroatom-linked compound, or a pharmaceutically acceptable salt thereof.

Furthermore, when L=S in the above heteroatom lined heterocycles, heterocycles (LXXV) and (LXXVI) shown in FIG. 11 can be oxidized in a selective manner with m-chloroperbenzoic acid to provide the sulfoxide compound (L=SO). The sulfur atom can be further oxidized with additional m-chloroperbenzoic acid, or with hydrogen peroxide in acetic acid, as described by Zask et al., J. Med. Chem. 33:1418-1423 (1990), to provide the sulfone compound (L=SO₂).

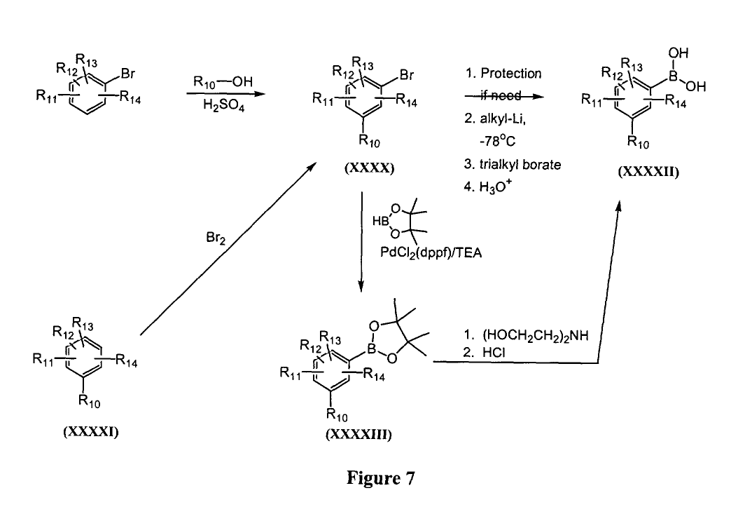

Various methods can be used to prepare intermediates and/or precursors used in the synthesis of compounds of the invention. One class of such intermediate are the boronic acid precursors of Ar₁, such as compound (XX). One representative set of methods for the synthesis of precursor compound (XX) are shown in FIG. 7. Many substituted aromatic halide compounds within the scope of Formula (XXXX), and methods for their synthesis are known to those of ordinary skill in the art, in the literature and/or are commercially available, but other such compounds must be synthesized. In one method for synthesizing such compounds, a reactive alcohol, such as R₁₀—OH (R₁₀ is defined hereinabove), especially secondary or tertiary alcohols, are utilized in an electrophilic aromatic substitution reaction to substitute R₁₀ onto the aromatic ring of a substituted or unsubstituted aryl halide, via an acid promoted alkylation reaction to give aryl compounds of class (XXXX). A useful acid utilized in such reactions include sulfuric acid in a suitable solvent, such as, for example, dichloromethane; another useful acid for this type of reaction is trifluoroacetic acid, either neat or diluted in a suitable solvent. Illustrative examples of useful R₁₀—OH include, but are not limited to, 3°alcohols, such as, adamantanol, methylcyclohexanol, t-butyl alcohol, and the like. Alternatively, an aromatic ring containing a desired R₁₀ substituent can be available in the art [e.g., aryl (XXXXI)], or available via other aromatic substitution reactions, such as Friedel Crafts Acylations, etc. The aromatic ring can then be halogenated, such as, for example, with bromine, iodine, or equivalent agents, to provide a precursor halide compound for boronation. The details of examples of preparations of materials such as the brominated intermediates shown below are given in the examples.

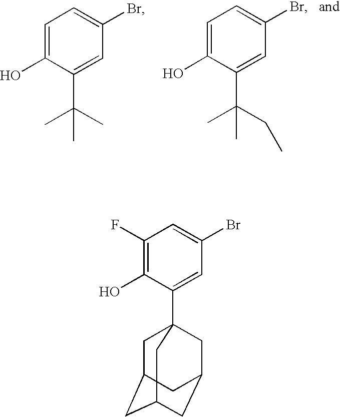

At this point, a substituent group or groups can be protected as needed. The hydroxyl group is one group that is particularly beneficial to protect, for example with a t-butyldimethylsilyl protecting group, to facilitate the subsequent boronation step. The aryl bromide or iodide can then undergo a metal exchange reaction with an alkyl-lithium, such as, n-butyl lithium or t-butyl lithium at a depressed temperature, such between −80° C. to −45° C. The aryl lithium is subsequently allowed to react with a trialkylborate, such as, for example triisopropylborate, trimethylborate and the like, and after hydrolysis gives boronic acid (XXXXII). Alternatively, the boronic acid can be prepared by another method which may be better suited for the presence of sensitive group(s) on the ring. The aryl bromide or iodide can be converted to a pinacol borane (XXXXIII) via Pd catalyzed reaction and subsequently hydrolyzed via methods known in the art, such as, (HOCH₂CH₂)₂NH/HCl, and the like.

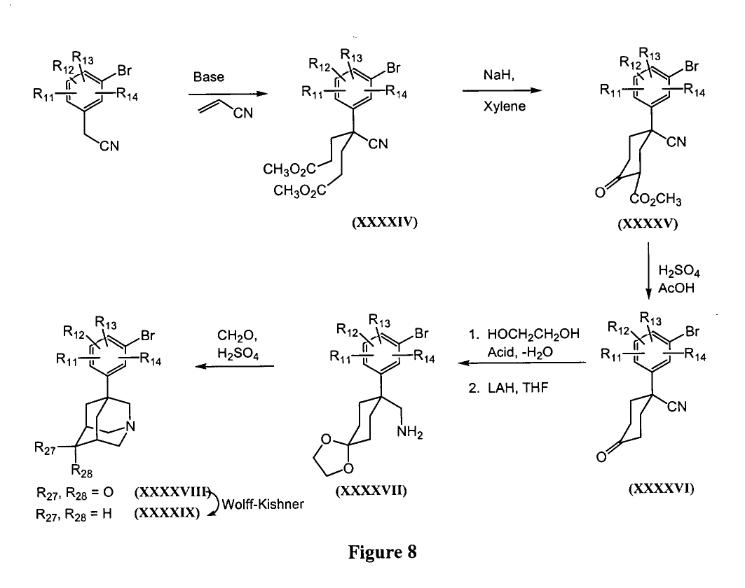

Another set of methods for preparing intermediates having nitrogen substituted adamantyl groups are shown in FIG. 8. Phenyl acetonitrile can be used with acrylonitrile in the presence of a base, such as, triton B, in an alcoholic solvent to give diester (XXXXIV). Cyclization can be executive through the use of a base, one particularly good base was NaH, in xylene to give cyclohexanone (XXXXV) followed by acid promoted decarboxylation to give a new cyclohexanone (XXXXVI). The cyclohexanone is protected, for example, as a 1,3-dioxolane, and the nitrile is reduced to amine (XXXXVII) with lithium aluminum hydride in THF. Azaadamantanone (XXXXVIII) can be prepared from amine (XXXXVII) via a double Mannich reaction in a similar manner as described by Black in Synthesis, 1981, 829-830. The carbonyl of azaadamantanone (XXXXVIII) can subsequently be reduced via methods known in the art, such as, for example, hydrazine/KOH/triglyme, and the like, to give azaadamantane (XXXXIX).

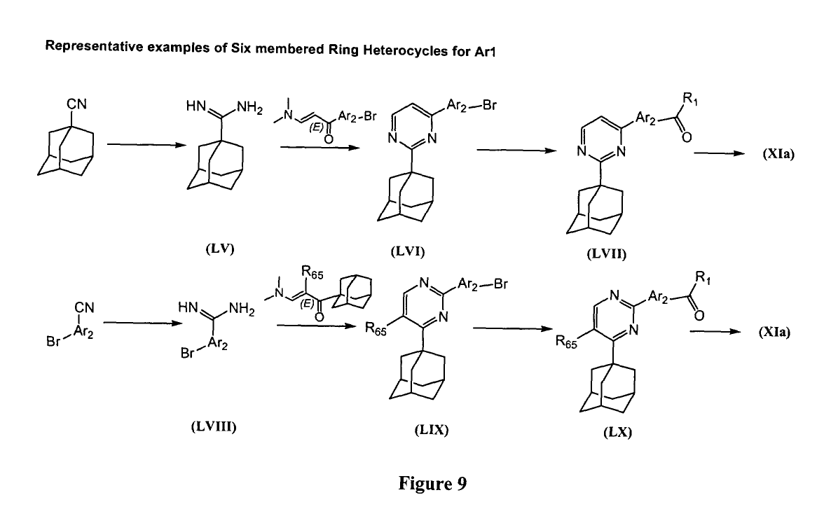

Another set of methods for preparing intermediates for compounds comprising Ar₁ compounds of interest are shown in FIG. 9, specifically six-membered heterocycles, such as pyrimidines. A nitrile can be used, such as R₉—CN, wherein R₉ has the meaning defined hereinabove, and therefore R₉ is incorporated in a pyrimidine through the nitrile as shown in FIG. 9. By way of illustration a specific example is shown in FIG. 9 starting with adamantanenitrile. The nitrile is converted to an imidate using HCl/EtOH and subsequently reacted with ammonia in EtOH to give amidine (LV). The amidine is cyclized in a manner know in the art to give pyrimidine (LVI). By selecting a group on Ar₂ that can be converted into a ketone or formyl, or a group that can be modified into a ketone or formyl group then biaryl (LVII) can be obtained and subsequently to (XIa). As an example, a bromine attached to Ar₂ can be converted to a formyl group through an aryl lithium intermediate. In a similar manner pyrimidine (LV) can be prepared. By selecting the appropriate R₆₅ then groups can be introduced on the pyrimidine ring, examples include, but limited to, methyl, formyl, hydroxyl, —CH₂OH, and the like.

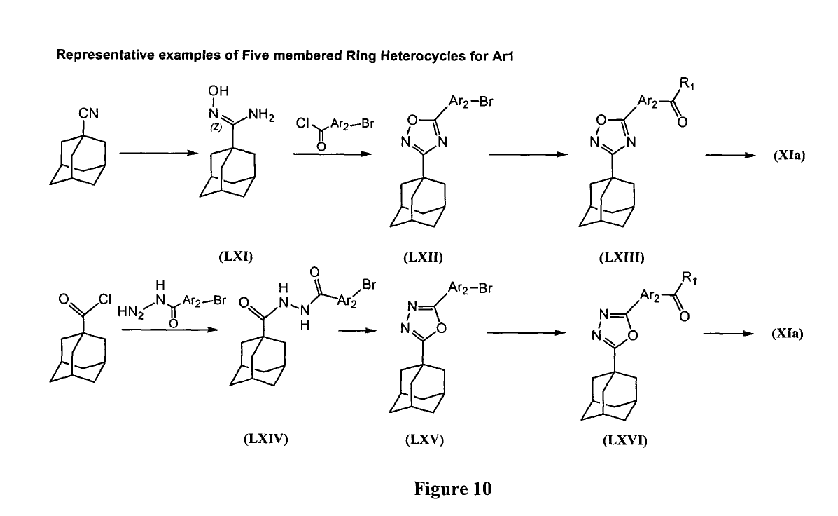

A set of methods for preparing desirable intermediates are shown in FIG. 10, specifically five-membered heterocycles, such as 1,2,4-and 1,3,4-oxadiazoles. A nitrile can be used, such R₉—CN, wherein R₉ has the meaning defined hereinabove, and therefore R₉ can be incorporated in an oxadiazole such as those shown in FIG. 10. By way of illustration a specific example is shown in FIG. 10 starting with adamantanenitrile. The nitrile can be converted to amidoxime (LXI) using hydroxylamine in EtOH with heat. The amidoxime is then acylated with an acid chloride and cyclized in a manner know in the art to give oxadiazole (LXII). By selecting a group on Ar₂ such as a ketone or formyl, or a group that can be modified into a ketone or formyl group then biaryl (LXIII) can be obtained and subsequently converted to (XIa). As an example, a bromine attached to Ar₂ can be converted to a formyl group through an aryl lithium intermediate. In a different manner oxadiazole (LXVI) can be prepared. This method involves the preparation of a diacyl hydrazine, such as (LXIV). This can be prepared via an acid chloride or carboxylic acid coupling to an acyl hydrazine. The diacylhydrazine (LXIV) is cyclized in the presence of tosylchloride in pyridine with heat to give oxadiazole (LXV). In a similar manner as described hereinabove, oxadiazole (LXVI) can be obtained and subsequently converted to (XIa).

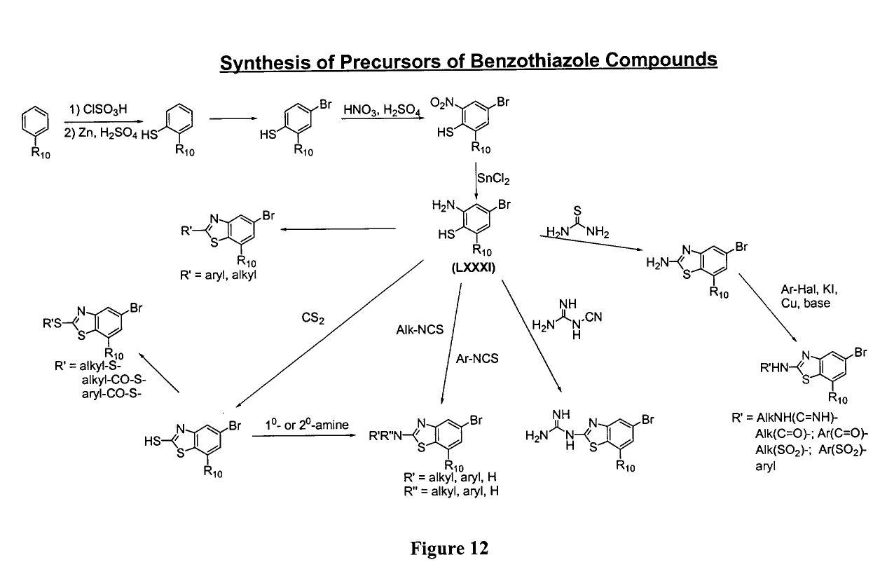

In certain embodiments described herein, the compounds of the invention have Ar₁ groups which comprise benzoxazole, benzothiazole, or benzimidazole groups. FIG. 12 illustrates group of synthetic approaches to precursors of such benzoxazole, benzothiazole, or benzimidazole compounds in general, and benzothiazole compounds in particular. FIG. 12 illustrates a reaction sequence in which a benzene ring having a desired R₁₀ substituent can be transformed, via a sequence of sulfonation, reduction, halogenation, nitration, and reduction to produce a 6-substituted-2-Amino-4-bromo-benzenethiol intermediate (LXXXI) that can be a precursor, via condensation reactions with variously substituted analogs of carboxylic acids, to produce a wide variety of substituted brominated benzothiazole compounds.

The references listed below provide relevant examples and experimental procedures for analogs of the reactions illustrated in FIG. 12, and are hereby incorporated herein by reference for their teachings of such experimental procedures. 1) Hansch et al.: J. Am. Chem. Soc. 70 (1948) 1561; 2) Patent, Consolidation Coal Co., U.S. Pat. No. 3,461,168, (1966); 3) M. H. Elmagdi et al.: Phosphorus, Sulfur, Silicon, Relat. Elem. 82 (1993) 195; 4) L. Racane et al.: Heterocycles 55 (2001) 2085; 5) C. A. Mathis: Bioorg. Med. Chem. Lett. 12 (2002) 295; 6) Tetrahedron Lett. 42 (2001) 2201; 7) R. D. Schoenwald et al.: J. Med. Chem. 27 (1984) 810; 8) J. D'Amico: J. Org. Chem. 26 (1961) 3436; 9) D. J. Brown et al.: Aust. J. Chem. 32 (1979) 2713; 10) P. R. Blakemore et al: Syn. Lett. (1998) 26; 11) F. Roulleau et al.: Tetrahedron Lett. 24 (1983) 719; 12) E. E. Gilbert: J. Heterocycle. Chem. 6 (1969) 483; 13) J. Garin et al.: J. Heterocycl. Chem. 28 (1991) 359;14) S. P. Sing et al.: Indian J. Chem., Sect. B 22 (1983) 370; 15) Patent, Am. Cyanamid Co., U.S. Pat. No. 2,575,614, (1950);16) Z. -G. Li et al.: J. Chem. Soc., Synop. 11 (2001) 470; 17) T. Kiatagawa et al.: Chem. Pharm. Bull. 49 (2001) 335; 18) J. S. Yadav et al.: Tetrahedron Lett. 39 (1998) 3259; 19) R. M. Scarboroughet al.: Bioorg. Med. Chem. Lett. 11 (2001) 1805; 20) M. A. El-Sherbeny: Arzneim. Forsch. 50 (2000) 848. Analogous reaction sequences can often be employed by those of ordinary skill in the art to produce analogous benzoxazole and benzimidazole precursors of Ar₁, either when appropriately substituted phenols or aniline starting materials are commercially available, or when the appropriate precursor phenols or anilines are available from synthetic procedures available in the literature.

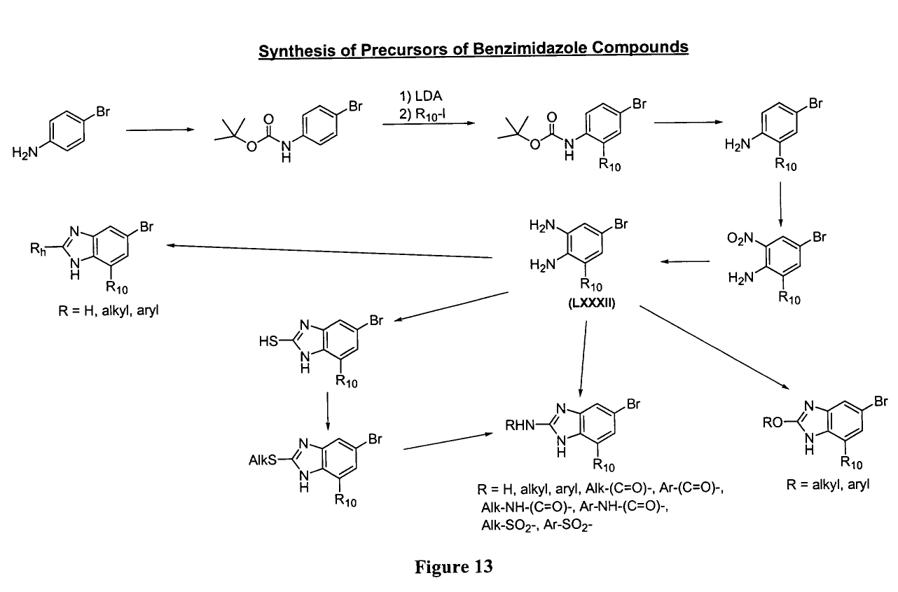

Certain bis-amino aromatics that are desirable for synthesizing precursors of Ar₁ that comprise benzimidazole rings are not readily commercially available. Therefore, the invention provides for the synthesis of many desirably substituted bis-amino aromatics via the reaction sequence illustrated in FIG. 13, which involves a directed lithiation reaction that can be carried out in the presence of a bromo substituent on an aromatic ring. The central 3-substituted-5-Bromo -benzene-1,2-diamine intermediate (LXXXII) can be condensed with analogs of carboxylic acids to form the desired benzimidazole rings.

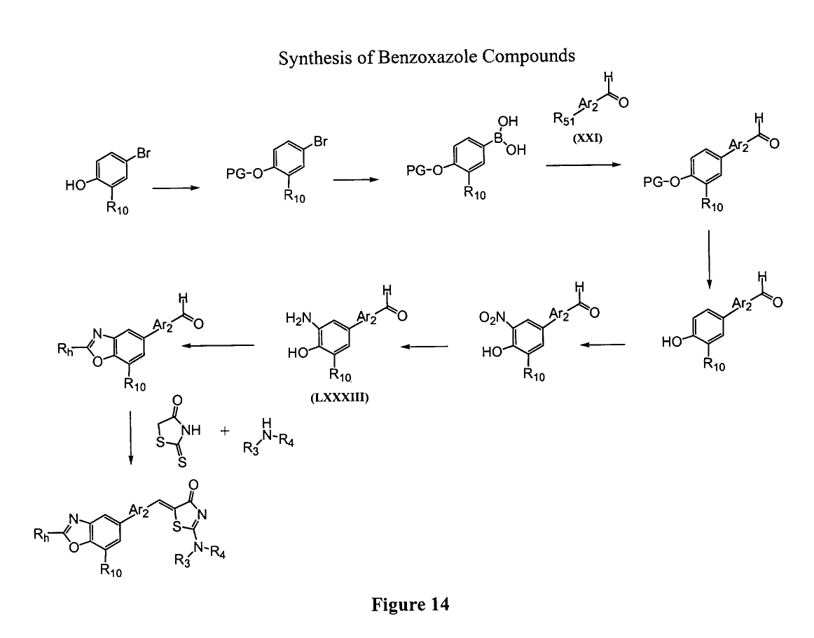

It is also possible in alternative synthetic strategies to functionalize appropriate biaryl intermediates that already comprise both Ar₁ and Ar₂ radicals, so as to incorporate a desired benzothiazole, benzimidazole, or benzoxazole ring. A generic example of such a synthesis, as applied to the synthesis of benzoxazoles, is illustrated in FIG. 14. A desirably substituted and protected bromophenol precursor of Ar₁ can be transformed to a boronic acid derivative suitable for Suzuki coupling, and coupled to a precursor of Ar₂, then the phenol deprotected, followed by nitration and reduction, to provide the desired amino phenol comprising a carbonyl group (LXXXIII). Compound (LXXXIII) can then be condensed with a carboxylic acid analog to form the desired benzoxazole ring, then the carbonyl group condensed with a five membered herterocycle as taught elsewhere herein, to form the desired final compounds of the invention.

A specific example of a benzoxazole synthesis is described in example 160. The references listed below provide further relevant experimental procedures for analogs of benzoxazoles, and are hereby incorporated herein by reference for their teachings of such experimental procedures; 1) K. Arakawa et al.: Chem. Pharm. Bull. 45 (1997) 1984; 2) J. H. Musser et al.: J. Med. Chem. 28 (1985) 1255; 3) Y. Katsura et al.: Chem. Pharm. Bull. 40 (1992) 1424; 4) I. N. Houpis et al.: J. Org. Chem. 58 (1993) 3176; 5) M. Kawase et al.: Heterocycles 48 (1998) 2103; 6) W. Kantlehner et al.: Liebig's Ann. Chem. (1982) 507; 7) Y. Ito et al.: J. Organomet. Chem. 131 (1977) 121; 8) E. -S. A. Ibrahim et al.: J. Heterocycl. Chem. 19 (1982) 761; 9) Acheson et al.: J. Chem. Soc. (1956) 4727; 10) F. Haviv et al.: J. Med. Chem. 31 (1988) 1719; 11) E. S. Lazer et al.: J. Med. Chem. 37 (1994) 913; and 12) R. W. DeSimone et al.: Bioorg. Med. Chem. Lett. 10 (2000) 2723.

The various organic group transformations utilized herein can be performed by a number of procedures other than those described above. References for other synthetic procedures that can be utilized for the synthetic steps leading to the compounds disclosed herein can be found in, for example, Smith, M. and March, J., Advanced Organic Chemistry, 5^{th} Edition, Weiley-Interscience (2001); or Larock, R. C., Comprehensive Organic Transformations, A Guide to Functional Group Preparations, Wiley, Inc. (1999), both incorporated herein by reference.

### Using the Compositions

The compounds described herein can be used effectively to prevent, alleviate or otherwise treat diseases of uncontrolled proliferation in mammals, including humans, such as cancer or precancerous diseases. Therefore, in certain embodiments, the invention relates to methods of treatment for a disease of uncontrolled cellular proliferation, wherein the method comprises administering to a mammal diagnosed as having a disease of uncontrolled cellular proliferation a compound of the invention in an amount that is effective to treat the disease of uncontrolled cellular proliferation.

The disease of uncontrolled cellular proliferation treated can be a carcinoma, lymphoma, leukemia, or sarcoma. The types of cancer treated by methods of the invention include but are not limited to Hodgkin's Disease, meyloid leukemia, polycystic kidney disease, bladder cancer, brain cancer, head and neck cancer, kidney cancer, lung cancer, myeloma, neuroblastoma/glioblastoma, ovarian cancer, pancreatic cancer, prostate cancer, skin cancer, liver cancer, melanoma, colon cancer, cervical carcinoma, breast cancer, epithelial cancer, and leukemia.

The effectiveness of the methods for treating the diseases of uncontrolled cellular proliferation can vary as a function of several variables, including the specific nature of disease or cancer, the details of the method of administration of the compound, the exact structure of the compounds administered, and other factors which are known to those of ordinary skill in the art. Therefore, one can screen the compounds of the invention for activity with respect to a selected disease of uncontrolled cellular proliferation.

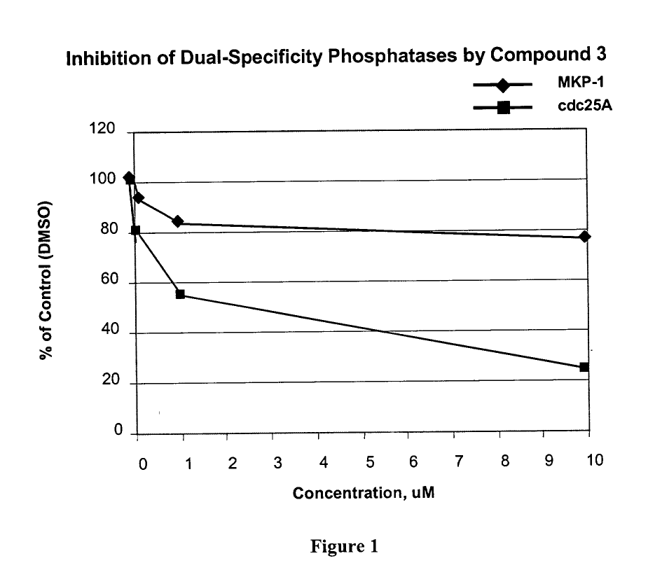

Compounds of the invention can function as inhibitors of Cdc 25-type phosphatase enzymes, which are overexpressed and significantly involved in uncontrolled cell growth and transformation in many types of cancer. Therefore, one method of in vivo screening of the compounds for anti-cancer activity is to test the activity of a particular compound for the ability to inhibit Cdc 25-type phosphatases. The results of one such test are shown in Example 161 below, and FIG. 1.

Compounds of the present invention have been found to be potent compounds in a number of biological assays, both in vitro and in vivo, that correlate to, or are representative of, human diseases. For example, the biological activity of the compounds provided herein can be measured by testing the compounds of the invention for their ability to kill or inhibit the growth of a panel of different human tumor cell lines. It is well known in the art that one or more known tumor cell lines used to test antitumor activity. Tumor cell lines that can be employed for such tests include but are not limited to known cell lines such as:

For Leukemia: CCRF—CEM, HL-60 (TB), K-562, MOLT-4, RPMI-8226, and SR. Lung Cancer: A549/ATCC, EKVX, HOP-62, HOP-92, NCI-H226, NCI-H23, NCI-H322M, NCI-H460, and NCI-H522.

Colon Cancer: COLO 205, HCC-2998, HCT-116, HCT-15, HT-29, KM-12, and SW-620.

CNS Cancer: SF-268, SF-295, SF-539, SNB-19, SNB-75, and U-251.

Melanoma: LOX-IMVI, MALME-3M, M-14, SK-MEL-2, SK-MEL-28, SK-MEL-5, UACC-257, and UACC-62.

Ovarian Cancer: IGR-OVI, OVCAR-3, OVCAR-4, OVCAR-5, OVCAR-8, and SK-OV-3.

Renal Cancer: 786-0, A-498, ACHN, CAKI-1, RXF-393, RXF-631, SN12C, TK10, and U0-31.

Prostate Cancer: PC-3 and DU-145.

Breast Cancer: MCF 7, MCF7/ADR-RES, MDA-MB-231/ATCC, HS578T, MDA-MB-435, MDA-N, BT-549, and T-47D.

This anti-cancer activity screening assay provides data regarding the general cytotoxicity of an individual compound. In particular, as described in the examples herein, active anticancer compounds can be identified by applying the compounds at a concentration of about 10 uM to one or more human tumor cell line cultures, such as for example leukemia, lung cancer, colon cancer, CNS cancer, melanoma, ovarian cancer, renal cancer, prostate cancer, breast cancer, or pancreatic cancer, so as to inhibit cell growth of the tumor cells. In some embodiments, the compounds of the invention are considered to be active for the treatment of cancer if, when they are applied to a culture of one of the above cancer cell lines at a concentration of about 10 uM, for a period of at least about 5 days, the growth of the cancer cells is inhibited, or the cancers cells killed to the extent of about 50% or more, as compared to a control not comprising the compound of the invention.

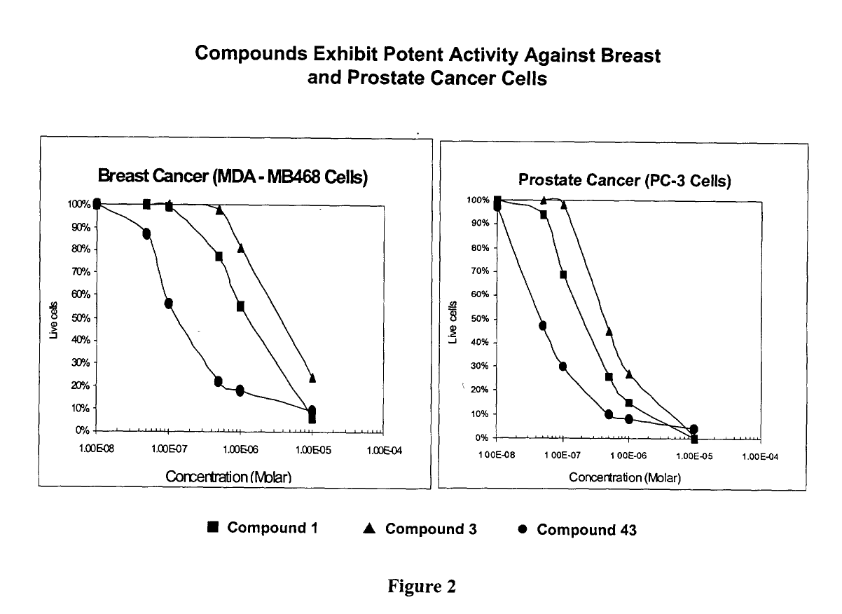

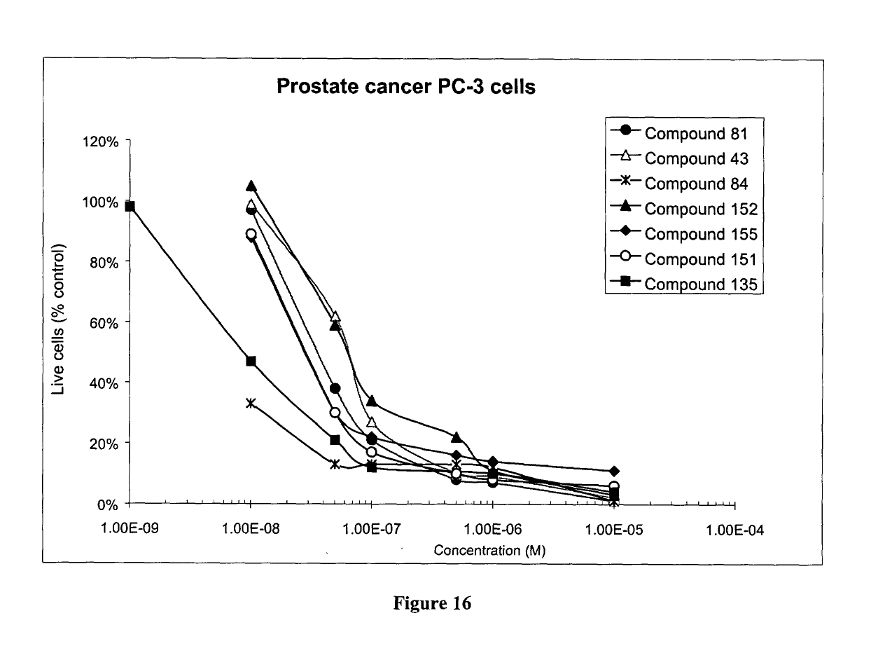

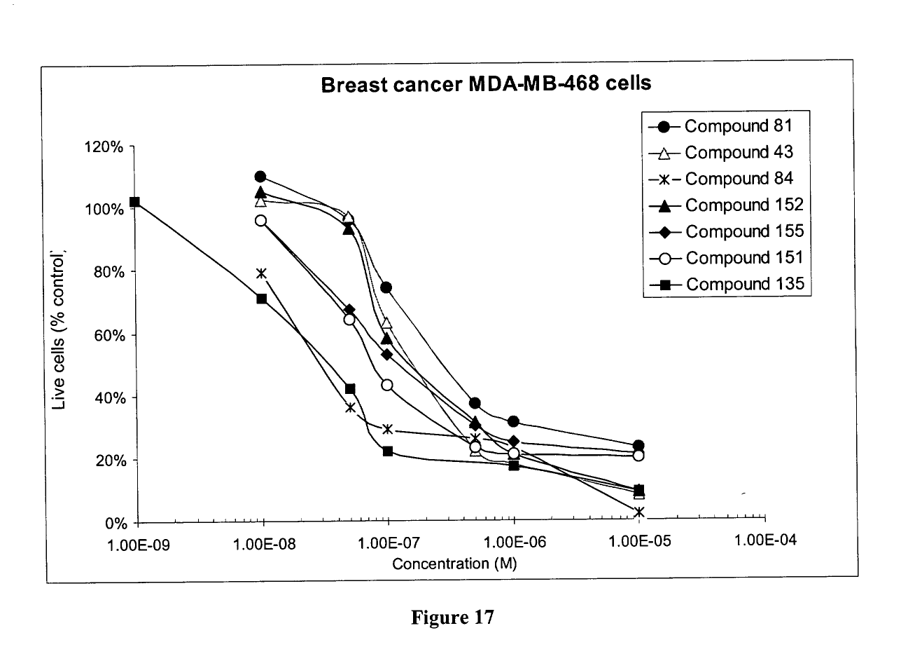

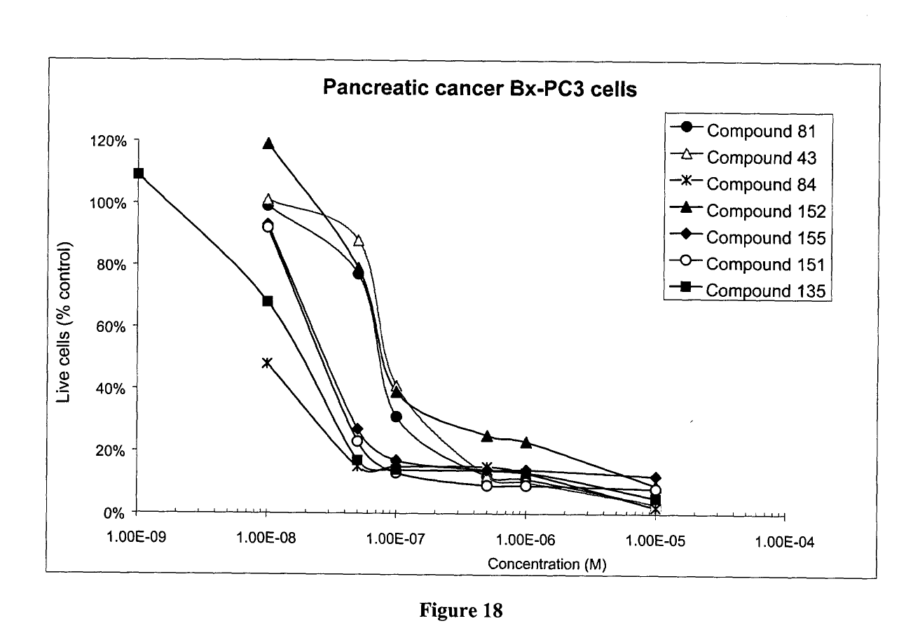

The anti-cancer activity of some of the compounds described herein have been tested in one type of such in vitro assay, as described in the examples herein, using a microculture assay with 3-(4,5-dimethylthiazol-2-yl)-2,5-diphenyltetrazolium bromide (“MTT”). This assay has an advantage over in vivo assay in that results are obtained within a week as opposed to several months. The assay can be carried out in 96-well microtiter plates. The MTT assay is based on the production of a dark blue formazan product by dehydrogenase in the mitochondria of live tumor cells after exposure to drug for 6 days [M. C. Alley, D. A. Scudiero, A. Monks, M. L. Hursey, M. J. Czerwinski, D. L. Fine, B. J. Abbout, J. G. Mayo, R. H. Shoemaker and M. R. Boyd, Cancer Res., 48, 589, 1988]. Thus, only live cells are stained and can be measured at 595 nm. Anti-cancer activity can be reported as percent of the tumor cell growth in the presence of compound at a defined dose compared to control/vehicle treated tumor cells. The results of some such screening assays are given in Examples 162a and 162b, and in FIGS. 2, 3, and 15-18.

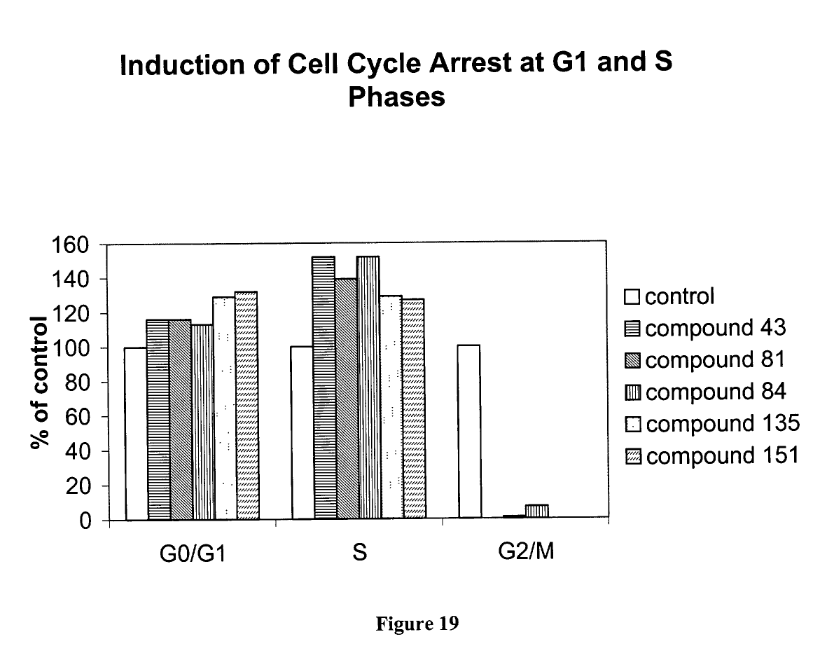

In a related experiment, detailed in Example 163, and FIG. 19, the activity of several of the compounds of the invention was tested using a culture of the human prostate cancer cell line PC-3, and the activity of the tested compounds for delaying and/or arresting the growth of the prostate cancer cells at a particular stage of cell development was demonstrated. The results of the experiment, shown in FIG. 19, provide evidence that compounds discloses herein are effective to delay and/or arrest cell growth at the G₀/G₁ or S phases of cell growth, so as to prevent the maturation of the cells to the G₂/M phases of cell growth. The results obtained in the experiments can be related to the activity of the compounds as inhibitors of Cdc 25 phosphatases.

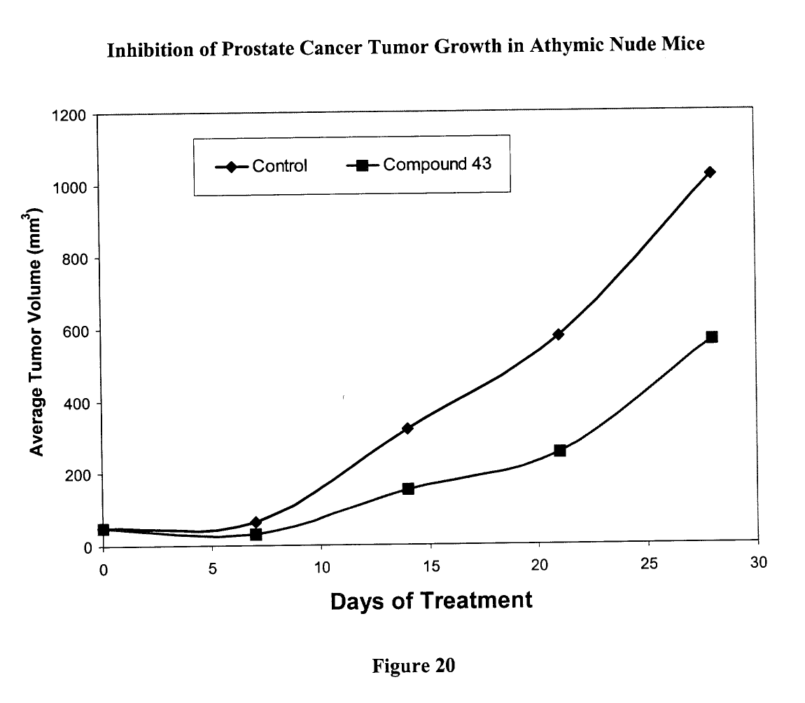

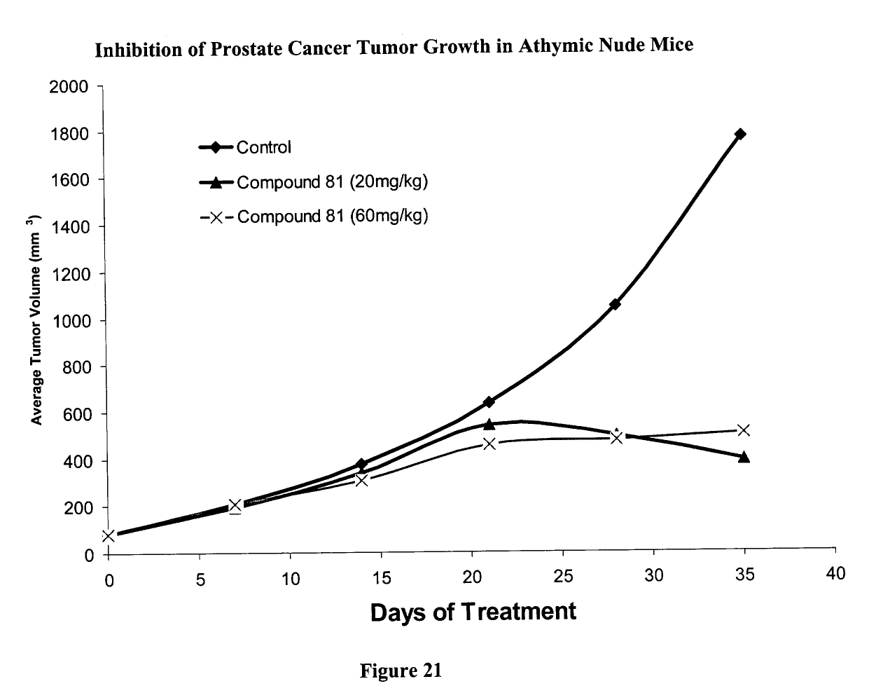

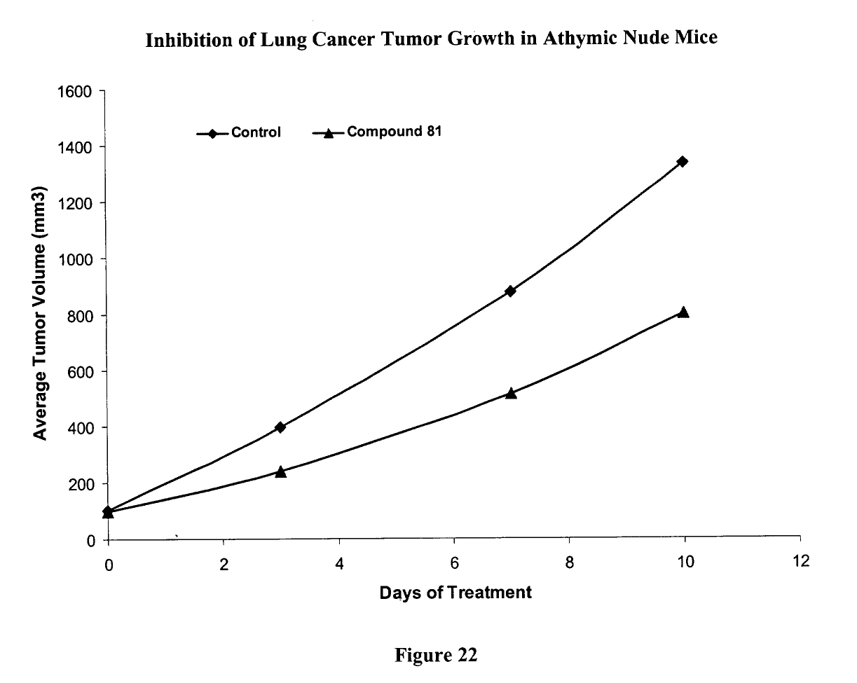

The compounds disclosed herein can be used to treat diseases of uncontrolled cellular proliferation in representative animal models, such as, athymic nude mice inoculated with human tumor cell lines. Example 164 and FIGS. 20-22 describe the results of in-vivo testing of compounds 43 and 81 of the invention with respect to prostate and non-small cell lung cancer, and show that compounds 43 and 81 significantly slowed the growth of solid human prostate cancer tumors, and that compound 81 significantly slowed the growth of non-small cell lung cancer, in athymic nude mice.

The compounds disclosed herein can be either used singularly, or plurally in mixtures of one or more compounds, isomers, or enantiomers, and in pharmaceutical compositions thereof for the treatment of mammalian diseases, particularly those diseases related to humans. Compounds disclosed herein and compositions thereof can be administered by various methods including, for example, orally, intravenously, enterally, parenterally, topically, nasally, vaginally, opthalinically, sublingually or by inhalation for the treatment of diseases related to uncontrolled proliferative diseases such as,

Routes of administration and dosages known in the art can be found in Comprehensive Medicinal Chemistry, Volume 5, Hansch, C. Pergamon Press, 1990; incorporated herein by reference. The compositions can also be used as regulators in diseases of uncontrolled proliferation. The composition can be useful in the treatment of polycystic kidney disease and cancers such as, carcinomas, lymphomas, leukemias, and sarcomas. A representative but non-limiting list of cancers is lymphoma, Hodgkin's Disease, myeloid leukemia, bladder cancer, brain cancer, head and neck cancer, kidney cancer, lung cancers such as small cell lung cancer and non-small cell lung cancer, myeloma, neuroblastoma/glioblastoma, ovarian cancer, pancreatic cancer, prostate cancer, skin cancer, liver cancer, melanoma, colon cancer, cervical carcinoma, breast cancer, and epithelial cancer. Compounds disclosed herein can be used for the treatment of inflammatory diseases such as osteoarthritis, rheumatoid arthritis, Crohn's Disease, pulmonary fibrosis, and Inflammatory Bowel Disease. Compounds disclosed herein can also be used for the treatment of precancer conditions such as cervical and anal dysplasias, other dysplasias, severe dysplasias, hyperplasias, atypical hyperplasias, and neoplasias.

Although the compounds described herein can be administered as pure chemicals either singularly or plurally, it is preferable to present the active ingredient as a pharmaceutical composition. Thus another embodiment of the invention is the use of a pharmaceutical composition comprising one or more compounds and/or a pharmaceutically acceptable salt thereof, together with one or more pharmaceutically acceptable carriers thereof and, optionally, other therapeutic and/or prophylactic ingredients. The carrier(s) should be “acceptable” in the sense of being compatible with the other ingredients of the composition and not overly deleterious to the recipient thereof.

The compounds of the invention are preferably present in the pharmaceutical composition in an amount effective to treat a disease of uncontrolled cellular proliferation, such as the various cancers and precancerous conditions described herein.

It will be further appreciated that the amount of the compound, or an active salt or derivative thereof (i.e. a prodrug), required for effective use in treatment of a disease of uncontrolled cellular proliferation, such as the various cancers and precancerous conditions described herein, will vary not only with the particular compound and/or salt selected but also with the route of administration, the nature of the condition being treated, and the age and condition of the patient. An effective amount of a compound provided herein is a substantially nontoxic but sufficient amount of the compound to provide a clinically useful degree inhibition of the growth or progression of the disease of uncontrolled cellular proliferation.

Though it is not possible to specify a single predetermined pharmaceutically effective amount of the compounds of the invention, and/or their pharmaceutical compositions, for each and every disease condition to be treated, determining such pharmaceutically effective amounts are within the skill of, and ultimately at the discretion of an attendant physician or clinician of ordinary skill. In some embodiments, the active compounds of the invention are administered to achieve peak plasma concentrations of the active compound of from typically about 0.1 to about 100 μM, about 1 to 50 μM, or about 2 to about 30 μM. This can be achieved, for example, by the intravenous injection of a 0.05 to 5% solution of the active ingredient, optionally in saline, or orally administered as a bolus containing about 0.5-500 mg of the active ingredient. Desirable blood levels can be maintained by continuous infusion to provide about 0.01-5.0 mg/kg/hr or by intermittent infusions containing about 0.4-15 mg/kg of the active compounds of the invention.

Pharmaceutical compositions include those suitable for oral, enteral, parental (including intramuscular, subcutaneous and intravenous), topical, nasal, vaginal, ophthalinical, sublingually or by inhalation administration. The compositions can, where appropriate, be conveniently presented in discrete unit dosage forms and can be prepared by any of the methods well known in the art of pharmacy. Such methods include the step of bringing into association the active compound with liquid carriers, solid matrices, semi-solid carriers, finely divided solid carriers or combination thereof, and then, if necessary, shaping the product into the desired delivery system.

When desired, the above-described compositions can be adapted to provide sustained release of the active ingredient employed, e.g., by combination thereof with certain hydrophilic polymer matrices, e.g., comprising natural gels, synthetic polymer gels or mixtures thereof.

The compounds of the invention can have oral bioavailability as exhibited by blood levels after oral dosing, either alone or in the presence of an excipient. Oral bioavailability allows oral dosing for use in chronic diseases, with the advantage of self-administration and decreased cost over other means of administration. Pharmaceutical compositions suitable for oral administration can be presented as discrete unit dosage forms such as hard or soft gelatin capsules, cachets or tablets each containing a predetermined amount of the active ingredient; as a powder or as granules; as a solution, a suspension or as an emulsion. The active ingredient can also be presented as a bolus, electuary or paste. Tablets and capsules for oral administration can contain conventional excipients such as binding agents, fillers, lubricants, disintegrants, or wetting agents. The tablets can be coated according to methods well known in the art., e.g., with enteric coatings.

Oral liquid preparations can be in the form of, for example, aqueous or oily suspensions, solutions, emulsions, syrups or elixirs, or can be presented as a dry product for constitution with water or other suitable vehicle before use. Such liquid preparations can contain conventional additives such as suspending agents, emulsifying agents, non-aqueous vehicles (which can include edible oils), or one or more preservative.

The compounds can also be formulated for parenteral administration (e.g., by injection, for example, bolus injection or continuous infusion) and can be presented in unit dose form in ampules, pre-filled syringes, small bolus infusion containers or in multi-does containers with an added preservative. The compositions can take such forms as suspensions, solutions, or emulsions in oily or aqueous vehicles, and can contain formulatory agents such as suspending, stabilizing and/or dispersing agents. Alternatively, the active ingredient can be in powder form, obtained by aseptic isolation of sterile solid or by lyophilization from solution, for constitution with a suitable vehicle, e.g., sterile, pyrogen-free water, before use.

For topical administration to the epidermis, the compounds can be formulated as ointments, creams or lotions, or as the active ingredient of a transdermal patch. Suitable transdermal delivery systems are disclosed, for example, in Fisher et al. (U.S. Pat. No. 4,788,603, incorporated herein by reference) or Bawas et al. (U.S. Pat. Nos. 4,931,279, 4,668,504 and 4,713,224; all incorporated herein by reference). Ointments and creams can, for example, be formulated with an aqueous or oily base with the addition of suitable thickening and/or gelling agents. Lotions can be formulated with an aqueous or oily base and will in general also contain one or more emulsifying agents, stabilizing agents, dispersing agents, suspending agents, thickening agents, or coloring agents. The active ingredient can also be delivered via iontophoresis, e.g., as disclosed in U.S. Pat. Nos. 4,140,122, 4383,529, or 4,051,842; incorporated herein by reference.

Compositions suitable for topical administration in the mouth include unit dosage forms such as lozenges comprising active ingredient in a flavored base, usually sucrose and acacia or tragacanth; pastilles comprising the active ingredient in an inert base such as gelatin and glycerin or sucrose and acacia; mucoadherent gels, and mouthwashes comprising the active ingredient in a suitable liquid carrier.

When desired, the above-described compositions can be adapted to provide sustained release of the active ingredient employed, e.g., by combination thereof with certain hydrophilic polymer matrices, e.g., comprising natural gels, synthetic polymer gels or mixtures thereof.

The pharmaceutical compositions according to the invention can also contain other adjuvants such as flavorings, coloring, antimicrobial agents, or preservatives.

It will be further appreciated that the amount of the compound, or an active salt or derivative thereof, required for use in treatment will vary not only with the particular salt selected but also with the route of administration, the nature of the condition being treated and the age and condition of the patient and will be ultimately at the discretion of the attendant physician or clinician.

In general, one of skill in the art understands how to extrapolate in vivo data obtained in a model organism, such as an athymic nude mice inoculated with human tumor cell lines, to another mammal, such as a human. These extrapolations are not simply based on the weights of the two organisms, but rather incorporate differences in metabolism, differences in pharmacological delivery, and administrative routes. Based on these types of considerations, a suitable dose will, in alternative embodiments, typically be in the range of from about 0.5 to about 10 mg/kg/day, or from about 1 to about 20 mg/kg of body weight per day, or from about 5 to about 50 mg/kg/day.

The desired dose can conveniently be presented in a single dose or as divided doses administered at appropriate intervals, for example, as two, three, four or more sub-doses per day. The sub-dose, as necessary by one skilled in the art, can itself be further divided, e.g., into a number of discrete loosely spaced administrations.

One skilled in the art will recognize that dosage and dosage forms outside these typical ranges can be tested and, where appropriate, be used in the methods of this invention.

### Combinations with Other Active Agents

According to another aspect of the invention, pharmaceutical compositions of matter useful for the treatment of cancer are provided that contain, in addition to the aforementioned compounds, an additional therapeutic agent. Such agents can be chemotherapeutic agents, ablation or other therapeutic hormones, antineoplastic agents, monoclonal antibodies useful against cancers and angiogenesis inhibitors. The following discussion highlights some agents in this respect, which are illustrative, not limitative. A wide variety of other effective agents also can be used.

Among hormones which can be used in combination with the present inventive compounds, diethylstilbestrol (DES), leuprolide, flutamide, cyproterone acetate, ketoconazole and amino glutethimide.

Among antineoplastic and anticancer agents that can be used in combination with the inventive compounds, 5-fluorouracil, vinblastine sulfate, estramustine phosphate, suramin and strontium-89. Other chemotherapeutics useful in combination and within the scope of the present invention are buserelin, chlorotranisene, chromic phosphate, cisplatin, cyclophosphamide, dexamethasone, doxorubicin, estradiol, estradiol valerate, estrogens conjugated and esterified, estrone, ethinyl estradiol, floxuridine, goserelin, hydroxyurea, melphalan, methotrexate, mitomycin and prednisone.

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as can be applied to the essential features hereinbefore set forth, and as follows in the scope of the appended claims.

The following examples are given merely to illustrate the invention and are not intended to be limiting in any manner:

### EXAMPLES

The following Examples (which are non-continuously numbered from 1 to 160) report representative examples the synthetic procedures used to synthesize various species of compounds that are within the scope of the invention. Each compound may be referred to elsewhere herein, in shorthand form, by its example number. For example, 5-[3-(3-Adamantan-1-yl-4-hydroxy-5-fluoro-phenyl) benzylidene]-2-morpholin-4-yl-thiazol-4-one, whose synthesis is reported in Example 1, may be referred to elsewhere herein as “Compound 1.”

Examples 161-164 report results relating to the biological activity of the compounds of the invention.

### Example 1

### 5-[3-(3-Adamantan-1-yl-4-hydroxy-5-fluoro-phenyl) benzylidene]-2-morpholin-4-yl-thiazol-4-one

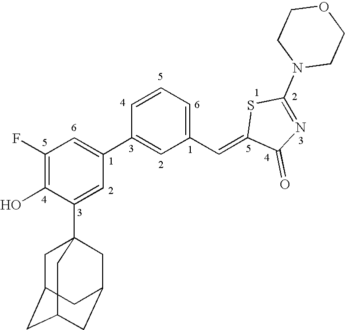

A solution of anhydrous toluene (300 mL), morpholine (0.96 mL), acetic acid (0.34 mL), 5-[3-(3-adamantan-1-yl-4-hydroxy-5-fluoro-phenyl) benzaldehyde (3.5 g, 10 mmol) and rhodanine (1.47 g, 11 mmol) was heated at reflux overnight under an argon atmosphere. The reaction mixture was cooled to room temperature, and the resulting crystalline compound was filtered, washed with toluene and ethanol/water. The off-white solid was dried under high vacuum to afford 3.75 g (73%) of 5-[3-(3-adamantan-1-yl-4-hydroxy-5-fluoro-phenyl) benzylidene]-2-morpholin-4-yl-thiazol-4-one, mp 293-295° C. ¹H NMR (300 MHz; DMSO-d₆): δ 1.72 (s, 6 H), 2.02 (s, 3 H), 2.13 (s, 6 H), 3.63-3.65 (m, 2 H), 3.71-3.74 (m, 4 H), 3.91 (t, J=4.2 Hz, 2 H), 7.21 (s, 1 H), 7.39 (dd, J₁=1.5 Hz, J₂=11.4 Hz, 1 H), 7.49 (d, J=5.1 Hz, 1 H), 7.51 (s, 1 H), 7.62-7.66 (m, 1 H), 7.74 (s, 1 H, 7.84 (s, 1 H), 9.58 (d, J=2.7 Hz, 1 H. ¹³C NMR (300 MHz; DMSO-d₆): 28.6, 36.7, 37.1 (d, J=2.3 Hz), 48.6, 48.7, 66.6, 66.7, 111.5 (d, J=19.7 Hz), 120.3, 127.5, 127.7, 128.0, 128.7, 129.8, 129.9, 130.4, 134.6, 139.4, 140.7, 143.5 (d, J=14.9 Hz), 152.6 (d, J=234 Hz), 174.5, 179.3. MS: Expected: 518; Found: 519 (M+H), Expected: 518; Found: 517 (M−H).

The intermediate 3-(3-adamantan-1-yl-4-hydroxy-5-fluoro-phenyl) benzaldehyde was prepared as follows:

### a. 3-(3-Adamantan-1-yl-4-hydroxy-5-fluoro-phenyl) benzaldehyde

To a solution of 3-(3-adamantan-1-yl-4-(t-butyldimethylsilyloxy)-5-fluoro-phenyl) benzaldehyde (2.48 g, 5.34 mmol) in anhydrous THF (60 mL) under an atmosphere of argon cooled to 0° C. was added dropwise a 1.0 M solution of tetrabutyl ammonium fluoride in THF (5.88 mL, 5.88 mmol). After the starting material was consumed as determined by TLC, the mixture was poured into a slurry of ice water. The mixture was diluted with ethyl acetate, separated and the aqueous layer was further extracted with ethyl acetate. The combined organics were washed successively with water and brine, dried over anhydrous magnesium sulfate, filtered, and evaporated. The resulting product was stirred in hexane, filtered and dried under reduced pressure to give 1.48 g (80%) of 3-(3-adamantan-1-yl-4-hydroxy-5-fluoro-phenyl) benzaldehyde as a yellow powder.

### b. 3-(3-Adamantan-1-yl-4-(t-butyldimethylsilyloxy)-5-fluoro-phenyl) benzaldehyde

A mixture of 3-adamantan-1-yl-4-(t-butyldimethylsilyloxy-5-fluoro bromobenzene (14.00 g, 31.89 mmol), 3-formylphenylboronic acid (5.74 g, 38.26 mmol) and sodium carbonate (10.14 g, 95.67 mmol) in toluene: ethanol (4:1, 800 mL) and water (33 mL) was degassed with argon for 45 minutes. Tetrakis(triphenylphosphine) palladium(0) (3.67 g, 3.19 mmol) was added and the mixture heated at reflux under argon for 5.5 hours. The solution was cooled to room temperature, diluted with ethyl acetate and washed successively with water and brine, dried over anhydrous magnesium sulfate, filtered and evaporated. The residue was purified on silica gel (eluent: hexane: ethyl acetate, 97:3) to give 11.34 g of 3-(3-adamantan-1-yl-4-hydroxy-5-fluoro-phenyl) benzaldehyde (77%).

### c. 3-Adamantan-1-yl-4-(t-butyldimethylsilyloxy-5-fluoro bromobenzene

To a solution of 3-adamantan-1-yl-4-hydroxy-5-fluoro bromobenzene (18.90 g, 58.15 mmol) and DMAP (7 mg, 0.05 mmol) in anhydrous DMF (120 mL) and triethylamine (6.47 g, 63.96 mmol, 8.92 mL) was added t-butyldimethylsilyl chloride (9.64 g, 63.96 mmol). The resulting mixture was allowed to stir for 17 hours poured into water, and extracted with diethyl ether (twice). The combined organics were washed successively with water and brine, dried over anhydrous magnesium sulfate, filtered, and evaporated. The residue was purified on silica gel (eluent:hexane:ethyl acetate, 9:1) to give 23.97 g (94%) of 3-adamantan-1-yl-4-(t-butyldimethylsilyloxy-5-fluoro bromobenzene as a white powder.

### d. 3-Adamantan-1-yl-4-hydroxy-5-fluoro bromobenzene

To a mixture of 3-fluoro-4-hydroxy-bromobenzene (19.10 g, 100 mmol) and 1-adamantanol (15.20 g, 100 mmol) in CH₂Cl₂ (100 mL) under an atmosphere of argon was added sulfuric acid (10 mL) dropwise over 3 minutes at room temperature. After stirring for 22 hours, the resulting mixture was poured into water and carefully neutralized with solid NaHCO₃ and extracted with CH₂Cl₂ (twice). The combined organics were washed brine and dried (MgSO₄). The mixture was filter, evaporated and the residue purified on silica gel (hexane) to give 14.62 g (45%) of 3-adamantan-1-yl-4-hydroxy-5-fluoro bromobenzene as a yellow solid.

### Example 2

### 5-[3-(3-Adamantan-1-yl-4-hydroxy-phenyl) benzylidene]-2-morpholin-4-yl-thiazol-4-one

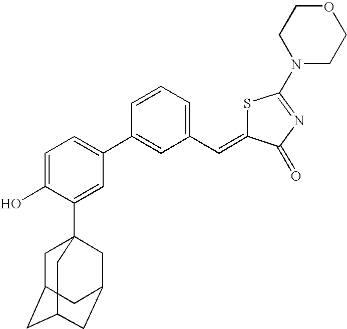

Prepared in a manner similar to that described in Example 1 using 3-(3-adamantan-1-yl-4-hydroxy-phenyl) benzaldehyde, mp 325-328° C. ¹H NMR (300 MHz; DMSO-d₆): δ 1.75 (s, 6 H), 2.05 (s, 3 H), 2.15 (s, 6 H), 3.60-3.80 (m, 6 H), 3.90-3.98 (m, 2 H), 6.88 (d, J=8.1 Hz, 1 H), 7.30-7.43 (m, 2 H), 7.50-7.55 (m, 2 H), 7.59-7.65 (m, 1 H), 7.76 (s, 1 H), 7.83 (s, 1 H), 9.55 (s, 1 H). MS: Expected: 500; Found: 501 (M+H), & (M+Na), Expected: 500; Found: 499 (M−H).

The intermediate 3-(3-adamantan-1-yl-4-hydroxy-phenyl) benzaldehyde was prepared as follows:

a. 3-(3-Adamantan-1-yl-4-hydroxy-phenyl) benzaldehyde.

To a solution of 3-(3-adamantan-1-yl-4-(t-butyldimethylsilyloxy)-phenyl) benzaldehyde (10.00 g, 22.39 mmol) in anhydrous THF (80 mL) under an atmosphere of argon cooled to 0° C. was added dropwise a 1.0 M solution of tetrabutyl ammonium fluoride in THF (24.6 mL, 24.6 mmol). After the starting material was consumed as determined by TLC, the mixture was poured into a slurry of ice water. The mixture was diluted with ethyl acetate, separated and the aqueous layer was further extracted with ethyl acetate. The combined organics were washed successively with water and brine, dried over anhydrous magnesium sulfate, filtered, and evaporated. The resulting product was stirred in hexane, filtered and dried under reduced pressure to give 7.01 g (94%) of 3-(3-adamantan-1-yl-4-hydroxy-phenyl) benzaldehyde as a yellow powder.

### b. 3-(3-Adamantan-1-yl-4-(t-butyldimethylsilyloxy)-phenyl) benzaldehyde

A mixture of 3-adamantan-1-yl-4-(t-butyldimethylsilyloxy-bromobenzene (32.56 g, 77.24 mmol), 3-formylphenylboronic acid (13.90 g, 92.70 mmol) and sodium carbonate (20.47 g, 193.10 mmol) in toluene: ethanol (4:1, 600 mL) and water (60 mL) was degassed with argon for 45 minutes. Tetrakis(triphenylphosphine)palladium(0) (4.46 g, 3.86 mmol) was added and the mixture heated at reflux under argon overnight. The solution was cooled to room temperature, diluted with ethyl acetate and washed successively with water and brine, dried over anhydrous magnesium sulfate, filtered and evaporated. The residue was purified on silica gel (eluent: hexane: ethyl acetate, 95:5) to give 26.86 g (78%) of 3-(3-adamantan-1-yl-4-(t-butyldimethylsilyloxy)-phenyl) benzaldehyde as an oil that solidified on standing.

### c. 3-[3-Adamantan-1-yl-4-(t-butyldimethylsilyloxy)]-1-bromobenzene

To a solution of 3-adamantan-1-yl-4-hydroxy-bromobenzene (18.90 g, 58.15 mmol) and DMAP (80 mg, 6.51 mmol) in anhydrous DMF (200 mL) and triethylamine (16.47 g, 162.70 mmol, 22.7 mL) at 0° C. was added t-butyldimethylsilyl chloride (9.64 g, 63.96 mmol). After 17 hours, the resulting mixture was poured into water and extracted with ethyl acetate (twice). The combined organics were washed successively with water and brine, dried over anhydrous magnesium sulfate, filtered, and evaporated. The resulting solid was suspended into hexane residue was purified on silica gel (eluent: hexane: ethyl acetate, 9:1) to give 46.2 g (84%) of 3-adamantan-1-yl-4-(t-butyldimethylsilyloxy-bromobenzene as a yellowish powder

### d. 2-Adamantan-1-yl-4-bromophenol

To a mixture of 4-bromophenol (34.60 g, 200 mmol) and 1-adamantanol (30.45 g, 200 mmol) in 100 mL of anhydrous CH₂Cl₂ at room temperature was added dropwise over 10-15 minutes concentrated H₂SO₄ (11 mL). After 1.5 hours a thick suspension resulted and the reaction was allowed to continue for a total of 24 hours. The suspension was carefully poured into ice water and neutralized with solid NaHCO₃. The resulting layers were separated and the aqueous layer extracted with CH₂Cl₂ (2×). The combined organics were washed with brine, dried (MgSO₄) and filtered. The solvent was removed under reduced pressure and the resulting solid was purified on silica gel (hexane: ethyl acetate 85:15), the impure fractions were further purified by recrystallization from hexane and the two lots combined to give 45.2 g (74%) of 2-adamantan-1-yl-4-bromophenol.

### Example 3

### 5-[3-(3-Adamantan-1-yl-4-hydroxy-phenyl) benzylidene]-2-piperidin-1-yl-thiazol-4-one

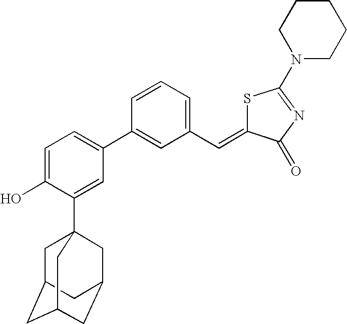

Prepared in a manner similar to that described in Example 1 using 3-(3-adamantan-1-yl-4-hydroxy-phenyl) benzaldehyde (example 2a), rhodanine and piperidine, mp 311-312° C. ¹H NMR (300 MHz; DMSO-d₆): δ 1.60-1.75 (m, 12 H, 2.03 (s, 3H), 2.14 (s, 6H), 3.60 (broad s, 2 H, 3.89 (m, 2 H, 6.86 (d, J=8.1 Hz, 1 H, 7.12-7.26 (m, 1 H, 7.34-7.39 (m, 2 H, 7.49 (d, J=4.8 Hz, 2 H, 7.59-7.64 (m, 1 H), 7.71 (s, 1 H, 7.81 (s, 1 H, 9.53 (s, 1 H. ¹³C NMR (300 MHz; DMSO-d₆): 21.1, 23.4, 25.1, 25.8, 28.5, 36.3, 36.6, 49.1, 49.7, 116.8, 124.7, 125.2, 126.9, 127.0, 128.0, 128.7, 128.8, 129.5, 129.7, 134.3, 135.7, 135.8, 137.2, 141.4, 156.1, 173.0, 179.1. MS: Expected: 498; Found: 499 (M+H), 521 (M+Na); Expected: 498; Found: 497 (M−H).

### Example 4

### 5-[4-(3-Adamantan-1-yl-4-hydroxy-phenyl) benzylidene]-2-piperidin-1yl-thiazol-4-one

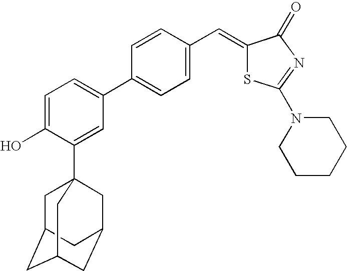

Prepared in a manner similar to that described in Example 1 using 4-(3-adamantan-1-yl-4-hydroxy-phenyl) benzaldehyde, rhodanine and piperidine, mp 325-328° C. ¹H NMR (300 MHz; DMSO-d₆): δ 1.60-1.73 (m, 12 H, 2.03 (s, 3H), 2.12 (s, 6H), 3.61 (s, 2 H, 3.87-3.90 (m, 2 H, 6.86 (d, J=8.7 Hz, 1 H, 7.36-7.39 (m, 2 H, 7.62-7.71 (m, 5H), 9.59 (s, 1 H. MS: Expected: 498; Found: 499 (M+H), 521 (M+Na); Expected: 498; Found: 497 (M−H).

The intermediate 4-(3-adamantan-1-yl-4-hydroxy-phenyl) benzaldehyde was prepared in a similar manner as described in Example 2 using 4-formylphenyl boronic acid in Step 2b.

### Example 5

### 5-[4-(3-Adamantan-1-yl-4-hydroxy-3-fluoro-phenyl) benzylidene]-2-piperidin-1-yl-thiazol-4-one

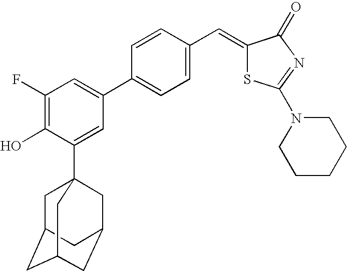

Prepared in a manner similar to that described in Example 1 using 4-(3-adamantan-1-yl-4-hydroxy-5-fluoro-phenyl) benzaldehyde, rhodanine and piperidine, mp 247-250° C. ¹H NMR (300 MHz; DMSO-d₆): δ 1.60-1.75 (m, 12 H, 2.04 (s, 3H), 2.12 (s, 6H), 3.61 (broad s, sH), 3.86-3.90 (m, 2 H, 7.22 (s, 1 H, 7.43 (dd, J=11.7 Hz, J=2.1 Hz, 1 H, 7.63 (s, 1 H, 7.65 (d, J=7.2 Hz, 2 H, 7.74 (d, J=8.4 Hz, 2 H, 9.64 (s, 1 H.

The intermediate 4-(3-adamantan-1-yl-4-hydroxy-5-fluoro-phenyl) benzaldehyde was prepared in a similar manner as described in Example 1 using 4-formylphenyl boronic acid in Step 1b.

### Example 6

### 5-[4-(2-Hydroxy-5-adamantan-1-yl-phenyl) benzylidene]-2-piperidin-1-yl-thiazol-4-one

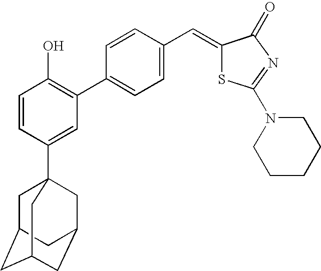

Prepared in a manner similar to that described in Example 1 using 4-(2-hydroxy-5-adamantan-1-yl-phenyl) benzaldehyde, rhodanine and piperidine, mp 312-314° C. ¹H NMR (300 MHz; DMSO-d₆): δ 7.84 (s, 1 H, 7.61 (m, 4H), 7.25 (m, 2 H, 6.94 (d, J=8.4 Hz, 1 H, 4.03 (m, 2 H, 3.59 (m, 2 H, 2.09 (brs, 3H), 1.92 (broad d, 6H), 1.77 (brs, 6H).

The intermediate 4-(2-hydroxy-5-adamantan-1-yl-phenyl) benzaldehyde was prepared in a similar manner as described in Example 2 using 4-formyl phenyl boronic acid in step 2b and 2-bromo-phenol in step 2d.

### Example 8

### 5-[3-(3-Adamantan-1-yl-4-hydroxy-phenyl) benzylidene]-2-(4-methyl-piperazin-1-yl)-thiazol-4-one

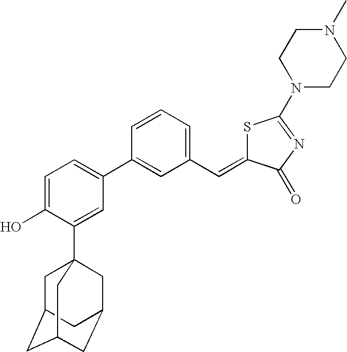

Prepared in a manner similar to that described in Example 1 using 3-(3-adamantan-1-yl-4-hydroxy-phenyl) benzaldehyde (example 2a), rhodanine and N-methylpiperazine, mp 291-294° C. ¹H NMR (300 MHz; DMSO-d₆):, 1.73 (s, 6 H), 2.03 (s, 3 H), 2.13 (s, 6 H), 2.23 (s, 3 H), 2.40-2.52 (m, 4 H), 3.64 (t, J=4.2 Hz, 2 H), 3.91 (t, J=4.2 Hz, 2 H), 6.86 (d, J=8.4 Hz, 1 H), 7.35-7.43 (m, 2 H), 7.47-7.55 (m, 2 H), 7.60-7.63 (m, 1 H), 7.72 (s, 1 H), 7.81 (s, 1 H), 9.54 (s, 1 H). MS: Expected: 513; Found: 514 (M+H), 536 (M+Na); Expected: 513; Found: 512 (M−H).

### Example 9

### 5-[3-(3-Adamantan-1-yl-4-hydroxy-5-fluoro-phenyl) benzylidene]-2-(4-methyl-piperazin-1-yl)-thiazol-4-one

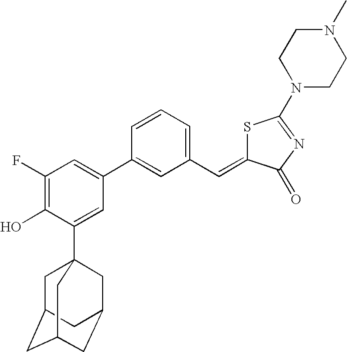

Prepared in a manner similar to that described in Example 1 using 3-(3adamantan-1-yl-4-hydroxy-5-fluoro-phenyl) benzaldehyde (example 1a), rhodanine and N-methylpiperazine, mp 226-228° C. ¹H NMR (300 MHz; DMSO-d₆):, 1.74 (s, 6H), 2.05 (s, 3H), 2.14 (s, 6H), 2.84 (s, 3H), 3.50 (s, 5H), 3.93 (s, 3H), 7.25 (s, 1 H, 7.43 (dd, J=1.8 Hz, J=11.7 Hz, 1 H, 7.56 (d, J=4.5 Hz, 1 H, 7.57 (s, 1 H, 7.72-7.70 (m, 1 H, 7.83 (s, 1 H, 7.91 (s, 1 H, 9.65 (s, 1 H. MS: Expected: 531; Found: 532 (M+H), 554 (M+Na); Expected: 531; Found: 530 (M−H).

### Example 10

### 5-[3-(3-Adamantan-1-yl-4-hydroxy-phenyl) benzylidene]-2-diethylamino-thiazol-4-one

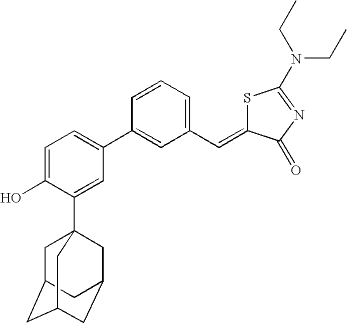

Prepared in a manner similar to that described in Example 1 using 3-(3-adamantan-1-yl-4-hydroxy-phenyl) benzaldehyde (example 2a), rhodanine and diethylamine, mp 275-277° C. ¹H NMR (300 MHz; DMSO-d₆): 1.21 (t, J=7.2 Hz, 3 H), 1.63 (t, J=7.2 Hz, 23H), 1.73 s, 6 H), 2.04 (s, 3 H), 2.14 (s, 6 H), 3.58 (q, J=7.2 Hz, 2 H), 3.73 (q, J=7.2 Hz, 2 H), 6.86 (d, J=8.4 Hz, 1 H), 7.25-7.45 (m, 2 H), 7.47-7.55 (m, 2 H), 7.60-7.63 (m, 1 H), 7.72 (s, 1 H), 7.81 (s, 1 H), 9.54 (s, 1 H). MS: Expected: 486; Found: 487 (M+H); Expected: 486; Found: 485 (M−H).

### Example 12

### 5-[3-(3-Adamantan-1-yl-4-hydroxy-phenyl) benzylidene]-2-pyrrolidine-1-yl-thaizol-4-one

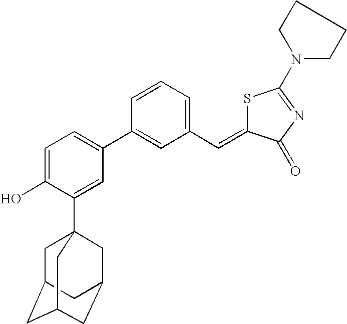

Prepared in a manner similar to that described in Example 1 using 3-(3-adamantan-1-yl-4-hydroxy-phenyl) benzaldehyde (example 2a), rhodanine and pyrrolidine, mp 243-245° C. ¹H NMR (300 MHz, DMSO-d₆): δ 1.73 (s, 6 H), 1.90-2.04 (m, 7 H), 2.14 (s, 6 H), 3.62 (t, J=6.3 Hz, 2 H), 3.69 (t, J=6.3 Hz, 2 H), 6.86 (d, J=8.7 Hz, 1 H), 7.36 (d, J=8.7 Hz, 1 H), 7.40 (s, 1 H), 7.43-7.55 (m, 2 H), 7.62 (d, J=6.3 Hz, 1 H), 7.70 (s, 1 H), 7.80 (s, 1 H), 9.55 (s, 1 H).

### Example 13

### 5-[3-(3-Adamantan-1-yl-4-hydroxy-5-fluoro-phenyl) benzylidene]-2-pyrrolidine-1-yl-thiazol-4-one-thiazol-4-one

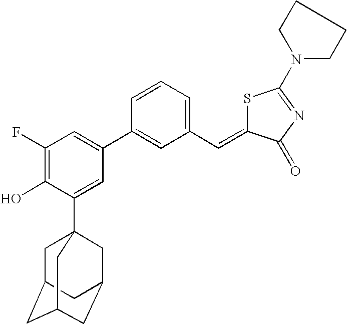

Prepared in a manner similar to that described in Example 1 using 3-(3-adamantan-1-yl-4-hydroxy-5-fluoro-phenyl) benzaldehyde (example 1a), rhodanine and pyrrolidine, mp 309-311° C. ¹H NMR (300 MHz, DMSO-d₆): δ 1.73 (s, 6 H), 1.96-2.03 (m, 7 H), 2.13 (s, 6 H), 3.59 (t, J=6.3 Hz, 2 H), 3.70 (t, J=6.3 Hz, 2 H), 7.20 (s, 1 H), 7.32 (dd, J₁=2.1 Hz, J₂=11.4 Hz, 1 H), 7.44-7.50 (m, 2 H), 7.57-7.61 (m, 1 H), 7.68 (s, 1 H), 7.78 (s, 1 H), 9.47 (d, J=2.7 Hz, 1 H). MS: Expected: 502; Found: 503 (M+H); Expected: 502; Found: 501 (M−H).

### Example 14

### 5-[3-(3-Adamantan-1-yl-4-hydroxy-phenyl) benzylidene]-2-azepan-1-yl-thiazol-4-one

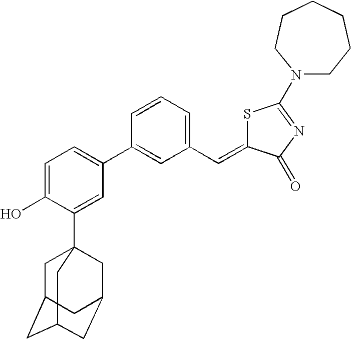

Prepared in a manner similar to that described in Example 1 using 3-(3-adamantan-1-yl-4-hydroxy-phenyl) benzaldehyde (example 2a), rhodanine and hexamethyleneimine, mp 321-324° C. ¹H NMR (300 MHz, DMSO-d₆): δ 1.55 (brs, 4 H), 1.70-1.85 (m, 10 H), 2.05 (s, 3 H), 2.15 (s, 6 H), 3.70 (t, J=6.0 Hz, 2 H), 3.88 (t, J=6.0 Hz, 2 H), 6.88 (d, J=8.1 Hz, 1 H), 7.35-7.43 (m, 2 H), 7.50-7.54 (m, 2 H), 7.60-7.67 (m, 1 H), 7.73 (s, 1 H), 7.83 (s, 1 H), 9.56 (s, 1 H). MS: Expected: 512; Found: 513 (M+H); Expected: 512; Found: 511 (M−H).

### Example 16

### 5-[3-(3-Adamantan-1-yl-4-hydroxy-phenyl) benzylidene]-2-azocan-1-yl-thiazol-4-one

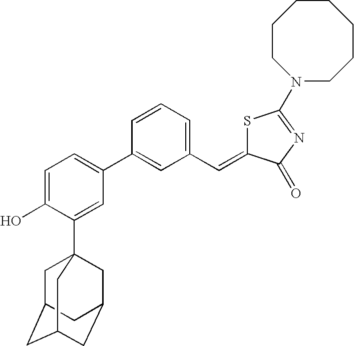

Prepared in a manner similar to that described in Example 1 using 3-(3-adamantan-1-yl-4-hydroxy-phenyl) benzaldehyde (example 2a), rhodanine and heptamethyleneimine, mp 284-286° C. ¹H NMR (300 MHz, DMSO-d₆): δ 1.36-1.64 (m, 6 H), 1.70-1.90 (m, 10 H), 2.03 (s, 3 H), 2.13 (s, 6 H), 3.65 (t, J=6.0 Hz, 2 H), 3.80 (t, J=6.0 Hz, 2 H), 6.86 (d, J=8.4 Hz, 1 H), 7.32-7.42 (m, 2 H), 7.51(d, J=4.2 Hz, 2 H), 7.60-7.675(m, 1 H), 7.71 (s, 1 H), 7.80 (s, 1 H), 9.54 (s, 1 H). MS: Expected: 526; Found: 527 (M+H); Expected: 526; Found: 525 (M−H).

### Example 18

### 5-[3-(2-Hydroxy-3-nitro-5-adamantan-1-yl-phenyl) benzylidene]-2-piperidin-1-yl-thiazol-4-one

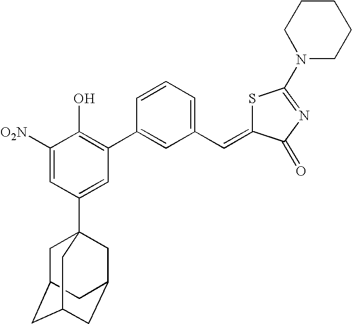

Prepared in a manner similar to that described in Example 1 using 3-(2-hydroxy-3-nitro-5-adamantan-1-yl-phenyl) benzaldehyde, rhodanine and piperidine, mp 209-211° C. ¹H NMR (300 MHz, CDCl₃): δ 1.77 (brm, 6 H), 1.94 (brd, 6 H), 2.14 (brs, 3 H), 3.57 (brs, 2 H), 4.03 (m, 2 H), 7.5-7.62 (m, 3 H), 7.70 (d, J=2.4 Hz, 1 H), 7.72 (s, 1 H), 7.86 (s, 1 H), 8.09 (d, J=2.4 Hz, 1 H), 11.02 (s, 1 H).

The intermediate 3-(2-hydroxy-3-nitro-5-adamantan-1-yl-phenyl) benzaldehyde was prepared as follows:

### a. 3-(2-hydroxy-3-nitro-5adamantan-1-yl-phenyl)

Prepared in a similar manner to that described in Example 1b, using 4-Adamantan-1-yl-2-bromo-6-nitro-phenol.

### b. 4-Adamantan-1-yl-2-bromo-6-nitro-phenol

To a solution of 4-Adamantan-1-yl-2-bromo-phenol (10 g, 32.5 mmol) in CH₂Cl₂ (500 mL) cooled to 0° C. under an atmosphere of argon was added dropwise nitronium tetrafluoroborate (81.4 mL of 0.5M in sulfolane, 163 mmol) over 1 hour. The mixture was poured into H₂O, extracted with CH₂Cl₂, and evaporated. Water was added to the residue, and then the mixture was filtered to give 10.4 g (91%) of 4-Adamantan-1-yl-2-bromo-6-nitro-phenol as a yellow powder.

### c. 4-Adamantan-1-yl-2-bromo-phenol

Prepared in a similar manner to that described in Example 1d, using 2-bromophenol and 1-adamantanol.

### Example 19

### 5-[3-(3-Adamantan-1-yl-4-hydroxy-5-fluoro-phenyl) benzylidene]-2-[4-(3-trifluoromethyl-phenyl)-piperazon-1-yl]-thiazol-4-one

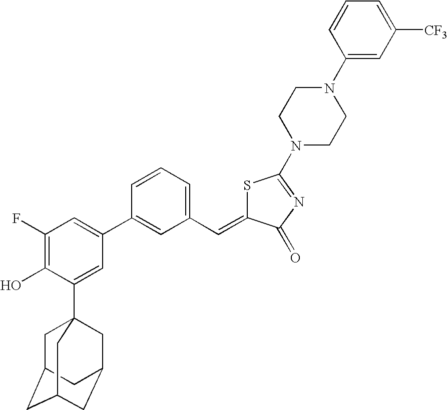

Prepared in a manner similar to that described in Example 1 using 3-(3-adamantan-1-yl-4-hydroxy-5-fluoro-phenyl) benzaldehyde (example 1a), rhodanine and 4-(3-trifluoromethylphenyl)piperazine, mp 291-293° C. ¹H NMR (300 MHz, DMSO-d₆): δ 1H NMR (300 MHz; in ppm, DMSO-d6): δ 1.74 (s, 6H), 2.05 (s, 3H), 2.14 (s, 6H), 3.42-3.46 (m, 4H), 3.77-3.81 (m, 2 H, 4.04-4.08 (m, 2 H, 7.12 (d, J=7.8 Hz, 1 H, H, 7.88 (s, 1 H, 9.59 (d, J=2.7 Hz, 1 H. MS: Expected: 661; Found: 662 (M+H); Expected: 661; Found: 660 (M−H).

### Example 20

### 5-[3-(3-Adamantan-1-yl-4-hydroxy-5-fluoro-phenyl) benzylidene]-2[benzyl-(2-dimethylamino-ethyl)-amino]-thiazol-4-one

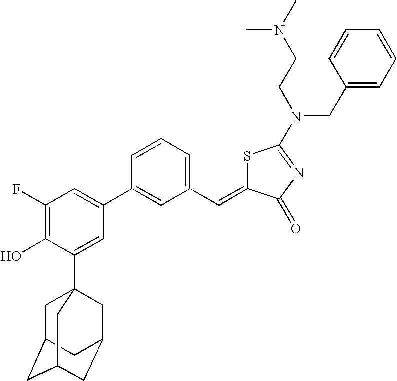

Prepared in a manner similar to that described in Example 1 using 3-(3-adamantan-1yl-4-hydroxy-5-fluoro-phenyl) benzaldehyde (example 1a), rhodanine and N′-benzyl-N,N-dimethylethylendiamine. mp 242-246° C. ¹H NMR (300 MHz, DMSO-d6): δ 1.67-1.74 (2 signals integrating to 6H), 1.98-2.11 (2 signals integrating to 9H), 2.81-2.87 (2 signals integrating to 6H), 3.43 (t, J=6.3 Hz, 2 H), 3.99 (t, J=6.3 Hz, 2 H, 4.86-5.01 (2 signals integrating to 2 H, 7.23 (s, 1 H, 7.36-7.43 (m, 6 H), 7.51-7.58 (m, 2 H), 7.66-7.74 (m, 1 H), 7.84-7.93 (m, 2 H), 9.62 (s, 1 H). MS: Expected: 609; Found: 610 (M+H); Expected: 609; Found: 608 (M−H).

### Example 21

### 5-[3-(3-Adamantan-1-yl-4-hydroxy-5-fluoro-phenyl)benzylidene]-2benzylamino-thiazol-2-benzylamino-thiazol-4-one

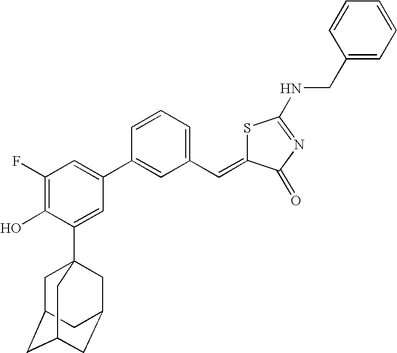

Prepared in a manner similar to that described in Example 1 using 3-(3-adamantan-1-yl-4-hydroxy-5-fluoro-phenyl)benzaldehyde (example 1a), rhodanine and benzylamine ¹H NMR (300 MHz; in ppm, DMSO-d₆): δ 1.73 (s, 6 H), 2.05 (s, 3 H), 2.13 (s, 6 H), 4.74 (d, J=5.7 Hz, 2 H), 7.22 (s, 1 H), 7.27-7.43 (m, 6 H), 7.46-7.58 (m, , 2 H), 7.66 (d, J=7.5 Hz, 1 H), 7.72 (s, 1 H), 7.82 (s, 1 H), 9.58 (d, J=2.7 Hz, 1 H, 10.09 (t, J=6.0 Hz, 1 H. MS: Expected: 538; Found: 539 (M+H); Expected: 538; Found: 537 (M−H).

### Example 22

### 5-[3-(5-Adamantan-1-yl-[1,3,4]-oxadiazol-2-yl)benzylidene]-2-piperidin-1-yl-thiazol-4-one

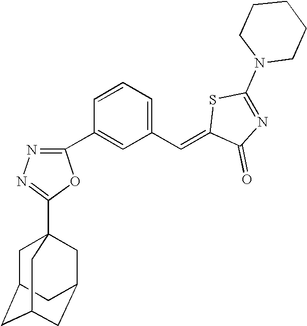

Prepared in a manner similar to that described in Example 1 using 3-(5-adamantan-1-yl-[1,3,4]-oxadiazol-2-yl)benzaldehyde, rhodanine and piperidine, mp 219-221° C. ¹H NMR (300 MHz, DMSO-d₆): δ 1.65-1.82 (m, 12 H), 2.11 (brs, 9 H), 3.60-3.70 (m, 2 H), 3.90-4.00 (m, 2 H), 7.68 (t, J=7.8 Hz, 1 H), 7.74 (s, 1 H), 7.81 (doublet of multiplets, 1 H), 8.00 (dt, J=7.8 Hz, J₂=1.2 Hz, 1 H), 8.22-8.25 (m, 1 H).

The intermediate 3-(5-adamantan-1-yl-[1,3,4]-oxadiazol-2-yl)benzaldehyde was prepared as followed:

### a. 3-(5-adamantan-1-yl-[1,3,4]-oxadiazol-2-yl)benzaldehyde

To a solution of 2-adamantan-1-yl-5-(bromo-phenyl)-[1,3,4]oxadiazole (1.55 g, 4.31 mmol) in anhydrous THF (30 mL) cooled to −78° C. was added dropwise under argon n-BuLi (2.5 M solution in hexane, 4.73 mmol, 1.90 mL. After 15 minutes DMF (0.67 mL) was added and the solution stirred for 15 min then quenched with 1N HCl and extracted with ethylacetate. The organic layer was further washed with water and brine, dried over magnesium sulfate, filtered and evaporated. The residue was chromatographed on silica gel (eluent 20% ethylacetate in hexane) to give 1.3 g (68%) of 3-(5-adamantan-1-yl-[1,3,4]-oxadiazol-2-yl)benzaldehyde.

### b. 2-adamantan-1-yl-5-(bromo-phenyl)-[1,3,4]oxadiazole

To a solution of 4-bromo-benzoic acid N′-(adamantan-1-carbonyl)-hydrazide (2.20 g, 5.83 mmol) in pyridine (30 mL) was added under argon p-toluenesulfonic chloride (2.22 g, 11.66 mmol) and the solution was refluxed for 24 hrs. After cooling to room temperature the solvent was removed under reduced pressure. The residue was dissolved in ethylacetate and washed successively with water, aqueous ammonium chloride and brine, dried over magnesium sulfate, filtered and evaporated to give 1.83 g (96%) of 2-adamtantan-1-yl-5-(bromo-phenyl)-[1,3,4]oxadiazole.

### c. 4-bromo-benzoic acid N′-(adamantan-1-carbonyl)-hydrazide

To a solution of 3-bromobenzoic hydrazide (4.87 g, 22.67 mmol) in dichloromethane (100 mL) was added triethylamine (4.74 mL, 34.01 mmol) and the solution cooled to 0° C. 1-Adamantanecarbonyl chloride (4.50 g, 22.67 mmol) dissolved in dichloromethane (25 mL) was dropwise added to the reaction mixture. The solution was allowed to slowly warmed to room temperature. The solution was filtered and washed with water and evaporated to give 6.65 g (78%) of 4-bromo-benzoic acid N′-(adamantan-1-carbonyl)-hydrazide.

### Example 24

### 5-[3-(3-Adamantan-1-yl-4-hydroxy-5-fluoro-phenyl)benzylidene]-2-(2-morpholino-1-yl-ethylamino)-thiazol-4-one

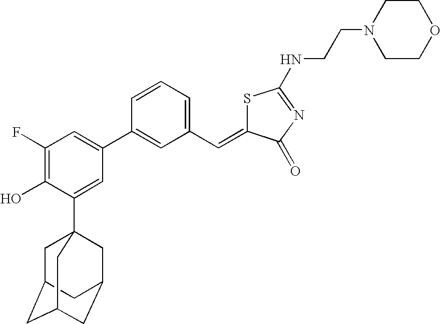

Prepared in a manner similar to that described in Example 1 using 3-(3-adamantan-1-yl-4-hydroxy-5-fluoro-phenyl)benzaldehyde (example 1a), rhodanine and 4-(2-aminoethyl)morpholine mp 180-193° C. ¹H NMR (300 MHz; in ppm, DMSO-d₆): δ 1.74 (s, 6 H), 2.05 (s, 3 H), 2.14 (s, 6 H), 3.20-3.40 (m, 6 H), 3.74-3.85 (m, 6 H), 7.22 (s, 1 H), 7.38 (dd, J₁=11.7 Hz, J₂=2.1 Hz, 1 H), 7.48 (d, J=7.8 Hz, 1), 7.56 (t, J=7.8 Hz, 1 H), 7.67 (d, J=7.8 Hz, 1 H), 7.73 (s, 1 H), 7.82 (s, 1 H), 9.60 (d, 2.7 Hz, 1 H), 9.98 (s, 1 H). MS: Expected: 561; Found: 562 (M+H); Expected: 561; Found: 560 (M−H).

### Example 26

### 5-[3-(3-Benzoyl-4-hydroxy-phenyl)benzylidene]-2-piperidin-1-yl-thiazol-4-one

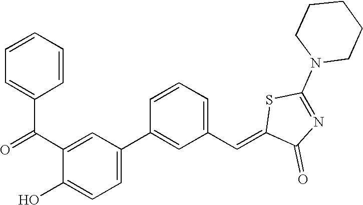

Prepared in a manner similar to that described in Example 1 using 3-(3-benzoyl-4-hydroxy-phenyl)benzaldehyde, rhodanine and piperidine, mp 135-138° C. ¹H NMR (300 MHz, DMSO-d₆): δ 1.64 (broad s, 6H), 3.56 (broad s, 2 H, 3.88 (broad s, 2 H, 7.10 (d, J=8.4 Hz, 1 H, 7.50-7.55 (m, 4H), 7.61-7.68 (m, 4H), 7.76-7.83 (m, 4H), 10.51 (s, 1 H. MS: Expected: 468; Found: 469 (M+H); Expected: 468; Found: 467 (M−H).

The intermediate 3-(3-benzoyl-4-hydroxy-phenyl)benzaldehyde was prepared as followed:

### a. 3-(3-benzoyl-4-hydroxy-phenyl)benzaldehyde

5-Bromo-2-hydroxy-benzophenone (1.0 g, 3.61 mmol), 3-formylphenylboronic acid (1.2 eq., 4.33 mmol, 650 mg) and sodium carbonate (3 eq., 10.83 mmol, 1.15 g) were added to 15 ml of toluene/ethanol/water 8:2:1 and the solution was degassed with argon gas for 20 min. Tetrakis(triphenylphosphine)palladium (0) (0.1 eq., 0.36 mmol, 416 mg) was added and the mixture was refluxed overnight. The mixture was separated between water and ethyl acetate. The organic phase was washed with brine, dried with sodium sulfate, filtered and evaporated. The residue was chromatographed on silica gel (hexane/ethyl acetate, 85:15) to give 0.70 g (45%) of 3-(3-benzoyl-4-hydroxy-phenyl)benzaldehyde. ¹H-NMR (300 MHz, CDCl₃): δ 7.20 (d, 1 H, J=8.4 Hz), 7.52-7.66 (m, 4 H), 7.70-7.84 (m, 6 H), 7.96 (t, 1 H, J=1.8 Hz), 10.05 (s, 1 H), 12.05 (s, 1 H).

### Example 27

### 5-[5-(3-Adamantan-1-yl-4-hydroxy-phenyl)-6-methoxy-pyridin-3-yl]-2-morpholin-4-yl-thiazol-4-one

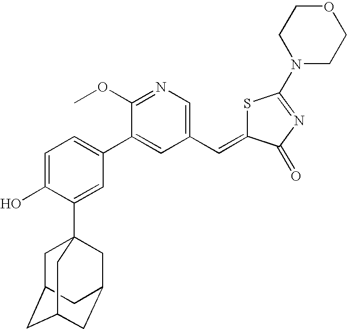

Prepared in a manner similar to that described in Example 1 using 5-(3-adamantan-1-yl-4-hydroxy-phenyl)-6-methoxy-pyridyl-3-carboxaldehyde, rhodanine and morpholine, mp 230-231° C. ¹H NMR (300 MHz, DMSO-d₆): δ 1.73 (brs, 6 H), 2.03 (brs, 3 H), 2.11 (brs, 6 H), 3.66 (brs, 2 H), 3.73 (brs, 4 H), 3.93 (s, 3 H), 3.94 (s, 2 H), 6.85 (d, J=7.8 Hz, 1 H), 7.29 (d, J=7.8 Hz, 1 H), 7.31 (s, 1 H), 7.87 (d, J=2.7 Hz, 1 H), 8.40 (d, J=2.4 Hz, 1 H), 9.60 (s, 1 H).

The intermediate 5-(3-adamantan-1-yl-4-hydroxy-phenyl)-6-methoxy-pyridyl-3-carboxaldehyde was prepared as followed:

### a. 5-(3-adamantan-1-yl-4-hydroxy-phenyl)-6-methoxy-pyridyl-3-carboxaldehyde

Prepared in a similar manner to that used in Example 1a, using 5-[3-Adamantan-1-yl-4-(-tert-butyldimethylsilanoxy)-phenyl]-6-methoxy-pyridine-3-carbaldehyde. ¹H NMR (300 MHz, CDCl₃): 1.78 (br. s, 6 H), 2.08 (br. s, 3 H), 2.17 (br. s, 6 H), 4.07 (s, 3 H), 6.87 (d, J=8.4 Hz, 1 H), 7.28 (dd, J₁=2.1 Hz, J₂=8.4 Hz, 1 H), 7.35 (m, 2 H), 8.05 (d, J=2.4 Hz, 1 H), 8.56 (d, J=2.7 Hz, 1 H), 8.58 (s, 1 H), 10.00 (s, 1 H).

### b. 5-[3-Adamantan-1-yl-4-(tert-butyldimethylsilanoxy)-phenyl]-6-methoxy-pyridine-3-carbaldehyde

A mixture of 3-Adamantan-1-yl-4-(tert-butyldimethylsilanyloxy)-boronic acid (20 g, 52.7 mmol), 5-Bromo-6-methoxy-pyridine-3-carbaldehyde (9.5 g, 44 mmol) and sodium carbonate (14 g, 132 mmol) in toluene: ethanol (4:1, 300 mL) and water (30 mL) was degassed with argon for 45 minutes. Tetrakis(triphenylphosphine)palladium(0) (1.5 g, 1.32 mmol) was added and the mixture heated at reflux under argon for 16 hours. The solution was cooled to room temperature, diluted with ethyl acetate and washed successively with water and brine, dried over anhydrous magnesium sulfate, filtered and evaporated. The residue was purified on silica gel (eluent: hexane: ethyl acetate, 92:8) to give 18.1 g (86%) 5-[3-Adamantan-1-yl-4-(tert-butyldimethylsilanoxy)-phenyl]-6-methoxy-pyridine-3-carbaldehyde.

### c. 3-Adamantan-1-yl-4-(tert-butyldimethylsilanyloxy)-boronic acid

To a solution of n-BuLi (142 mL of 2.5 M, 356 mmol), in anhydrous THF (500 mL) cooled to −78 C under an atmosphere of argon was added dropwise a solution of 3-[3-Adamantan-1-yl-4-(t-butyldimethylsilyloxy)]-1-bromobenzene (example 2c) (100 g, 237 mmol) in anhydrous THF (500 mL) over 1 h. Mixture stirred at −78 C for 1 h, then triisopropyl borate (164 mL, 712 mmol) was added dropwise over 40 min at −78 C. Warmed to 0 C, then mixture was quenched with aqueous NH₄Cl, extracted with ethyl acetate (twice). The combined organic layers were washed with brine, dried over magnesium sulfate, filtered, and evaporated to give 77 g (84%) of 3-Adamantan-1-yl-4-(tert-butyldimethylsilanyloxy)-boronic acid as a white powder. Used directly in next step

### Example 29

### 5-[3-(3-Adamantan-1-yl-4-hydroxy-5-benzoyl-phenyl)benzylidene]-2-piperidin-1-yl-thiazol-4-one

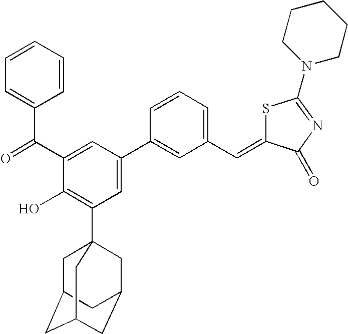

Prepared in a manner similar to that described in Example 1 using 3-(3-adamantan-1-yl-4-hydroxy-5-benzoyl-phenyl)benzaldehyde, rhodanine and piperidine, mp 238° C. ¹H NMR (300 MHz, DMSO-d₆): δ 1.68 (brs, 6 H), 1.79 (brs, 6 H), 2.10 (brs, 3 H), 2.23 (brs, 6 H), 3.59 (brt, 2 H), 3.91 (brt, 2 H), 7.53-7.80 (m, 12 H), 12.72 (s, 1 H).

The intermediate 3-(3-adamantan-1-yl-4-hydroxy-5-benzoyl-phenyl)benzaldehyde was prepared as followed:

### a. 3-(3-adamantan-1-yl-4-hydroxy-5-benzoyl-phenyl)benzaldehyde

3′-Adamantan-1-yl-5′-benzoyl-4′-methoxymethoxy-biphenyl-3-carbaldehyde (870 mg, 1.81 mmol) was dissolved in tetrahydofuran/methanol (1:1, 20 mL). 10% Sulfuric acid (5 ml) was added and the reaction was set to reflux. After 2 hrs the mixture was poured into ice/water, solid sodium bicarbonate was added and the mixture was extracted with ethyl acetate. The combined organic phases were washed with brine, dried with sodium sulfate, filtered and evaporated.

### b. 3′-Adamantan-1-yl-5′-benzoyl-4′-methoxymethoxy-biphenyl-3-carbaldehyde

3-Adamantan-1-yl-5-bromo-2-methoxymethoxy-benzophenone (1.0 g, 2.20 mmol), 3-formylphenylboronic acid (2.64 mmol, 395 mg) and sodium carbonate (6.6 mmol, 700 mg) were added to 15mL of toluene/ethanol/water 8:2:1 and the solution degassed with argon gas for 20 min. Tetrakis(triphenylphosphine)palladium (0) (0.1 eq., 0.22 mmol, 254 mg) was added and the mixture was refluxed overnight. The mixture was separated between water and ethyl acetate. The organic phase was washed with brine, dried with sodium sulfate, filtered and evaporated. The residue was chromatographed on silica gel (hexane/ethyl acetate, 85:15) to give 0.87 g (82%) of 3′-Adamantan-1-yl-5′-benzoyl-4′-methoxymethoxy-biphenyl-3-carbaldehyde.

### c. 3-Adamantan-1-yl-5-bromo-2-methoxymethoxy-benzophenone

3-Adamantan-1-yl-5-bromo-2-hydroxy-benzophenone (6.31 g, 15.34 mmol) was dissolved in anhydrous dichloromethane (100 mL). 4-Dimethylaminopyridine (1.5 mmol, 190 mg) and N,N-diisopropylethylamine (92 mmol, 16 mL) were added followed by methoxymethyl chloride (46 mmol, 3.5 ml). The mixture was stirred overnight at room temperature. Ethyl acetate was added to the mixture and the organic phase was washed with 0.5N HCl, then saturated sodium bicarbonate followed by brine. The organic phase was dried with sodium sulfate, filtered and evaporated to give 6.65 g (92 %) of 3-Adamantan-1-yl-5-bromo-2-methoxymethoxy-benzophenone

### d. 3-Adamantan-1-yl-5-bromo-2-hydroxy-benzophenone

5-Bromo-2-hydroxy-benzophenone (10.0 g, 36.09 mmol) and adamantane-1-ol (5.50 g, 36.09 mmol) were dissolved in dichloromethane (150 mL). Conc. sulfuric acid (1 eq., 2 mL) was added and the reaction was stirred under reflux for 2 days. Solid sodium bicarbonate was added, and the mixture was separated between water and dichloromethane. The organic phase was dried with sodium sulfate, filtered and evaporated. The crude material was purified by silica gel chromatography (hexane/ethyl acetate, 99:1) to give 6.31 g (43%) of 3-Adamantan-1-yl-5-bromo-2-hydroxy-benzophenone.

### Example 31

### 5-[3-(3-Adamantan-1-yl-4-hydroxy-5-fluoro-phenyl)benzylidene]-2piperazin-1-yl-thiazol-4-one

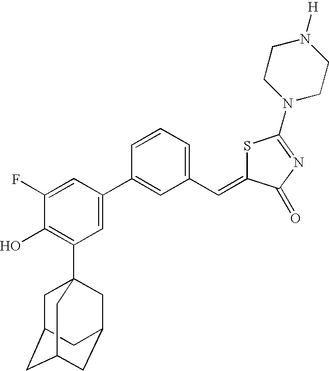

Prepared in a manner similar to that described in Example 1 using 3-(3-adamantan-1-yl-4-hydroxy-5-fluoro-phenyl)benzaldehyde (example 1a), rhodanine and piperazine, mp 230-233° C. ¹H NMR (300 MHz, DMSO-d₆): δ 1.73 (s, 6 H), 2.05 (s, 3 H), 2.14 (s, 6 H), 3.05-3.09 (m, 4 H), 3.70-3.74 (m, 2 H), 3.96-4.02 (m, 2 H), 7.23 (s, 1 H), 7.42 (dd, J₁=1.2 Hz, J₂=11.7 Hz, 1 H), 7.53 (d, J=4.8 Hz, 1 H), 7.54 (s, 1 H), 7.66-7.70 (m, 1 H), 7.77 (s, 1 H), 7.87 (s, 1 H), 9.61 (brs, 1 H). MS: Expected: 517; Found: 518 (M+H); Expected: 517; Found: 516 (M−H).

### Example 33

### 5-[3-(3-Adamantan-1-yl-4-hydroxy-6-methyl-phenyl)benzylidene]-2-morpholin-4-yl-thiazol-4-one

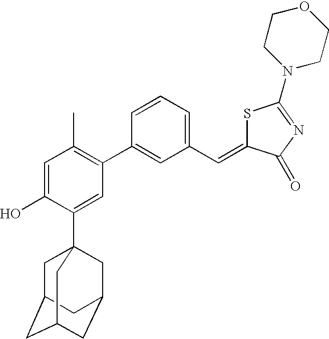

Prepared in a manner similar to that described in Example 1 using 3-(3-adamantan-1-yl-4-hydroxy-6-methyl-phenyl)benzaldehyde, rhodanine and morpholine, mp 327-329° C. ¹H NMR (300 MHz, DMSO-d₆): δ 1.68 (s, 6 H), 1.98 (s, 3 H), 2.05 (s, 6 H), 2.15 (s, 3 H), 3.60-3.78 (m, 6 H), 3.88-3.92 (m, 2 H), 6.68 (s, 1 H), 6.90 (s, 1 H), 7.35 (dd, J₁=7.2 Hz, J₂=1.8 Hz, 1 H), 7.46-7.55 (m, 3 H), 7.70 (d, J=1.5 Hz, 1 H), 9.32 (d, J=1.8 Hz, 1 H). MS: Expected: 514; Found: 515 (M+H); Expected: 514; Found: 513 (M−H).

### Example 35

### 5-[3-(3-Adamantan-1-yl-5-methoxy-phenyl)benzylidene]-2-morpholin-4-yl-thiazol-4-one

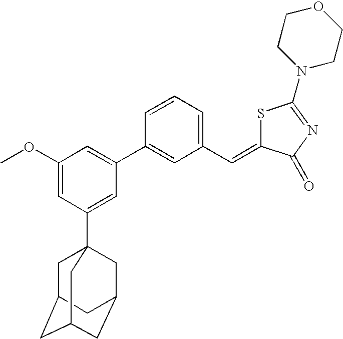

Prepared in a manner similar to that described in Example 1 using 3-(3-adamantan-1-yl-5-methoxy-phenyl)benzaldehyde, rhodanine and morpholine, mp 234-238° C. ¹H NMR (300 MHz, CDCl₃): δ 1.80 (brm, 6 H), 1.98 (brd, 6 H), 2.12 (brs, 3 H), 3.63 (t, J=5.1 Hz, 2 H), 3.83 (q, J=5.7 Hz, 4 H), 3.88 (s, 3 H), 4.10 (t, J=5.4 Hz, 2 H), 6.95 (m, 2 H), 7.20 (dd, J=1.5 Hz, 1 H), 7.50 (d, J=0.9 Hz, 1 H), 7.51 (d, J=1.5 Hz, 1 H), 7.60 (m, 1 H), 7.75 (s, 1 H), 7.90 (s, 1 H).

### Example 36

### 5-[6-(3-Adamantan-1-yl-4-t-butyldimethylsilyloxy-phenyl)-pyridin-2-yl]-2-morpholin-4-yl-thiazol-4-one

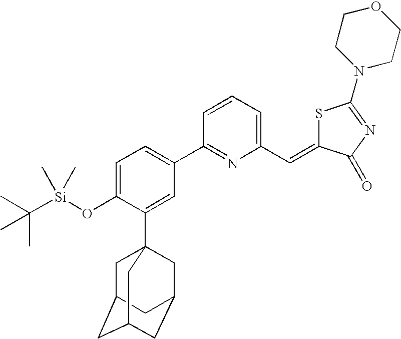

Prepared in a manner similar to that described in Example 1 using 6-(3-adamantan-1-yl-4-t-butyldimethylsilyloxy-phenyl)-pyridin-2-carboxaldehyde, rhodanine and morpholine, mp 265-268° C. ¹H NMR (300 MHz, DMSO-d₆): δ 0.39 (s, 6 H), 1.05 (s, 9 H), 1.75 (s, 6 H), 2.07 (s, 3 H), 2.16 (s, 6 H), 3.60-3.80 (m, 6 H), 3.90-3.98 (m, 2 H), 6.97 (d, J=8.4 Hz, 1 H), 7.69 (d, J=7.8 Hz, 1 H), 7.75 (s, 1 H), 7.79-7.85 (m, 2 H), 7.94 (t, J=7.8 Hz, 1 H), 8.09 (s, 1 H).

The intermediate 6-(3-adamantan-1-yl-4-t-butyldimethylsilyloxy-phenyl)-pyridin-2-carboxaldehyde was prepared in a similar manner as described in example 2b using 3-(3-Adamantan-1-yl-4-(t-butyldimethylsilyloxy)-1-phenylboronic acid (example 27c) and 6-bromopyridine-2-carboxyaldehyde.

### Example 38

### 5-[3-(3-Adamantan-1-yl-phenyl)-phenyl-3-yl]-2-morpholin-4-yl-thiazol-4-one

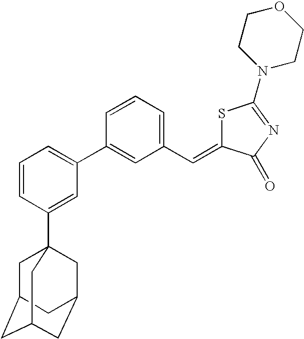

Prepared in a manner similar to that described in Example 1 using 3-(3-adamantan-1-yl-phenyl)benzaldehyde, rhodanine and morpholine, mp 252-254° C. ¹H NMR (300 MHz, DMSO-d₆): δ 1.77 (brs, 6 H), 1.95 (brd, 6 H), 2.08 (brs, 3 H), 3.69 (m, 2 H), 3.74 (m, 4 H), 3.94 (m, 2 H), 7.38-7.48 (m, 2 H), 7.52 (dt, J=6.9, 2.1 Hz, 1 H), 7.61(m, 2 H), 7.67 (s, 1 H), 7.74 (m, 1 H), 7.80 (s, 1 H), 7.94 (s, 1 H).

### Example 39

### 5-[6-(3-Adamantan-1-yl-4-hydroxy-phenyl)-pyridin-2-yl]-2-morpholin-4-yl-thiazol-1-one

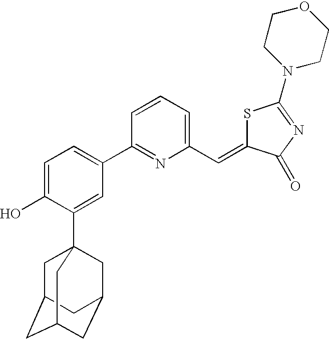

Prepared in a manner similar to that described in Example 1 using 6-(3-adamantan-1-yl-4-hydroxy-phenyl)-pyridin-2-carboxaldehyde, rhodanine and morpholine, mp 339-343° C. ¹H NMR (300 MHz, DMSO-d₆): δ 1.74 (s, 6 H), 2.06 (s, 3 H), 2.17 (s, 6 H), 3.60-3.80 (m, 6 H), 3.90-3.98 (m, 2 H), 6.92 (d, J=8.4 Hz, 1 H), 7.65 (d, J=7.5 Hz, 1 H), 7.71-7.85 (m, 3 H), 7.91 (t, J=7.8 Hz, 1 H), 8.00 (d, J=1.8 Hz, 1 H), 9.77 (s, 1 H). MS: Expected: 501; Found: 502 (M+H); Expected: 501; Found: 500 (M−H).

### Example 40

### 5-[6-(3-Adamantan-1-yl-4-hydroxy-phenyl)-pyridin-3-yl]-2-morpholin-4-yl-thiazol-1-one

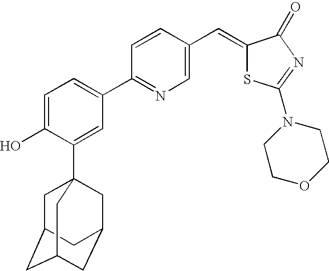

Prepared in a manner similar to that described in Example 1 using 6-(3-adamantan-1-yl-4-hydroxy-phenyl)-pyridin-3-carboxaldehyde, rhodanine and morpholine, mp 313-316° C. ¹H NMR (300 MHz, DMSO-d₆): δ 1.76 (s, 6 H), 2.06 (s, 3 H), 2.13 (s, 6 H), 3.69-3.80 (m, 6 H), 3.90-3.97 (m, 2 H), 6.89 (d, J=8.1 Hz, 1 H), 7.70 (s, 1 H), 7.80 (dd, J₁=8.4 Hz, J₂=1.8 Hz, 1 H), 7.98 (d, J=1.5 Hz, 2 H), 8.00 (d, J=2.1 Hz, 1 H), 8.86 (s, 1 H), 9.83 (s, 1 H). MS: Expected: 501; Found: 502 (M+H); Expected: 501; Found: 500 (M−H).

The intermediate 6-(3-adamantan-1-yl-4-hydroxy-phenyl)-pyridin-3-carboxaldehyde was prepared in a similar manner to that described in example 27 using 6-bromo-pyridine-3-carbaldehyde in step b.

### Example 41

### 5-[4-(2-Adamantan-1-yl-pyrimidin-4-yl)-benzylidene]-2-morpholin-4-yl-thiazol-4-one

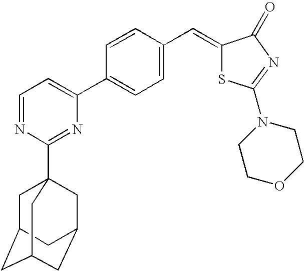

Prepared in a manner similar to that described in Example 1 using 4-(2-adamantan-1-yl-pyrimidin-4-yl)benzaldehyde, rhodanine and morpholine, mp 283-285° C. ¹H NMR (300 MHz, DMSO-d₆): δ 1.78 (s, 6 H), 2.09 (s, 9 H), 3.67-3.81 (m, 6 H), 3.92-3.97 (m, 2 H), 7.74 (s, 1 H), 7.82 (d, J=8.1, 2 H), 7.94 (d, J=5.4, 1 H), 8.37 (d, J=8.1, 2 H), 8.86 (d, J=5.4, 1 H). MS: Expected: 486; Found: 487 (M+H).

The intermediate 4-(2-adamantan-1-yl-pyrimidin-4-yl)benzaldehyde was prepared as followed:

### a. 4-(2-adamantan-1-yl-pyrimidin-4-yl)benzaldehyde

To a solution of 4-(2-adamantan-1-yl-pyrimidin-4-yl)-bromobenzene (4.47 g, 12.13 mmol) in THF (100 mL) cooled to −78° C. was added under argon n-BuLi (2.5 M in hexane, 5.34 mL, 13.34 mmol). After 15 minutes DMF was added dropwise and the reaction allowed warm to room temperature and stirred for 1 hr. The reaction was quenched with 1N HCl (20 mL) and extracted with ethylacetate. The organic layer was further washed with water and brine, dried over magnesium sulfate, filtered and evaporated. The residue was chromatographed on silica gel to give 1.85 g (48%) of 4-(2-adamantan-1-yl-pyrimidin-4-yl)benzaldehyde.

### b. 4-(2-adamantan-1-yl-pyrimidin-4-yl)-bromobenzene

To a solution of adamantane-1-carboxamidine (3.93 g, 22.04 mmol), 1-(4-bromophenyl)-3-(dimethylamino)-2-propene-1-one (5.60 g, 22.04 mmol) in ethanol (125 mL) was added NaOEt (3.74 g, 55.1 mmol) and the reaction mixture refluxed for 48 hrs. The solution was diluted with ethylacetate and washed successively with aqueous ammonium chloride and brine, dried over magnesium sulfate, filtered and evaporated. The residue was chromatographed on silica gel to afford 4.5 g (55%) of 4-(2-adamantan-1-yl-pyrimidin-4-yl)-bromobenzene.

### Example 42

### 5-[3-(3-Adamantan-1-yl-5-hydroxy-phenyl)benzylidene]-2-morpholin-4-yl-thiazol-4-one

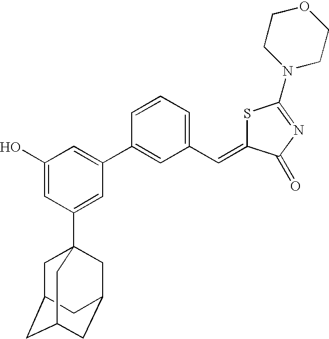

To a solution of 5-[3-(3-Adamantan-1-yl-5-methoxy-phenyl)benzylidene]-2-morpholin-4-yl-thiazol-4-one (example 35)(87 mg, 0.169 mmol) in CH₂Cl₂ (10 mL) cooled to −78 C under an atmosphere of argon was added dropwise a solution of BBr₃ (0.128 mL, 1.35 mmol) in CH₂Cl₂ (10 mL) over 0.5 h. Mixture was stirred at room temperature for 16 h, then poured into H₂O and extracted with CH₂Cl₂ (twice). The combined organic layers were washed successively with a saturated solution of NaHCO₃ and brine, dried over magnesium sulfate, and evaporated. The residue was purified by reverse phase HPLC (65% B isocratic, 35% A; B=30% THF, 70% Acetonitrile, 0.02% TFA, A=H₂O, 0.02% TFA) to give 46 mg (54%) 5-(3′-Adamatan-1-yl-5′-hydroxy-biphenyl-3-ylmethylene)-2-morpholin-4-yl-thiazol-4-one as a white powder. mp 328-331° C. ¹H NMR (300 MHz, DMSO-d₆): δ 1.75 (brs, 6 H), 1.90 (brd, 6 H), 2.07 (brs, 3 H), 3.68 (m, 2 H), 3.74 (m, 4 H), 3.94 (m, 2 H), 6.79 (t, J=1.8 Hz, 1 H), 6.87 (t, J=1.8 Hz, 1 H), 7.10 (t, J=1.8 Hz, 1 H), 7.58 (m, 2 H), 7.65 (m, 1 H), 7.78 (s, 1 H), 7.86 (s, 1 H), 9.45 (s, 1 H).

### Example 43

### 5-[3-(3-Adamantan-1-yl-4-hydroxy-5-fluoro-phenyl)benzyl]-2-morpholin-4-yl-thiazol-4-one

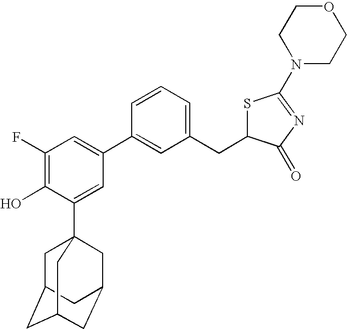

A solution of toluene (80 mL), morpholine (0.31 mL, 3.53 mmol), acetic acid (0.11 mL, 1.93 mmol) and 5-(3′-Adamantan-1-yl-5′-fluoro-4′-hydroxy-biphenyl-3-ylmethyl)-2-thioxo-thiazol-4-one (1.5 g, 3.21 mmol) was heated at reflux for 16 hours under an argon atmosphere. The mixture was cooled to 0° C. and filtered to give 1.06 g (64%) of 5-(3′-Adamantan-1-yl-5′-fluoro-4′-hydroxy-biphenyl-3-methyl)-2-morpholin-4-yl-thiazol-4-one as a white powder. mp 227-229° C. ¹H NMR (300 MHz, CDCl₃): δ 1.80 (s, 6 H), 2.10 (s, 3 H), 2.17 (s, 6 H), 3.01 (dd, J₁=10.8 Hz, J₂=14.1 Hz, 1 H), 3.48 (t, J=5.1 Hz, 2 H), 3.67-3.75 (m, 5 H), 3.94-4.00 (m, 2 H), 4.53, (dd, J₁=3.3 Hz, J₂=10.8 Hz, 1 H), 5.41 (d, J=6.9 Hz, 1 H), 7.14-7.22 (m, 3 H), 7.32-7.43 (m, 3 H), MS: Expected: 520; Found: 521 (M+H); Expected: 520; Found: 519 (M−H).

The intermediate 5-(3′-Adamantan-1-yl-5′-fluoro-4′-hydroxy-biphenyl-3ylmethyl)-2-thioxo-thiazolidin-4-one was prepared as followed:

### a. 5-(3′-Adamantan-1-yl-5′-fluoro-4′-hydroxy-biphenyl-3-ylmethyl)-2-thioxo-thiazolidin-4-one

To a solution of 5-(3′-Adamantan-1-yl-5′-fluoro-4′-hydroxy-biphenyl-3-ylmethylene)-2-thioxo-thiazolidin-4-one (5 g, 10.75 mmol) in anhydrous pyridine (9.4 mL) and THF (50 mL) under an atmosphere of argon was added LiBH₄ (11.83 mL of 2 M in THF, 23.66 mmol). The resulting mixture was heated at reflux for 5 hours. The mixture was cooled, quenched by dropwise addition of 1.0 N HCl then with ethyl acetate (twice). The combined organic layers were washed successively with 1.0 N HCl, water and brine, dried over anhydrous magnesium sulfate, filtered, and evaporated. The residue was purified on silica gel (eluent: hexane: ethyl acetate, 5:1) to give 4.45 g (87%) of 5-(3′-Adamantan-1-yl-5′-fluoro-4′-hydroxy-biphenyl-3-ylmethyl)-2-thioxo-thiazolidin-4-one.

### b. 5-(3′-Adamantan-1-yl-5′-fluoro-4′-hydroxy-biphenyl-3-ylmethylene)-2thioxo-thiazolidin-4-one

A solution of anhydrous toluene (500 mL), aniline (2.0 mL, 22 mmol), acetic acid (0.69 mL, 12 mmol), 5-[3-(3-adamantan-1-yl-4-hydroxy-5-fluoro-phenyl)benzaldehyde (7.0 g, 20 mmol) (example 1a) and rhodanine (2.66 g, 20 mmol) was heated at reflux for 16 hours under an argon atmosphere. The mixture was cooled to 0° C, then filtered to give 8.2 g (89%) of 5-(3′-Adamantan-1-yl-5′-fluoro-4′-hydroxy-biphenyl-3-ylmethylene)-2-thioxo-thiazolidin-4-one as a yellow powder.

### Example 44

### 5-[6-(3-Phenyl-4-methoxy-phenyl)-pyridin-2-yl]-2-morpholin-4-yl-thiazol-4-one

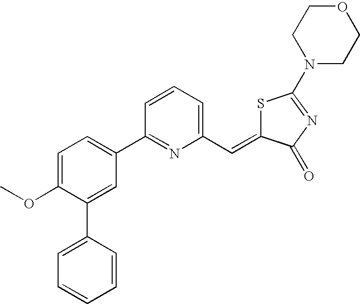

Prepared in a manner similar to that described in Example 1 using 6-(3-Phenyl-4-methoxy-phenyl)-pyridin-2-carboxaldehyde, rhodanine and morpholine, mp 214-217° C. ¹H NMR (300 MHz, DMSO-d₆): δ 3.19 (brs, 2 H), 3.51 (brs, 2 H), 3.67 (brs, 2 H), 3.83 (s, 3 H), 3.87 (brs, 2 H), 7.25 (d, J=8.7 Hz, 1 H), 7.34-7.48 (m, 3 H), 7.52 (s, 1 H), 7.54 (d, J=1.8 Hz, 1 H), 7.69 (d, J=7.5 Hz, 1 H), 7.71 (s, 1 H), 7.90-8.02 (m, 2 H), 8.12 (dd, J₁=8.7 Hz, J₂=2.4 Hz, 1 H), 8.33 (d, J=2.4 Hz, 1 H). MS: Expected: 457; Found: 458 (M+H).

### Example 45

### 5-[3-(3-Adamantan-1-yl-4-hydroxy-5-fluoro-phenyl)benzylidene]-2-(cis-2,6-dimethylmorpholin-4-yl)-thiazol-4-one

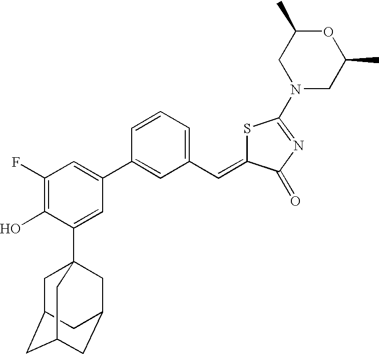

Prepared in a manner similar to that described in Example 1 using 3-(3-adamantan-1-yl-4-hydroxy-5-fluoro-phenyl)benzaldehyde, rhodanine and 2,6-dimethyl-morpholine, mp 287-289° C. ¹H NMR (300 MHz, DMSO-d₆): δ 1.12-1.18 (m, 6 H), 1.74 (s, 6 H), 2.05 (s, 3 H), 2.15 (s, 6 H), 3.60-3.77 (m, 3 H), 3.98-4.09 (m, 3 H), 7.24 (s, 1 H), 7.43 (dd, J₁=11.7 Hz, J₂=2.1 Hz, 1 H), 7.54 (d, J=7.54 Hz, 1 H), 7.55 (s, 1 H, 7.66-7.70 (m, 1 H), 7.76 (s, 1 H), 7.88 (s, 1 H), 9.60 (d, J=2.4 Hz, 1 H).

### Example 48

### 5-[3-(3-Adamantan-1-yl-4-hydroxy-phenyl)-4-methoxy-benzylidene]-2-morpholin-4-yl-thiazol-4-one

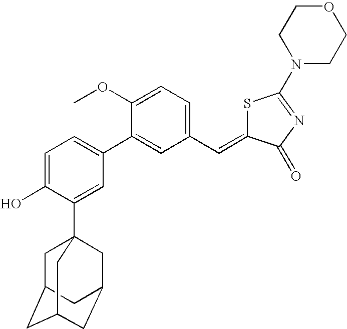

Prepared in a manner similar to that described in Example 1 using 3-(3-adamantan-1-yl-4-hydroxy-phenyl)-4-methoxy-benzaldehyde, rhodanine and morpholine, mp 339-343° C. ¹H NMR (300 MHz, DMSO-d₆): δ 1.73 (brs, 6 H), 2.03 (brs, 3 H), 2.11 (brs, 6 H), 3.64 (m, 2 H), 3.73 (m, 4 H), 3.83 (s, 3 H), 3.91 (m, 2 H), 6.81 (d, J=8.1 Hz, 1 H), 7.22 (m, 3 H), 7.54 (m, 2 H), 7.68 (s, 1 H), 9.43 (s, 1 H).

### Example 50

### 5-[3-(3-Adamantan-1-yl-4-hydroxy-5-fluoro-phenyl)benzylidene]-2-(trans-2,6-dimethylmorpholin-4-yl)-thiazol-4-one

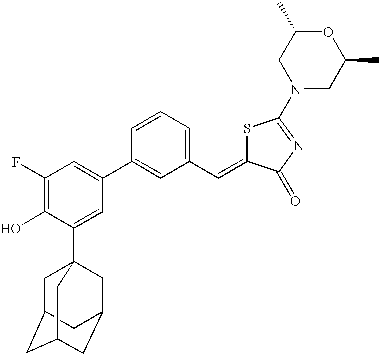

Prepared in a manner similar to that described in Example 1 using 3-(3-adamantan-1-yl-4-hydroxy-5-fluoro-phenyl)-benzaldehyde, rhodanine and trans-2,6-dimethylmorpholine, mp 262-264° C. ¹H NMR (300 MHz, DMSO-d₆): δ 1.13-1.17 (m, 6 H), 1.74 (s, 6 H), 2.05 (s, 3 H), 2.14 (s, 6 H), 1.93 (dd, J=10.8 Hz, J=13.2 Hz, 1 H), 3.14 (dd, J=12.9 Hz, J=10.8 Hz, 1 H), 3.62-3.70 (m, 2 H), 3.73 (d, J=12.9 Hz, 1 H), 4.50 (d, J=12.9 Hz, 1 H), 7.24 (s, 1 H), 7.43 (dd, J=11.7 Hz, J=2.1 Hz, 1 H), 7.52-7.55 (m, 2 H), 7.65-7.70 (m, 1 H), 7.75 (s, 1 H), 7.87 (s, 1 H), 9.59 (s, 1 H). MS: Expected: 546; Found: 547 (M+H).

### Example 51

### 5-[3-(3-Adamantan-1-yl-4-hydroxy-5-fluorophenyl)benzylidene]-2-methylsulfanyl-thiazol-4-one

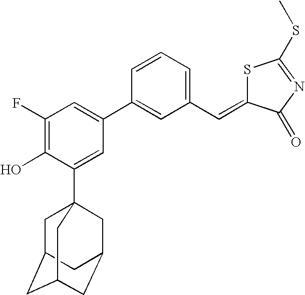

To a suspension of 5-[3-(3-adamantan-1-yl-4-hydroxy-5-fluorophenyl)benzylidene]-2-thioxo-thiazol-4-one (0.465 g, 1.0 mmol) and DIEA (0.21 mL, 1.2 mmol) in EtOH (7 mL) was added iodomethane (0.10 mL, 1.60 mmol). The mixture was stirred at RT (23 hrs) and the resulting mixture was then poured in water. After stirring for 1 hour, the product was filtered, washed with EtOH and stirred in EtOH for 1 hr and filtered. The solid was further purified by preparative HPLC to give 253 mg (53%) of 5-[3-(3-Adamantan-1-yl-4-hydroxy-5-fluorophenyl)benzylidene]-2-methylsulfanyl-thiazol-4-one. mp 270-271° C. ¹H NMR (300 MHz, DMSO-d₆): δ 1.74 (s, 6H), 2.05 (s, 3H), 2.14 (s, 6H), 2.82 (s, 3H), 7.24 (s, 1 H, 7.42 (dd, J₁=2.1 Hz, J₂=11.7 Hz, 1 H, 7.53-7.59 (m, 2 H, 7.71-7.75 (m, 1 H, 7.92 (s, 1 H, 7.95 (s, 1 H, 9.61 (d, J=2.4 Hz, 1 H. ¹³C NMR (75 MHz, DMSO-d₆): ppm 15.5, 28.4, 36.5, 36.9 (d, J=2.3 Hz), 111.3 (d, J=20.8 Hz), 119.9, 126.3, 127.7, 128.4, 128.7, 129.3 (d, J=7.1 Hz), 129.8, 133.7, 135.1, 139.1, 140.4, 143.3 (d, J=14.8 Hz), 152.2 (d, J=234 Hz), 178.7, 193.1. Expected: 479; Found: 480 (M+H); Expected: 479; Found: 478 (M−H).

The intermediate 5-[3-(3-adamantan-1-yl-4-hydroxy-5-fluorophenyl)benzylidene]-2-thioxo-thiazol-4-one was prepared in a similar as in example 1 using 5-[3-(3-adamantan-1yl-4-hydroxy-5-fluoro-phenyl)benzaldehyde (example 1a) and rhodanine.

### Example 52

### 5-[3-(3-Adamantan-1-yl-4-hydroxy-5-fluorophenyl)benzylidene]-2-hydroxyamino-thiazol-4-one

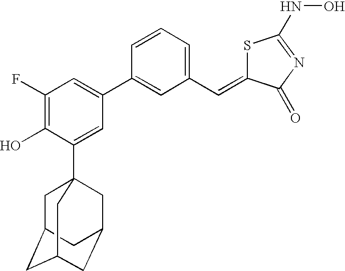

To a stirred mixture of 5-[3-(3-adamantan-1-yl-4-hydroxy-5-fluorophenyl)benzylidene]-2-methylsulfanyl-thiazol-4-one (example 51) (0.300 g, 0.63 mmol) and hydroxylamine hydrochloride (0.048 g, 0.69 mmol) at RT under argon was added t-BuOK (78 mg, 0.69 mmol) and the resulting mixture heated at reflux for 7 hours. The mixture was cooled to room temperature, diluted with ethyl acetate and water, separated and the aqueous layer washed once with ethyl acetate. The combined organics were washed successively with water and brine, dried over anhydrous magnesium sulfate, filtered and evaporated. The crude product was further purified by prep HPLC (isocratic; 30% A: 70% B, A is water with 0.02% TFA and B is 30% THF in acetonitrile with 0.02% TFA) and after evaporation afforded 5-[3-(3-adamantan-1-yl-4-hydroxy-5-fluorophenyl)benzlidene]-2-hydroxyamino-thiazol-4-one as a yellow solid (89 mg, 31%); mp 238-240; ¹H NMR (300 MHz; in ppm, DMSO-d₆): δ 1.73 (s, 6H), 2.05 (s, 3H), 2.14 (s, 6H), 7.26 (s, 1 H, 7.42 (dd, J₁=1.5 Hz, J₂=11.7 Hz, 1 H), 7.49-7.58 (m, 2 H), 7.63 (s, 1 H), 7.65-7.68 (m, 1 H), 7.84 (s, 1 H), 9.59 (d, J=2.7 Hz, 1 H), 10.92 (s, 1 H), 12.06 (s, 1 H). MS: Expected: 464; Found: 465 (M+H); Expected: 464; Found: 463 (M−H).

### Example 53

### 5-[3-(3-Adamantan-1-yl-4-hydroxy-5-fluoro-phenyl)benzylidene]-2-(2-(S)-carboxy-1-yl)-thiazol-4-one

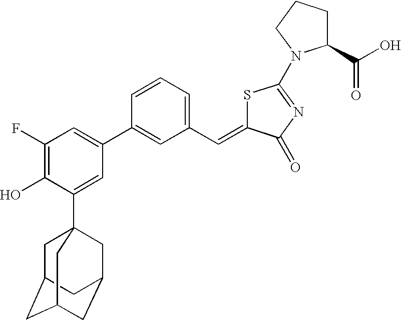

Prepared in a manner similar to that described in Example 52 using 5-[3-(3-adamantan-1-yl-4-hydroxy-5fluorophenyl)benzylidene]-2-methylsulfanyl-thiazol-4-one (example 51) and (S)-proline, mp 195-198° C. ¹H NMR (300 MHz, DMSO-d₆): δ 1.74 (s, 6 H), 2.05 (s, 6 H), 2.14 (s, 6 H), 2.31-2.43 (m, 1 H), 3.66-3.88 (m, 2 H), 4.58-4.69 (m, 1 H), 7.25 (s, 1 H), 7.43 (dd, J₁=2.1 Hz, J₂=11.7 Hz, 1 H), 7.55 (d, J=4.8 Hz, 2 H), 7.64-7.72 (m, 1 H), 7.76 (s, 1 H), 7.88 (s, 1 H), 9.59 (d, J=2.4 Hz, 1 H), 13.09 (brs, 1 H). MS: Expected: 546; Found: 547 (M+H), Expected: 546; Found: 545 (M−H).

### Example 57

### 5-[3-(3-Adamantan-1-yl-4-carboxyethyl-phenyl)-benzylidene]-2-pyrrolidine-1-yl-thiazol-4-one

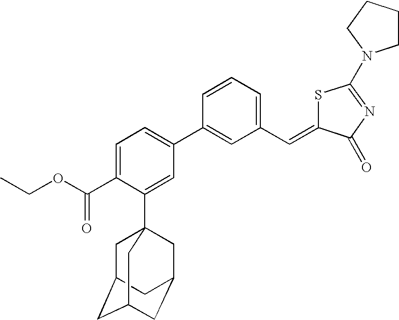

Prepared in a manner similar to that described in Example 1 using 3-(3-adamantan-1-yl-4-carboxyethyl-phenyl)benzaldehyde, rhodanine and pyrrolidine, mp 193° C. ¹H NMR (300 MHz, DMSO-d₆): δ 1.42 (t, J=7.2 Hz, 3 H), 1.77 (brs, 6 H), 2.12 (brm, 6 H), 2.14 (brs, 6 H), 3.63 (t, 2 H), 3.89 (t, 2 H), 4.41 (q, J=7.2 Hz, 2 H), 7.35-7.62 (m, 6 H), 7.73 (d, J=7.2 Hz, 1 H), 7.87 (s, 1 H).

The intermediate 5-[3-(3-adamantan-1-yl-4-carboxyethyl-phenyl)-benzaldehyde was prepared as followed:

### a. 5-[3-(3-adamantan-1-yl-4-carboxyethyl-phenyl)-benzaldehyde

A mixture of trifluoro-methanesulfonic acid 3-adamatan-1-yl-3′-formyl-biphenyl-3-yl ester (1.81 g, 3.89 mmol) and triethylamine (1.1 mL, 7.78 mmol) in a mixture of dimethylformamide:ethanol (4:1, 93 mL) was degassed with argon for 1 h. Tetrakis(triphenylphosphine)palladium(0) (1.35 g, 1.17 mmol) was added and the vessel was pressurized with carbon monoxide to 55 psi, and heated to 70 C for 16 hours. The solution was cooled to room temperature, depressurized, poured into H₂O and extracted with ethyl acetate (twice). The combined organic layers were washed successively with water and brine, dried over anhydrous magnesium sulfate, filtered and evaporated. The residue was purified on silica gel (eluent: hexane: ethyl acetate, 95:5) to give 798 mg (53%) of 3-Adamantan-1-yl-3′-formyl-biphenyl-4-carboxylic acid ethyl ester.

### b. Trifluoro-methanesulfonic acid 3-adamatan-1-yl-3′-formyl-biphenyl-3-yl ester.

A mixture of 3-(3-Adamantan-1-yl-4-hydroxy-phenyl)benzaldehyde (2.58 g, 7.76 mmol) (example 2a), dimethylaminopyridine (95 mg, 0.776 mmol), and pyridine (1.9 mL, 23.3 mmol) in CH₂Cl₂ was cooled to −78 C under an atmosphere of argon, then trifluoromethanesulfonic anhydride (1.6 mL, 9.31 mmol) was added dropwise over 1 h. The mixture was stirred at room temperature for 1 h, then was poured into water and extracted with CH₂Cl₂ (twice). The combined organic layers were washed successively with a saturated solution of NaHCO₃, water and brine, dried over anhydrous magnesium sulfate, filtered and evaporated. The residue was purified on silica gel (eluent: hexane:ethyl acetate, 9:1) to give 3.35 g (95%) of trifluoro-methanesulfonic acid 3-adamatan-1-yl-3′-formyl-biphenyl-3-yl ester.

### Example 59

### 5-[5-(3-Adamantan-1-yl-4-hydroxy-5-fluoro-phenyl)-thiophene-2-yl-methylene]-2-pyrrolidine-1-yl-thiazol-4-one

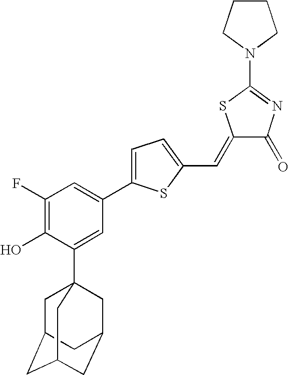

Prepared in a manner similar to that described in Example 1 using 5-(3-adamantan-1-yl-4-hydroxy-5-fluoro-phenyl)-thiophene-2-carboxaldehyde, rhodanine and pyrrolidine, mp 312-315° C. ¹H NMR (300 MHz, DMSO-d₆): 1.73 (s, 6 H), 1.91-2.08 (m, 7 H), 2.11 (s, 6 H), 3.62 (t, J=6.6 Hz, 2 H), 3.69 (t, J=6.3 Hz, 2 H), 7.22 (s, 1 H), 7.41 (d, J=11.7 Hz, 1 H), 7.83 (s, 1 H), 7.98 (s, 1 H), 8.08 (s, 1 H), 9.54 (d, J=2.7 Hz, 1 H). Expected: 508; Found: 509 (M+H); Expected: 508; Found: 507 (M−H).

### Example 61

### 5-[3-(3-Adamantan-1-yl-4-hydroxy-5-fluoro-phenyl)-4-dimethylamino-benzylidene]-2-pyrrolidin-1-yl-thiazol-4-one

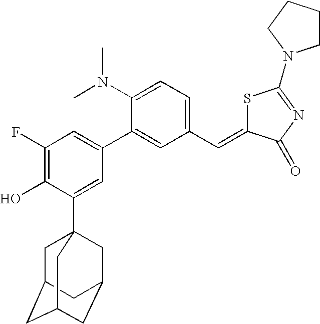

Prepared in a manner similar to that described in Example 1 using 3-(3-adamantan-1-yl-4-hydroxy-5-fluoro-phenyl)-4-dimethylamino-benzaldehyde, rhodanine and pyrrolidine, mp 294-298° C. ¹H NMR (300 MHz, DMSO-d₆): δ 1.72 (s, 6 H), 1.92-2.04 (m, 7 H), 2.11 (s, 6 H), 2.57 (s, 6 H), 2.57 (s, 6 H), 3.59 (t, J=6 Hz, 1 H), 3.67 (t, J=6.3 Hz, 1 H), 7.07 (d, J=8.7 Hz, 1 H), 7.11 (s, 1 H), 7.27 (dd, J₁=11.7 Hz, J₂=1.8 Hz, 1 H), 7.40 (d, J=2.4 Hz, 1 H), 7.45 (dd, J₁=8.4 Hz, J₂=1.8 Hz, 1 H), 7.57 (s, 1 H), 9.47 (d, J=2.4 Hz, 1 H). Expected: 545; Found: 546 (M+H); Expected: 545; Found: 544 (M−H).

### Example 62

### 5-[3-(3-Adamantan-1-yl-4-hydroxy-5-fluoro-phenyl)-4-trifluoromethoxy-benzylidene]-2-morpholin-4-yl-thiazol-4-one

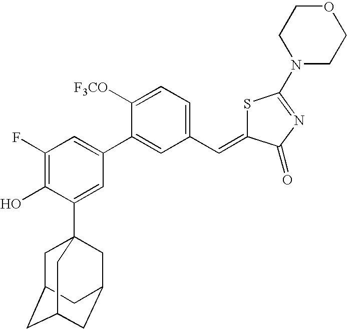

Prepared in a manner similar to that described in Example 1 using 3-(3-adamantan-1-yl-4-hydroxy-5-fluoro-phenyl)-4-trifluoromethoxy-benzaldehyde, rhodanine and morpholine, mp 238° C. ¹H NMR (300 MHz, DMSO-d₆): δ 1.71 (s, 6 H), 2.03 (s, 3 H), 2.09 (s, 6 H), 3.61-3.67 (m, 2 H), 3.68-3.75 (m, 4 H), 3.89-3.95 (m, 2 H), 7.07 (s, 1 H), 7.23 (dd, J₁=11.4 Hz, J₂=2.1 Hz, 1 H), 7.55 (dd, J₁=8.4 Hz, J₂=2.4 Hz, 1 H), 7.67 (dd, J₁=8.7 Hz, J₂=2.4 Hz, 1 H), 7.74 (s, 1 H), 7.78 (d, J=2.4 Hz, 1 H), 9.72 (s, 1 H). Expected: 602; Found: 603 (M+H); Expected: 602; Found: 601 (M−H).

### Example 64

### 5-[3-(3-Fluoro-4-hydroxy-phenyl)-benzylidene]-2-morpholin-4-yl-thiazol-4-one

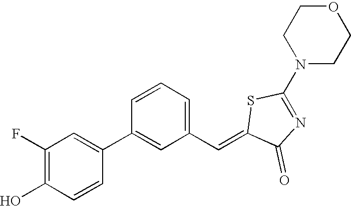

Prepared in a manner similar to that described in Example 1 using 3-(3-fluoro-4-hydroxy-phenyl)-benzaldehyde, rhodanine and morpholine, mp 233-235° C. ¹H NMR (300 MHz, DMSO-d₆): δ 3.62-3.78 (m, 6 H), 3.92 (t, J=4.8 Hz, 1 H), 7.04 (t, J=9 Hz, 1 H), 7.37 (dd, J₁=8.4 Hz, J₂=1.8 Hz, 1 H), 7.49-7.57 (m, 3 H), 7.67 (td, J₁=4.5 Hz, J₂=1.5 Hz, 1 H), 7.72 (s, 1 H), 7.84 (s, 1 H), 10.08 (s, 1 H. Expected: 384; (M+H); Expected: 384; Found: 383 (M−H).

### Example 66

### 5-[5-(3-Adamantan-1-yl-4-hydroxy-5-fluoro-phenyl)-pyridin-3-yl]-2-morpholin-4-yl-thiazol-4-one

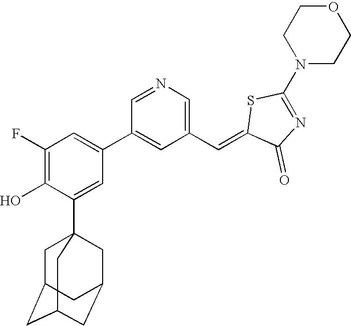

Prepared in a manner similar to that described in Example 1 using 5-(3-adamantan-1-yl-4-hydroxy-5-fluoro-phenyl)-pyridin-3-carboxaldehyde, rhodanine and morpholine, mp 310-317° C. ¹H NMR (300 MHz, DMSO-d₆): δ 1.73 (s, 6 H), 2.04 (s, 3 H), 2.14 (s, 6 H), 3.65-3.76 (m, 6 H), 3.89-3.96 (m, 2 H), 7.26 (s, H), 7.52 (dd, J₁=1.8 Hz, J₂=10.8 Hz, 1 H), 7.77 (s, 1 H), 8.20-8.23 (m, 1 H), 8.75 (d, J=2.1, 1 H), 8.85 (d, J=2.1, 1 H), 9.73 (d, J=2.7, 1 H). Expected: 519; Found: 520 (M+H); Expected: 519; Found: 518 (M−H).

### Example 68

### 5-[6-(3-Phenyl-4-hydroxy-phenyl)-pyridin-2-yl]-2-morpholin-4-yl-thiazol-4-one

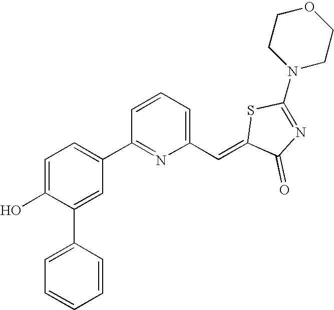

Prepared in a manner similar to that described in Example 1 using 6-(3-phenyl-4-hydroxy-phenyl)-pyridin-2-carboxaldehyde, rhodanine and morpholine, mp 292-295° C. ¹H NMR (300 MHz, DMSO-d₆): δ 3.27 (brs, 2 H), 3.56 (brs, 2 H), 3.73 (brs, 2 H), 3.93 (brs, 2 H), 7.13 (d, J=8.4 Hz, 1 H), 7.41 (t, J=6.5 Hz, 1 H), 7.50 (t, J=7.4 Hz, 2 H), 7.65 (d, J=7.5 Hz, 1 H), 7.72 (t, J=4.2 Hz, 1 H), 7.76 (s, 1 H), 7.99 (d, J=4.2 Hz, 2 H), 8.02 (d, J=1.8 Hz, 1 H), 8.39 (d, J=1.8 Hz, 1 H), 10.08 (s, 1 H). MS: Expected: 443; Found: 444 (M+H), Expected: 443; Found: 442 (M−H).

### Example 69

### 5-[3-(3-Adamantan-1-yl-4-hydroxy-5-fluoro-phenyl)benzylidene]-2-(pyrrolidin-1-yl amino)-thiazol-4-one

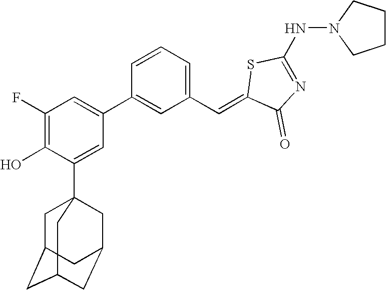

Prepared in a manner similar to that described in Example 52 using 5-[3-(3-adamantan-1-yl-4-hydroxy-5-fluorophenyl)benzylidene]-2-methylsulfanyl-thiazol-4-one (example 51) and 1-aminopyrrolidine, mp 293-296° C. ¹H NMR (300 MHz, DMSO-d₆): δ 1.69-1.83 (m, 10 H), 2.05 (s, 3 H), 2.14 (s, 6 H), 2.86 (m, 4 H), 7.23 (s, 1 H), 7.42 (dd, J₁=2.1 Hz, J₂=11.7 Hz, 1 H), 7.48-7.59 (m, 2 H), 7.63-7.90 (m, 2 H), 7.84 (s, 1 H), 9.59 (s, 1 H), 11.91 (brs, 1 H). MS: Expected: 517; Found: 518 (M+H), Expected: 517; Found: 516 (M−H).

### Example 70

### 5-[3-(3-Adamantan-1-yl-4-hydroxy-5-fluorophenyl)benzylidene]-2-(N-guanidinyl)-thiazol-4-one

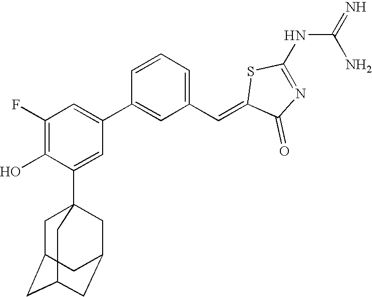

Prepared in a manner similar to that described in Example 52 using 5-[3-(3-adamantan-1-yl-4-hydroxy-5-fluorophenyl)benzylidene]-2-methylsulfanyl-thiazol-4-one (example 51) and guanidine, mp 288-290° C. ¹H NMR (300 MHz, DMSO-d₆): δ 1.73 (s, 6 H), 2.05 (s, 3 H), 2.13 (s, 6 H), 7.22(s, 1 H), 7.39 (dd, J₁=1.8 Hz, J₂=11.7 Hz, 1 H), 7.46-7.55 (m, 3 H), 7.62 (d, J=7.5 Hz, 1 H), 7.69 (s, 1 H), 7.81 (s, 1 H), 8.34 (brs, 1 H), 9.58 (d, J=2.7 Hz, 1 H). Expected: 490; Found: 491 (M+H).

### Example 71

### 5-[3-(3-Adamantan-1-yl-4-hydroxy-5-fluorophenyl)benzylidene]-2-methoxyamino-thiazol-4-one

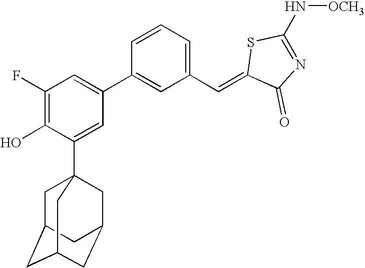

Prepared in a manner similar to that described in Example 52 using 5-[3-(3-adamantan-1-yl-4-hydroxy-5-fluorophenyl)benzylidene]-2-methylsulfanyl-thiazol-4-one (example 51) and O-methyl hydroxylamine, mp 286-288° C. ¹H NMR (300 MHz, DMSO-d₆): δ 1.73 (s, 6 H), 2.05 (s, 3 H), 2.14 (s, 6 H), 3.80 (s, 3 H), 7.24 (s, 1 H), 7.42 (dd, J₁=1.8 Hz, J₂=11.7 Hz, 1 H), 7.49-7.58 (m, 2 H), 7.66-7.70 (m, 2 H), 7.83 (s, 1 H), 9.59 (d, J=2.7 Hz, 1 H), 12.25 (brs, 1 H). Expected: 478; Found: 479 (M+H).

### Example 72

### 5-[3-(3-Adamantan-1-yl-4-hydroxy-5-fluorophenyl)benzylidene]-2-semicarbazid-1-yl-thiazol-4-one

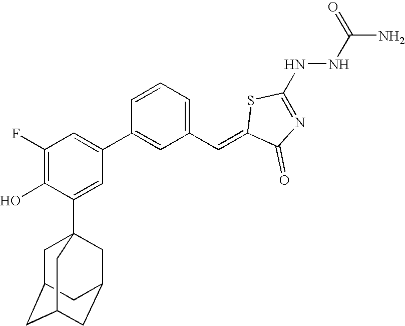

Prepared in a manner similar to that described in Example 52 using 5-[3-(3-adamantan-1-yl-4-hydroxy-5-fluorophenyl)benzylidene]-2-methylsulfanyl-thiazol-4-one (example 51) and semicarbazide, mp 234-236° C. ¹H NMR (300 MHz, DMSO-d₆): δ 1.73 (s, 6 H), 2.05 (s, 3 H), 2.14 (s, 6 H), 6.12 (brs, 2 H), 7.23 (s, H), 7.39-7.46 (m, 1 H), 7.49-7.59 (m, 2 H), 7.68 (d, J=9.3 Hz, 1 H), 7.69 (s, 1 H), 7.85 (s, 1 H), 8.91 (s, 1 H), 9.59 (s, 1 H). Expected: 506; Found: 507 (M+H).

### Example 73

### 5-[3-(3-Adamantan-1-yl-4-hydroxy-5-fluorophenyl)benzylidene]-2-(morpholin-4-yl-amino)-thiazol-4-one

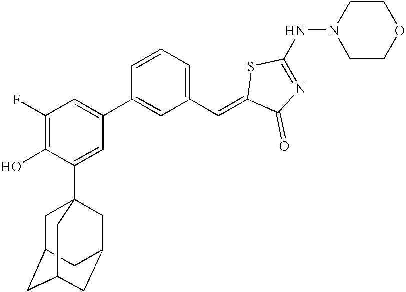

Prepared in a manner similar to that described in Example 52 using 5-[3-(3-adamantan-1-yl-4-hydroxy-5-fluorophenyl)benzylidene]-2-methylsulfanyl-thiazol-4-one (example 51) and 4-amino-morpholine, mp 304-309° C. ¹H NMR (300 MHz, DMSO-d₆): δ 1.73 (s, 6 H), 2.05 (s, 3 H), 2.14 (s, 6 H), 2.72-2.77 (m, 4 H), 3.67-3.71 (m, 4 H), 7.22 (s, 1 H), 7.39 (dd, J₁=11.7 Hz, J₂=1.5 Hz, 1 H), 7.49-7.59 (m, 2 H), 7.65 (d, J=7.2 Hz, 1 H), 7.68 (s, 1 H), 7.82 (s, 1 H), 9.60 (d, J=2.7 Hz, 1 H), 12.03 (s, 1 H). Expected: 533; Found: 534 (M+H).

### Example 74

### 5-[3-(3-Adamantan-1-yl-4-hydroxy-5-fluorophenyl)benzylidene]-2-hydrazino-thiazol-4-one

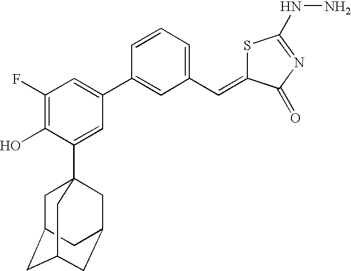

Prepared in a manner similar to that described in Example 52 using5-[3-(3-adamantan-1-yl-4-hydroxy-5-fluorophenyl)benzylidene]-2-methylsulfanyl-thiazol-4-one (example 51) and hydrazine, mp 303-308° C. ¹H NMR (300 MHz, DMSO-d₆): δ 1.74 (s, 6 H), 2.05 (s, 3 H), 2.15 (s, 6 H), 4.57 (s, 1 H), 7.21 (s, 1 H), 7.39 (dd, J₁=11.7 Hz, J₂=1.8 Hz, 1 H), 7.42 (s, 1 H), 7.51-7.57 (m, 3 H), 7.80 (s, 1 H), [8.60 (brs), 9.40 (brs), 3 H]. Expected: 463; Found: 464 (M+H).

### Example 76

### 5-[3-(3-Adamantan-1-yl-4-hydroxy-5-fluoro-phenyl)benzylidene]-2-(2-(R)-carboxy-1-yl)-thiazol-4-one

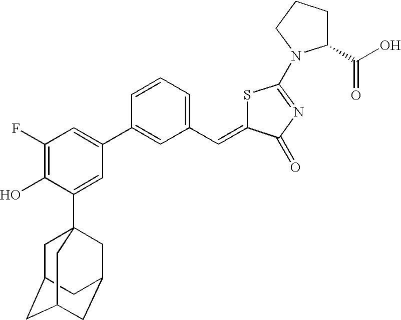

Prepared in a manner similar to that described in Example 52 using 5-[3-(3-adamantan-1-yl-4-hydroxy-5-fluorophenyl)benzylidene]-2-methylsulfanyl-thiazol-4-one (example 51) and (D)-proline, mp 164-168° C. ¹H NMR (300 MHz, DMSO-d₆): δ 1.74 (s, 6 H), 1.99-2.22 (m, 12 H), 2.32-2.44 (m, 1 H), 3.66-3.87 (m, 2 H), 4.59-4.71 (m, 1 H), 7.21-7.29 (m, 1 H), 7.37-7.48 (m, 1 H), 7.48-7.60 (m, 2 H), 7.65-7.72 (m, 1 H), 7.77 (s, 1 H), 7.82-7.96 (m, 1 H), 9.60 (d, J=2.7 Hz, 1 H), 13.07 (s, 1 H). Expected: 546; Found: 547 (M+H); Expected: 546; Found: 545 (M−H).

### Example 77

### 5-[2-(3-Adamantan-1-yl-4-hydroxy-phenyl)-benzylidene]-2-morpholin-4-yl-thiazol-4-one

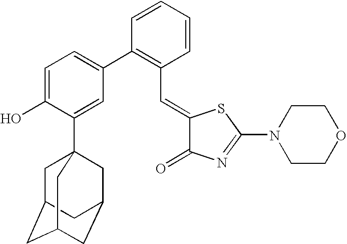

Prepared in a manner similar to that described in Example 1 using 2-(3-adamantan-1-yl-4-hydroxy-phenyl)-benzaldehyde, rhodanine and morpholine, mp 321-324° C. 1H NMR (300 MHz, DMSO-d₆): δ 1.71( s, 6 H), [2.01 (s), 2.06 (s), 9 H], 3.64-3.75 (m, 6 H), 3.92 (m, 2 H), 6.86 (d, J=8.7 Hz, 1 H), 6.96-7.00 (m, 2 H), 7.41-7.50 (m, 3 H), 7.51 (s, 1 H), 7.62-7.66 (m, 1 H), 9.59 (s, 1 H).

### Example 78

### 5-[3-(3-Adamantan-1-yl-4-hydroxy-phenyl)-4,6-dimethoxy-benzylidene]-2-pyrrolidin-4-yl-thiazol-4-one

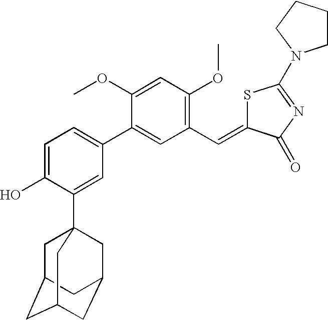

Prepared in a manner similar to that described in Example 1 using 3-(3-adamantan-1-yl-4-hydroxy-phenyl)-4,6-dimethoxy-benzaldehyde, rhodanine and pyrrolidine, mp 343-345° C. 1H NMR (300 MHz, DMSO-d₆): δ 1.73 (s, 6 H), 1.9-2.06 (m, 7 H), 2.13 (s, 6 H), 3.54-3.61 (m, 2 H), 3.62-3.72 (m, 2 H), 3.89 (s, 3 H), 3.96 (s, 3 H), 6.79 (d, J=7.2 Hz, 1 H), 6.80 (s, 1 H), 7.19-7.25 (m, 2 H), 7.39 (s, 1 H), 7.85 (s, 1 H), 9.39 (s, 1 H).

### Example 81

### 5-[3-(3-Adamantan-1-yl-4-hydroxy-5-fluoro-phenyl)benzyl]-2-pyrrolidin-4-yl-thiazol-4-one

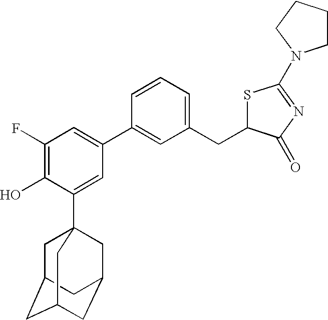

Prepared in a manner similar to that described in Example 1 using 5-[3-(3-adamantan-1-yl-4-hydroxy-5-fluoro-phenyl)benzyl]-2-thioxo-thiazol-4-one, and pyrrolidine, mp 152-154° C. ¹H NMR (300 MHz, DMSO-d₆): δ 1.73 (s, 6 H), 1.84-1.97 (m, 4 H), 2.04 (s, 3H), 2.13 (s, 6 H), 2.83 (dd, J₁=10.8 Hz, J₂=14.1 Hz, 1 H, 3.37 (t, J=6.0 Hz, 2 H, 3.50 (dd, J₁=10.2 Hz, J₂=14.4 Hz, 1 H, 3.56 (t, J=6.0 Hz, 2 H), 4.77 (dd, J₁=3.6 Hz, J₂=10.2 Hz, 1 H), 7.14-7.22 (m, 2 H), 7.29-7.38 (m, 2 H), 7.42-7.48 (m, 1 H), 7.51 (s, 1 H), 9.49 (d, J=2.7 Hz, 1 H). Expected: 504; Found: 505 (M+H); Expected: 504; Found: 503 (M−H).

The intermediate 5-[3-(3-adamantan-1-yl-4-hydroxy-5-fluoro-phenyl)benzyl]-2-thioxo-thiazol-4-one was prepared by the reduction 5-[3-(3-adamantan-1-yl-4-hydroxy-5-fluoro-phenyl)benzylidene]-2-thioxo-thiazol-4-one using LiBH₄/pyridine in THF heated to reflux in a manner similar to that described by Giles, et. al., Tetrahedron 2000, 56, 4531-4537.

### Example 82

### 5-[5-(3,3-Dimethyl-2,3-dihydro-benzofuran-5-yl)-pyridin-3-yl methylene]-2-morpholin-4-yl-thiazol-4-one

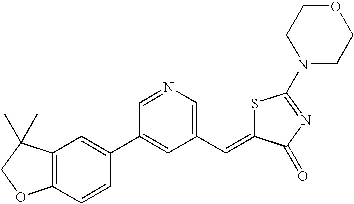

Prepared in a manner similar to that described in Example 1 using 5-(3,3-dimethyl-2,3-dihydro-benzofuran-5-yl)-pyridin-3-carboxaldehyde, rhodanine and morpholine, mp 230-233° C. ¹H NMR (300 MHz, DMSO-d₆): δ 1.34 (s, 6 H), 3.65-3.75 (m, 6 H), 3.93 (t, J=4.5 Hz, 2 H), 4.27 (s, 2 H), 6.90 (d, J=8.7 Hz, 1 H), 7.51 (dd, J1=8.7 Hz, J2=2.1 Hz, 1 H), 7.66 (d, J=2.1 Hz, 1 H), 7.74 (s, 1 H), 8.18 (t, J=2.1 Hz, 1 H), 8.74 (d, J=2.1 Hz, 1 H), 8.86 (d, J=2.1 Hz, 1 H). Expected: 421; Found: 422 (M+H).

### a. 5-(3,3-dimethyl-2,3-dihydro-benzofuran-5-yl)-pyridin-3-carboxaldehyde

To a degassed solution of 3,3-dimethyl-2,3-dihydro-benzofuran-5-boronic acid (1.2 g, 6.25 mmol), 5-bromo-pyridine-3-carbaldehyde (0.97 g, 5.21 mmol) and sodium carbonate (1.38 g, 13.03 mmol) in a mixture of toluene: ethanol (4:1, 50 mL) and water (5 mL) was added tetrakistriphenylphosphine Palladium (0) (300 mg, 0.26 mmol) and the reaction was heated at reflux overnight. After cooling the solution was diluted with ethyl acetate and washed successively with water and brine, dried over magnesium sulfate, filtered and evaporated. The residue was chromatographed on silica gel (eluent: 5% ethylacetate in hexane) to give 0.94 g (71%) of 5-(3,3-dimethyl-2,3-dihydro-benzofuran-5-yl)-pyridin-3-carboxaldehyde.

### c. 3,3-dimethyl-2,3-dihydro-benzofuran-5-boronic acid

The compound was prepared using a procedure analogous to that reported in example 27c above.

### d. 5-bromo-3,3-dimethyl-2,3-dihydro-benzofuran

A mixture of 4-bromo-2-(2-chloro-1,1-dimethyl-ethyl)-1-methoxy-benzene (65 g, 0.234 mol), pyridine hydrochloride (121.8 g, 1.054 mol) and quinoline (110.67 mL, 0.936 mol) was refluxed at 164° C.-167° C. under argon for 5 hrs. After cooling to room temperature the reaction mixture was treated with ice-cold 6N HCl and extracted twice with ether. The organic layers were combined, dried (Mg₂SO₄), filtered and evaporated. The residue was purified on silica gel (10% ethyl acetate in hexane) to give 52 g of 5-bromo-3,3-dimethyl-2,3-dihydro-benzofuran (98%). ¹H NMR (300 MHz; CDCl₃): 1.32 (s, 6 H), 4.23 (s, 2 H), 6.67 (d, J=8.1 Hz, 1 H), 7.19 (m, 2 H).

### e. 4-bromo-2-(2-chloro-1,1-dimethyl-ethyl)-1-methoxy-benzene

Sulfuric acid (2 mL, 0.033 mol) was added dropwise under argon to 4-bromoanisole (14.6 mL, 0.117 mol). The mixture was warmed to 40-43° C. (warm water bath) and 3-chloro-2-methyl propene was added dropwise in 4 equal portions over 2 hrs. After 2 hrs at 40-43° C. the solution was diluted with dichloromethane and washed successively with water, saturated aqueous NaHCO₃, water and brine, dried (Mg₂SO₄), filtered and evaporated. The residue was crystallized from hexanes to give 14.1 g of 4-bromo-2-(2-chloro-1,1-dimethyl-ethyl)-1-methoxy-benzene. The mother liquor was further purified on silica gel (10% ethyl acetate in hexane) to afford additional 4.8 g of product. 58 % yield. ¹H NMR (300 MHz; CDCl₃): 1.43 (s, 6 H), 3.82 (s, 3 H), 3.93 (s, 2 H), 6.75 (dd, J=2.4 Hz and 7.2 Hz, 1 H), 7.32 (m, 2 H).

### Example 84

### 5-[3-(3-Adamantan-1-yl-4-hydroxy-phenyl)-5-methoxy-6-hydroxy-benzylidene]-2-morpholin-4-yl-thiazol-4-one

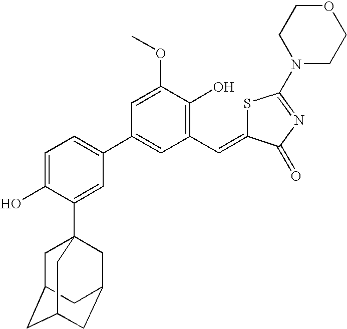

Prepared in a manner similar to that described in Example 1 using 3-(3-adamantan-1-yl-4-hydroxy-phenyl)-5-methoxy-6-hydroxy-benzaldehyde, rhodanine and morpholine, mp 310-312° C. ¹H NMR (300 MHz, DMSO-d₆): δ 1.75 (s, 1 H, [2.04 (s), 2.16 (s) 9H], 3.66 (s, 2 H, 3.74 (s, 4H), 3.92 (s, 5H), 6.86 (d, J=8 Hz, 1 H, 7.22 (s, 2 H, 7.34 (d, J=10 Hz, 2 H, 7.98 (s, 1 H, 9.43 (s, 1 H, 9.57 (s, 1 H. Expected: 546; Found: 547 (M+H), Expected: 546; Found: 545 (M−H).

### Example 86

### 5-[3-(3-Adamantan-1-yl-4-hydroxy-phenyl)-6-methoxy-benzylidene]-2-morpholin-4-yl-thiazol-4-one

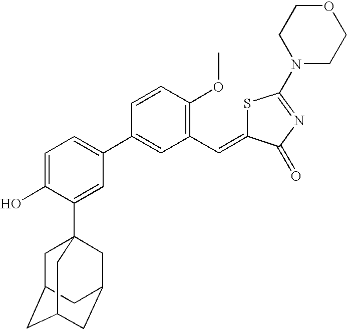

Prepared in a manner similar to that described in Example 1 using 3-(3-adamantan-1-yl-4-hydroxy-phenyl)-6-methoxy-benzaldehyde, rhodanine and morpholine, mp 355-360° C. ¹H NMR (300 MHz, DMSO-d₆): δ 1.75 (s, 6 H), [2.04 (s), 2.15 (s) 9 H], 3.64-3.65 (m, 2 H), 3.73 (m, 4 H), 3.91 (s, 5 H), 7.62-7.65 (m, 2 H), 7.92 (s, 1 H), 9.47 (s, 1 H). Expected: 530; Found: 531 (M+H), Expected: 530; Found: 529 (M−H).

### Example 89

### 5-[5-(3-Adamantan-1-yl-4-hydroxy-phenyl)-1H-indol-3-yl methylene]-2-morpholin-4-yl-thiazol-4-one

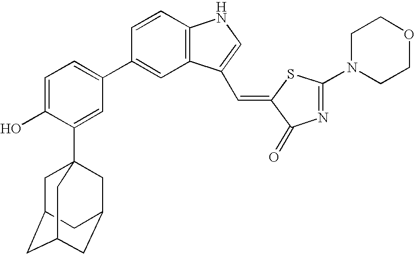

Prepared in a manner similar to that described in Example 1 using 5-(3-adamantan-1-yl-4-hydroxy-phenyl)-1H-indol-3-carboxaldehyde, rhodanine and morpholine, mp 312-315° C. ¹H NMR (300 MHz, DMSO-d₆): δ 1.76 (s, 6 H), 2.08 (s, 3 H), 2.17 (s, 6H), 3.68-4.00 (m, 8H), 6.86 (d, J=8 Hz, 1 H), 7.38-7.54 (m, 4H), 7.83 (s, 1 H), 8.05 (s, 1 H), 8.1 (s, 1 H), 9.36 (s, 1 H), 9.36 (s, 1 H).

The intermediate 5-(3-adamantan-1-yl-4-hydroxy-phenyl)-1H-indol-3-carboxaldehyde was prepared in a similar manner to that described in example 27 using 6-bromoindole-3-carbaldehyde in step b.

### Example 91

### 5-[3-(3-Adamantan-1-yl-4-hydroxy-phenyl)-4-fluoro-benzylidene]-2-morpholin-4-yl-thiazol-4-one

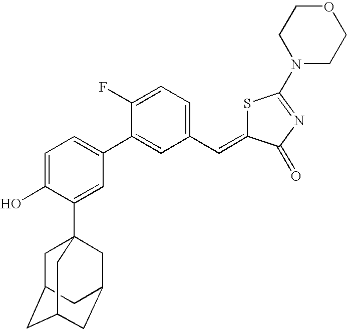

Prepared in a manner similar to that described in Example 1 using 3-(3-adamantan-1-yl-4-hydroxy-phenyl)-4-fluoro-benzaldehyde, rhodanine and morpholine, mp 338-343° C. ¹H NMR (300 MHz, DMSO-d₆): δ (DMSO-d₆), 1.74 (s, 6 H), 2.04 (s, 3H), 2.12 (s, 6H), 3.67 (d, J=5 Hz, 2 H, 3.74 (d, J=5 Hz, 4 H), 3.91-3.96 (m, 2 H), 6.89 (d, J=8 Hz, 1 H), 7.26-7.31 (m, 2 H), 7.41 (dd, J₁=12 Hz, J₂=8.4 Hz, 1 H), 7.56-7.62 (m, 1 H), 7.75 (s, 1 H), 7.76-7.77 (m, 1 H), 9.65 (s, 1 H).

### Example 93

### 5-[3-(3-Adamantan-1-yl-4-hydroxy-phenyl)-4-(morpholino-4-yl methyl)-benzylidene]-2-morpholin-4-yl-thiazol-4-one

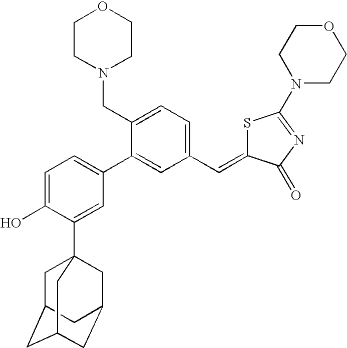

Prepared in a manner similar to that described in Example 1 using 3-(3-adamantan-1-yl-4-hydroxy-phenyl)-4-(morpholino-4-yl methyl)-benzaldehyde, rhodanine and morpholine, mp 304-305° C. ¹H NMR (300 MHz, DMSO-d₆): δ 1.71 (br. m, 6 H), 2.01 (br. s, 3 H), 2.09 (br s, 6 H), 2.29 (brs, 4 H)., 3.53 (m, 4 H), 3.63 (m, 2 H, 3.71 (m, 4 H), 3.91 (brt, 2 H), 6.82 (d, J=8.4 Hz, 1 H), 7.08 (dd, J₁=1.5, J₂=8.4 Hz, 1 H), 7.18 (d, J=2.1 Hz, 1 H, 7.45 (s, 1 H, 7.50-7.60 (m, 2 H), 7.67 (s, 1 H), 9.46 (s, 1 H).

### Example 95

### 5-[5-(3-t-Butyl-4-hydroxy-phenyl)-pyridin-3-yl]-2-morpholin-4-yl-thiazol-4-one

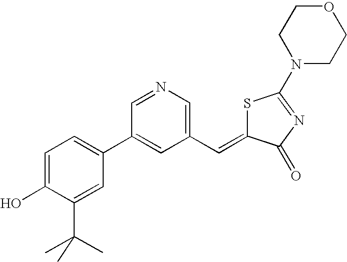

Prepared in a manner similar to that described in Example 1 using 5-(3-t-butyl-4-hydroxy-phenyl)-pyridin-3-carbaldehyde, rhodanine and morpholine, mp 231-234° C. ¹H NMR (300 MHz, DMSO-d₆): δ 1.39 (s, 9 H), 3.65-3.75 (m, 6 H), 3.92 (t, J=4.5 Hz, 2 H), 6.92 (d, J=8.1 Hz, 1 H), 7.41 (dd, J₁=8.1 Hz, J₂=1.5 Hz, 1 H), 7.46 (s, 1 H), 7.75 (s, 1 H), 8.12 (s, 1 H), 8.71 (d, J=1.5 Hz, 1 H), 8.80 (d, J=1.5 Hz, 1 H), 9.73 (s, 1 H). Expected: 423; Found: 424 (M+H); Expected: 423; Found: 422 (M−H), 458 (M+Cl⁻).

### Example 97

### 5-[5-(3-Adamantan-1-yl-4,5-methylenedioxy-phenyl)-pyridin-3-yl]-2-morpholin-4-yl-thiazol-4-one

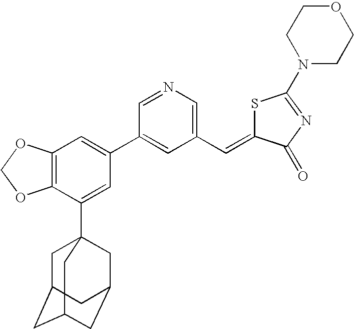

Prepared in a manner similar to that described in Example 1 using 5-(3-adamantan-1-yl-4,5-methylenedioxy-phenyl)-pyridin-3-carboxaldehyde, rhodanine and morpholine, mp 255-258° C. ¹H NMR (300 MHz, DMSO-d₆): δ 1.72 (s, 6 H), 2.03 (s, 9 H), 3.65-3.75 (m, 6 H), 3.92 (t, J=4.5 Hz, 2 H), 6.04 (s, 2 H), 7.04 (d, J=1.8 Hz, 1 H), 7.24 (d, J=1.5 Hz, 1 H), 7.75 (s, 1 H), 8.17 (s, 1 H), 8.74 (d, J=2.1 Hz, 1 H), 8.82 (d, J=2.1 Hz, 1 H). Expected: 529; Found: 530 (M+H).

### Example 98

### 5-[3-(3-Adamantan-1-yl-4-hydroxy-phenyl)-6-hydroxy-benzylidene]-2-morpholin-4-yl-thiazol-4-one

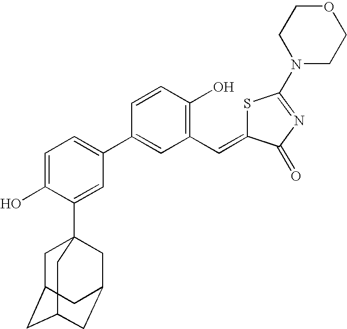

Prepared in a manner similar to that described in Example 1 using 3-(3-adamantan-1-yl-4-hydroxy-phenyl)-6-hydroxy-benzaldehyde, rhodanine and morpholine, mp 325-330° C. ¹H NMR (300 MHz, DMSO-d₆): δ 1.72 (s, 6H), 2.02 (s, 3 H), 2.13 (s, 6 H), 3.64(s, 2 H), 3.72(s, 4 H), 3.91 (s, 2 H), 6.82 (d, J=8 Hz, 1 H), 6.98 (d, J=8 Hz, 1 H), 7.24 (d, J=8 Hz, 1 H), 7.30 (s, 1 H), 7.49 (d, J=8 Hz, 1 H), 7.59 (s, 1 H), 7.94 (s, 1 H), 9.43 (s, 1 H), 10.39 (s, 1 H).

### Example 99

### 5-[3-(3-[1-Cyano-4-oxo-cyclohexyl]-4-methoxy-phenyl)benzylidene]-2-morpholin-4yl-thiazol-4-one

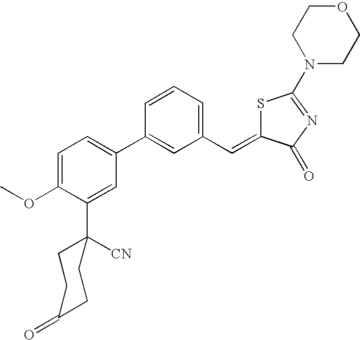

Prepared in a manner similar to that described in Example 1 using 3-(3-[1-cyano-4-oxo-cyclohexyl]-4-methoxy-phenyl)benzaldehyde, rhodanine and morpholine, mp 260-262° C. ¹H NMR (300 MHz, CDCl₃): δ 2.5-2.7 (m, 6 H), 2.85-3.0 (m, 2 H), 3.67 (t, J=4.8 Hz, 2 H), 3.83 (m, 4 H), 3.99 (s, 3 H), 4.10 (m, 2 H), 7.10 (d, J=8.4 Hz, 1 H), 7.49 (s, 1 H), 7.51 (s, 1 H), 7.56 (m, 1 H), 7.61 (dd, J₁=2.1, J₂=8.4 Hz, 1 H), 7.71 (s, 2 H), 7.87 (s, 1 H).

The intermediate 3-(3-[1-cyano-4-oxo-cyclohexyl]-4-methoxy-phenyl)benzaldehyde was prepared as followed:

### a. 3-(3-[1-cyano-4-oxo-cyclohexyl]-4-methoxy-phenyl)benzaldehyde

Prepared in a similar manner to that described in Example 1b, using 1-(5-bromo-2-methoxy-phenyl)-4-oxo-cyclohexanecarbonitrile.

### b. 1-(5-Bromo-2-methoxy-phenyl)-4-oxo-cyclohexanecarbonitrile

A solution of 5-(5-Bromo-2-methoxy-phenyl)-5-cyano-2-oxo-cyclohexanecarboxylic acid methyl ester (17.54 g, 47.89 mmol) in acetic acid (360 mL) and H₂SO₄ (180 mL of 10% solution in water) under an atmosphere of argon was heated at reflux for 16 h. The mixture was cooled and extracted with toluene (twice). The combined organic layers were neutralized with a saturated Na₂CO₃ solution, then washed successively with a saturated Na₂CO₃ solution, water and brine, dried over magnesium sulfate, filtered and evaporated to give 11.08 g (75%) 1-(5-Bromo-2-methoxy-phenyl)-4-oxo-cyclohexanecarbonitrile as a white powder.

### c. 5-(5-Bromo-2-methoxy-phenyl)-5-cyano-2-oxo-cyclohexanecarboxylic acid methyl ester

A mixture of 4-(5-Bromo-2-methoxy-phenyl)-4-cyano-heptanedioic acid dimethyl ester (46.4 g, 116 mmol) and sodium hydride (2.8 g, 116 mmol) in xylene (200 mL) under an atmosphere of argon was heated at reflux for 16 hours. The reaction mixture was quenched with 10% acetic acid, the organic layer was separated and washed successively with a saturated NaHCO₃ solution, water and brine, dried over anhydrous magnesium sulfate, filtered and evaporated. The residue was purified on silica gel (eluent: hexane:ethyl acetate, 86:14) to give 30.2 g (75%) of 5-(5-Bromo-2-methoxy-phenyl)-5-cyano-2-oxo-cyclohexanecarboxylic acid methyl ester as a white solid.

### d. 4-(5-Bromo-2-methoxy-phenyl)-4-cyano-heptanedioic acid dimethyl ester

A mixture of (5-Bromo-2-methoxy-phenyl)-acetonitrile (25 g, 110 mmol) and methyl acrylate (30.8 mL, 342 mmol) in tert-butanol (50 mL) was heated at reflux under an atmosphere of argon. To this mixture a solution of Triton B (11 mL of 40% in MeOH) in tert-butanol (30 mL) was added rapidly, and the resulting mixture was heated at reflux for 16 hours. The solvent was evaporated and the residue was dissolved in CH₂Cl₂ and washed successively with 0.2 N HCl and water, dried over anhydrous magnesium sulfate, filtered and evaporated to give 46.4 g (105% crude yield) of 4-(5-Bromo-2-methoxy-phenyl)-4-cyano-heptanedioic acid dimethyl ester, which was used as this in the next step.

### Example 100

### 5-{[3-(6-Oxo-1-aza-tricyclo[3.3.1.1^{3,7}]decan-3-yl)-4-methoxy-phenyl]-benzylidene}-2-morpholin-4-yl-thiazol-4-one

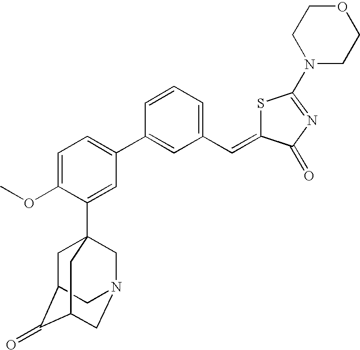

Prepared in a manner similar to that described in Example 1 using 3-(6-oxo-1-aza-tricyclo[3.3.1.1^{3,7}]decan-3-yl)-4-methoxy-phenyl]-benzaldehyde, rhodanine and morpholine, mp 167-170° C. ¹H NMR (300 MHz, DMSO-d₆): δ 2.38 (d, J=13.2 Hz, 2 H), 2.67 (d, J=12.0 Hz, 2 H), 3.08 (d, J=12.6 Hz, 2 H), 3.53 (s, 2 H), 3.67 (m, 2 H), 3.73 (m, 4 H), 3.85 (s, 3 H), 3.92 (t, J=4.8 Hz, 2 H), 7.11 (d, J=9.0 Hz, 1 H), 7.39 (d, J=1.8 Hz, 1 H), 7.54 (s, 1 H), 7.55 (s, 1 H), 7.58 (dd, J₁=1.8, J₁=8.7 Hz, 1 H), 7.69 (m, 1 H), 7.75 (s, 1 H), 7.88 (s, 1 H).

The intermediate [3-(6-oxo-1-aza-tricyclo[3.3.1.1^{3,7}]decan-3-yl)-4-methoxy-phenyl]-benzaldehyde was prepared as followed:

### a. [3-(6-oxo-1-aza-tricyclo[3.3.1.1^{3,7}]decan-3-yl)-4-methoxy-phenyl]-benzaldehyde

Prepared in a similar manner to that described in Example 1b, using 7-(5-bromo-2-methoxy-phenyl)-1-aza-tricyclo[3.3.1.1.^{3,7}]decan-3-one.

### b. 7-(5-Bromo-2-methoxy-phenyl)-1-aza-tricyclo[3.3.1.1.^{3,7}]decan-3-one

A solution of paraformaldehyde (2.2 g, 72.4 mmol) in 2% H₂SO₄/water (500 mL) was heated to reflux under an atmosphere of argon. To this was added dropwise a solution of C-[8-(5-Bromo-2-methoxy-phenyl)-1,4-dioxa-spiro[4.5]dec-8-yl]-methylamine (5.16 g, 14.5 mmol) in ethanol (150 mL). The resulting solution was heated at reflux for 16 hours, then was cooled and extracted with CH₂Cl₂ (twice). The combined organic layers were washed with 1.0 N HCl. The combined aqueous layers were neutralized by addition of 10 M NaOH solution, then extracted with CH₂Cl₂. This organic layer was washed with brine, dried over anhydrous magnesium sulfate, filtered, and evaporated to give 2.7 g (55%) 7-(5-Bromo-2-methoxy-phenyl)-1-aza-tricyclo[3.3.1.1.^{3,7}]decan-3-one as an orange oil.

### c. C-[8-(5-Bromo-2-methoxy-phenyl)-1,4-dioxa-spiro[4.5]dec-8-yl]-methylamine

A solution of lithium aluminum hydride (506 mg, 13.3 mmol) in anhydrous THF (70 mL) was cooled to 0 C under an atmosphere of argon. To this was added dropwise a solution of 8-(5-Bromo-2-methoxy-phenyl)-1,4-dioxa-spiro[4.5]decane-8-carbonitrile (4.7 g, 13.3 mmol) in anhydrous THF (20 mL). The resulting mixture was heated at reflux for 5 hours. H₂O (0.53 mL), 20% NaOH in water (0.4 mL) and H₂O (1.9 mL) were sequentially added to the mixture, which was then dried with anhydrous sodium sulfate, and filtered. The filter cake was bulk washed with THF for 10 minutes, then refiltered. The combined filtrates were evaporated to give 5.2 g (110% crude yield) of C-[8-(5-Bromo-2-methoxy-phenyl)-1,4-dioxa-spiro[4.5]dec-8-yl]-methylamine, which was used as this in the next step

### d. 8-(5-Bromo-2-methoxy-phenyl)-1,4-dioxa-spiro[4.5]decane-8-carbonitrile

To a solution of 1-(5-Bromo-2-methoxy-phenyl)-4-oxo-cyclohexanecarbonitrile (example 99b) (8.0 g, 25.9 mmol) and ethylene glycol (5 mL, 89 mmol) in toluene (30 mL) under an atmosphere of argon was added p-toluenesulfonic acid (246 mg, 1.295 mmol). The resulting mixture was heated at reflux for 16 hours, using a Dean-Stark trap to remove water. The solution was cooled and poured into a saturated solution of NaHCO₃, and extracted with ethyl acetate (twice). The combined organic layers were washed successively with water and brine, dried over anhydrous magnesium sulfate, filtered and evaporated. The residue was recrystallized from ethanol to give 4.84 g (53%) of 8-(5-Bromo-2-methoxy-phenyl)-1,4-dioxa-spiro[4.5]decane-8-carbonitrile as a white powder.

### Example 104

### 5-[3-(3-Adamantan-1-yl-4-hydroxy-phenyl)-4,6-dihydroxy-benzylidene]-2-morpholin-4yl-thiazol-4-one

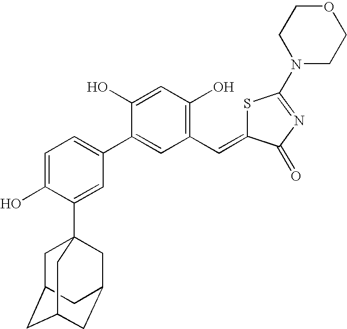

Prepared in a manner similar to that described in Example I using 3-(3-adamantan-1-yl-4-hydroxy-phenyl)-4,6-dihydroxy-benzaldehyde, rhodanine and morpholine, mp 306-310° C. ¹H NMR (300 MHz, DMSO-d₆): δ 1.73 (s, 6 H), 2.03 (s, 3 H), 2.11 (s, 6 H), 3.59-3.63 (m, 2 H), 3.70-3.73 (m, 4 H), 3.87-3.91 (m, 2 H), 6.58 (s, 1 H), 6.77 (d, J=8.4 Hz, 1 H), 7.19-7.25 (m, 2 H), 7.30 (s, 1 H), 7.91 (s, 1 H), 9.29 (s, 1 H), 10.11 (s, 1 H), 10.31 (s, 1 H). Expected: 532;Found 533 (M+H), Expected: 532; Found 531 (M−H).

### Example 106

### 5-[1-(3′-Adamantan-1-yl-4′-hydroxy-biphenyl-4-yl)-1H-pyrrol-2-ylmethylene]2-pyrrolidin-4-yl-thiazol-4-one

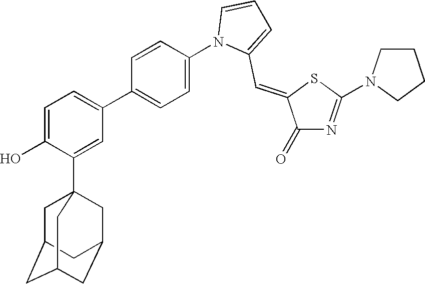

Prepared in a manner similar to that described in Example 1 using 1-(3′-adamantan-1-yl-4′-hydroxy-biphenyl-4yl)-1H-pyrrol-2-carboxaldehyde, rhodanine and pyrrolidine, mp 336-340° C. ¹H NMR (300 MHz, DMSO-d₆): δ 1.75 (s, 6 H), 1.95-2.06 (m, 7 H), 2.15 (s, 6 H), 3.59-3.70 (m, 4 H), 6.51 (t, J=3.3 Hz, 1 H), 6.72-6.74 (m, 1 H), 6.89 (d, J=9 Hz, 1 H), 7.24 (s, 1 H), 7.34-7.43 (m, 5 H), 7.76 (d, J=9 Hz, 2 H), 9.58 (s, 1 H).

The intermediate 1-(3′-adamantan-1-yl-4′-hydroxy-biphenyl-4-yl)-1H-pyrrol-2-carboxaldehyde was prepared in a similar manner to that described in example 27 using 1-(4-bromophenyl)-1H-pyrrole-2-carbaldehyde in step b.

### Example 107

### 5-[3-(3-Adamantan-1-yl-4-hydroxy-phenyl)-4-hydroxy-5-methoxy-benzylidene]-2-morpholin-4yl-thiazol-4-one

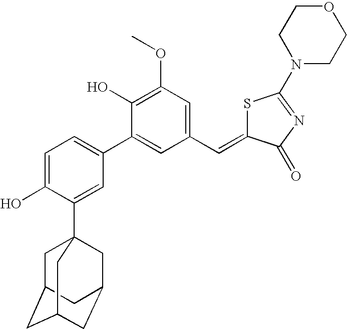

Prepared in a manner similar to that described in Example 1 using 3-(3-adamantan-1-yl-4-hydroxy-phenyl)-4-hydroxy-5-methoxy-benzaldehyde, rhodanine and morpholine, mp 335-338° C. ¹H NMR (300 MHz, DMSO-d₆): δ 1.73 (s, 6 H), 2.03 (s, 3 H), 2.11 (s, 6 H), 3.63-3.67 (m, 2 H), 3.70-3.75 (m, 4 H), 3.90(s, 5 H), 6.81 (d, J=8.4 Hz, 1 H), 7.16-7.17 (m, 2 H), 7.26 (dd, J1=9 Hz, J2=1.8 Hz, 1 H), 7.32 (d, J=2 Hz, 1 H), 7.65 (s, 1 H), 9.27 (s, 1 H), 9.41 (s, 1 H).

### Example 108

### 5-[3-(3-Adamantan-1-yl)-4-hydroxy-5-fluoro-phenyl]-benzylidene]-2-azetidin-1-yl-thiazol-4-one

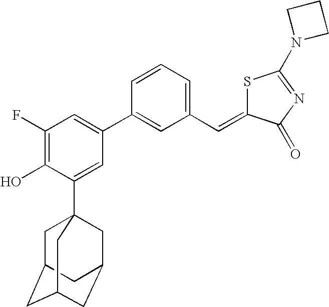

Prepared in a manner similar to that described in Example 52 using 5-[3-(3-adamantan-1yl-4-hydroxy-5-fluorophenyl)benzylidene]-2-methylsulfanyl-thiazol-4-one (example 51) and azetidine, mp 154-157° C. ¹H NMR (300 MHz, DMSO-d₆): δ 1.74 (s, 6H), 2.05 (s, 3H), 2.14 (s, 6H), 2.45-2.53 (m, 2H), 4.31 (t, J=5.1 Hz, 4H), 7.23 (s, 1H), 7.42 (dd, J=2.1 Hz, J=12.0 Hz, 1H), 7.49-7.56 (m, 2H), 7.64-7.68 (m, 1H), 7.71 (s, 1H), 7.84 (s, 1H), 9.59 (d, J=2.7 Hz, 1H). Expected: 488; Found: 489 (M+H); Expected: 488; Found: 487 (M−H).

### Example 110

### 5-[3-(3-Adamantan-1-yl-4-hydroxy-phenyl)-4-hydroxy-5-methoxybenzylidene]-2-pyrrolidin-1-yl-thiazol-4-one

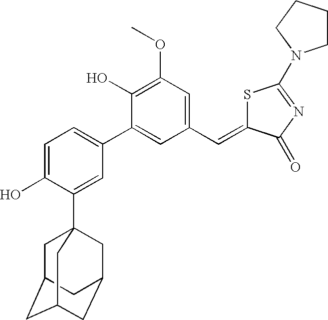

Prepared in a manner similar to that described in Example 1 using 3-(3-adamantan-1-yl-4-hydroxy-phenyl)-4-pyrrolidin-1-ylmethyl benzaldehyde, rhodanine and pyrrolidine, mp 353-355° C. ¹H NMR (300 MHz, DMSO-d₆): δ (DMSO-d6), 1.73 (s, 6 H), 1.95-2.04 (m, 7 H), 2.13 (s, 6 H), 3.60 (t, J=6 Hz, 2 H), 3.68 (t, J=6 Hz, 2 H), 3.89 (s, 3 H), 6.81 (d, J=9 Hz, 1 H), 7.16-7.18 (m, 2 H), 7.29-7.32 (m, 2 H), 7.61 (s, 1 H), 9.26 (s, 1 H), 9.42 (s, 1 H).

### Example 113

### 5-[3-(3-Adamantan-1-yl-4-hydroxy-phenyl)-benzylidene]-2-(2-hydroxymethyl-pyrrolidin-1-yl)-thiazol-4-one

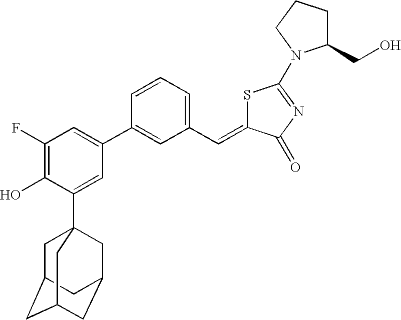

Prepared in a manner similar to that described in Example 52 using 5-[3-(3-adamantan-1-yl-4-hydroxy-5-fluorophenyl)benzylidene]-2-methylsulfanyl-thiazol-4-one (example 51) and (L)-prolinol. ¹H NMR (300 MHz, DMSO-d₆): δ 1.73 (s, 6H), 2.04 (s, 6H), 2.14 (s, 6H), 3.50-3.95 (m, 5H), 3.96-4.05 (m, 1H), 4.19-4.27 (m, 1H), 7.24 (s, 1H), 7.42 (dd, J=1.5 Hz, J=11.7 Hz, 1H), 7.53 (d, J=5.1 Hz, 2H), 7.64-7.70 (m, 1H), 7.72 (s, 1H), 7.87 (s, 1H), 9.60 (s, 1H). Expected: 532; Found: 533 (M+H); Expected: 532; Found: 531 (M−H).

### Example 116

### 5-[5-(3-Adamantan-1-yl-4-hydroxy-5-fluoro-phenyl)-pyridin-3-ylmethyl]-2-pyrrolidin-1-yl-thiazol-4-one

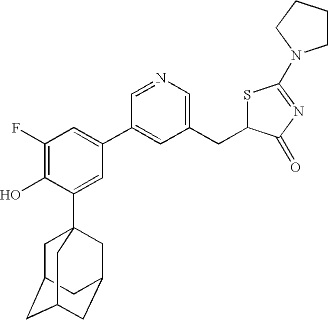

Prepared in a manner similar to that described in Example 52 using 5-[5-(3-adamantan-1-yl-5-fluoro-4-hydroxy-phenyl)-pyridin-3-ylmethyl]-2-methylsulfanyl-thiazol-4-one and pyrrolidine. 1H NMR (300 MHz, DMSO-d₆): δ 1.74 (s, 6H), 1.88-1.94 (m, 4H), 2.05 (s, 3H), 2.14 (s, 6H), 3.11 (dd, J=9.3 H, J=14.1 Hz, 1H), 3.34-3.42 (m, 2H), 3.48-3.60 (m, 3H), 4.88 (dd, J=4.5 Hz, J=9.0 Hz, 1H), 7.26 (s, 1H), 7.54 (dd, J=1.8 Hz, J=11.4 Hz, 1H), 8.31 (s, 1H), 8.52 (s, 1H), 8.87 (s, 1H), 9.82 (s, 1H). Expected: 505; Found: 506 (M+H); Expected: 505; Found: 504 (M−H).

### Example 117

### 5-[3-(3-Adamantan-1-yl-4-hydroxy-5-fluoro-phenyl)-benzylidene]-2-(cis-4-hydroxy-2(R)-carboxy-pyrrolidine-1-yl)-thiazol-4-one

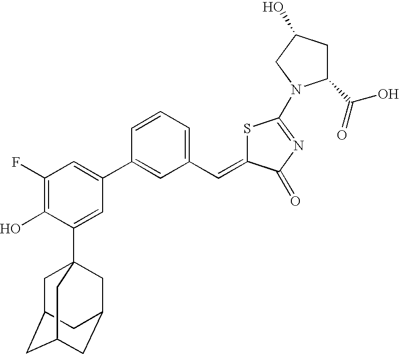

Prepared in a manner similar to that described in Example 52 using 5-[3-(3-adamantan-1-yl-4-hydroxy-5-fluorophenyl)benzylidene]-2-methylsulfanyl-thiazol-4-one (example 51) and cis-4-hydroxy-(D)-proline. ¹H NMR (300 MHz, DMSO-d₆): δ 1.73 (s, 6H), 2.04 (s, 6H), 2.14 (s, 6H), 3.50-3.95 (m, 5H), 3.96-4.05 (m, 1H), 4.19-4.27 (m, 1H), 7.24 (s, 1H), 7.42 (dd, J₁=1.5 Hz, J₂=11.7 Hz, 1H), 7.53 (d, J=5.1 Hz, 2H), 7.64-7.70 (m, 1H), 7.72 (s, 1H), 7.87 (s, 1H), 9.60 (s, 1H). Expected: 532; Found: 533 (M+H); Expected: 532; Found: 531 (M−H).

### Example 118

### See Example 162

### Example 119

### See Example 161

### Example 120

### 5-[5-(3-Adamantan-1-yl-4-hydroxy-phenyl)-6-methoxy-pyridin-3-ylmethyl]-2-morpholin-4-yl-thiazol-4-one

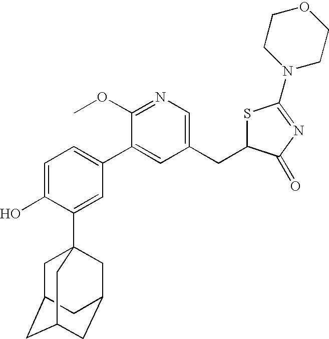

Prepared in a manner similar to that described in Example 43 using 5-[5-(3-Adamantan-1-yl-4-hydroxy-phenyl)-6-methoxy-pyridin-3-ylmethyl]-2-thioxo-thiazolidin-4-one and morpholine. mp 175-177° C. ¹H NMR (300 MHz, DMSO-d₆): δ 1.73 (br. s, 6 H), 2.04 (br. s, 3 H), 2.10 (br. s, 6 H), 2.95 (dd, J₁=9.3 Hz, J₂=13.8 Hz, 1 H), 3.40-3.80 (m, 8 H), 3.84 (s, 3 H), 4.79 (dd, J₁=4.2, J₂=8.7 Hz, 1H), 6.80 (d, J=8.4 Hz, 1 H), 7.22 (d, J=8.4 Hz, 1 H), 7.24 (s, 1 H), 7.59 (d, J=2.4 Hz, 1 H), 7.93 (d, J=2.4 Hz, 1 H), 9.46 (s, 1 H).

### Example 121

### 5-(3-Adamantan-1-yl-4-hydroxy-[1,1′;3′,1″]terphenyl-5′-ylmethylene)-2-morpholin-4-yl-thiazol-4-one

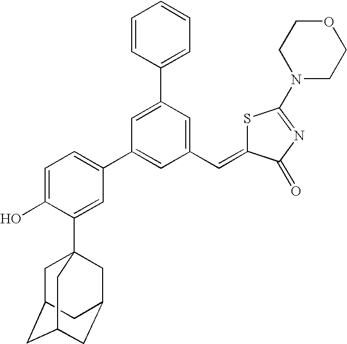

Prepared in a manner similar to that described in Example 1 using 3-Adamantan-1-yl-4-hydroxy-[1,1′;3′,1″]terphenyl-5′-carbaldehyde, rhodanine and morpholine. mp 315-320° C. ¹H NMR (300 MHz, DMSO-d₆): δ 1.76 (s, 6 H), 2.06 (s, 3 H), 2.17 (s, 6H), 3.66-3.79 (m, 6 H), 3.92-3.99 (m, 2 H), 6.90 (d, J=9.0 Hz, 1 H), 7.38-7.56 (m, 5 H), 7.73-7.87 (m, 6 H), 9.54 (s, 1 H).

### Example 122

### 5-(5′-Adamantan-1-yl-3′-fluoro-4′-hydroxy-biphenyl-3-ylmethylene)-2-thiomorpholin-4-yl-thiazol-4-one

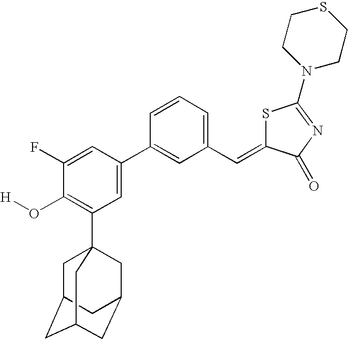

Prepared in a manner similar to that described in Example 1 using 5-[3-(3-adamantan-1-yl-4-hydroxy-5-fluoro-phenyl)benzaldehyde, rhodanine and thiomorpholine. mp 180-184° . ¹H NMR (300 MHz, DMSO-d₆): δ 1.73 (s, 6 H), 2.04 (s, 3 H), 2.14 (s, 6 H), 2.73-2.83 (m, 4 H), 3.86-3.94 (m, 2 H), 4.13-4.20 (m, 2H), 7.23 (s, 1 H), 7.42 (dd, J₁=1.8 Hz, J₂=11.7 Hz, 1 H), 7.51-7.56 (m, 2 H), 7.63-7.71 (m, 1 H), 7.76 (s, 1 H), 7.87 (s, 1 H), 9.59 (br s, 1 H).

### Example 123

### 5-[4′-Hydroxy-3′-(1-methyl-cyclohexyl)-biphenyl-3-ylmethylene]-2-morpholin-4-yl-thiazol-4-one

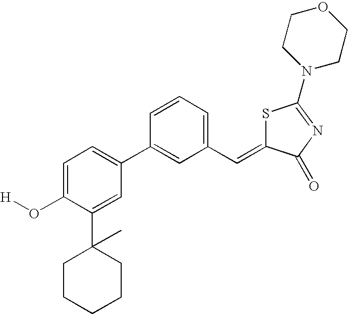

Prepared in a similar manner to that described in Example 1 using 4′-Hydroxy-3′-(1-methyl-cyclohexyl)-biphenyl-3-carbaldehyde, rhodanine, and morpholine. mp 264-267° C. ¹H NMR (300 MHz, DMSO-d₆): δ 1.28 (s, 3 H), 1.30-1.70 (m, 8 H), 2.17-2.27 (m, 2 H), 3.63-3.73 (m, 6 H), 3.91 (brs, 2 H), 6.87 (d, J=8.1 Hz, 1 H ), 7.34 (d, J=8.4 Hz, 1 H ), 7.44 (s, 1 H), 7.49-7.55 (m, 2 H), 7.59-7.65 (m, 1 H), 7.70-7.79 (m, 2 H), 9.54 (s, 1 H).

The intermediate 4′-Hydroxy-3′-(1-methyl-cyclohexyl)-biphenyl-3-carbaldehyde was prepared as followed:

### a. 4′-Hydroxy-3′-(1-methyl-cyclohexyl)-biphenyl-3-carbaldehyde

Prepared in a similar manner to that described in Example 1b, using 4-Bromo-2-(1-methyl-cyclohexyl)-phenol.

### b. 4-Bromo-2-(1-methyl-cyclohexyl)-phenol

Prepared in a similar manner to that described in Example 1 d, using 4-bromophenol and 1-methylcyclohexanol. ¹H NMR (300 MHz, CDCl₃): 1.29 (s, 3 H), 1.40-1.60 (m, 6 H), 1.60-1.75 (m, 2 H), 2.00-2.12 (m, 2 H), 5.23 (br. s, 1 H), 6.52 (d, J=8.7 Hz, 1 H), 7.12 (dd, J₁=2.1 Hz, J₂=8.4 Hz, 1 H), 7.35 (d, J=2.7 Hz, 1 H).

### Example 124

### 5-[1-(3′-Adamantan-1-yl-4′-hydroxy-biphenyl-3-yl)-ethylidene]-2-morpholin-4yl-thiazol-4-one

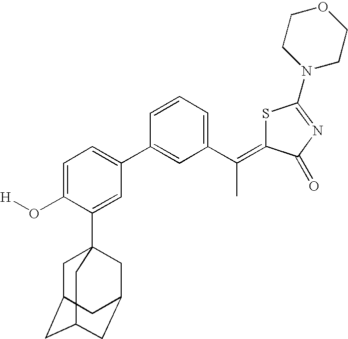

Prepared in a manner similar to that described in Example 1 using 1-(3′-Adamantan-1-yl-4′-hydroxy-biphenyl-3yl)-ethanone, rhodanine and morpholine. mp 324-325° C. ¹H NMR (300 MHz, DMSO-d₆): δ 1.74 (br. s, 6 H), 2.05 (br. s, 3 H), 2.13 (br. s, 6 H), 2.68 (s, 3 H), 3.41 (br. m, 2 H), 3.63 (br. m, 4 H), 3.82 (br. m, 2 H), 6.86 (d, J=9.3 Hz, 1H), 7.30 (m, 3H), 7.47 (t, J=8.1 Hz, 1H), 7.58(m, 2 H), 9.51 (s, 1H),

The intermediate 1-(3′-Adamantan-1-yl-4′-hydroxy-biphenyl-3-yl)-ethanone was prepared as followed:

### a. 1-(3′-Adamantan-1-yl-4′-hydroxy-biphenyl-3-yl)-ethanone

Prepared in a similar manner to that described in Example 1c, using 1-[3′-Adamantan-1-yl-4′-(tert-butyl-dimethylsilanoxy)-biphenyl-3-yl]-ethanone. ¹H NMR (300 MHz; DMSO): 1.78 (br. s, 6 H), 2.09 (br. s, 3 H), 2.17 (br. s, 6 H), 2.67 (s, 3 H), 6.90 (d, J=9.0 Hz, 1 H), 7.38 (m, 2 H), 7.57 (t, J=7.8 Hz, 1 H), 7.84 (d, J=8.1 Hz, 1 H), 7.88 (d, J=7.8 Hz, 1 H), 8.08 (s, 1 H), 9.53 (s, 1 H).

### b. 1-[3′-Adamantan-1-yl-4′-(tert-butyl-dimethylsilanoxy)-biphenyl-3-yl]-ethanone

A mixture of 1-[3′-Adamantan-1-yl-4′-(tert-butyl-dimethylsilanoxy)-biphenyl-3-yl]-ethanol (0.3 g, 0.648 mmol) and MnO₂ (676 mg, 7.78 mmol) in CH₂Cl₂ was heated at reflux under an atmosphere of argon for 16 hours. The mixture was cooled, filtered through celite, and evaporated to give 300 mg (100%) 1-[3′-Adamantan-1-yl-4′-(tert-butyl-dimethylsilanoxy)-biphenyl-3-yl]-ethanone. ¹H NMR (300 MHz; DMSO): 0.38 (s, 6 H), 1.07 (s, 9 H), 1.79 (br. s, 6 H), 2.09 (br. s, 3 H), 2.17 (br. s, 6 H), 2.65 (s, 3 H), 6.89 (dd, J₁=1.2 Hz, J₂=8.4 Hz, 1 H), 7.32 (m, 1 H), 7.48 (m, 2 H), 7.74 (d, J=7.5 Hz, 1 H), 7.86 (d, J=7.5 Hz, 1 H), 8.13 (s, 1 H).

### c. 1-[3′-Adamantan-1-yl-4′-(tert-butyl-dimethylsilanoxy)-biphenyl-3-yl]-ethanol

A solution of 3-(3-Adamantan-1-yl-4-hydroxy-phenyl)benzaldehyde (4 g, 9.0 mmol) (example 2a) in anhydrous THF (100 mL) was cooled to 0° C. then methyl magnesium bromide (9.9 mL of 1.0 M solution) was added dropwise over 10 minutes. The mixture was stirred at room temperature for 16 hours, then the reaction was quenched with H₂O and extracted with ethyl acetate (twice). The combined organic layers were washed successively with a saturated NH₄Cl solution and brine, dried over anhydrous magnesium sulfate, filtered and evaporated. The residue was purified on silica gel (eluent: hexane:ethyl acetate, 95:5) to give 2 g (48%) 1-[3′-Adamantan-1-yl-4′-(tert-butyl-dimethylsilanoxy)-biphenyl-3-yl]-ethanol. ¹H NMR (300 MHz; DMSO): 0.37 (s, 6 H), 1.06 (s, 9 H), 1.55 (s, 3 H), 1.78 (br. s, 6 H), 2.09 (br. s, 3 H), 2.16 (br. s, 6 H), 4.96 (q, J=6.6 Hz, 1 H), 6.86 (d, J=8.1 Hz, 1 H), 7.28 (m, 2 H), 7.39 (t, J=7.5 Hz, 1 H), 7.45 (m, 2 H), 7.43 (s, 1 H).

### Example 125

### 5-(3′-Adamantan-1-yl-5′-fluoro-4′-hydroxy-biphenyl-3-ylmethylene)-2-(4,5-dihydro-thiazol-2-ylamino)-thiazol-4-one

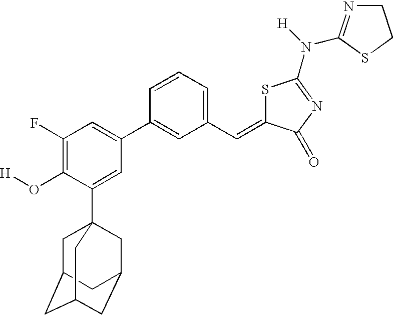

Prepared in a manner similar to that described in Example 52 using 5-[3-(3-adamantan-1-yl-4-hydroxy-5-fluorophenyl)benzylidene]-2-methylsulfanyl-thiazol-4-one (example 51) and 2-amino-2-thiazoline. mp 308-310OC. ¹H NMR (300 MHz, DMSO-d₆): δ 1.74 (s, 6 H), 2.05 (s, 3 H), 2.14 (s, 6 H), 3.43 (t, J=8.7 Hz, 2 H), 3.79 (t, J=8.1 Hz, 2 H), 7.23 (s, 1 H), 7.41 (dd, J₁=2.1 Hz, J₂=11.7 Hz, 1 H), 7.48-7.58 (m, 2 H), 7.64-7.68 (m, 1 H), 7.79 (s, 1 H), 7.86 (s, 1 H), 9.58 (d, J=3.0 Hz, 1 H), 10.3 (br s, 1 H).

### Example 126

### 5-[4,4′-Dihydroxy-5-methoxy-3′-(1-methyl-cyclohexyl)-biphenyl-3-ylmethylene]-2-morpholin-4-yl-thiazol-4-one

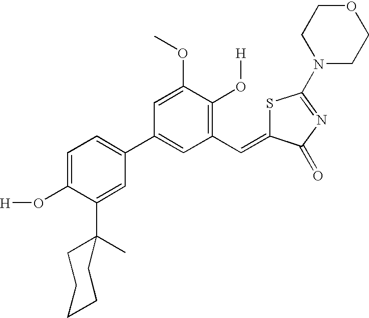

Prepared in a manner similar to that described in Example 1 using 4,4′-Dihydroxy-5-methoxy-3′-(1-methyl-cyclohexyl)-biphenyl-3-carbaldehyde, rhodanine and morpholine. mp 297-299° C. ¹H NMR (300 MHz, DMSO-d₆): δ 1.29 (s, 3 H), 1.40-1.67 (m, 8 H), 2.24 (t, J=11.1 Hz, 2 H), 3.60-3.73 (m, 6 H), 3.91 (s, 5 H), 6.85 (d, J=8.1 Hz, 1 H), 7.19 (s, 2 H), 7.29 (d, J=8.4 Hz, 1 H), 7.38 (s, 1 H), 7.98 (s, 1 H), 9.41 (s, 1 H), 9.55 (s, 1 H).

The intermediate 4,4′-Dihydroxy-5-methoxy-3′-(1-methyl-cyclohexyl)-biphenyl-3-carbaldehyde was prepared as followed:

### a. 4,4′-Dihydroxy-5-methoxy-3′-(1-methyl-cyclohexyl)-biphenyl-3-carbaldehyde

Prepared in a similar manner to that described in Example 1c, using 4′-(tert-Butyl-dimethylsilanyloxy)-4-hydroxy-5-methoxy-3(1-methyl-cyclohexyl)-biphenyl-3-carbaldehyde. ¹H NMR (300 MHz; DMSO): 1.29 (s, 3 H), 1.30-1.60 (m, 6 H), 1.60-1.75 (m, 2 H), 2.16 (m, 2 H), 3.91 (s, 3 H), 6.84 (d, J=8.1 Hz, 1 H), 7.28 (dd, J₁=1.5 Hz, J₂=8.1 Hz, 1 H), 7.36 (s, 3 H), 9.42 (s, 1 H), 10.23 (s, 1 H), 10.29 (s, 1 H).

### b. 4′-(tert-Butyl-dimethylsilanyloxy)-4-hydroxy-5-methoxy-3′-(1-methyl-cyclohexyl)-biphenyl-3-carbaldehyde

Prepared in a similar manner to that described in Example 1b, using 4-(tert-Butyl-dimethylsilanoxy)-3-(1-methyl-cyclohexyl)-boronic acid and 5-Bromo-2-hydroxy-3-methoxy-benzaldehyde.

### c. 4-(tert-Butyl-dimethylsilanoxy)-3-(1-methyl-cyclohexyl)-boronic acid

To a solution of n-BuLi (15.6 mL of 2.5 M, 39.12 mmol), in anhydrous THF (75 mL) cooled to −78° C. under an atmosphere of argon was added dropwise a solution of [4-Bromo-2-(1-methyl-cyclohexyl)-phenoxy]-tert-butyl-dimethylsilane (10 g, 26.1 mmol) in anhydrous THF (75 mL) over 1 h. Mixture stirred at −78 C for 1 h, then triisopropyl borate (18 mL, 78.2 mmol) was added dropwise over 40 min at −78 C. Warmed to 0 C, then mixture was quenched with aqueous NH₄Cl and extracted with ethyl acetate (twice). The combined organic layers were washed with brine, dried over magnesium sulfate, filtered, and evaporated to give 9.27 g (100%) of 4-(tert-Butyl-dimethylsilanoxy)-3-(1-methyl-cyclohexyl)-boronic acid as a thick oil. Used directly in next step.

### d. [4-Bromo-2-(1-methyl-cyclohexyl)-phenoxy]-tert-butyl-dimethylsilane

To a solution of 4-Bromo-2-(1-methyl-cyclohexyl)-phenol (19.4 g, 72 mmol) (example 123 b) and DMAP (260 mg, 2.16 mmol) in anhydrous DMF (130 mL) and triethylamine (8.0 mL, 79.3 mmol) was added t-butyldimethylsilyl chloride (11.95 g, 79.3 mmol). The resulting mixture was allowed to stir for 2 hours then poured into water, and extracted with ethyl acetate (twice). The combined organics were washed successively with water and brine, dried over anhydrous magnesium sulfate, filtered, and evaporated to give 27.1 g (98%) of [4-Bromo-2-(1-methyl-cyclohexyl)-phenoxy]-tert-butyldimethylsilane.

### Example 127

### 5-[4,4′-Dihydroxy-5-methoxy-3′-(1-methyl-cyclohexyl)-biphenyl-3-ylmethylene]-2-dimethylamino-thiazol-4-one

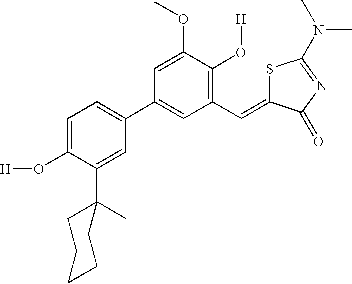

Prepared in a manner similar to that described in Example 1 using 4,4′-Dihydroxy-5-methoxy-3′-(1-methyl-cyclohexyl)-biphenyl-3-carbaldehyde (example 126a), rhodanine and dimethylamine. mp 277-279° C. ¹H NMR (300 MHz, DMSO-d₆): δ 1.29 (s, 3 H), 1.40-1.67 (m, 8 H), 2.25 (t, J=11.7 Hz, 2 H), 3.21 (s, 3 H), 3.29 (s, 3 H), 3.91 (s, 3 H), 6.85 (d, J=8.1 Hz, 1 H), 7.20 (s, 2 H), 7.31 (d, J=8.1 Hz, 1 H), 7.41 (s, 1 H), 7.95 (s, 1 H), 9.42 (s, 1 H), 9.53 (s, 1 H).

### Example 128

### 5-(3′-tert-Butyl-4,4′-dihydroxy-5-methoxy-biphenyl-3-ylmethylene)-2-morpholin-4-yl-thiazol-4-one

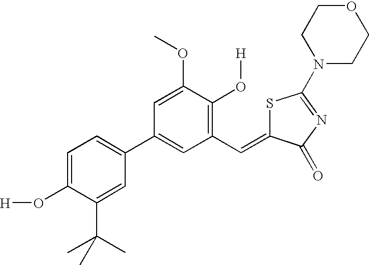

Prepared in a manner similar to that described in Example 1 using 3′-tert-Butyl-4,4′-dihydroxy-5-methoxy-biphenyl-3-carbaldehyde, rhodanine and morpholine. mp 286-289° C. ¹H NMR (300 MHz, DMSO-d₆): δ 1.40 (s, 9 H), 3.60-3.72 (m, 6 H), 3.91 (s, 5 H), 6.85 (d, J=7.8 Hz, 1 H), 7.14-7.26 (m, 2 H), 7.30 (d, J=8.4 Hz, 1 H), 7.38 (s, 1 H), 7.97 (s, 1 H), 9.47 (s, 1 H), 9.55 (s, 1 H).

This intermediate 3′-tert-Butyl-4,4′-dihydroxy-5-methoxy-biphenyl-3-carbaldehyde was prepared in a similar manner to that described in Example 1, starting with the bromination of commercially available 2-tert-Butyl-phenol with pyridinium tribromide.

### Example 129

### 5-(3′-tert-Butyl-4,4′-dihydroxy-5-methoxy-biphenyl-3-ylmethylene)-2-dimethylamino-thiazol-4-one

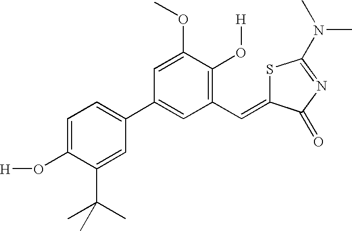

Prepared in a manner similar to that described in Example 1 using 3′-tert-Butyl-4,4′-dihydroxy-5-methoxy-biphenyl-3-carbaldehyde, rhodanine and dimethylamine. mp 272-274° C. ¹H NMR (300 MHz, DMSO-d₆): δ 1.40 (s, 9 H), 3.21 (s, 3H), 3.29 (s, 3H), 3.91 (s, 3 H), 6.85 (d, J=8.1 Hz, 1 H), 7.12-7.26 (m, 2 H), 7.32 (d, J=8.1 Hz, 1 H), 7.40 (s, 1 H), 7.94 (s, 1 H), 9.47 (s, 1 H), 9.53 (s, 1 H).

### Example 130

### 5-(3′-Cyclohexyl-4′-hydroxy-biphenyl-3-ylmethylene)-2-morpholin-4-yl-thiazol-4-one

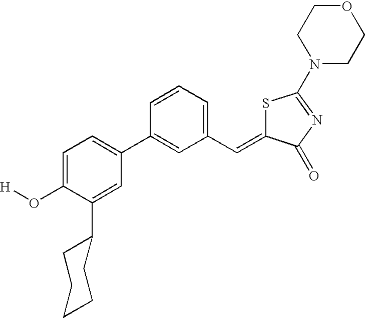

Prepared in a manner similar to that described in Example 1 using 3′-Cyclohexyl-4′-hydroxy-biphenyl-3-carbaldehyde, rhodanine and morpholine. mp 267-271° C. ¹H NMR (300 MHz, DMSO-d₆): δ 1.18-1.55 (m, 5 H), 1.65-1.85 (m, 5 H), 2.82-2.97 (m, 1 H), 3.60-3.78 (m, 6 H), 3.86-3.97 (m, 2 H), 6.88 (d, J=8.7 Hz, 1 H), 7.33 (dd, J=1.2 Hz, J=8.4 Hz, 1 H), 7.42 (s, 1 H), 7.46-7.56 (m, 2 H), 7.59-7.68 (m, 1 H), 7.75 (s, 1 H), 7.83 (s, 1 H), 9.50 (s, 1 H).

The intermediate 3′-cyclohexyl-4′-hydroxy-biphenyl-3-carbaldehyde was prepared in a similar manner to that described in Example 1, starting with the bromination of commercially available 2-cyclohexyl-phenol with pyridinium tribromide.

### Example 131

### 5-(3′-sec-Butyl-4′-hydroxy-biphenyl-3-ylmethylene)-2-morpholin-4-yl-thiazol-4-one

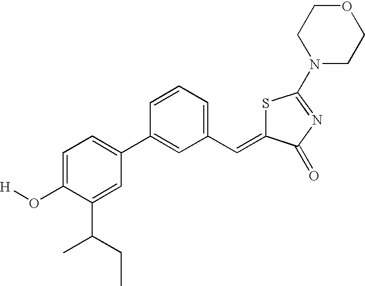

Prepared in a manner similar to that described in Example 1 using 3′-sec-Butyl-4′-hydroxy-biphenyl-3-carbaldehyde, rhodanine and morpholine. mp 228-230° C. ¹H NMR (300 MHz, DMSO-d₆): δ 0.82 (t, J=6.9 Hz, 3 H), 1.19 (d, J=7.2, 3 H), 1.45-1.80 (m, 2 H), 3.04 (m, 1 H), 3.60-3.80 (m, 6 H), 3.87-3.98 (m, 2 H), 7.28-7.37 (m, 1 H), 7.40 (s, 1 H), 7.45-7.57 (m, 2 H), 7.57-7.69 (m, 1 H), 7.75 (s, 1 H), 7.82 (s, 1 H), 9.47 (s, 1 H).

This intermediate 3′-sec-Butyl-4′-hydroxy-biphenyl-3-carbaldehyde was prepared in a similar manner to that described in Example 1, starting with the bromination of commercially available 2-sec-Butyl-phenol with pyridinium tribromide.

### Example 132

### 5-[4′-Hydroxy-3′-(1-methyl-cyclohexyl)-biphenyl-3-ylmethylene]-2-pyrrolidin-1-yl-thiazol-4-one

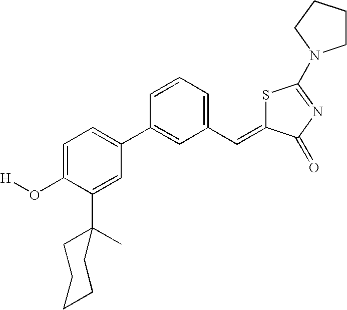

Prepared in a manner similar to that described in Example 1 using 4′-Hydroxy-3′-(1-methyl-cyclohexyl)-biphenyl-3-carbaldehyde, rhodanine and pyrrolidine. mp 278-280° C. ¹H NMR (300 MHz, DMSO-d₆): δ 1.31 (s, 3 H), 1.34-1.77 (m, 8 H), 1.93-2.10 (m, 4 H), 2.07-2.29 (m, 2 H), 3.60-3.77 (m, 4 H), 6.90 (d, J=8.7 Hz, 1 H), 7.37 (dd, J₁=2.1 Hz, J₂=8.1 Hz, 1 H), 7.48 (d, J=2.1 Hz, 1 H), 7.51-7.66 (m, 3 H), 7.12 (s, 1 H), 7.80 (s, 1 H), 9.57 (s, 1 H).

### Example 133

### 5-[4′-Hydroxy-3′-(1-methyl-cyclohexyl)-biphenyl-3-ylmethyl]-2-pyrrolidin-1-yl-thiazol-4-one

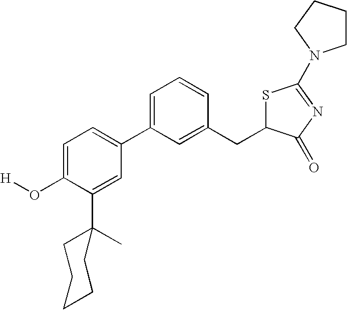

Prepared in a manner similar to that described in Example 43 using 5-[4′-Hydroxy-3′-(1-methyl-cyclohexyl)-biphenyl-3-ylmethylene]-2-thioxo-thiazolidin-4-one and pyrrolidine. mp 109-110° C. ¹H NMR (300 MHz, DMSO-d₆): δ 1.31 (s, 3 H), 1.38-1.60 (br. m, 6 H), 1.60-1.75 (br. m, 2 H), 1.92 (br. m, 4 H), 2.19 (br. m, 2 H), 2.89 (dd, J₁=10.8 Hz, J₂=14.4 Hz, 1 H), 3.35-3.60 (m, 8 H), 4.37 (dd, J₁=0.6 Hz, J₂=4.8 Hz, 1 H), 4.75 (dd, J=3.9, 10.5 Hz, 1 H), 6.86 (d, J=8.1 Hz, 1 H), 7.16 (d, J=7.2 Hz, 1 H), 7.28 (dd, J₁=1.5 Hz, J₂=8.7 Hz, 1H), 7.34 (d, J=7.5 Hz, 1H), 7.40 (m, 2H), 7.45 (s, 1H), 9.46 (s, 1H).

### Example 134

### 5-(3′-Adamantan-1-yl-4′-hydroxy-biphenyl-3-ylmethyl)-2-pyrrolidin-1-yl-thiazol-4-one

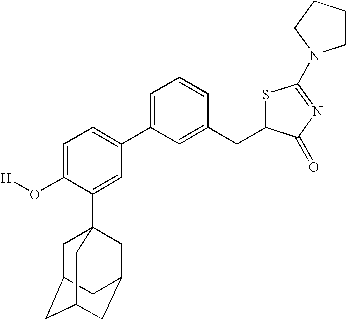

Prepared in a manner similar to that described in Example 43 using 5-(3′-Adamantan-1-yl-4′-hydroxy-biphenyl-3-ylmethyl)-2-thioxo-thiazolidin-4-one and pyrrolidine. mp 153-154° C. ¹H NMR (300 MHz, DMSO-d₆): δ 1.75 (br. s, 6 H), 1.92 (br. m, 4 H), 2.05 (br. s, 3 H), 2.13 (br. s, 6 H), 2.89 (dd, J₁=10.5 Hz, J₂=14.1 Hz, 1H), 3.40-3.50 (m, 2 H), 3.50-3.61 (m, 2H), 4.37 (t, J=5.1 Hz, 1H), 4.76 (dd, J₁=3.9 Hz, J₂=10.8 Hz, 1 H), 6.84 (d, J=7.8 Hz, 1H), 7.16 (d, J=7.5 Hz, 1 H), 7.26-7.36 (m, 3 H), 7.41 (d, J=7.8 Hz, 1 H), 7.46 (s, 1 H), 9.45 (s, 1 H).

### Example 135

### 5-(3′-Adamantan-1-yl-4′-hydroxy-biphenyl-3-ylmethyl)-2-morpholin-4-yl-thiazol-4-one

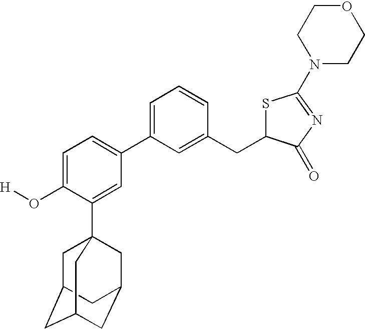

Prepared in a manner similar to that described in Example 43 using 5-(3′-Adamantan-1-yl-4′-hydroxy-biphenyl-3-ylmethyl)-2-thioxo-thiazolidin-4-one and morpholine. mp 129-131° C. ¹H NMR (300 MHz, DMSO-d₆): δ 1.74 (br. s, 6 H), 2.05 (br. s, 3 H), 2.13 (br. s, 6 H), 2.92 (dd, J₁=10.5 Hz, J₂=14.4 Hz, 1 H), 3.40-3.50 (m, 4 H), 3.56-3.64 (m, 4 H), 3.78 (m, 2 H), 4.37 (t, J=4.8 Hz, 1 H), 4.79 (dd, J₁=3.9, J₂=10.2 Hz, 1H), 6.84 (d, J=8.1 Hz, 1 H), 7.15 (d, J=7.5 Hz, 1 H), 7.26-7.35 (m, 3 H), 7.41 (d, J=7.8 Hz, 1 H), 7.45 (s, 1 H), 9.45 (s, 1 H).

### Example 136

### 5-(3′-Adamantan-1-yl-4′-hydroxy-biphenyl-3-ylmethyl)-2-dimethylamino-thiazol-4-one

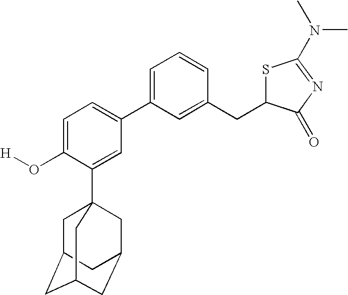

Prepared in a manner similar to that described in Example 43 using 5-(3′-Adamantan-1-yl-4′-hydroxy-biphenyl-3-ylmethyl)-2-thioxo-thiazolidin-4-one and dimethylamine. mp 243-246° C. ¹H NMR (300 MHz, DMSO-d₆): δ 1.74 (br. s, 6 H), 2.05 (br. s, 3 H), 2.13 (br. s, 6 H), 2.89 (dd, J₁=10.5 Hz, J₂=13.8 Hz, 1 H), 3.05 (s, 3 H), 3.19 (s, 3 H), 3.49 (dd, J₁=3.9 Hz, J₂=13.8 Hz), 4.78 (dd, J₁=3.9 Hz, J₂=10.2 Hz, 1 H), 6.85 (d, J=8.4 Hz, 1 H), 7.15 (d, J=7.2 Hz, 1H), 7.26-7.35 (m, 3 H), 7.41 (d, J=7.8 Hz, 1 H), 7.46 (s, 1 H), 9.44 (s, 1 H).

### Example 137

### 5-(3′-Cyclohexyl-4′-hydroxy-biphenyl-3-ylmethyl)-2-morpholin-4-yl-thiazol-4-one

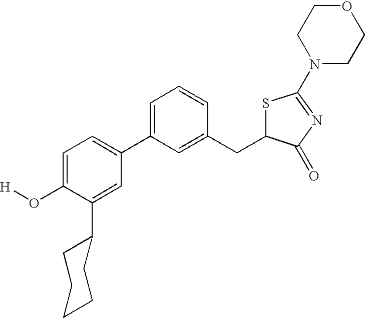

Prepared in a manner similar to that described in Example 43 using 5-(3′-Cyclohexyl-4′-hydroxy-biphenyl-3-ylmethyl))-2-thioxo-thiazolidin-4-one and morpholine. mp 100-108° C. ¹H NMR (300 MHz, DMSO-d₆): δ 1.30-1.45 (br. m, 6 H), 1.71-1.82 (m, 5 H), 1.99 (t, J=6.6 Hz, 1 H), 2.92 (dd, J₁=10.5 Hz, J₂=14.1 Hz, 1 H), 3.42-3.48 (m, 3 H), 3.58-3.66 (m, 4 H), 3.76-3.82 (m, 2 H), 4.84 (dd, J₁=4.2 Hz, J₂=10.5 Hz, 1 H ), 6.87 (d, J=8.4 Hz, 1 H), 7.15 (d, J=7.2 Hz, 1 H), 7.26 (dd, J₁=8.4, J₂=2.1 Hz, 1 H), 7.29-7.38 (m, 2 H), 7.41 (d, J=7.5 Hz, 1 H), 7.47 (s, 1 H), 9.542 (s, 1 H).

### Example 138

### 5-[4′-Hydroxy-4,5-dimethoxy-3′-(1-methyl-cyclohexyl)-biphenyl-3-ylmethyl]-2-morpholin-4-yl-thiazol-4-one

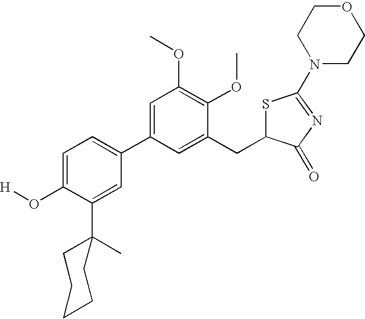

Prepared in a manner similar to that described in Example 43 using 5-[4′-Hydroxy-4,5-dimethoxy-3′-(1-methyl-cyclohexyl)--biphenyl-3-ylmethyl]-2-thioxo-thiazolidin-4-one and morpholine. mp 210-213° C. ¹H NMR (300 MHz, DMSO-d₆): δ 1.30 (s, 3 H), 1.38-1.60 (br. m, 6 H), 1.60-1.75 (br. m, 2 H), 2.18 (br. m, 2 H), 2.84 (dd, J₁=11.1 Hz, J₂=_{14.1} Hz, 1H), 3.45 (m, 2 H), 3.53 (dd, J₁=4.5 Hz, J₂=14.1 Hz, 1 H), 3.63 (m, 4 H), 3.75 (s, 3 H), 3.80 (m, 2 H), 3.87 (s, 3 H), 4.71 (dd, J₁=4.2 Hz, J₂=11.1 Hz, 1 H), 6.84 (d, J=8.4 Hz, 1 H), 6.97 (s, 1 H), 7.03 (s, 1H), 7.25 (d, J=8.1 Hz, 1H), 7.34 (s, 1 H), 9.41 (s, 1 H).

### Example 139

### 5-[4′-Hydroxy-4,5-dimethoxy-3′-(1-methyl-cyclohexyl)-biphenyl-3-ylmethyl]-2-pyrrolidin-1-yl-thiazol-4-one

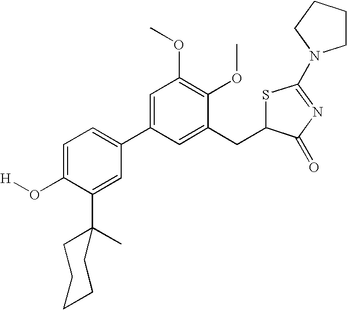

Prepared in a manner similar to that described in Example 43 using 5-[4′-hydroxy-4,5-dimethoxy-3′-(1-methyl-cyclohexyl)-biphenyl-3-ylmethyl]-2-thioxo-thiazolidin-4-one and pyrrolidine. mp 227-230° C. ¹H NMR (300 MHz, DMSO-d₆): δ 1.30 (s, 3 H), 1.38-1.60 (br. m, 6 H), 1.60-1.75 (br. m, 2 H), 1.93 (m, 4 H), 2.18 (br. m, 2 H), 2.80 (dd, J₁=11.4 Hz, J₂=13.8 Hz, 1 H), 3.38 (m, 2 H), 3.53 (dd, J₁=4.2 Hz, J₂=14.1 Hz, 1 H), 3.58 (m, 2 H), 3.75 (s, 3 H), 3.87 (s, 3 H), 4.66 (dd, J₁=4.2 Hz, J₂=11.4 Hz, 1 H), 6.84 (d, J=8.4 Hz, 1 H), 6.98 (s, 1 H), 7.03 (s, 1 H), 7.26 (d, J=8.1 Hz, 1H), 7.34 (s, 1 H), 9.41 (s, 1 H).

### Example 140

### 2-Dimethylamino-5-[4′-hydroxy-4,5-dimethoxy-3′-(1-methyl-cyclohexyl)-biphenyl-3-ylmethyl]-thiazol-4-one

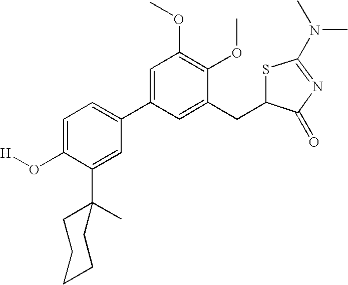

Prepared in a manner similar to that described in Example 43 using 5-[4′-hydroxy-4,5-dimethoxy-3′-(1-methyl-cyclohexyl)-biphenyl-3-ylmethyl]-2-thioxo-thiazolidin-4-one and dimethylamine. mp 187-188° C. ¹H NMR (300 MHz, DMSO-d₆): δ 1.30 (s, 3 H), 1.38-1.60 (br. m, 6 H), 1.60-1.75 (br. m, 2 H), 2.18 (m, 2 H), 2.84 (dd, J₁=11.1 Hz, J₂=14.1 Hz, 1H), 3.06 (s, 3 H), 3.20 (s, 3 H), 3.52 (dd, J₁=3.9 Hz, J₂=13.8 Hz, 1 H), 3.75 (s, 3 H), 3.87 (s, 3 H), 4.70 (dd, J₁=4.2, J₂=11.1 Hz, 1 H), 6.84 (d, J=8.1 Hz, 1 H), 6.98 (d, J=1.5 Hz, 1 H), 7.03 (d, J=1.5 Hz, 1 H), 7.26 (dd, J₁=1.5 Hz, J₂=8.1 Hz, 1 H), 7.34 (d, J=1.8 Hz, 1 H), 9.41 (s, 1 H).

### Example 141

### 5-[4′-Hydroxy-3′-(1-methyl-cyclohexyl)-biphenyl-3-ylmethyl]-2-morpholin-4-yl-thiazol-4-one

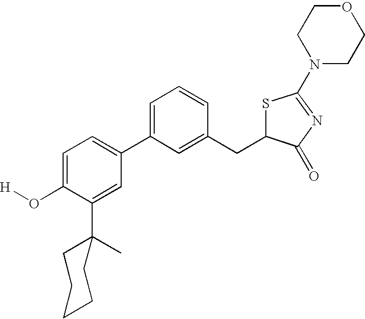

Prepared in a manner similar to that described in Example 43 using 5-[4′-Hydroxy-3′-(1-methyl-cyclohexyl)-biphenyl-3-ylmethyl]-2-thioxo-thiazolidin-4-one and morpholine. mp 98-99° C. ¹H NMR (300 MHz, DMSO-d₆): δ 1.30 (s, 3 H), 1.38-1.60 (br. m, 6 H), 1.60-1.75 (br. m, 2 H), 2.19 (br. m, 2 H), 2.93 (dd, J₁=10.2 Hz, J₂=14.4 Hz, 1H), 3.46 (m, 2 H), 3.59 (m, 4 H), 3.78 (t, J=4.5 Hz, 1 H), 4.79 (dd, J₁=3.9 Hz, J₂=9.9 Hz, 1 H), 6.86 (d, J=8.4 Hz, 1 H), 7.16 (d, J=6.9 Hz, 1 H), 7.28 (dd, J₁=2.1 Hz, J₂=8.4 Hz, 1 H), 7.34 (d, J=7.5 Hz, 1 H), 7.36-7.42 (m, 2 H), 7.44 (s, 1 H), 9.46 (s, 1 H).

### Example 142

### 2-Dimethylamino-5-[4′-hydroxy-3′-(1-methyl-cyclohexyl)-biphenyl-3-ylmethyl]-thiazol-4-one

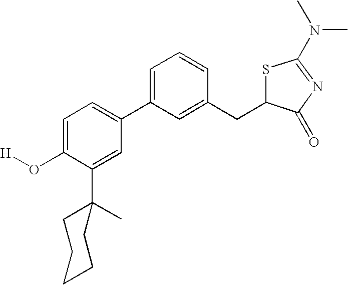

Prepared in a manner similar to that described in Example 43 using 5-[4′-hydroxy-3′-(1-methyl-cyclohexyl)-biphenyl-3-ylmethyl]-2-thioxo-thiazolidin-4-one and dimethylamine. mp 108-110° C. ¹H NMR (300 MHz, DMSO-d₆): δ 1.31 (s, 3 H), 1.38-1.60 (br. m, 6 H), 1.60-1.75 (br. m, 2 H), 2.19 (br. m, 2 H), 2.91 (dd, J₁=10.5 Hz, J₂=14.1 Hz, 1 H), 3.05 (s, 3 H), 3.19 (s, 3 H), 3.49 (dd, J₁=4.2 Hz, J₂=14.4 Hz, 1 H), 4.78 (dd, J₁=4.2, J₂=10.5 Hz, 1 H), 6.86 (d, J=8.1 Hz, 1 H), 7.16 (d, J=7.2 Hz, 1 H), 7.29 (d, J=7.8 Hz, 1 H), 7.34 (d, J=7.5 Hz, 1 H), 7.38-7.42 (m, 2 H), 7.45 (s, 1 H), 9.46 (s, 1 H).

### Example 143

### 5-[3′-(1,1-Dimethyl-propyl)-4′-hydroxy-biphenyl-3-ylmethyl]-2-pyrrolidin-1-yl-thiazol-4-one

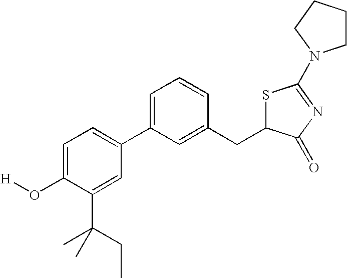

Prepared in a manner similar to that described in Example 43 using 5-[3′-(1,1-Dimethyl-propyl)-4′-hydroxy-biphenyl-3-ylmethyl]-2-thioxo-thiazolidin-4-one (example 151a) and pyrrolidine. mp 216-219° C. ¹H NMR (300 MHz, DMSO-d₆): δ 0.62 (t, J=7.5 Hz, 3 H), 1.35 (s, 6 H), 1.90 (m, 6 H), 2.89 (dd, J₁=10.8 Hz, J₂=14.1 Hz, 1 H), 3.38 (m, 2 H), 3.50 (dd, J₁=3.9 Hz, J₂=13.8 Hz, 1 H), 3.57 (m, 2 H), 4.76 (dd, J₁=3.9 Hz, J₂=10.5 Hz, 1 H), 6.84 (d, J=8.1 Hz, 1 H), 7.16 (d, J=7.5 Hz, 1 H), 7.19 (dd, J₁=2.4 Hz, J₂=7.5 Hz, 1 H), 7.30-7.36 (m, 2 H), 7.41 (d, J=7.8 Hz, 1 H), 7.46 (s, 1 H), 9.44 (s, 1 H).

### Example 144

### 5-(3′-Cyclopentyl-4′-hydroxy-biphenyl-3-ylmethyl)-2-pyrrolidin-1-yl-thiazol-4-one

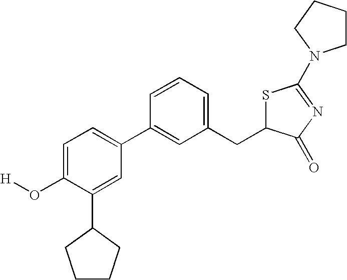

Prepared in a manner similar to that described in Example 43 using 5-(3′-Cyclopentyl-4′-hydroxy-biphenyl-3-ylmethyl)-2-thioxo-thiazolidin-4-one and pyrrolidine. mp 99-105° C. ¹H NMR (300 MHz, DMSO-d₆): δ 1.63-1.78 (brd. m, 6 H), 1.89-2.01 (Brd. m, 6 H), 2.89 (dd, J₁=10.5 Hz, J₂=14.1 Hz, 1 H), 3.23-3.31 (m, 2H), 3.52 (dd, J₁=3.6 Hz, J₂=14.1 Hz, 1 H), 3.57 (brd. m, 3 H), 4.76 (dd, J₁=3.9 Hz, J₂=10.5, 1 H ), 6.86 (d, J=8.7 Hz, 1 H), 7.16 (d, J=7.2 Hz, 1 H), 7.27 (dd, J₁=8.1 Hz, J₂=1.8 Hz, 1 H), 7.32 (d, J=7.5 Hz, 1 H), 7.37 (d, J=1.8 Hz, 1 H), 7.42 (d, J=7.5 Hz, 1 H), 7.46 (s, 1 H), 9.41 (s, 1 H).

The intermediate 5-(3′-cyclopentyl-4′-hydroxy-biphenyl-3-ylmethyl)-2-thioxo-thiazolidin-4-one was prepared in a manner similar to that described in Example 151, starting with the bromination of commercially available 2-cyclopentyl-phenol with pyridinium tribromide.

### Example 145

### 5-[3′-(1,1-Dimethyl-propyl)-4′-hydroxy-biphenyl-3-ylmethylene]-2-pyrrolidin-1-yl-thiazol-4-one

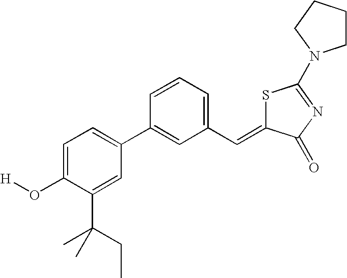

Prepared in a manner similar to that described in Example 1 using 3′-(1,1-Dimethyl-propyl)-4′-hydroxy-biphenyl-3-carbaldehyde (example 151c), rhodanine and pyrrolidine. mp 282-283° C. ¹H NMR (300 MHz, DMSO-d₆): 0.64 (t, J=7.8 Hz, 3 H), 1.38 (s, 6 H), 1.91 (q, J=7.8 Hz, 2 H), 2.01 (m, 4H), 3.62 (t, 2 H), 3.71 (t, 2 H), 6.88 (d, J=8.1 Hz, 1 H), 7.38 (dd, J₁=2.1 Hz, J₂=7.8 Hz, 1 H), 7.42 (d, J=2.1 Hz, 1 H), 7.50-7.60 (m, 2 H), 7.64 (m, 1 H), 7.72 (s, 1 H), 7.82 (s, 1 H), 9.55 (s, 1 H).

### Example 146

### 5-[3′-(1,1-Dimethyl-propyl)-4′-hydroxy-biphenyl-3-ylmethylene]-2-morpholin-4-yl-thiazol-4-one

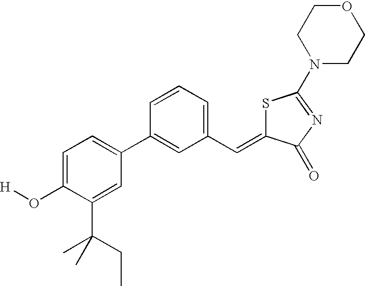

Prepared in a manner similar to that described in Example 1 using 3′-(1,1-Dimethyl-propyl)-4′-hydroxy-biphenyl-3-carbaldehyde (example 151c), rhodanine and morpholine. mp 174-176° C. ¹H NMR (300 MHz, DMSO-d₆): δ 0.63 (t, J=7.8 Hz, 3 H), 1.37 (s, 6 H), 1.88 (q, J=7.8 Hz, 2 H), 3.68 (m, 2 H), 3.75 (m, 4 H), 3.94 (m, 2 H), 6.88 (d, J=8.1 Hz, 1 H), 7.38 (dd, J₁=1.8 Hz, J₂=8.1 Hz, 1 H), 7.40 (s, 1 H), 7.50-7.60 (m, 2 H), 7.64 (m, 1 H), 7.76 (s, 1 H), 7.83 (s, 1 H), 9.55 (s, 1 H).

### Example 147

### 5-[4,4′-Dihydroxy-5-methoxy-3′-(1-methyl-cyclohexyl)-biphenyl-3-ylmethyl]-2-pyrrolidin-1-yl-thiazol-4-one

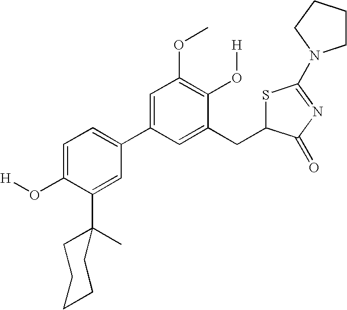

Prepared in a manner similar to that described in Example 43 using: 5-[4,4′-Dihydroxy-5-methoxy-3′-(1-methyl-cyclohexyl)-biphenyl-3-ylmethyl]-2-thioxo-thiazolidin-4-one and pyrrolidine. mp 204-206° C. ¹H NMR (300 MHz, DMSO-d₆): δ 1.30 (s, 3 H), 1.40-1.60 (m, 6 H), 1.70 (m, 2 H), 1.93 (m, 4 H), 2.17 (m, 2 H), 2.69 (dd, J₁=11.4 Hz, J₂=13.8 Hz, 1 H), 3.16 (d, 2 H), 3.50-3.65 (m, 3 H), 3.86 (s, 3 H), 4.69 (dd, J₁=3.6 Hz, J₂=11.1 Hz, 1 H), 6.82 (d, J=8.4 Hz, 1 H), 6.89 (d, J=2.1 Hz, 1 H), 6.97 (d, J=2.1 Hz, 1 H), 7.21 (dd, J₁=2.1 Hz, J₂=8.4 Hz, 1 H), 7.32 (d, J=2.1 Hz, 1 H), 8.76 (s, 1 H), 9.32 (s, 1 H).

### Example 148

### 5-[4,4′-Dihydroxy-5-methoxy-3′-(1-methyl-cyclohexyl)-biphenyl-3-ylmethyl]-2-morpholin-4-yl-thiazol-4-one

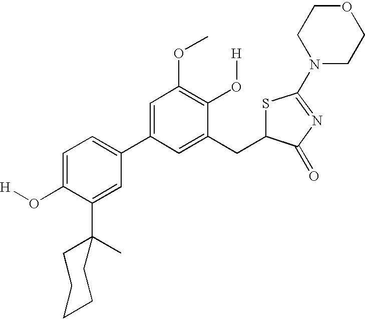

Prepared in a manner similar to that described in Example 43 using: 5-[4,4′-Dihydroxy-5-methoxy-3′-(1-methyl-cyclohexyl)-biphenyl-3-ylmethyl]-2-thioxo-thiazolidin-4-one and morpholine. mp 221-223° C. ¹H NMR (300 MHz, DMSO-d₆): δ 1.30 (s, 3 H), 1.40-1.60 (m, 6 H), 1.70 (m, 2 H), 2.17 (m, 2 H), 2.73 (dd, J=11.1 Hz, J₂=13.8 Hz, 1H), 3.16 (d, 2 H), 3.45 (m, 2 H), 3.55 (dd, J₁=3.9 Hz, J₂=14.1 Hz, 1 H), 3.63 (m, 4 H), 3.81 (m, 2 H), 3.85 (s, 3 H), 4.72 (dd, J₁=4.2 Hz, J₂=11.1 Hz, 1 H), 6.82 (d, J=8.4 Hz, 1 H), 6.88 (d, J=1.8 Hz, 1 H), 6.97 (d, J=2.1 Hz, 1 H), 7.20 (dd, J₁=2.1 Hz, J₂=8.1 Hz, 1 H), 7.31 (d, J=2.1 Hz, 1 H), 8.77 (s, 1 H), 9.32 (s, 1 H).

### Example 149

### 5-[4′-Hydroxy-4-methoxy-3′-(1-methyl-cyclohexyl)-biphenyl-3-ylmethyl]-2-pyrrolidin-1-yl-thiazol-4-one

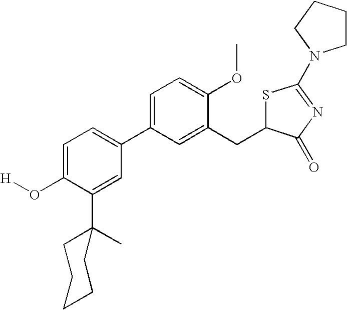

Prepared in a manner similar to that described in Example 43 using 5-[4′-Hydroxy-4-methoxy-3′-(1-methyl-cyclohexyl)-biphenyl-3-ylmethyl]-2-thioxo-thiazolidin-4-one and pyrrolidine. mp 121-122° C. ¹H NMR (300 MHz, DMSO-d₆): δ 1.30 (s, 3 H), 1.40-1.60 (m, 6 H), 1.69 (m, 2 H), 1.92 (m, 4 H), 2.17 (m, 2 H), 2.75 (dd, J₁=11.4 Hz, J₂=13.2 Hz, 1 H), 3.38 (m, 2H), 3.43 (dd, J₁=3.9 Hz, J₂=13.2 Hz, 1 H), 3.58 (m, 2 H), 3.82 (s, 3 H), 4.67 (dd, J₁=3.9 Hz, J₂=11.1 Hz, 1 ), 6.83 (d, J=8.4 Hz, 1H), 7.02 (d, J=8.4 Hz, 1 H), 7.22 (d, J=8.4 Hz, 1 H), 7.36 (m, 3 H), 9.36 (s, 1 H).

### Example 150

### 5-[4′-Hydroxy-5-methoxy-3′-(1-methyl-cyclohexyl)-biphenyl-3-ylmethyl]-2-pyrrolidin-1-yl-thiazol-4-one

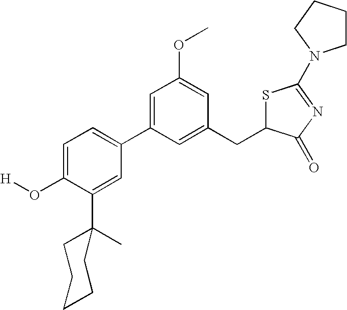

Prepared in a manner similar to that described in Example 43 using 5-[4′-Hydroxy-5-methoxy-3′-(1-methyl-cyclohexyl)-biphenyl-3-ylmethyl]-2-thioxo-thiazolidin-4-one and pyrrolidine. mp 88-91° C. ¹H NMR (300 MHz, DMSO-d₆): δ 1.30 (s, 3 H), 1.40-1.60 (m, 6 H), 1.69 (m, 2 H), 1.92 (m, 4 H), 2.17 (m, 2 H), 2.83 (t, J=12.6 Hz, 1H), 3.39 (m, 2 H), 3.48 (d, J=12.3 Hz, 1 H), 3.58 (m, 2 H), 3.79 (s, 3 H), 4.76 (d, J=6.9 Hz, 1 H), 6.78 (s, 1 H), 6.85 (d, J=8.4 Hz, 1 H), 6.92 (s, 1 H), 7.03 (s, 1 H), 7.28 (d, J=8.1 Hz, 1 H), 7.38 (s, 1 H), 9.46 (s, 1 H).

### Example 151

### 5-[3′-(1,1-Dimethyl-propyl)-4′-hydroxy-biphenyl-3-ylmethyl]-2-morpholin-4-yl-thiazol-4-one

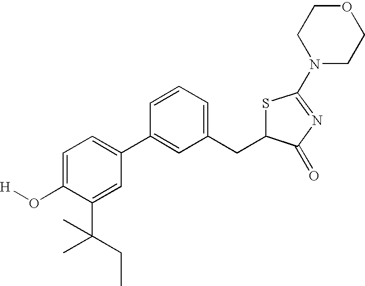

A solution of toluene (50 mL), morpholine (0.124 mL, 1.43 mmol), acetic acid (0.082 mL, 1.43 mmol) and 5-[3′-(1,1-Dimethyl-propyl)-4′-hydroxy-biphenyl-3-ylmethyl]-2-thioxo-thiazolidin-4-one (0.5 g, 1.29 mmol) was heated at reflux overnight under an argon atmosphere. After cooling the solvent removed by distillation. The residue was purified on silica gel (eluent: ethyl acetate) to give 476 mg (84%) of 5-[3′-(1,1-Dimethyl-propyl)-4′-hydroxy-biphenyl-3-ylmethyl]-2-morpholin-4-yl-thiazol-4-one. mp 74-77° C. ¹H NMR (300 MHz, DMSO-d₆): δ 0.62 (t, J=6.9 Hz, 3 H), 1.35 (s, 6 H), 1.87 (q, J=6.9 Hz, 2 H), 2.83 (dd, J₁=10.8 Hz, J₂=13.2 Hz, 1 H), 3.46 (m, 2H), 3.59 (m, 5 H), 3.78 (m, 2 H), 4.80 (dd, J₁=3.0 Hz, J₂=9.6 Hz, 1H), 6.85 (d, J=8.1 Hz, 1 H), 7.16 (d, J=7.5 Hz, 1 H), 7.33 (m, 3 H), 7.42 (d, J=7.5 Hz, 1 H), 7.46 (s, 1 H), 9.45 (s, 1 H).

The intermediate 5-[3′-(1,1-Dimethyl-propyl)-4′-hydroxy-biphenyl-3-ylmethyl]2-thioxo-thiazolidin-4-one was prepared as followed:

### a. 5-[3′-(1,1-Dimethyl-propyl)-4′-hydroxy-biphenyl-3-ylmethyl]-2thioxo-thiazolidin-4-one

To a solution of 5-[3′-(1,1-Dimethyl-propyl)-4′-hydroxy-biphenyl-2-ylmethylene]-2-thioxo-thiazolidin-4-one (2.36 g, 6.15 mmol) in anhydrous pyridine (30 mL, 369 mmol) and THF (150 mL) under an atmosphere of argon was added LiBH₄ (15.4 mL of 2 M in THF, 30.76 mmol). The resulting mixture was heated at reflux for 16 h. The reaction mixture was cooled and quenched by dropwise addition of 1.0 N HCl and extracted with ethyl acetate. The organic layer was washed successively with 1.0 N HCl, water and brine, dried over anhydrous magnesium sulfate, filtered, and evaporated. The residue was purified on silica gel (eluent:hexane:ethyl acetate, 4:1) to give 2.02 g (85%) of 5-[3′-(1,1-Dimethyl-propyl)-4′-hydroxy-biphenyl-3-ylmethyl]-2-thioxo-thiazolidin-4-one. Used as this in the next step.

### b. 5-[3′-(1,1-Dimethyl-propyl)-4′-hydroxy-biphenyl-2-ylmethylene]-2-thioxo-thiazolidin-4-one

A solution of anhydrous toluene (120 mL), aniline (0.136 mL), acetic acid (0.085 mL), 3′-(1,1-Dimethyl-propyl)-4′-hydroxy-biphenyl-3-carbaldehyde (2.0 g, 7.45 mmol) and rhodanine (1.09 g, 8.2 mmol) was heated at reflux overnight under an argon atmosphere. The toluene was removed by distillation and the product crystallized from ethanol/water to give 2.09 g (73%) of 5-[3′-(1,1-dimethyl-propyl)-4′-hydroxy-biphenyl-2-ylmethylene]-2-thioxo-thiazolidin-4-one as a yellow solid. ¹H NMR (300 MHz; CDCl₃): δ 0.63 (t, J=7.5 Hz, 3 H), 1.37 (s, 6 H), 1.88 (q, J=7.5 Hz, 2 H), 6.88 (d, J=8.4 Hz, 1 H), 7.39 (m, 2 H), 7.48 (d, J=7.8 Hz, 1 H), 7.58 (t, J=8.1 Hz, 1 H), 7.70 (d, J=7.5 Hz, 1 H), 7.76 (s, 1 H), 7.80 (s, 1 H), 9.57 (s, 1 H).

### c. 3′-(1,1-Dimethyl-propyl)-4′-hydroxy-biphenyl-3-carbaldehyde

To a solution of 4′-(tert-Butyldimethylsilanyloxy)-3′-(1,1-dimethyl-propyl)-biphenyl-3-carbaldehyde (9.46 g, 25.8 mmol) in anhydrous THF (400 mL) under an atmosphere of argon cooled to 0° C. was added dropwise a 1.0 M solution of tetrabutyl ammonium fluoride in THF (31 mL, 30.96 mmol). After 1 hr the mixture was poured into a slurry of ice water and extracted with ethylacetate twice. The combined organic layers were washed successively with water and brine, dried over anhydrous magnesium sulfate, filtered, and evaporated. The resulting product was stirred in hexane, filtered and dried under reduced pressure to give 5.82 g (84%) of 3′-(1,1-dimethyl-propyl)-4′-hydroxy-biphenyl-3-carbaldehyde as a white powder. ¹H NMR (300 MHz; CDCl₃): δ 0.71 (t, J=7.5 Hz, 3 H), 1.43 (s, 6 H), 1.91 (q, J=7.5 Hz, 2 H), 5.03 (s, 1 H), 6.77 (d, J=8.4 Hz, 1 H), 7.34 (dd, J₁=2.4 Hz, J₂=8.1 Hz, 1 H), 7.46 (d, J=2.4 Hz, 1 H), 7.58 (t, J=7.8 Hz, 1 H), 7.81 (m, 1 H), 7.83 (m, 1 H), 8.05 (t, J=1.8 Hz, 1 H), 10.09 (s, 1 H).

### d. 4′-(tert-Butyldimethylsilanyloxy)-3′-(1,1-dimethyl-propyl)-biphenyl-3-carbaldehyde

A mixture of [4-Bromo-2-(1,1-dimethyl-propyl)-phenoxy]-tert-butyldimethylsilane (10.00 g, 27.98 mmol), 3-formylphenylboronic acid (4.6 g, 30.8 mmol) and sodium carbonate (8.9 g, 83.94 mmol) in a mixture of toluene: ethanol (4:1, 200 mL) and water (20 mL) was degassed with argon for 45 minutes. Tetrakis(triphenylphosphine)palladium(0) (970 mg, 0.84 mmol) was added and the mixture heated at reflux under argon for 16 hours. The solution was cooled to room temperature, diluted with ethyl acetate and washed successively with water and brine, dried over anhydrous magnesium sulfate, filtered and evaporated. The residue was purified on silica gel (eluent: hexane:ethyl acetate, 97:3) to give 9.46 g (92%) of 4-(tert-Butyldimethylsilanyloxy)-3′(1,1-dimethyl-propyl)-biphenyl-3-carbaldehyde as a clear oil. ¹H NMR (300 MHz; CDCl₃): δ 0.35 (s, 6 H), 0.68 (t, J=7.5 Hz, 3 H), 1.04 (s, 9 H), 1.40 (s, 6 H), 1.90 (q, J=7.5 Hz, 2 H), 6.89 (d, J=8.7 Hz, 1 H), 7.33 (dd, J=2.4, 8.4 Hz, 1 H), 7.47 (d, J=2.4 Hz, 1 H), 7.56 (t, J=7.5 Hz, 1 H), 7.79 (m, 2 H), 8.04 (t, J=1.8 Hz, 1 H), 10.07 (s, 1 H).

### e. [4-Bromo-2-(1,1-dimethyl-propyl)-phenoxy]-tert-butyldimethylsilane

To a solution of 4-Bromo-2-(1,1-dimethyl-propyl)-phenol (15.2 g, 61 mmol) and DMAP (223 mg, 1.83 mmol) in anhydrous DMF (300 mL) and triethylamine (9.4 mL, 67 mmol) was added t-butyldimethylsilyl chloride (10 g, 67 mmol). The resulting mixture was allowed to stir for 17 hours then poured into water and extracted with ethyl acetate (twice). The combined organics were washed successively with water and brine, dried over anhydrous magnesium sulfate, filtered, and evaporated to give 22 g (100%) of [4-Bromo-2-(1,1-dimethyl-propyl)-phenoxy]-tert-butyldimethylsilane as a clear oil. ¹H NMR (300 MHz; CDCl₃): δ 0.20 (s, 6 H), 0.53 (t, J=7.5 Hz, 3 H), 0.91 (s, 9 H), 1.20 (s, 6 H), 1.73 (q, J=7.5 Hz, 2 H), 6.56 (d, J=8.4 Hz, 1 H), 7.05 (dd, J₂=2.1 Hz, J₂=8.4 Hz, 1 H), 7.19 (d, J=2.1 Hz, 1 H).

### f. 4-Bromo-2-(1,1-dimethyl-propyl)-phenol

To a solution of 2-(1,1-dimethyl-propyl)-phenol (10 g, 61 mmol) in CH₂Cl₂ under an atmosphere of argon was added pyridinium tribromide (21 g, 67 mmol). After stirring at room temperature for 2 hours, the resulting mixture was poured into 1.0 N HCl and extracted with CH₂Cl₂ (twice). The combined organics were washed with water, then brine and dried (MgSO₄). The mixture was filtered and evaporated to give 15.2 g (100%) of 4-Bromo-2-(1,1-dimethyl-propyl)-phenol. ¹H NMR (300 MHz; CDCl₃): δ 0.67 (t, J=7.5 Hz, 3 H), 1.34 (s, 6 H), 1.84 (q, J=7.5 Hz, 2 H), 4.84 (s, 1 H), 6.53 (d, J=8.4 Hz, 1 H), 7.16 (dd, J=2.1, 8.4 Hz, 1 H), 7.29 (d, J=2.1 Hz, 1 H).

### Example 152

### 5-[3′-(1,1-Dimethyl-propyl)-4-fluoro-4′-hydroxy-biphenyl-3-ylmethylene]-2-morpholin-4-yl-thiazol-4one

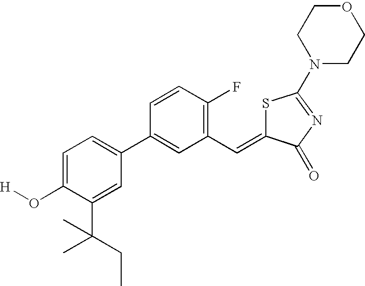

Prepared in a manner similar to that described in Example 1 using 3′-(1,1-Dimethyl-propyl)-4-fluoro-4′-hydroxy-biphenyl-3-carbaldehyde, rhodanine and morpholine. mp 246-247° C. ¹H NMR (300 MHz, DMSO-d₆): δ 0.64 (t, J =7.5 Hz, 3 H), 1.37 (s, 6 H), 1.88 (q, J=7.5 Hz, 2 H), 3.68 (m, 2 H), 3.74 (m, 4 H), 3.94 (m, 2 H), 6.89 (d, J=9.0 Hz, 1 H), 7.36 (m, 2 H), 7.42 (d, J=8.4 Hz, 1 H), 7.60-7.80 (m, 3 H), 9.58 (s, 1 H).

### Example 153

### 5-{5-[3-(1,1-Dimethyl-propyl)-4-hydroxy-phenyl]-thiophen-2-ylmethylene}-2-morpholin-4-yl-thiazol-4-one

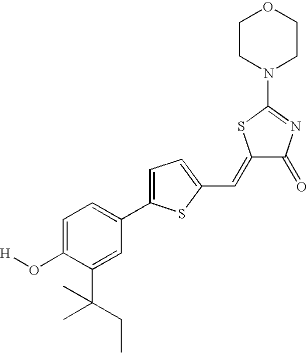

Prepared in a manner similar to that described in Example 1 using 5-[3-(1,1-Dimethyl-propyl)-4-hydroxy-phenyl]-thiophen-2-carbaldehyde, rhodanine and morpholine. mp 322-323° C. ¹H NMR (300 MHz, DMSO-d₆): δ 0.62 (t, J=7.5 Hz, 3 H), 1.35 (s, 6 H), 1.89 (q, J=7.5 Hz, 2 H), 3.60-3.80 (m, 6 H), 3.92 (m, 2 H), 6.84 (d, J=8.4 Hz, 1 H), 7.40 (m, 2 H), 7.46 (d, J=3.9 Hz, 1 H), 7.57 (dd, J=0.6, 3.9 Hz, 1 H), 7.83 (d, J=0.6 Hz, 1 H), 9.81 (s, 1 H).

### Example 154

### 5-[3′-(1,1-Dimethyl-propyl)-5′-fluoro-4′-hydroxy-biphenyl-3-ylmethylene]-2-morpholin-4-yl-thiazol-4-one

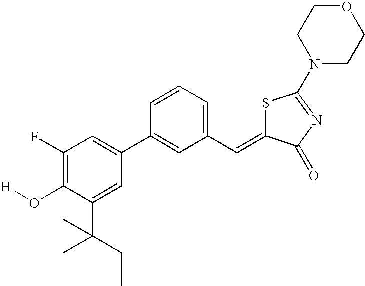

Prepared in a manner similar to that described in Example 1 using 3′-(1,1-Dimethyl-propyl)-5′-fluoro-4′-hydroxy-biphenyl-3-carbaldehyde, rhodanine and morpholine. mp 206-208° C. ¹H NMR (300 MHz, DMSO-d₆): δ 0.64 (t, J=7.5 Hz, 3 H), 1.38 (s, 6 H), 1.89 (q, J=7.5 Hz, 2 H), 3.68 (m, 2 H), 3.75 (m, 4 H), 3.94 (m, 2 H), 7.26 (s, 1 H), 7.44 (dd, J₁=1.8 Hz, J₂=11.4 Hz, 1 H), 7.55 (s, 1 H), 7.57 (s, 1 H), 7.69 (m, 1 H), 7.77 (s, 1 H), 7.89 (s, 1 H), 9.61 (d, J=2.7 Hz, 1 H).

### Example 155

### 5-{6-[3-(1,1-Dimethyl-propyl)-4-hydroxy-phenyl]-pyridin-2ylmethyl}--2-morpholin-4-yl-thiazol-4-one

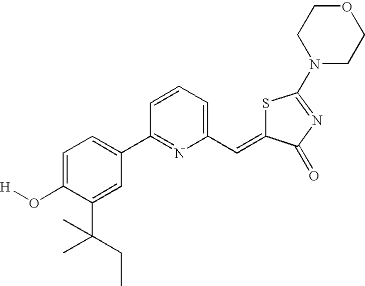

Prepared in a manner similar to that described in Example 43 using 5-{6-[3-(1,1-Dimethyl-propyl)-4-hydroxy-phenyl]-pyridin-2-ylmethyl}-2-thioxo-thiazolidin-4-one and morpholine. mp 240-242° C. 1H NMR (300 MHz, DMSO-d₆): δ 0.62 (t, J=7.5 Hz, 3 H), 1.38 (s, 6 H), 1.88 (q, J=7.5 Hz, 2 H), 3.15 (dd, J₁=11.7 Hz, J₂=16.5 Hz, 1 H), 3.49 (m, 2 H), 3.66 (m, 4 H), 3.75 (dd, J₁=3.0 Hz, J₂=16.2 Hz, 1 H), 3.84 (m, 2 H), 4.70 (dd, J₁=3.0 Hz, J₂=11.7 Hz, 1 H), 6.85 (d, J=8.7 Hz, 1 H), 7.19 (d, J=6.6 Hz, 1 H), 7.71 (m, 3 H), 7.98 (d, J=2.4 Hz, 1 H), 9.66 (s, 1 H).

### Example 156

### 5-{4-[3-(1,1-Dimethyl-propyl)-4-hydroxy-phenyl]-1H-pyrrol-2-ylmethylene}-2-morpholin-4-yl-thiazol-4-one

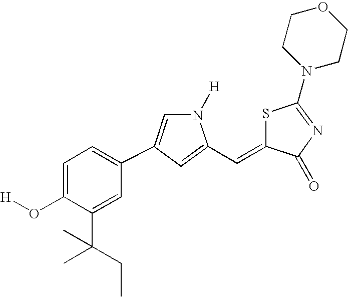

Prepared in a manner similar to that described in Example 1 using 4-[3-(1,1-Dimethyl-propyl)-4-hydroxy-phenyl]-1H-pyrrol-2-carbaldehyde, rhodanine and morpholine. mp 314° C. ¹H NMR (300 MHz, DMSO-d₆): δ 0.61 (t, J=7.5 Hz, 3 H), 1.34 (s, 6 H), 1.85 (q, J=7.5 Hz, 2 H), 3.67 (m, 2 H), 3.72 (m, 4 H), 3.89 (m, 2 H), 6.66 (s, 1 H), 6.75 (d, J=7.8 Hz, 1 H), 7.24 (m, 2 H), 7.44 (s, 1 H), 7.48 (s, 1 H), 11.63 (s, 1 H).

### Example 157

### 5-{5-[3-(1,1-Dimethyl-propyl)-4-hydroxy-phenyl]-furan-2-ylmethyl}-2-morpholin-4-yl-thiazol-4-one

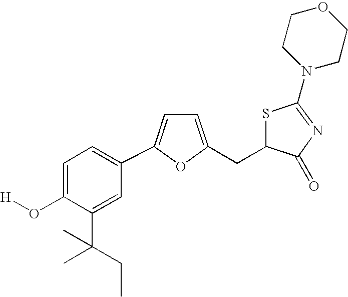

Prepared in a similar manner to that described in Example 43, using 5-{5-[3[(1,1-Dimethyl-propyl)-4-hydroxy-phenyl]-furan-2-ylmethyl}-2-thioxo-thiazolidin-4-one and morpholine. mp 195-196° C. ¹H NMR (300 MHz, DMSO-d₆): δ 0.59 (t, J=7.5 Hz, 3 H), 1.32 (s, 6 H), 1.85 (q, J=7.5 Hz, 2 H), 3.06 (dd, J₁=9.9 Hz, J₂=15.9 Hz, 1 H), 3.46 (dd, J₁=3.9 Hz, J₂=15.9 Hz, 1 H), 3.49 (m, 2 H), 3.62 (m, 4 H), 3.81 (m, 2 H), 4.67 (dd, J₁=3.9 Hz, J₂=9.6 Hz, 1 H), 6.24 (d, J=3.0 Hz, 1 H), 6.55 (d, J=3.0 Hz, 1 H), 6.78 (d, J=8.1 Hz, 1 H), 7.29 (dd, J₁=2.1,J₂=8.1 Hz, 1 H), 7.36 (d, J=2.1 Hz, 1 H), 9.50 (s, 1 H).

The intermediate 5-{5-[3[(1,1-Dimethyl-propyl)-4-hydroxy-phenyl]-furan-2-ylmethyl}-2-thioxo-thiazolidin-4-one was prepared as followed:

### a. 5-{5-[3[(1,1-Dimethyl-propyl)-4-hydroxy-phenyl]-furan-2-ylmethyl}-2-thioxo-thiazolidin-4-one

Prepared in a similar manner to that described in Example 43a, using 5-{5-[3[(1,1-Dimethyl-propyl)-4-hydroxy-phenyl]-furan-2-ylmethylene}-2-thioxo-thiazolidin-4-one. ¹H NMR (300 MHz; CDCl₃): 0.68 (t, J=7.5 Hz, 3 H), 1.40 (s, 6 H), 1.88 (q, J=7.5 Hz, 2 H), 3.30 (dd, J₁=9.6 Hz, J₂=15.6 Hz, 1 H), 3.60 (dd, J₁=3.6 Hz, J₂=15.0 Hz, 1 H), 4.69 60 (dd, J₁=3.6 Hz, J₂=9.6 Hz, 1 H), 6.21 (d, J=3.3 Hz, 1 H), 6.40 (d, J=3.3 Hz, 1 H), 6.68 (d, J=8.1 Hz, 1 H), 7.32 (dd, J=2.1 Hz, J₂=8.1 Hz, 1 H), 7.46 (d, J=2.1 Hz, 1 H), 9.76 (br. s, 1 H).

### b. 5-{5-[3[(1,1-Dimethyl-propyl)-4-hydroxy-phenyl]-furan-2-ylmethylene}-2-thioxo-thiazolidin-4-one

Prepared in a similar manner to that described in Example 43b, using 5-[3-(1,1-Dimethyl-propyl)-4-hydroxy-phenyl]-furan-2-carbaldehyde. ¹H NMR (300 MHz; CDCl₃): 0.65 (t, J=7.5 Hz, 3 H), 1.39 (s, 6 H), 1.89 (q, J=7.5 Hz, 2 H), 6.92 (d, J=8.1 Hz, 1 H), 7.06 (d, J=3.9 Hz, 1 H), 7.27 (d, J=3.9 Hz, 1 H), 7.53 (dd, J₁=2.1 Hz, J₂=8.1 Hz, 1 H), 7.64 (d, J=2.1 Hz, 1 H), 9.95 (s, 1 H), 13.61 (br. s, 1 H).

### c. 5-[3-(1,1-Dimethyl-propyl)-4-hydroxy-phenyl]-furan-2-carbaldehyde

Prepared in a similar manner to that described in Example 1a, using 5-[4-(tert-Butyldimethylsilanoxy)-3-(1,1-dimethyl-propyl)-phenyl]-furan-2-carbaldehyde. ¹H NMR (300 MHz; CDCl₃): 0.68 (t, J=7.5 Hz, 3 H), 1.41 (s, 6 H), 1.90 (q, J=7.5 Hz, 2 H), 5.67 (s, 1 H), 6.71 (d, J=3.6 Hz, 1 H), 6.82 (d, J=8.1 Hz, 1 H), 7.33 (d, J=3.6 Hz, 1 H), 7.59 (dd, J₁=2.1 Hz, J₂=8.1 Hz, 1 H), 7.64 (d, J=2.1 Hz, 1 H), 9.58 (s, 1 H).

### d. 5-[4-(tert-Butyldimethylsilanoxy)-3-(1,1-dimethyl-propyl)-phenyl]-furan-2-carbaldehyde

Prepared in a similar manner to that described in Example 1b, using 4-(tert-Butyldimethylsilanoxy)-3-(1,1-dimethyl-propyl)-boronic acid (2.00 g, 6.2 mmol) and 5-Bromo-2-furaldehyde (1.2 g, 6.82 mmol) to give 2.45 g (100 .%) of 5-[4-(tert-Butyldimethylsilanoxy)-3-(1,1-dimethyl-propyl)-phenyl]-furan-2-carbaldehyde as a clear oil. Used directly in next step.

### c. 4-(tert-Butyldimethylsilanoxy)-3-(1,1-dimethyl-propyl)-boronic acid

To a solution of n-BuLi (50.3 mL of 2.5 M, 125.85 mmol), in anhydrous THF (150 mL) cooled to −78 C under an atmosphere of argon was added dropwise a solution of [4-Bromo-2-(1,1-dimethyl-propyl)-phenoxy]-tert-butyldimethylsilane (30 g, 83.9 mmol) in anhydrous THF (150 mL) over 1 h. Mixture stirred at −78 C for 1 h, then triisopropyl borate (58 mL, 251.7 mmol) was added dropwise over 40 min at −78 C. Warmed to 0° C., and the mixture was quenched with aqueous NH₄Cl, extracted with ethyl acetate (twice). The combined organic layers were washed with brine, dried over magnesium sulfate, filtered, and evaporated to give 20.56 g (76%) of 4-(tert-Butyldimethylsilanoxy)-3-(1,1-dimethyl-propyl)-boronic acid as a white powder. Used directly in next step.

### Example 158

### 5-{5-[3-(1,1-Dimethyl-propyl)-4-hydroxy-phenyl]-thiophen-2-ylmethyl}-2-morpholin-4-yl-thiazol-4-one

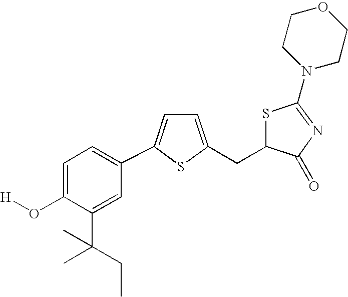

Prepared in a manner similar to that described in Example 43 using 5-{5-[3-(1,1-Dimethyl-propyl)-4-hydroxy-phenyl]-thiophen-2-ylmethyl}-2-thioxo-thiazolidin4-one and morpholine. mp 200-202° C. ¹H NMR (300 MHz, DMSO-d₆): 0.60 (t, J=7.5Hz, 3 H), 1.32 (s, 6 H), 1.84 (q, J=7.5Hz, 2 H), 3.50 (m, 3 H), 3.65 (m, 4 H), 3.78 (m, 2 H), 4.70 (dd, J=3.9, 9.0 Hz, 1 H), 6.78 (d, J=9.0 Hz, 1 H), 6.86 (d, J=3.6 Hz, 1 H), 7.09 (d, J=3.6 Hz, 1 H), 7.24 (m, 2 H).

### Example 159

### 5-[3′-(1,1-Dimethyl-propyl)-5-fluoro-4′-hydroxy-biphenyl-3-ylmethylene]-2-morpholin-4-yl-thiazol-4-one

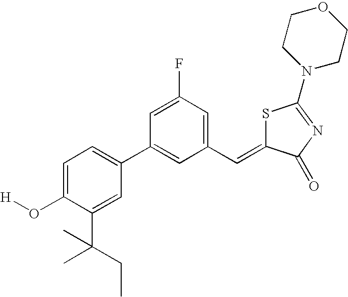

Prepared in a manner similar to that described in Example 1 using 3′-(1,1-Dimethyl-propyl)-5-fluoro-4′-hydroxy-biphenyl-3-carbaldehyde and morpholine. mp 210-212° C. ¹H NMR (300 MHz, DMSO-d₆): 0.69 (t, J=7.2 Hz, 3 H), 1.43 (s, 6 H), 1.93 (q, J=7.2 Hz, 2 H), 3.81 (m, 6 H), 4.00 (m, 2 H), 6.94 (d, J=8.7 Hz, 1 H), (d, J=9.6 Hz, 1 H), 7.48 (m, 2 H), 7.57 (d, J=10.2 Hz, 1 H), 7.77 (s, 1 H), 7.82 (s, 1 H), 9.71 (s, 1 H).

### Example 160

### 5-{3-[7-(1,1-Dimethyl-propyl)-benzoxazol-5-yl]-benzyl}-2-morpholin-4-yl-thiazol-4one

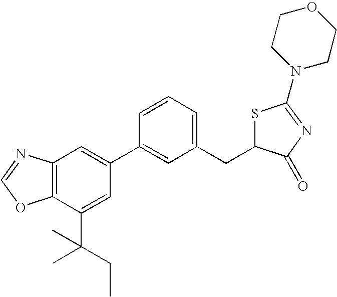

A solution of 5-[5′-Amino-3′-(1,1-dimethyl-propyl)-4′-hydroxy-biphenyl-3-ylmethyl]-2-morpholin-4-yl-thiazol-4-one (40 mg, 8.8×10⁻⁵ mol) in triethylorthoformate (5 mL, 30 mmol) was heated to 100° C. under an atmosphere of argon for 16 hours. The mixture was cooled, extracted with ethyl acetate, washed successively with water and brine, dried over anhydrous magnesium sulfate, filtered and evaporated. The residue was purified on silica gel (eluent: ethyl acetate) to give 24 mg (59%) of 5-{3-[7-(1,1-Dimethyl-propyl)-benzooxazol-5-yl]-benzyl}-2-morpholin-4-yl-thiazol-4-one. ¹H NMR (300 MHz; DMSO): 0.63 (t, J=6.9 Hz, 3 H), 1.45 (s, 6 H), 1.88 (q, J=6.9 Hz, 2 H), 2.97 (dd, J₁=10.2, J₂=14.1 Hz, 1 H), 3.44 (m, 2 H), 3.57 (m, 5 H), 3.77 (m, 2 H), 4.84 (dd, J₁=4.2, J₂=10.5 Hz, 1 H), 7.25 (d, J=7.5 Hz, 1 H), 7.39 (t, J=7.5 Hz, 1 H), 7.45 (s, 1 H), 7.57 (d, J=8.1 Hz, 1 H), 7.62 (s, 1 H). 7.87 (s, 1 H), 8.75 (s, 1 H). MS: Expected: 463; Found: 464 (M+1).

The intermediate 5-[5′-Amino-3′-(1,1-dimethyl-propyl)-4′-hydroxy-biphenyl-3-ylmethyl]-2-morpholin-4-yl-thiazol-4-one was prepared as followed:

### a. 5-[5′-Amino-3′-(1,1-dimethyl-propyl)-4′-hydroxy-biphenyl-3-ylmethyl]-2-morpholin-4-yl-thiazol-4-one

A mixture of 5-[3′-(1,1-dimethyl-propyl)-4′-hydroxy-5′nitro-biphenyl-3-ylmethyl]-2-morpholin-4-yl-thiazol-4-one (118 mg, 0.244 mmol), sodium hydrogen phosphite (0.305 mL of 2.4 M aqueous solution, 0.732 mmol) and Pd/C (12 mg) in anhydrous DMF (10 mL) was heated at 60° C. for 3 hours. The reaction mixture was cooled, filtered through celite, then extracted with ethyl acetate, washed successively with water and brine, dried over anhydrous magnesium chloride, filtered and evaporated to give 60 mg (54%) of 5-[5′-Amino-3′-(1,1-dimethyl-propyl)-4′-hydroxy-biphenyl-3-ylmethyl]-2-morpholin-4-yl-thiazol-4-one.

### b. 5-[5′-Amino-3′-(1,1-dimethyl-propyl)-4′-hydroxy-biphenyl-3-ylmethyl]-2-morpholin-4-thiazol-4-one

Prepared in a similar manner to that used in Example 43 using 5-[3′-(1,1-Dimethyl-propyl)-4′-hydroxy-5′nitro-biphenyl-3-ylmethyl]-2-thioxo-thiazolidin-4-one and morpholine.

### c. 5-[3′-(1,1-Dimethyl-propyl)-4′-hydroxy-5′-nitro-biphenyl-3-ylmethyl]-2-thioxo-thiazolidin-4-one

Prepared in a similar manner to that used in Example 43 using 5-[3′-(1,1-Dimethyl-propyl)-4′-hydroxy-5′nitro-biphenyl-3-ylmethylene]-2-thioxo-thiazolidin-4-one, lithium borohydride and pyridine in THF.

### d. 5-[3′-(1,1-Dimethyl-propyl)-4′-hydroxy-5′-nitro-biphenyl-3-ylmethylene]-2-thioxo-thiazolidin-4-one

Prepared in a similar manner to that used in Example 43, using 3′-(1,1-Dimethyl-propyl)-4′-hydroxy-5′-nitro-biphenyl-3-carbaldehyde, rhodanine, and aniline.

### e. 3′-(1,1-Dimethyl-propyl)-4′-hydroxy-5′-nitro-biphenyl-3-carbaldehyde

To a mixture of nitronium tetrafluoroborate (594 mg, 4.47 mmol) in CH₂Cl₂ (10 mL) cooled to 0° C. under an atmosphere of argon was added dropwise a solution of 3′-(1,1-Dimethyl-propyl)-4′-hydroxy-biphenyl-3-carbaldehyde (example 151) (1.0 g, 3.72 mmol) in CH₂Cl₂ (10 mL). The mixture was stirred at 0° C. for 1 hour, then at room temperature for 3 hours. The reaction mixture was quenched with water, extracted into ethyl acetate (twice), washed successively with a saturated solution of NaHCO₃, water and brine, dried over anhydrous magnesium sulfate, filtered and evaporated to give 1.08 g (93%) 3′-(1,1-Dimethyl-propyl)-4′-hydroxy-5′-nitro-biphenyl-3-carbaldehyde. ¹H NMR (300 MHz; CDCl₃): 0.70 (t, J=6.9 Hz, 3 H), 1.47 (s, 6 H), 1.99 (q, J=6.9 Hz, 2 H), 7.65 (t, J=7.5 Hz, 1 H), 7.66 (d, J=2.4 Hz, 1H), 7.83 (m, 1 H), 7.89 (m, 1H), 8.06 (t, J=1.8 Hz, 1 H), 8.27 (d, J=2.4 Hz, 1 H), 10.11 (s, 1 H), 11.59 (s, 1 H).

### Example 161

### In Vitro Screening of Cancer Drug Candidates, Inhibition of Cdc25a.

The phosphatase assay was performed in a 96-well microtiter plate using recombinant human enzyme Cdc25A purchased from Upstate Biotechnology (Lake Placid, N.Y.). Stock solutions of the test compounds were prepared in DMSO. Five μl of suitable dilutions of the compounds were added to the assay. The final volume of the assay was 100 μl. Twenty units of Cdc25A were pre-incubated with the test compounds at 37° C. for 10 min in the reaction mixture containing 100 mM Tris-HCl, pH 8.2, 40 mM NaCl, 1 mM DTT and 20% glycerol. The reaction was initiated by addition of the enzyme substrate 3-O-methylfluorescein (OMFP; Sigma, St. Louis, Mo.) at a final concentration of 40 μM and incubated at room temperature for 1 hour. This substrate allows fluorometric determination of enzyme activity. OMFP is readily metabolized to the fluorescent O-methylfluorescein by the enzyme Cdc25A, thus the absorbance of each reaction sample was determined at 477 nm using a plate reader (SpectraMax 340, Molecular Devices, Sunnyvale, Calif.). The reaction was linear over the time used in the experiments and was directly proportional to both the enzyme and substrate concentration. Inhibition of Cdc25A activity by test compounds was calculated as a percentage of the solvent control. Results for a representative compound (compound 3) of the invention are shown in Figure One.

### Example 162a

### In Vitro Testing of Cancer Drug Candidates, Human Cancer Cell Based Assays.

### Materials and Methods

The following human cancer cell lines were used to detect anti-cancer activity in the compounds of the invention.

The breast cancer cell line MDA-MB468 served to detect anti-breast cancer activity.

The prostate cancer cell line PC-3 was used to detect anti-lung cancer activity

The non-small-cell lung cancer cell line A549 was used to detect anti-lung cancer activity

The pancreatic cancer cell line BX-PC-3 was used to detect anti-pancreatic cancer activity.

Cell lines were purchased from American Type Culture Collection (ATCC).

### Culture Conditions

The cancer cell cultures were grown as recommended by the ATTC manuals. A549 cells and BX-PC-3 were grown in DME Dulbecco's modified Eagle's medium containing 4500 mg/L glucose; 4 mM L-glutamine; 10 U/ml Pen-G; 10 mcg/ml medium and 10% fetal calf serum (FCS). PC-3 and MDA-MB468 cells were grown in RPMI medium 1640 containing 2 mM L-glutamine; 10 U/ml Pen-G; 10 mcg/ml Streptomycin and 10% FCS. Cells were kept at 6% CO₂ and 37° C. Cells were seeded on day zero in 96-well format tissue culture plates at suitable densities the day before starting treatment, in the media indicated above.

### Treatment

On day one, the compounds of the invention were added to the culture media of growing cells, containing 10% FCS. The cell media contained the compounds of the invention at one of six concentrations: 1×10⁸, 5×10⁻⁸, 1×10⁻⁷, 5×10⁻⁷, 1×10⁻⁶, and 1×10⁻⁵M. 0.1% DMSO was used as vehicle control, and never exceeded 0.1% final concentration. On day four the media was removed and replaced with fresh media containing the compounds of the invention and FCS.

### MTT Assay

The assay is based on the cleavage of the yellow tetrazolium salt MTT to purple formazan crystals by dehydrogenase activity in active mitochondria. This conversion only occurs in living cells with intact/functional mitochondria.

### Procedure

On day five 10 μl of 5 mg/ml MTT dye are added to each well containing a cell culture. Cells are incubated for additional 4 hours at 6% CO₂ and 37° C. Reaction is then stopped by adding 100 μl/well of a solubilization solution consisting of 10% Sodium Dodecyl Sulfate (SDS) and 10 mM HCl On day 6 the formazan crystals formed are solubilized and the resulting colored solution is quantified using a scanning multiwell spectrophotometer at a wavelength of 595 nm.

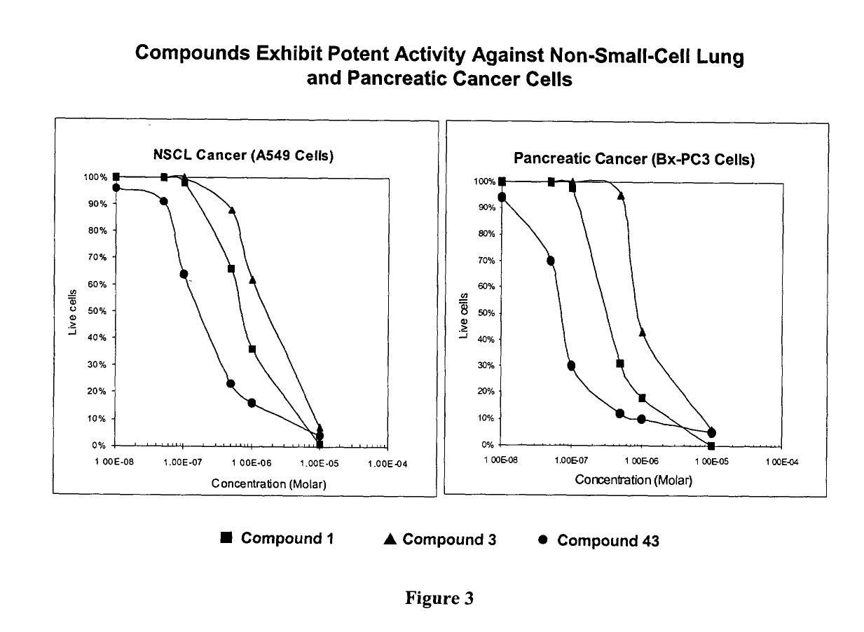

Representative results for compounds 1, 3, and 43 are shown in FIGS. 2 and 3. As can be seen, compounds 1, 3, and 43, when administered in concentrations in the range of 10⁻⁷-10⁻⁵ M or higher, kill significant percentages of the cells of breast cancer, prostate cancer, lung cancer, and pancreatic cancer cultures.

### Example 162b

### In Vitro Testing of Cancer Drug Candidates, Human Cancer Cell Based Assays

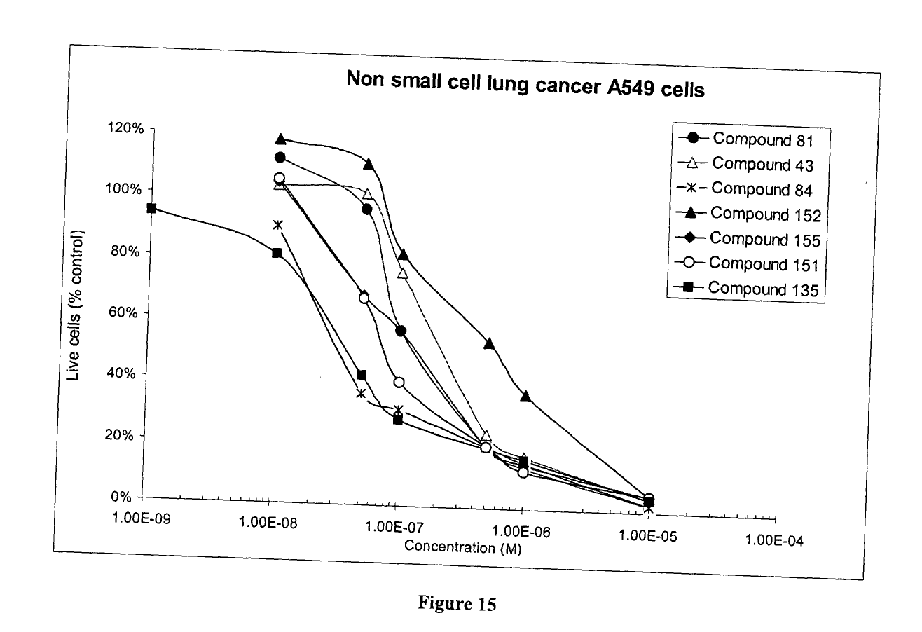

The procedure of Example 162a was employed to screen additional compounds of the invention for anti-cancer activity. The results are shown in FIGS. 15-18. As can be seen in the Figures, compounds 43, 81, 84, 135, 151, 152, and 155, when administered in concentrations in the range of 10⁻⁷-10⁻⁵ M or higher, kill significant percentages of the cells of breast cancer, prostate cancer, lung cancer, and pancreatic cancer cultures.

### Example 163

### Cdc25A Inhibitors Induce Cell Cycle Delay/Arrest at G1 and S Phases in Cancer Cell lines

### Assay Principle

DNA content is a marker of cellular maturity within the cell growth cycle. Cells in G_{0/1} phase have a diploid DNA content. When the cells enter S phase, DNA content increases in proportion to cell progression through S phase. Upon entering G₂ and later M phases the cells have twice the G_{0/1} phase DNA content. Whether a cell is in G_{0/1}, S or G₂/M phase, therefore, can be determined by measuring its DNA content, which can be experimentally estimated via the use of propidium iodide (PI), which specifically binds DNA and fluoresces. Measuring the DNA content of individual cells allows one to determine the cell cycle and whether cell arrest occurs in a particular phase of the cell cycle, such as a G1 or S phase.

### Materials and Methods

### Cell Culture

The human prostate cancer cell line (PC-3) purchased from American Type Culture Collection (ATCC) was grown in RPMI medium 1640 containing 2 mM L-glutamine, 10 U/ml Pen-G, 10 mcg/ml Streptomycin and 10% fetal calf serum (FCS) under conditions of 6% CO₂ and 37° C.

### Treatment with the Compounds

After being seeded for 2 days, PC-3 cells in exponential phase of growth were treated with the compounds at 0.1 μM for 18 hours. DMSO was used as vehicle control. Cells were harvested by trypsin/EDTA treatment.

### Cell DNA Content Measurements

After treatment with the compounds as described above, the treated cells were harvested, washed once with PBS, fixed by 70% ethanol overnight, and incubated with PI/RNase Staining Buffer (BD PharMingen) for 30 min. Data on the DNA content of each cell were estimated from their fluorescence as measured with a Becton Dickinson flow cytometer (FACScalibur), and analyzed with ModFit LT software (Verity Software House). Results were expressed as percentage of controls comprising DMSO, and are shown in FIG. 19.

The results provide evidence that compounds were effective to delay and/or arrest cell growth at the G₀/G₁ or S phases of cell growth, so as to prevent the maturation of the cells to the G₂/M phases of cell growth.

### Example 164

### Intraperitoneal Administration the Compounds of the Invention can Slow the Growth of Solid Prostate and Non-Small Cell Lung Cancer in Mice

### Animal and Tumor Growth and Preparation

Four to six week-old male athymic nude mice (Harlan) were housed under sterile conditions in a fixed 12-12-hr artificial light-dark cycle, and maintained on a standard rodent diet provided ad libitum. Animals were allowed two days to acclimate in this experimental environment prior to the initiation of the study.

Groups of animals were injected subcutaneously with tumor cells (H460 or PC3). Prior to the injection, the sterile tumor cells were at a logarithmic growth phase and were washed twice with PBS, counted and resuspended in sterile saline at 5-50 million cells per ml. As soon as solid tumors were discernible, the animals were sorted into treatment groups with equal average tumor volume. Animals were treated every other day intraperitoneally with a final volume of 5 ml/kg. Tumor volume was measured once a week for the duration of the study.

Using this protocol compounds 43 and 81 of the invention were tested in two different tumor models as follows:

Experiment I: Treatment of solid prostate cancer tumors

Study 1—Treatment groups(n=6/group):

1) control (sesame oil)

2) Compound 43 (20mg/kg)

Study 2—Treatment groups(n=6/group):

1) control (sesame oil)

2) Compound 81 (20mg/kg)

3) Compound 81 (60mg/kg)

Experiment II: Treatment of solid non-small-cell lung cancer tumors

Study 1—Treatment groups(n=6/group):

1) control (sesame oil)

2) Compound 81 (20mg/kg)

As can be seen in FIGS. 20-22, treatment of the cancerous nude mice with compounds 43 and 81 significantly retarded the growth of aggressive prostate and lung cancer tumors grown subcutaneously, indicating that these compounds have potential as anti-cancer therapeutics.

Throughout this application, various publications are referenced. The disclosures of these publications in their entireties are hereby incorporated by reference into this application.

It will be apparent to those skilled in the art that various modifications and variations can be made in the present invention without departing from the scope or spirit of the invention. Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

We claim:

1. A compound having the structure 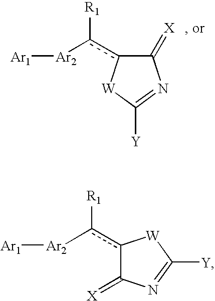
wherein:
a) Ar₁ has the structure 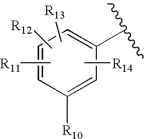
wherein R₁₀ is an organic radical having 1 to 12 carbon atoms, and R₁₁, R₁₂, R₁₃ and R₁₄ are independently selected from hydrogen, inorganic radicals, or organic radicals having 1 to 10 carbon atoms.
b) Ar₂ has 4 to 30 carbon atoms and is an aryl, substituted aryl, heteroaryl, or substituted heteroaryl radical;
c) R₁ is hydrogen, hydroxy, alkoxy, alkyl, or substituted alkyl;
d) - - - represents a bond present or absent;
e) W is —S— or —O—;
f) X is —S— or —O—; and
g) Y is an organic radical comprising 1 to 15 carbon atoms;
or a pharmaceutically acceptable salt thereof.

2. The compound of claim 1 wherein “- - - ” is present.

3. The compound of claim 1 wherein “- - - ” is absent.

4. The compound of claim 1 wherein R₁ is hydrogen.

5. The compound of claim 1 wherein W is —S—.

6. The compound of claim 1 wherein W is —O—.

7. The compound of claim 1 wherein X is —O—.

8. The compound of claim 1 wherein W is —S—, and X is —O—.

9. The compound of claim 8 wherein R₁ is hydrogen or an alkyl having 1 to 4 carbon atoms.

10. The compound of claim 1 wherein Y is an —S—R₂ or -0-R₂ radical wherein the R₂ radical comprises 1 to 10 carbon atoms.

11. The compound of claim 10 wherein R₂ is an alkyl, substituted alkyl, cycloalkyl, or substituted cycloalkyl radical.

12. The compound of claim 1 wherein Y is an —NR₃R₄ radical wherein R₃ and R₄ are independently selected from the group consisting of hydrogen, hydroxyl, amino, and an organic radical comprising 1 to 15 carbon atoms.

13. The compound of claim 12 wherein the organic radical is selected from the group consisting of alkoxy, substituted alkoxy, alkyl, substituted alkyl, cycloalkyl, substituted cycloalkyl, heterocyclic, substituted heterocyclic, amidine, substituted amidine, urea, substituted urea, amino, substituted amino, amide alkyl, amide substituted alkyl, amide aryl, amide substituted aryl, amide heteroaryl, amide substituted heteroaryl, acyl alkyl, and acyl substituted alkyl radicals.

14. The compound of claim 1 wherein Y has the formula

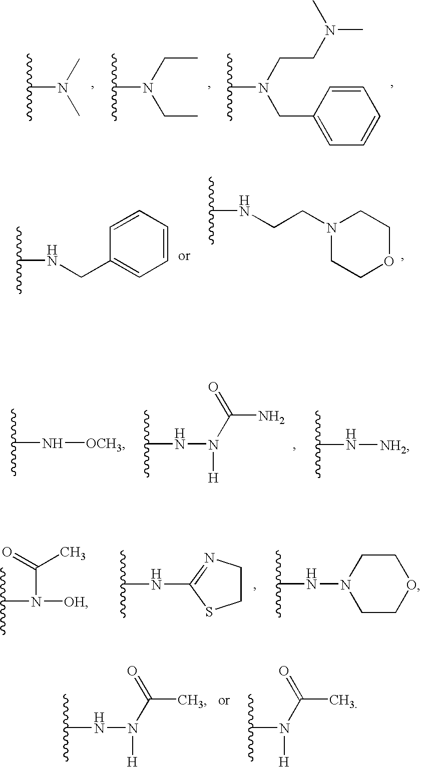

15. The compound of claim 1 wherein Y has the formula

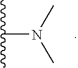

16. The compound of claim 1 wherein Y is an —NR₃R₄ radical and R₃ and R₄ together with the nitrogen form a heterocycle or substituted heterocycle comprising 1 to 15 carbon atoms.

17. The compound of claim 16 wherein the heterocyclic ring is saturated and has 5 or 6 ring atoms, and the remaining ring atoms optionally comprise one or more additional heteroatoms selected from nitrogen, oxygen, or sulfur.

18. The compound of claim 1 wherein Y has the structure

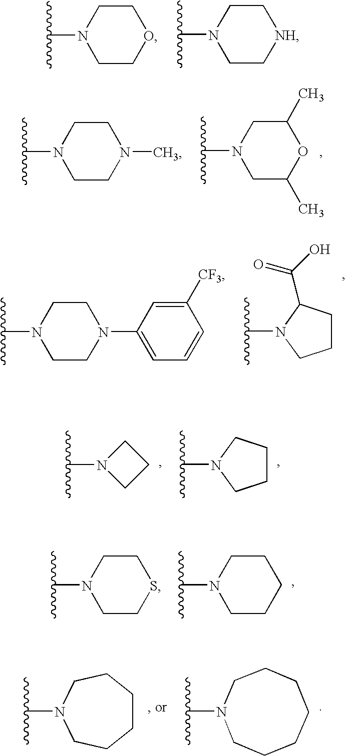

19. The compound of claim 1 wherein Y has the structure

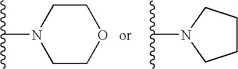

20. The compound of claim 1 wherein Ar₁ comprises from six to twenty carbon atoms.

21. The compound of claim 20 wherein Ar₁ has the structure 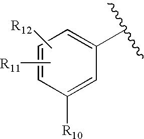
wherein R₁₁ and R₁₂, are independently selected from hydrogen, hydroxy, halides, or organic radicals having 1 to 4 carbon atoms.

22. The compound of claim 20 wherein Ar₁ has the structure 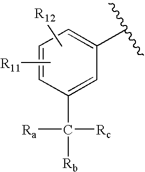
wherein R₁₁ and R₁₂ are independently selected from hydrogen, inorganic radicals, and organic radicals, and wherein Rₐ, R_{b}, and R_{c} are independently selected from hydrogen, inorganic, or organic radicals, with the proviso that no more than one of Rₐ, R_{b}, and R_{c} are hydrogen.

23. The compound of claim 22 wherein two or three of the Rₐ, R_{b}, and R_{c} radicals together form a bicyclic, polycyclic, heterocyclic, alicyclic, aryl, or heteroaryl ring.

24. The compound of claim 22 wherein Rₐ, R_{b}, and R_{c} are alkyls that each comprise 1 to 4 carbon atoms.

25. The compound of claim 22 wherein R₁₀ has the structure

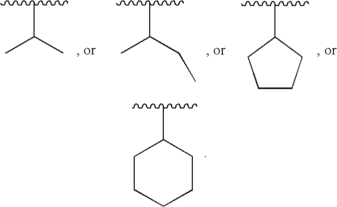

26. The compound of claim 22 wherein Rₐ, R_{b}, and R_{c} are independently selected from an alkyl, substituted alkyl, cycloalkyl, substituted alkyl, heterocyclic or substituted heterocyclic radical.

27. The compound of claim 21 wherein R₁₀ has the structure 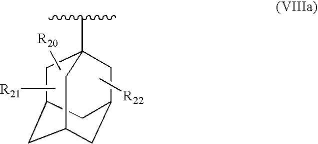
wherein R₂₀, R₂₁ and R₂₂ are independently hydrogen, a halogen, alkyl, hydroxy, carboxyl, alkylcarboxamide or dialkylcarboxamide radical.

28. The compound of claim 21 wherein R₁₀ has the structure

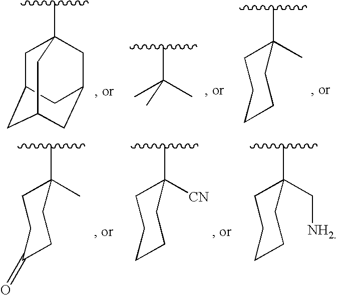

29. The compound of claim 21 wherein R₁₀ has the structure

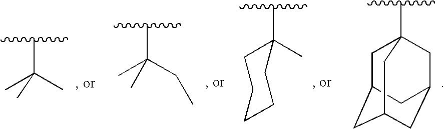

30. The compound of claim 20 wherein Ar₁ is benzoxazole group having the formula 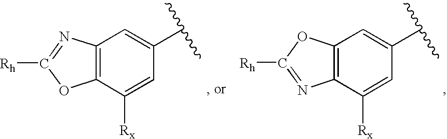
wherein Rₓ and Rₕ are independently selected from hydrogen, an inorganic radical, and an organic radical comprising 1 to 15 carbon atoms.

31. The compound of claim 20 wherein Ar₁ is benzoxazole group having the formula 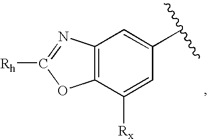
wherein Rₓ is an organic radical comprising 1 to 15 carbon atoms, and Rₕ is selected from hydrogen, and an organic radical comprising 1 to 4 carbon atoms.

32. The compound of claim 20 wherein Ar₁ is benzothiazole group having the formula 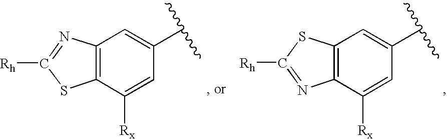
wherein Rₓ and Rₕ are independently selected from hydrogen, an inorganic radical, and an organic radical comprising 1 to 15 carbon atoms.

33. The compound of claim 20 wherein Ar₁ is benzimidazole group having the formula 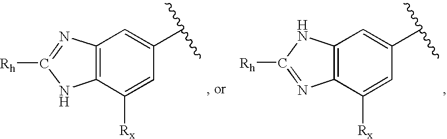
wherein Rₓ and Rₕ are independently selected from hydrogen, an inorganic radical, and an organic radical comprising 1 to 15 carbon atoms.

34. The compound of claim 1 wherein Ar₁ has the structure

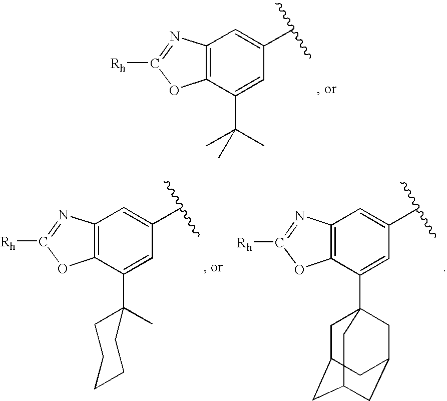

35. The compound of claim 1 wherein Ar₁ has the structure

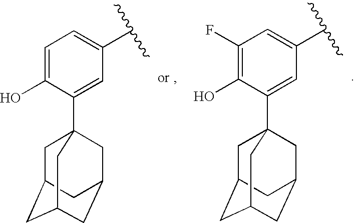

36. The compound of claim 1 wherein Ar₁ has the structure

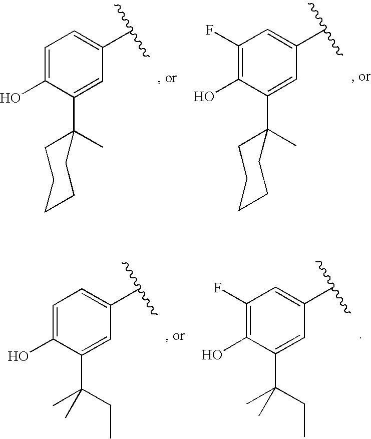

37. The compound of claim 1 wherein Ar₂ has from 6 to 20 carbon atoms.

38. The compounds of claim 37 wherein Ar₂ has the structure 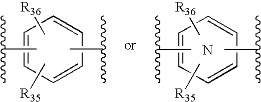
wherein R₃₄ and R₃₅ are independently selected from hydrogen, an inorganic, or an organic radical having from 1 to 12 carbon atoms.

39. The compounds of claim 38 wherein the organic radical is selected from the group consisting of an alkyl, substituted alkyl, alkenyl, substituted alkenyl, alkynyl, substituted alkynyl, cycloalkyl, substituted cycloalkyl, heterocyclic, substituted heterocyclic, alkoxy, substituted alkoxy, hydroxyl, acyl, amino, mono-substituted amino, di-substituted amino, carboxy, carboalkoxy, alkylcarboxamide, substituted alkylcarboxamide, dialkylcarboxamide, substituted dialkylcarboxamide, haloalkoxy, heteroaryl, substituted heteroaryl, aryl, and substituted aryl radical.

40. The compound of claim 1 wherein Ar₂ has the structure 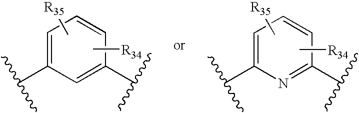
wherein R₃₄ and R₃₅ are independently selected from hydrogen, hydroxy, halogen, alkyl, haloalkyl, alkoxy, or haloalkoxy radicals.

41. The compounds of claim 1 wherein Ar₂ has the structure 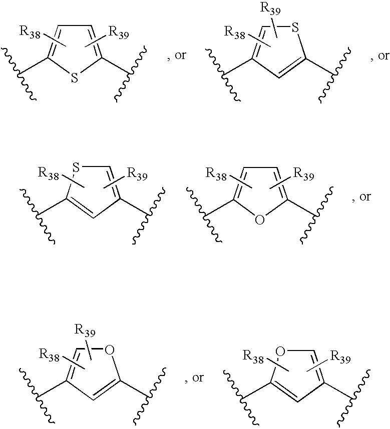
wherein the R₃₈ and R₃₉ radicals are independently selected from hydrogen, inorganic radicals, or organic radicals.

42. The compounds of claim 1 wherein Ar₂ has the structure

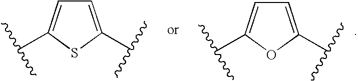

43. The compound of claim 16 wherein Ar₁ has the structure 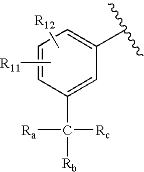
wherein R₁₁ and R₁₂ are independently selected from hydrogen, hydroxy, halogen, and organic radicals comprising 1 to four carbon atoms, and wherein Rₐ, R_{b}, and R_{c} are independently selected from hydrogen, an alkyl, substituted alkyl, cycloalkyl, substituted alkyl, heterocyclic or substituted heterocyclic radical; with the proviso that no more than one of Rₐ, R_{b}, and R_{c} are hydrogen.

44. The compound of claim 43 wherein R₁₁ is hydroxy.

45. The compound of claim 43 wherein two or three of the Rₐ, R_{b}, and R_{c} radicals together form a cycloalkyl, substituted cycloalkyl, bicyclic, polycyclic, heterocyclic, alicyclic, aryl, or heteroaryl ring radical.

46. The compound of claim 43 wherein W is —S—, and X is —O—, and R₁ is hydrogen.

47. The compound of claim 43 wherein Ar₂ has the structure 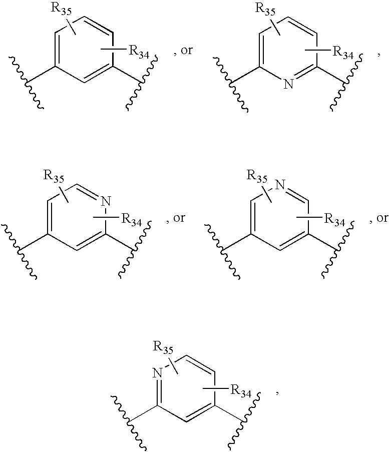
wherein R₃₄ and R₃₅ are independently selected from hydrogen, hydroxy, halogen, alkyl, haloalkyl, alkoxy, or haloalkoxy, and the alkyl, haloalkyl, alkoxy, or haloalkoxy radicals have from 1 to 4 carbon atoms; or 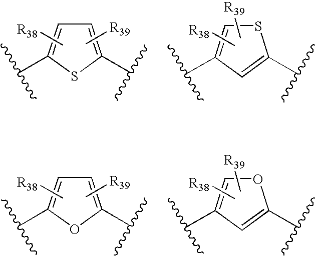
wherein the R₃₈ and R₃₉ radicals are independently selected from hydrogen, inorganic radicals, or organic radicals having 1 to 6 carbon atoms.

48. The compound of claim 1 wherein the inorganic radical is an amino, a hydroxy, or a halogen radical.

49. The compound of claim 1 wherein the compound, when applied to a cell culture of non-small cell lung cancer A549 cells, prostate cancer PC-3 cells, breast cancer MDA-MB-468 cells, or pancreatic cancer BX-PC3 cells at least twice over 5 days at a concentration of about 10 uM, the cancer cells are killed to the extent of at least about 50% as compared to a control not comprising the compound.

50. A pharmaceutical composition for treating a disease of uncontrolled cellular proliferation in mammals comprising one or more pharmaceutically acceptable carriers and one or more compounds of claim 1 in an amount that is effective to treat the disease of uncontrolled cellular proliferation, or a pharmaceutically acceptable salt thereof.

51. A method of treatment for a disease of uncontrolled cellular proliferation comprising administering to a mammal diagnosed as having a disease of uncontrolled cellular proliferation the compound of claim 1 that is effective to treat the disease of uncontrolled cellular proliferation, or a pharmaceutically acceptable salt thereof.

52. The method of claim 51, wherein the disease of uncontrolled cellular proliferation is cancer.

53. The method of claim 51, wherein the disease of uncontrolled cellular proliferation is carcinoma, lymphoma, leukemia, or sarcoma.

54. The method of claim 51, wherein the disease of uncontrolled cellular proliferation is selected from the group of Hodgkin's Disease, meyloid leukemia, polycystic kidney disease, bladder cancer, brain cancer, head and neck cancer, kidney cancer, lung cancer, myeloma, neuroblastoma/glioblastoma, ovarian cancer, pancreatic cancer, prostate cancer, skin cancer, liver cancer, melanoma, colon cancer, cervical carcinoma, breast cancer, epithelial cancer, and leukemia.

55. The method of claim 51 wherein the mammal is a human.

56. A pharmaceutical composition for modulating carbohydrate or lipid metabolism in mammals comprising one or more pharmaceutically acceptable carriers and an amount of one or more compounds of claim 1 or a pharmaceutically acceptable salt thereof.

57. A method of treatment for Type II diabetes, hyperglycemia, or obesity comprising administering to a mammal diagnosed as having diabetes, hyperglycemia, or obesity the compound of claim 1 that is effective to treat the diabetes, hyperglycemia, or obesity, or a pharmaceutically acceptable salt thereof.

58. A method of treatment for an inflammatory disease comprising administering to a mammal diagnosed as having an inflammatory disease a compound of claim 1 that is effective to treat the inflammatory disease, or a pharmaceutically acceptable salt thereof.

59. A pharmaceutical composition of claim 58 wherein the disease is osteoarthritis or rheumatoid arthritis.

60. A compound having the structure 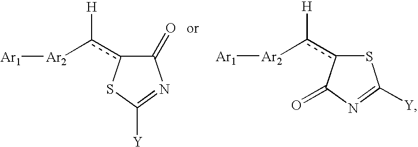
wherein:
a) Ar₁ has the structure 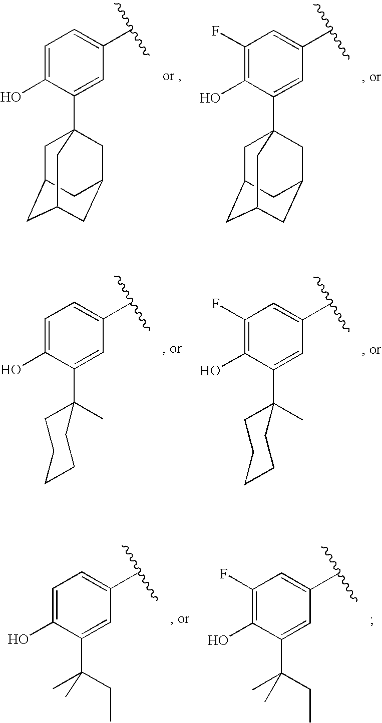
b) Ar₂ has the structure 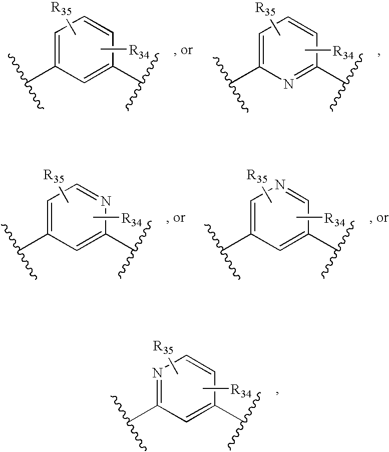
wherein R₃₄ and R₃₅ are independently selected from hydrogen, hydroxy, halogen, alkyl, haloalkyl, alkoxy, or haloalkoxy, and the alkyl, haloalkyl, alkoxy, or haloalkoxy radicals have from 1 to 4 carbon atoms; or the structure 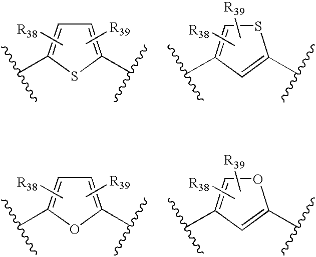
wherein the R₃₈ and R₃₉ radicals are independently selected from hydrogen, inorganic radicals, or organic radicals having 1 to 6 carbon atoms;
c) - - - represents a bond present or absent; and
d) Y has the structure 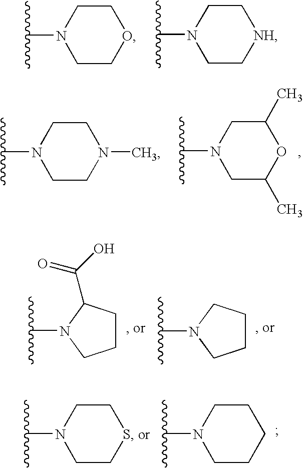
or a pharmaceutically acceptable salt thereof.

61. The compound of claim 60 wherein R₃₄,R₃₅, R₃₈, and R₃₉ are hydrogen.

62. A compound of the formula:
5-[3-(3-Adamantan-1-yl-4-hydroxy-5-fluoro-phenyl)benzylidene]-2-morpholin-4-yl-thiazol-4-one;
5-[3-(3-Adamantan-1-yl-4-hydroxy-phenyl)benzylidene]-2-piperidin-1yl-thiazol-4-one;
5-[3-(3-Adamantan-1-yl-4-hydroxy-5-fluoro-phenyl)benzyl]-2-morpholin-4-yl-thiazol-4-one;
5-[3-(3-Adamantan-1-yl-4-hydroxy-5-fluoro-phenyl)benzyl]-2-pyrrolidin-4-yl-thiazol-4-one;
5-[3-(3-Adamantan-1-yl-4-hydroxy-phenyl)-5-methoxy-6-hydroxy-benzylidene]-2-morpholin-4-yl-thiazol-4-one;
5-(3′-Adamantan-1-yl-4′-hydroxy-biphenyl-3-ylmethyl)-2-morpholin-4-yl-thiazol-4-one;
5-(3′-Adamantan-1-yl-4′-hydroxy-biphenyl-3-ylmethyl)-2dimethylamino-thiazol-4-one;
5-[4′-Hydroxy-3′-(1-methyl-cyclohexyl)-biphenyl-3-ylmethyl]-2-morpholin-4-yl-thiazol-4-one;
2-Dimethylamino-5-[4′-hydroxy-3′-(1-methyl-cyclohexyl)-biphenyl-3-ylmethyl]-thiazol-4-one;
5-[3′-(1,1-Dimethyl-propyl)-4′-hydroxy-biphenyl-3-ylmethyl]-2-pyrrolidin-1-yl-thiazol-4-one;
5-[3′-(1,1-Dimethyl-propyl)-4′-hydroxy-biphenyl-3-ylmethylene]-2-morpholin-4-yl-thiazol-4-one;
5-[4,4′-Dihydroxy-5-methoxy-3′-(1-methyl-cyclohexyl)-biphenyl-3-ylmethyl]-2-morpholin-4-yl-thiazol-4-one;
5-[4′-Hydroxy-4-methoxy-3′-(1-methyl-cyclohexyl)-biphenyl-3-ylmethyl]-2-pyrrolidin-1-yl-thiazol-4-one,
5-[3′-(1,1-Dimethyl-propyl)-4′-hydroxy-biphenyl-3-ylmethyl]-2-morpholin-4-yl-thiazol-4-one;
5-[3′-(1,1-Dimethyl-propyl)-4-fluoro-4′-hydroxy-biphenyl-3-ylmethylene]-2-morpholin-4-yl-thiazol-4-one;
5-[3′-(1,1-Dimethyl-propyl)-5′-fluoro-4′-hydroxy-biphenyl-3-ylmethylene]-2-morpholin-4-yl-thiazol-4-one;
5-{5-[3-(1,1-Dimethyl-propyl)-4-hydroxy-phenyl]-furan-2-ylmethyl}-2-morpholin-4-yl-thiazol-4-one;
5-{6-[3-(1,1-Dimethyl-propyl)-4-hydroxy-phenyl]-pyridin-2-ylmethyl}-2-morpholin-4-yl-thiazol-4-one;
5-[3′-(1,1-Dimethyl-propyl)-5-fluoro-4′-hydroxy-biphenyl-3-ylmethylene]-2-morpholin-4-yl-thiazol-4-one.
